(19) United States
(12) Patent Application Publication
(10) Pub. No.: US 2020/0031924 A1
(43) Pub. Date: Jan. 30, 2020

(54) Dosage Regimens of Lingo-1 Antagonists and Uses for Treatment of Demyelinating Disorders

(71) Applicant: Biogen MA Inc., Cambridge, MA (US)

(72) Inventors: Diego Cadavid, Concord, MA (US); Lei Xu, Wayland, MA (US); Lin Xu, Lexington, MA (US)

(21) Appl. No.: 16/316,428

(22) PCT Filed: Jul. 12, 2017
(86) PCT No.: PCT/US2017/041757

§ 371 Date: Jan. 9, 2019

Related U.S. Application Data
(60) Provisional application No. 62/362,032, filed on Jul. 13, 2016.
(60) Provisional application No. 62/394,094, filed on Sep. 13, 2016.
(60) Provisional application No. 62/412,582, filed on Oct. 25, 2016.
(60) Provisional application No. 62/486,217, filed on Apr. 17, 2017.
(60) Provisional application No. 62/512,510, filed on May 30, 2017.

(51) Int. Cl. C07K 16/28; A61K 39/395; A61K 45/06; A61P 25/00; A61K 9/00; G01N 33/68

(52) U.S. Cl. CPC C07K 16/2803; A61K 39/3955; A61K 45/06; A61P 25/00; A61K 2039/545; G01N 33/6854; C07K 2317/76; C07K 2317/565; A61K 9/0019

(57) ABSTRACT

Dosage regimens, methods, compositions and kits are described herein useful for detecting and/or treating a CNS demyelinating disease.

### RELATED APPLICATIONS

This application claims priority to U.S. Ser. No. 62/362,032 filed Jul. 13, 2016, U.S. Ser. No. 62/394,094 filed Sep. 13, 2016, U.S. Ser. No. 62/412,582 filed Oct. 25, 2016, U.S. Ser. No. 62/486,217 filed Apr. 17, 2017, and U.S. Ser. No. 62/512,510 filed May 30, 2017, the entire contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 12, 2017, is named B2047-7124WO_SL.txt and is 288,381 bytes in size.

### BACKGROUND OF THE INVENTION

Multiple sclerosis (MS) is an inflammatory disease of the brain and spinal cord characterized by recurrent foci of inflammation that lead to destruction of the myelin sheath. In many areas, nerve fibers are also damaged. Inflammatory activity in MS patients tends to be highest in the initial phase of disease.

Emerging data demonstrate that irreversible axonal loss occurs early in the course of MS. Transected axons fail to regenerate in the central nervous system (CNS); and therefore, early treatment aimed at suppressing MS lesion formation is of importance. As early as disease onset, axons are transected in lesions with active inflammation (Trapp et al. (1998) N Engl J Med 338: 278-285; Bjartmar et al. (2001) Curr Opin Neurol 14: 271-278; Ferguson et al. (1997) Brain 120: 393-399). The degree of demyelination is related to the degree of inflammation and the exposure of demyelinated axons to the inflammatory environment, as well as non-inflammatory mediators (Trapp et al. (1998) N Engl J Med 338: 278-285; Kornek et al. (2000) Am J Pathol 157: 267-276; Bitsch et al. (2000) Brain 123: 1174-1183). There is also destruction of oligodendrocytes and impaired remyelination in demyelinating lesions (Peterson et al. (2002) J Neuropathol Exp Neurol 61: 539-546; Chang et al. (2002) N Engl J Med 346: 165-173). A loss of oligodendrocytes leads to a reduction in the capacity to re-myelinate and may also result in the loss of trophic factors that support neurons and axons (Bjartmar et al. (1999) J Neurocytol 28: 383-395).

Optic neuritis, e.g., acute optic neuritis (AON), is characterized by inflammatory white matter lesions in the optic nerve. It is often associated with MS and is one the most common initial manifestations of the disease. AON causes structural and functional optic nerve damage (e.g., neuroaxonal injury and demyelination) that can result in permanent visual impairment for some patients (Cole, S. R. et al. Invest Ophtalmol Vis Sci (2000) 41(5):1017-1021; Mi, S. et al. CNS Drugs 2013: 27(7):493-503; Mangione C M et al. Arch Ophthalmol. (1988) 116(11):1496-1504). The current treatment for acute optic neuritis is high dose steroids which provides mostly symptomatic relief and fails to enhance CNS remyelination or provide neuroaxonal protection (Beck R W et al. N Engl J Med 1992 326:581-8).

Currently approved therapies for MS are primarily immunomodulatory, and typically do not have direct effects on CNS repair. Although some degree of axonal remyelination by oligodendrocytes takes place early during the course of MS, typically, in younger patients, the ability to repair the CNS eventually fails, leading to irreversible tissue injury and an increase in disease-related disabilities. Thus, there is a need for additional therapies that enhance remyelination and neuroaxonal protection in CNS demyelinating diseases, such as MS and optic neuritis.

### SUMMARY OF THE INVENTION

Disclosed herein are methods and compositions for treating or preventing CNS disorders, e.g., CNS demyelinating disorders, using a reparative agent (e.g., a LINGO-1 antagonist, e.g., an anti-LINGO-1 antibody molecule). In certain embodiments, the methods and compositions described herein include a reparative agent (e.g., a LINGO-1 antagonist) as a monotherapy, or in combination with an immunomodulatory agent. In certain embodiments, the reparative agent (e.g., a LINGO-1 antagonist) can be used to treat multiple sclerosis (MS), e.g., a progressive form of MS. In one embodiment, Applicants have identified an unexpected dose response relationship after administration of increasing doses of an anti-LINGO-1 antibody molecule, in the presence of an immunomodulatory agent, e.g., an Interferon (IFN)-β 1 molecule, to a subject with MS, e.g., a subject with a relapsing form of MS (e.g., relapse remitting MS (RRMS), or secondary progressive MS (SPMS)). For example, the dose response identified showed a non-monotonic or biphasic dose-effect relationship, showing an increased improvement response at lower cumulative drug exposure, followed by less improvement at higher cumulative drug levels. In some embodiments, MS subjects with less brain damage, e.g., less advanced forms of MS, showed a more pronounced improvement after treatment compared to subjects with more brain damage, e.g., more advanced form of MS. Thus, disclosed herein are dosage regimens that result in a selected total cumulative exposure (also referred to herein as “cumulative exposure”) to an anti-LINGO-1 antibody therapy; said dosage regimens result in improved therapeutic outcomes in MS subjects. Said dosage regimens can be tailored according to several parameters, including disease status, improvement response, subject's age, and diagnostic test used to evaluate treatment outcome. Accordingly, improved dosage regimens for treatment of CNS demyelinating diseases, such as MS, are disclosed. The methods, compositions and kits described herein can be useful for treating a CNS disorder, e.g., a CNS demyelinating disease.

Accordingly, in one aspect, the invention features a method of treating a CNS disorder, e.g., a CNS demyelinating disease (e.g., MS), in a subject (e.g., a subject in need of treatment). The method includes administering to the subject a reparative agent (e.g., a LINGO-1 antagonist), alone or in combination with an immunomodulatory agent, in an amount and/or at a frequency (e.g., at least one dosage regimen) sufficient to result in a selected cumulative exposure of the reparative agent in the subject, thereby treating the CNS disorder. In some embodiments, the selected cumulative exposure of the reparative agent in the subject is between about 20,000 μg*day/mL to about 55,000 μg*day/mL. In some embodiments, administration of the reparative agent, e.g., the anti-LINGO-1 antibody, molecule is continued until the selected cumulative exposure of the reparative agent is achieved. In other embodiments, administration of the reparative agent is discontinued once the selected cumulative exposure of the reparative agent is achieved.

In another aspect, the invention features a reparative agent, e.g., an anti-LINGO-1 antibody molecule, for use in treating a subject having multiple sclerosis (MS), wherein said reparative agent, e.g., anti-LINGO-1 antibody molecule, is administered alone or in combination with an immunomodulatory agent, in an amount and/or at a frequency (e.g., at least one dosage regimen) sufficient to result in a selected cumulative exposure of the reparative agent. In some embodiments, the selected cumulative exposure of the reparative agent in the subject is between about 20,000 μg*day/mL to about 55,000 μg*day/mL. In embodiments, administration of the reparative agent is continued until the selective cumulative exposure of the reparative agent is achieved. In other embodiments, administration of the reparative agent is discontinued once the selected cumulative exposure of the reparative agent is achieved.

In another aspect, the invention features a method of monitoring a subject (e.g., a subject having a CNS demyelinating disease (e.g., MS)) who is receiving a reparative agent, e.g., a LINGO-1 antagonist (e.g., an anti-LINGO antibody as described herein). The method includes acquiring a value for the level of the reparative agent in the subject at one or more time intervals, e.g., before, during, or after administration of the reparative agent. In one embodiment, the reparative agent is administered alone. In other embodiments, the reparative agent is administered in combination with an immunomodulatory agent. In embodiments, the value acquired is a measure of the cumulative exposure of the reparative agent in the subject, e.g., a selected cumulative exposure as described herein. In some embodiments, the selected cumulative exposure of the reparative agent in the subject is between about 20,000 μg*day/mL to about 55,000 μg*day/mL. In some embodiments, depending on the value acquired administration of the reparative agent is modified, such that a selected cumulative exposure (e.g., a selected cumulative exposure as described herein) of the reparative agent is achieved. In some embodiments, administration of the reparative agent, e.g., the anti-LINGO-1 antibody molecule, is continued until the selected cumulative exposure of the reparative agent molecule is achieved. In other embodiments, administration of the reparative agent is discontinued once the selected cumulative exposure of the reparative agent is achieved.

In some embodiments, the cumulative exposure of the reparative agent, e.g., the LINGO-1 antagonist (e.g., the anti-LINGO-1 antibody molecule), in the subject, is between about 12,000 μg*day/mL to about 106,000 μg*day/mL, between about 20,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 26). In other embodiments, the cumulative exposure of the reparative agent, in the subject, is between about 16,000 μg*day/mL to about 87,000 μg*day/mL, about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 87,000 μg*day/mL, about 28,000 μg*day/mL to about 63,000 μg*day/mL; typically between about 28,000 μg*day/mL to about 44,000 μg*day/mL. In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In one embodiment, a subject having any of the aforesaid cumulative exposures of the reparative agent shows a mean overall response score of about 0.1 to 0.4, e.g., about 0.1, 0.2, 0.3, or 0.4 as shown in FIG. 26, e.g., compared to a subject not receiving the reparative agent.

In some embodiments where the subject is an MS patient with RRMS, the cumulative exposure of the reparative agent, e.g., the LINGO-1 antagonist (e.g., the anti-LINGO-1 antibody molecule), in the subject, is between about 12,000 μg*day/mL to about 240,000 μg*day/mL, between about 20,000 μg*day/mL to about 166,000 μg*day/mL, between about 20,000 μg*day/mL to about 106,000 μg*day/mL, between about 20,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 28A). In other embodiments, the cumulative exposure of the reparative agent, in the subject, is between about 16,000 μg*day/mL to about 200,000 μg*day/mL, about 16,000 μg*day/mL to about 135,000 μg*day/mL, about 16,000 μg*day/mL to about 87,000 μg*day/mL, about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 200,000 μg*day/mL, about 28,000 μg*day/mL to about 135,000 μg*day/mL, about 28,000 μg*day/mL to about 87,000 μg*day/mL, about 28,000 μg*day/mL to about 63,000 μg*day/mL; typically between about 28,000 μg*day/mL to about 44,000 μg*day/mL. In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In some embodiments, the cumulative exposure of the reparative agent in the RRMS subject is between about 20,000 μg*day/mL to about 55,000 μg*day/mL, between about 20,000 μg*day/mL to about 35,000 μg*day/mL, or between about 35,000 μg*day/mL to about 55,000 μg*day/mL. In one embodiment, a subject having RRMS having any of the aforesaid cumulative exposures of the reparative agent shows a mean overall response score of about 0.1 to 0.4, e.g., about 0.1, 0.2, 0.3, or 0.4 as shown in FIG. 28A, e.g., compared to a subject not receiving the reparative agent.

In some embodiments where the subject is an MS patient with SPMS, the cumulative exposure of the reparative agent, e.g., the LINGO-1 antagonist (e.g., the anti-LINGO-1 antibody molecule), in the subject, is between about 12,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 28B). In other embodiments, the cumulative exposure of the reparative agent, in the subject, is between about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 63,000 μg*day/mL; typically between about 28,000 μg*day/mL to about 44,000 μg*day/mL. In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In one embodiment, a subject having SPMS having any of the aforesaid selected cumulative exposures of the reparative agent shows a mean overall response score of about −0.2 to 0.6, e.g., about 0.1, 0.2, 0.3, 0.4, 0.5, or 0.6 as shown in FIG. 28B, e.g., compared to a subject not receiving the reparative agent.

In some embodiments where the subject is an MS patient younger than 40 years old, the cumulative exposure of the reparative agent, e.g., the LINGO-1 antagonist (e.g., the anti-LINGO-1 antibody molecule), in the subject, is between about 12,000 μg*day/mL to about 106,000 μg*day/mL, between about 20,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 29A). In other embodiments, the cumulative exposure of the reparative agent, in the subject, is between about 16,000 μg*day/mL to about 87,000 μg*day/mL, about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 87,000 μg*day/mL, about 28,000 μg*day/mL to about 63,000 μg*day/mL; typically between about 28,000 μg*day/mL to about 44,000 μg*day/mL. In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In one embodiment, a subject under 40 years old having any of the aforesaid cumulative exposures of the reparative agent shows a mean overall response score of about 0 to 0.3, e.g., about 0.1, 0.2, or 0.3 as shown in FIG. 25A, e.g., compared to a subject not receiving the reparative agent.

In some embodiments where the subject is an MS patient 40 years old or older, the cumulative exposure of the reparative agent, e.g., the LINGO-1 antagonist (e.g., the anti-LINGO-1 antibody molecule), in the subject, is between about 12,000 μg*day/mL to about 106,000 μg*day/mL, between about 20,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 29B). In other embodiments, the cumulative exposure of the reparative agent, in the subject, is between about 16,000 μg*day/mL to about 87,000 μg*day/mL, about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 87,000 μg*day/mL, about 28,000 μg*day/mL to about 63,000 μg*day/mL; typically between about 28,000 μg*day/mL to about 44,000 μg*day/mL.

In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In one embodiment, a subject 40 years old or older having any of the aforesaid cumulative exposures of the reparative agent shows a mean overall response score of about 0 to 0.6, e.g., about 0.1, 0.2, 0.3, 0.4, 0.5, or 0.6 as shown in FIG. 29B, e.g., compared to a subject not receiving the reparative agent.

In some embodiments where the subject is an MS patient that shows an improved therapeutic outcome by a measure of upper extremity function, e.g., using a 9 Hole Peg Test (9HPT), the cumulative exposure of the reparative agent, e.g., the LINGO-1 antagonist (e.g., the anti-LINGO-1 antibody molecule), resulting in said improved therapeutic outcome in the subject, is between about 12,000 μg*day/mL to about 106,000 μg*day/mL, between about 20,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIGS. 30A and 30B). In other embodiments, the cumulative exposure of the reparative agent, in the subject, is between about 16,000 μg*day/mL to about 87,000 μg*day/mL, about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 87,000 μg*day/mL, about 28,000 μg*day/mL to about 63,000 μg*day/mL; typically between about 28,000 μg*day/mL to about 44,000 μg*day/mL. In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In one embodiment, a subject having any of the aforesaid cumulative exposures of the reparative agent shows an improved response score using a 9HPT test of about −1.5 to −2 as shown in FIGS. 30A and 30B, e.g., compared to a subject not receiving the reparative agent.

In some embodiments where the subject is an MS patient that shows an improved therapeutic outcome by a measure of short distance ambulatory function, e.g., using a Timed Walk of 25 Feet (T25FW), the cumulative exposure of the reparative agent, e.g., the LINGO-1 antagonist (e.g., the anti-LINGO-1 antibody molecule), resulting in said improved therapeutic outcome in the subject, is between about 12,000 μg*day/mL to about 55,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 31). In other embodiments, the cumulative exposure of the reparative agent, in the subject, is between about 16,000 μg*day/mL to about 44,000 μg*day/mL, and more typically, between about 16,000 μg*day/mL to about 28,000 μg*day/mL. In yet other embodiments, the cumulative exposure of the reparative agent, in the subject, is between about 28,000 μg*day/mL to about 44,000 μg*day/mL. In one embodiment, the cumulative exposure is between about 20,000 μg*day/mL to about 55,000 μg*day/mL. In another embodiment, the cumulative exposure is between about 20,000 μg*day/mL to about 35,000 μg*day/mL. In another embodiment, the cumulative exposure is between about 35,000 μg*day/mL to about 55,000 μg*day/mL. In one embodiment, a subject having any of the aforesaid cumulative exposures of the reparative agent shows an improved response score using a T25FW test of less than 1 as shown in FIG. 31, e.g., compared to a subject not receiving the reparative agent.

In one embodiment, the reparative agent administered is a LINGO-1 antagonist (e.g., an anti-LINGO antibody molecule as described herein), as a monotherapy or a combination therapy. In one embodiment, the reparative agent is an anti-LINGO-1 antibody molecule, which is administered in an amount ranging from about 0.3 mg/kg, about 1 mg/kg, about 3 mg/kg, about 5 mg/kg, about 10 mg/kg, about 30 mg/kg, about 60 mg/kg, about 100 mg/kg, or about 150 mg/kg; for example, between about 1 to 150 mg/kg, about 3 to 100 mg/kg, about 5 to 50 mg/kg, about 10 to 30 mg/kg, about 5 to 15 mg/kg, about 7 to 12 mg/kg, about 10 to 20 mg/kg, about 20 to 30 mg/kg, about 3 to 5 mg/kg; or typically, 3 mg/kg, 5 mg/kg, 10 mg/kg, 30 mg/kg, or 100 mg/kg. In other embodiments, the anti-LINGO-1 antibody molecule is administered as a fixed dose, e.g., about 150 mg to about 7500 mg, about 100 mg to about 7000 mg, about 200 mg to about 3500 mg, about 250 mg to about 3000 mg, about 300 mg to about 2000 mg, about 350 mg to about 1500 mg, about 500 mg to about 1000 mg, about 700 mg to about 900 mg, about 200 mg to about 350 mg, or about 7000 mg, about 5000 mg, about 2000 mg, about 700 mg, about 500 mg, about 350 mg, or about 200 mg. In some embodiments, the anti-LINGO-1 antibody molecule is administered at a fixed dose of about 700 mg to about 850 mg every four weeks, e.g., about 750 mg to about 800 mg every 4 weeks. In some embodiments, the anti-LINGO-1 antibody molecule is administered at a fixed dose of about 700 mg every four weeks, about 725 mg every four weeks, about 750 mg every four weeks, about 775 mg every four weeks, about 800 mg every four weeks, about 825 mg every four weeks, or about 850 mg every four weeks. In some embodiments, the anti-LINGO-1 antibody molecule is administered once a month, or every 4 weeks, e.g., intravenously. In some embodiments, the anti-LINGO-1 antibody molecule is administered at a fixed dose of about 750 mg every four weeks intravenously.

In certain embodiments, the reparative agent (e.g., a LINGO-1 antagonist, e.g., the anti-LINGO-1 antibody molecule) is administered at one or more of the following dosage regimens:

(i) a single dose of 100 mg/kg, or 7000 mg, e.g., administered intravenously;

(ii) one dose of 30 mg/kg, or 2000 mg, e.g., administered intravenously, every four weeks, to a total of 3 doses;

(iii) one dose of 10 mg/kg, 700 mg, or 750 mg, e.g., administered intravenously, every four weeks, to a total of 8 doses;

(iv) one dose of 3 mg/kg, or 200 mg, e.g., administered intravenously, every four weeks, to a total of 18 doses;

(v) one dose of 3-5 mg/kg, or 200-350 mg, e.g., administered subcutaneouly, every four weeks, to a total of 18 doses;

(vi) one dose of 10 mg/kg, or 750 mg, e.g., administered intravenously, every 12 weeks, up to a total of 8 doses; or

(vii) one dose of 30 mg/kg, e.g., administered intravenously, every 24 weeks, up to a total of 4 doses.

In one embodiment, the subject is administered the reparative agent at any of (i), (ii), (iii), (iv), (v), (vi) or (vii) until a selected cumulative exposure is achieved. In other embodiments, the subject is administered the reparative agent as a combination of one or more of (i), (ii), (iii), (iv), (v), (vi) or (vii) until a selected cumulative exposure is achieved. Any combination of one, two, three or more of (i), (ii), (iii), (iv), (v), (vi) or (vii) can be administered, e.g., until a selected cumulative exposure is achieved.

In some embodiments, administration of the reparative agent is continued until a selected cumulative exposure of the reparative agent is achieved, e.g., a selected cumulative exposure as described herein.

In some embodiments, administration of the reparative agent to the subject is intermittent. In one embodiment, administration of the reparative agent is discontinued once a subject achieves a selected cumulative exposure, e.g., a subject dosed at one or more of the dosage regimens described herein. Once the administration of the reparative agent is discontinued, the subject can be monitored for the reappearance of an MS symptom, e.g., using one or more of the neurological, disability, physical and cognitive tests described herein. If one or more MS symptoms reappear (e.g., the subject suffers a relapse or worsening of the disability), administration of the reparative agent is re-initiated, e.g., following a treatment cycle described herein. In one embodiment, the same treatment cycle is used before and after re-initiation. In other embodiments, a different treatment cycle is used before and after re-initiation. Thus, in some embodiments, administration of the reparative agent is intermittent between treatment cycles, but each treatment cycle is administered to achieve a selected cumulative exposure as described herein.

In embodiments, a dosage regimen includes one or more doses of a reparative agent administered in combination with (prior, concurrently or after), administration of a second therapeutic agent, e.g., one or more doses of immunomodulatory agent as described herein. In embodiments, a dosage regimen includes intermittent administration of one or more doses of a reparative agent in combination with a second therapeutic agent, e.g., one or more doses of immunomodulatory agent as described herein. In embodiments, a dosage regimen can have one or more periods of no administration of the therapeutic agent, e.g., drug holidays, in between treatment cycles.

In another embodiment, a treatment cycle comprises administration of the anti-LINGO-1 antibody molecule at 10 mg/kg once every 3 months for 2 years for a total of 8 doses.

In one embodiment, the subject receiving intermittent administration of the reparative agent has an accelerated disability.

In some embodiments, the dosage regimen, e.g., intermittent administration, comprises administration of the reparative agent to the subject for a fixed period of time, e.g., in a treatment cycle, followed by a cycle without treatment (also referred to herein as a “drug holiday”). In some embodiments, a treatment cycle with the anti-LINGO-1 antibody molecule comprises administration of the anti-LINGO-1 antibody molecule at 10 mg/kg every 4 weeks for between about 6 months and about 12 months, e.g., about 6 months, about 7 months, about 8 months, about 9 months, about 10 months, about 11 months, or about 12 months. In some embodiments, the anti-LINGO-1 antibody molecule is administered at a fixed dose of about 700 mg to about 850 mg every four weeks, e.g., about 750 mg to about 800 mg every 4 weeks. In some embodiments, the drug holiday between treatment cycles with the anti-LINGO-1 antibody molecule may be for a duration of between about 6 months and about 18 months or more, e.g., about 6 months, about 7 months, about 8 months, about 9 months, about 10 months, about 11 months, about 12 months, about 13 months, about 14 months, about 15 months, about 16 months, about 17 months, or about 18 months or more. A subject may receive more than one drug holidays, e.g., 2, 3, 4, 5, 6, or more drug holidays over the course of treatment with the anti-LINGO-1 antibody molecule. In some embodiments, the treatment cycles separated by the drug holidays may be continued for 3, 4, 5, 6, 7, 8 or more treatment cycles, or indefinitely. In some embodiments, the second treatment cycle is shorter than the first treatment cycle. In some embodiments, the second treatment cycle is longer than the first treatment cycle. In one embodiment, the second treatment cycle is the same length as the first treatment cycle. In one embodiment, the second drug holiday is the same length as the first drug holiday. In some embodiments, the second drug holiday is longer than the first drug holiday. In some embodiments, the second drug holiday is shorter than the first drug holiday.

In some embodiments, the method includes monitoring the subject for the development of an MS symptom once the administration of the reparative agent is discontinued. In one embodiment, the subject is evaluated using one, two, three or all of: (i) a neurological assessment, e.g., diffuse tensor imaging-radial diffusivity (DTI-RD, e.g., whole brain DTI); (ii) a measure of disability (e.g., EDSS); (iii) a measure of physical function; or (iv) a measure of cognitive function, e.g., as described herein. If a worsening in one or more of the disease parameters is identified, administration of the reparative agent is re-initiated. Administration of the reparative agent can be continued until an improvement of the symptoms occurs. In one embodiment, the one or more MS symptom include a worsening of one or more symptoms and/or disability in the subject, e.g., disease progression as described herein. In one embodiment, the subject experiences a relapse.

In some embodiments, the method includes selecting a subject suitable for treatment with the anti-LINGO-1 antibody molecule. In some embodiments, the subject suitable for treatment has, or is identified as having, 1, 2, 3, 4 or 5 of the following:

(i) A baseline Magnetization Transfer Ratio (MTR) in T2 lesion of less than or equal to about −0.25 to about −0.35, e.g., less than or equal to about −0.31 normalized MTR units (nMTRu);

(ii) A normalized MTR in T2 lesion of less than or equal to 0 nMTRu;

(iii) A baseline diffuse tensor imaging-radial diffusivity (DTI-RD) in T2 lesion of less than or equal to about 0.9×10⁻³ mm²/s to 1.0×10⁻³ mm²/s, e.g, less than or equal to about 0.95×10⁻³ mm²/s;

(iv) A DTI-RD in a value of less than or equal to 1.2×10⁻³ mm²/s; or

(v) A disease duration of less than or equal to about 15 years to about 35 years; e.g., less than or equal to about 20 years.

In some embodiments, the subject suitable for treatment has, or is identified as having, (i) of the above. In other embodiments, the subject suitable for treatment has, or is identified as having, (ii) of the above. In other embodiments, the subject suitable for treatment has, or is identified as having, (iii) of the above. In yet other embodiments, the subject suitable for treatment has, or is identified as having, (iv) of the above. In other embodiments, the subject suitable for treatment has, or is identified as having, (v) of the above. In embodiments, the subject suitable for treatment has, or is identified as having, (i) and (iii) of the above. In other embodiments, the subject suitable for treatment has, or is identified as having, (i) and (iv) of the above. In other embodiments, the subject suitable for treatment has, or is identified as having, (i) and (v) of the above. In yet other embodiments, the subject suitable for treatment has, or is identified as having, (ii) and (iii) of the above. In other embodiments, the subject suitable for treatment has, or is identified as having, (ii) and (iv) of the above. In other embodiments, the subject suitable for treatment has, or is identified as having, (ii) and (v) of the above. In other embodiments, the subject suitable for treatment has, or is identified as having, (iii) and (v) of the above. In other embodiments, the subject suitable for treatment has, or is identified as having, (iv) and (v) of the above. In other embodiments, the subject suitable for treatment has, or is identified as having, (i), (iii) and (v) of the above. In other embodiments, the subject suitable for treatment has, or is identified as having, (i), (iv) and (v) of the above. In other embodiments, the subject suitable for treatment has, or is identified as having, (ii), (iii) and (v) of the above. In other embodiments, the subject suitable for treatment has, or is identified as having, (ii), (iv) and (v) of the above. In yet other embodiments, the subject suitable for treatment has, or is identified as having, (i)-(v) of the above.

In some embodiments, the method includes administering a dosage regimen as described herein if the subject is identified as having 1, 2, 3, 4 or 5 of the above.

In some embodiments, the method includes administering a dosage regimen as described herein if the subject is identified as having (i) or (ii) of the above. In embodiments, the method further comprises administering the dosage regimen if the subject is identified as having (iii) or (iv) of the above, thus the subject is identified as having one of: (i) and (iii), (i) and (iv), (ii) and (iii), or (ii) and (iv) of the above. In embodiments, the method further comprises administering the dosage regimen if the subject is identified as having (v) of the above, thus the subject is identified as having (i) and (v) or (ii) and (v) of the above. In embodiments, the method further comprises administering the dosage regimen if the subject is identified as having (iii)-(v) or (iv)-(v) of the above, thus the subject is identified as having (i)-(v) of the above.

In yet other embodiments, the method includes administering a dosage regimen as described herein if the subject is identified as having (iii) or (iv) of the above. In embodiments, the method further comprises administering the dosage regimen if the subject is identified as having (v) of the above, thus the subject is identified as having (iii) and (v) or (iv) and (v) of the above.

In some embodiments, the method includes selecting a subject suitable for treatment with the anti-LINGO-1 antibody molecule. In some embodiments, the subject suitable for treatment has, or is identified as having, all of the following:

(i) A baseline Magnetization Transfer Ratio (MTR) in T2 lesion of less than or equal to about −0.31 normalized MTR units (nMTRu) or a normalized MTR in T2 lesion of less than or equal to 0 nMTRu;

(ii) A baseline diffuse tensor imaging-radial diffusivity (DTI-RD) in T2 lesion of less than or equal to about 0.95×10⁻³ mm²/s or a DTI-RD in a value of less than or equal to 1.2×10⁻³ mm²/s; and

(iii) A disease duration of less than or equal to about 20 years.

In some embodiments, the method includes administering an anti-LINGO-1 antibody molecule at a dose of about 10 mg/kg if the subject is identified as having (i)-(iii) of the above. In other embodiments, the method includes administering the anti-LINGO-1 antibody molecule at a fixed dose of about 750 mg if the subject is identified as having (i)-(iii) of the above.

In other embodiments, the method includes selecting a subject suitable for treatment with the anti-LINGO-1 antibody molecule. In some embodiments, the subject suitable for treatment has, or is identified as having, one or both of the following:

(i) A baseline MTR in T2 lesion of less than or equal to about −0.25 to −0.35, e.g., less than or equal to about −0.31 nMTRu; or

(ii) A disease duration of less than or equal to about 10 years to about 15 years; e.g., less than or equal to about 12 years.

In some embodiments, the subject suitable for treatment has, or is identified as having, (i) of the above. In other embodiments, the subject suitable for treatment has, or is identified as having, (ii) of the above. In embodiments, the subject suitable for treatment has, or is identified as having, (i) and (ii) of the above.

In some embodiments, the method includes administering a dosage regimen as described herein if the subject is identified as having 1 or 2 of the above.

In some embodiments, the method includes administering a dosage regimen as described herein if the subject is identified as having (i) of the above. In embodiments, the method further comprises administering the dosage regimen if the subject is identified as having (ii) of the above, thus the subject is identified as having (i) and (ii) of the above.

In other embodiments, the method includes administering a dosage regimen as described herein if the subject is identified as having (ii) of the above.

In other embodiments, the method includes selecting a subject suitable for treatment with the anti-LINGO-1 antibody molecule. In some embodiments, the subject suitable for treatment has, or is identified as having, one or both of the following:

(i) A baseline MTR in T2 lesion of less than or equal to about −0.15 to about −0.20, e.g., less than or equal to about −0.17 normalized MTR units (nMTRu); or

(ii) A baseline DTI-RD in T2 lesion of less than or equal to about 0.9×10⁻³ mm²/s to 1.0×10⁻³ mm²/s, e.g, less than or equal to about 0.98×10⁻³ mm²/s.

In some embodiments, the subject suitable for treatment has, or is identified as having, (i) of the above. In other embodiments, the subject suitable for treatment has, or is identified as having, (ii) of the above. In embodiments, the subject suitable for treatment has, or is identified as having, (i) and (ii) of the above.

In some embodiments, the method includes administering a dosage regimen as described herein if the subject is identified as having 1 or 2 of the above.

In other embodiments, the method includes administering a dosage regimen as described herein if the subject is identified as having (i) of the above. In embodiments, the method further comprises administering the dosage regimen if the subject is identified as having (ii) of the above, thus the subject is identified as having (i) and (ii) of the above.

In other embodiments, the method includes administering a dosage regimen as described herein if the subject is identified as having (ii) of the above.

In other embodiments, the method includes selecting a subject suitable for treatment with the anti-LINGO-1 antibody molecule. In some embodiments, the subject suitable for treatment has, or is identified as having, both of the following:

(i) A baseline MTR in T2 lesion of less than or equal to about −0.17 normalized MTR units (nMTRu); and

(ii) A baseline DTI-RD in T2 lesion of less than or equal to about 0.98×10⁻³ mm²/s.

In some embodiments, the method includes administering an anti-LINGO-1 antibody molecule at a dose of about 10 mg/kg if the subject is identified as having (i) and (ii) of the above. In other embodiments, the method includes administering the anti-LINGO-1 antibody molecule at a fixed dose of about 750 mg if the subject is identified as having (i) and (ii) of the above.

nMTRu are normalized MTR units. In order to directly compare data acquired at different centers, MTR values are mapped onto a normalized scale where 0 is the median MTR of normal gray matter and 1 is the median MTR of normal white matter. These reference values are measured for a particular scanner using the dummy run for the site, which is performed on a healthy subject. Using the reference values from the dummy run, a linear mapping function that translates MTR values from that scanner into calibrated MTR values on the normalized scale is constructed. On the normalized scale, MTR values in brain tissue, including non-acute lesions, typically range from about −0.5 to 1.5. MTR in acute lesions can be much lower. (See Brown R A, et al. Proc Intl Soc Mag Reson Med. 2011; 19:4082).

In other embodiments, the method includes selecting a subject suitable for treatment with the anti-LINGO-1 antibody molecule. In some embodiments, the subject suitable for treatment has, or is identified as having a DTI-RD in T2 lesion of less than about 0.7×10⁻³ mm²/s to about 0.8×10⁻³ mm²/s, e.g, less than about 0.732×10⁻³ mm²/s at baseline, e.g., prior to treatment. In some embodiments, the method further includes administering a dosage regimen as described herein if the subject is identified as having a DTI-RD in T2 lesion of less than 0.7×10⁻³ mm²/s to 0.8×10⁻³ mm²/s, e.g, less than about 0.732×10⁻³ mm²/s at baseline, e.g., prior to treatment.

In certain embodiments, the reparative agent administered is a LINGO-1 antagonist (e.g., an anti-LINGO antibody molecule as described herein). In some embodiment, the anti-LINGO antibody molecule is opicinumab (also referred to herein as “BIIB033”). In one embodiment, the anti-LINGO-1 antibody molecule comprises (i) a heavy chain comprising, consisting essentially of, or consisting of, the amino acid sequence of SEQ ID NO: 275, and (ii) a light chain comprising, consisting essentially of, or consisting of, the amino acid sequence of SEQ ID NO: 276.

In other embodiments, the reparative agent administered is a LINGO-1 antagonist (e.g., an anti-LINGO antibody as described herein) in combination with a second agent (e.g., an immunomodulatory agent as described herein). In one embodiment, the reparative agent is an anti-LINGO antibody (e.g., opicinumab), which is administered as a combination therapy in an amount ranging from about 1 mg/kg, about 3 mg/kg, about 5 mg/kg, about 10 mg/kg, about 30 mg/kg, about 60 mg/kg, or about 100 mg/kg; for example, between about 1 to 150 mg/kg, about 3 to 100 mg/kg, about 5 to 50 mg/kg, about 10 to 30 mg/kg, about 5 to 15 mg/kg, about 7 to 12 mg/kg, about 10 to 20 mg/kg, about 20 to 30 mg/kg, or 3 mg/kg, 5 mg/kg, 10 mg/kg, 30 mg/kg, or 100 mg/kg. In one embodiment, the reparative agent is administered as a combination therapy in an amount of about 10 mg/kg. In other embodiments, the reparative agent is administered as a combination therapy in an amount ranging from about 150 mg to about 7500 mg, about 100 mg to about 7000 mg, about 200 mg to about 3500 mg, about 250 mg to about 3000 mg, about 300 mg to about 2000 mg, about 350 mg to about 1500 mg, about 500 mg to about 1000 mg, about 700 mg to about 900 mg, about 200 mg to about 350 mg; or about 7000 mg, about 5000 mg, about 2000 mg, about 750 mg, about 700 mg, about 500 mg, about 350 mg, or about 200 mg. In one embodiment, the reparative agent is administered as a combination therapy in an amount of about 750 mg. In some embodiments, the anti-LINGO-1 antibody molecule is administered at a fixed dose of about 700 mg to about 850 mg every four weeks, e.g., about 750 mg to about 800 mg every 4 weeks. In some embodiments, the anti-LINGO-1 antibody molecule is administered at a fixed dose of about 700 mg every four weeks, about 725 mg every four weeks, about 750 mg every four weeks, about 775 mg every four weeks, about 800 mg every four weeks, about 825 mg every four weeks, or about 850 mg every four weeks. In one embodiment, the reparative agent is administered as a combination therapy in an amount of about 750 mg every four weeks. In some embodiments, the anti-LINGO-1 antibody molecule is administered once a month, or every 4 weeks, e.g., intravenously. In some embodiments, the anti-LINGO-1 antibody molecule is administered in combination with a second agent at a fixed dose of about 750 mg every four weeks intravenously. In one embodiment, the immunomodulatory agent is administered according to the standard of care for that agent, e.g., as described herein. In another embodiment, administration of the LINGO-antagonist allows for administration of a reduced amount of the immunomodulatory agent.

In one embodiment, the subject (e.g., the human) has, or is at risk of having, MS. In one embodiment, the subject is a human having, or is at risk of having, MS about 40 years old or older. In another embodiment, the subject is a human having, or is at risk of having, MS about 40 years old or less.

In one embodiment, the human subject has one or more symptoms associated with MS. In certain embodiments, the subject with MS is chosen from a human having one or more of: RRMS (e.g., quiescent RRMS, active RRMS), primary progressive MS (PPMS), or secondary progressive MS (SPMS), or active SPMS. In one embodiment, the subject has a relapsing form of MS. In one embodiment, the subject has RRMS or SPMS. In one embodiment, the subject with MS has SPMS. In one embodiment, the subject with MS has RRMS.

In some embodiments, the subject has a relapsing form of MS. For example, the subject has shown relapsing activity within 2 years, within 1.5 year, within 1 year, within 8 months, within 6 months, or within 3 months or less, prior to beginning treatment with the reparative agent, e.g., the anti-LINGO-1 antibody molecule.

In one aspect, the invention provides a method of treating SPMS in a subject. The method includes administering a reparative agent as described herein, e.g., an anti-LINGO-1 antibody molecule, in an amount and/or at a frequency (e.g., at least one dosage regimen) sufficient to result in a selected cumulative exposure, thereby treating the SPMS disorder. In some embodiments, the selected cumulative exposure is between about 20,000 μg*day/mL to about 35,000 μg*day/mL in the subject. In some embodiments, administration of the reparative agent, e.g., the anti-LINGO-1 antibody molecule, is continued until the selected cumulative exposure of the reparative agent molecule is achieved. In other embodiments, administration of the reparative agent is discontinued once the selected cumulative exposure of the reparative agent is achieved.

In another aspect, the invention features an anti-LINGO-1 antibody molecule for use in treating a subject having secondary progressive multiple sclerosis (SPMS), wherein said anti-LINGO-1 antibody molecule is administered in an amount and/or at a frequency (e.g., at least one dosage regimen) sufficient to result in a selected cumulative exposure of the anti-LINGO-1 antibody molecule. In some embodiments, the selected cumulative exposure of the anti-LINGO-1 antibody molecule is between about 20,000 μg*day/mL to about 35,000 μg*day/mL in the subject. In some embodiments, administration of the reparative agent, e.g., the anti-LINGO-1 antibody molecule, is continued until the selected cumulative exposure of the reparative agent molecule is achieved. In other embodiments, administration of the reparative agent is discontinued once the selected cumulative exposure of the reparative agent is achieved.

In some embodiments, the reparative agent (e.g., a LINGO-1 antagonist, e.g., the anti-LINGO-1 antibody molecule) is administered to the subject at one or more of the following dosage regimens:

(i) a single dose of 100 mg/kg, or 7000 mg, e.g., administered intravenously;

(ii) one dose of 30 mg/kg, or 2000 mg, e.g., administered intravenously, every four weeks, to a total of 3 doses;

(iii) one dose of 10 mg/kg, 700 mg, or 750 mg, e.g., administered intravenously, every four weeks, to a total of 8 doses;

(iv) one dose of 3 mg/kg, or 200 mg, e.g., administered intravenously, every four weeks, to a total of 18 doses;

(v) one dose of 3-5 mg/kg, or 200-350 mg, e.g., administered subcutaneouly, every four weeks, to a total of 18 doses

(vi) one dose of 10 mg/kg, or 750 mg, e.g., administered intravenously, every 12 weeks, up to a total of 8 doses; or

(vii) one dose of 30 mg/kg, e.g., administered intravenously, every 24 weeks, up to a total of 4 doses.

In yet another aspect, the invention provides a method of improving a disability, e.g., a walking disability, in a subject, e.g., a patient with MS. The method includes administering a reparative agent as described herein, e.g., an anti-LINGO-1 antibody molecule, in an amount and/or at a frequency (e.g., at least one dosage regimen) sufficient to result in a selected cumulative exposure in the subject, thereby improving the disability. In some embodiments, the selected cumulative exposure, is between about 20,000 μg*day/mL to about 35,000 μg*day/mL in the subject. In some embodiments, administration of the reparative agent, e.g., the anti-LINGO-1 antibody molecule, is continued until the selected cumulative exposure of the reparative agent molecule is achieved. In other embodiments, administration of the reparative agent is discontinued once the selected cumulative exposure of the reparative agent is achieved.

In another aspect, the invention features a reparative agent, e.g., an anti-LINGO-1 antibody molecule, for use in improving a disability, e.g., a walking disability, in a subject with MS, wherein said anti-LINGO-1 antibody molecule is administered in an amount and/or at a frequency (e.g., at least one dosage regimen) sufficient to result in a selected cumulative exposure of the reparative agent, e.g., the anti-LINGO-1 antibody molecule, in the subject. In some embodiments, the selected cumulative exposure, is between about 20,000 μg*day/mL to about 35,000 μg*day/mL in the subject. In some embodiments, administration of the reparative agent, e.g., the anti-LINGO-1 antibody molecule, is continued until the selected cumulative exposure of the reparative agent molecule is achieved. In other embodiments, administration of the reparative agent is discontinued once the selected cumulative exposure of the reparative agent is achieved.

In some embodiments, the reparative agent (e.g., a LINGO-1 antagonist, e.g., the anti-LINGO-1 antibody molecule) is administered to the subject at one or more of the following dosage regimens:

(i) a single dose of 100 mg/kg, or 7000 mg, e.g., administered intravenously;

(ii) one dose of 30 mg/kg, or 2000 mg, e.g., administered intravenously, every four weeks, to a total of 3 doses;

(iii) one dose of 10 mg/kg, 700 mg, or 750 mg, e.g., administered intravenously, every four weeks, to a total of 8 doses;

(iv) one dose of 3 mg/kg, or 200 mg, e.g., administered intravenously, every four weeks, to a total of 18 doses;

(v) one dose of 3-5 mg/kg, or 200-350 mg, e.g., administered subcutaneouly, every four weeks, to a total of 18 doses;

(vi) one dose of 10 mg/kg, or 750 mg, e.g., administered intravenously, every 12 weeks, up to a total of 8 doses; or

(vii) one dose of 30 mg/kg, e.g., administered intravenously, every 24 weeks, up to a total of 4 doses.

In some embodiments, the subject is naïve or previously treated with a disease modifying therapy. Exemplary disease-modifying therapies (DMTs) for MS, include oral therapies, such as Tecfidera®, Aubagio®, or Gilenya®; injectable therapies, such as Avonex®, Betaseron®, Copaxone®, Extavia®, Glatopa®, Plegridy®, or Rebif®; infusion therapies, such as Tysabri®, Lemtrada®, or Novantrone®. In one embodiment, the subject is previously treated with an IFNβ agent, e.g., Avonex®.

In some embodiments, the subject has a baseline EDSS score of 2-6, prior to beginning treatment with the reparative agent, e.g., the anti-LINGO-1 antibody molecule. In some embodiments, the subject has an EDSS score of about 2. In some embodiments, the subject has an EDSS score of about 3. In some embodiments, the subject has an EDSS score of about 4. In some embodiments, the subject has an EDSS score of about 5. In some embodiments, the subject has an EDSS score of about 6.

In some embodiments, the subject has a walking disability, prior to beginning treatment with the reparative agent, e.g., the anti-LINGO-1 antibody molecule

In some embodiments, the subject is evaluated by one or more (e.g., one, two, three or all) of:

(i) neurological assessment, e.g., diffuse tensor imaging-radial diffusivity (DTI-RD, e.g., whole brain DTI);

(ii) a measure of disability (e.g., EDSS, wherein a reduced score is indicative of improvement, and an increased score is indicative of worsening);

(iii) a measure of physical function, e.g., upper extremity function, e.g., using a 9 Hole Peg Test (9HPT dominant and non-dominant hands, wherein reduced time is indicative of improvement, and increased time is indicative of worsening), a measure of short distance ambulatory function, e.g., using a Timed Walk of 25 Feet (T25FW, wherein reduced time is indicative of improvement, and increased time is indicative of worsening), or a measure of long distance ambulatory function, e.g., using a 6 minute walk test (6 MW, wherein reduced time is indicative of improvement, and increased time is indicative of worsening);

(iv) a measure of cognitive function, e.g., PASAT-3, wherein an increased score is indicative of improvement, and a decreased score is indicative of worsening; or

(v) a measure of cognitive function, e.g. SDMT, wherein an increase score is indicative of improvement, and a decreased score is indicative of worsening.

Additional methodologies for evaluating the subject are disclosed herein below.

In one embodiment, the subject is an MS patient who is an improvement responder. As used herein, “an improvement responder” refers to a subject who has a confirmed improvement in any one or more components of, e.g., 9HPT (either hand), T25FW or PASAT. In one embodiment, an improvement responder shows ≥15% change from baseline in any of 9HPT (either hand), T25FW or PASAT, confirmed 3 months or later, or a change in EDSS, confirmed 3 months or later, or a combination of both.

In another aspect, the invention features a dosage formulation or dosage regimen, e.g., a combination of dosage regimens, that results in a selected cumulative exposure of the reparative agent, e.g., the LINGO-1 antagonist (e.g., the anti-LINGO-1 antibody molecule), in the subject, between about 12,000 μg*day/mL to about 106,000 μg*day/mL, between about 20,000 μg*day/mL to about 72,000 μg*day/mL; typically between about 20,000 μg*day/mL to about 55,000 μg*day/mL, and more typically, between about 20,000 μg*day/mL to about 35,000 μg*day/mL, between about 35,000 μg*day/mL to about 55,000 μg*day/mL (e.g., as determined by the total drug exposure over time, e.g., cumulative area under the curve (AUC) shown in FIG. 26). In other embodiments, the selected cumulative exposure of the reparative agent, in the subject, is between about 16,000 μg*day/mL to about 87,000 μg*day/mL, about 16,000 μg*day/mL to about 63,000 μg*day/mL; typically between about 16,000 μg*day/mL to about 44,000 μg*day/mL, and more typically, between about 16,000 μg*day/mL to about 28,000 μg*day/mL. In yet other embodiments, the selected cumulative exposure of the reparative agent, in the subject, is between about 28,000 μg*day/mL to about 87,000 μg*day/mL, about 28,000 μg*day/mL to about 63,000 μg*day/mL; typically between about 28,000 μg*day/mL to about 44,000 μg*day/mL. In one embodiment, the selected cumulative exposure is between about 20,000 μg*day/mL to about 55,000 μg*day/mL. In another embodiment, the selected cumulative exposure is between about 20,000 μg*day/mL to about 35,000 μg*day/mL. In another embodiment, the selected cumulative exposure is between about 35,000 μg*day/mL to about 55,000 μg*day/mL. In embodiments, the antibody molecule is instructed to be administered at one or more of the following dosage regimens:

(i) a single dose of 100 mg/kg, or 7000 mg, e.g., administered intravenously;

(ii) one dose of 30 mg/kg, or 2000 mg, e.g., administered intravenously, every four weeks, to a total of 3 doses;

(iii) one dose of 10 mg/kg, 700 mg, or 750 mg, e.g., administered intravenously, every four weeks, to a total of 8 doses;

(iv) one dose of 3 mg/kg, or 200 mg, e.g., administered intravenously, every four weeks, to a total of 18 doses;

(v) one dose of 3-5 mg/kg, or 200-350 mg, e.g., administered subcutaneouly, every four weeks, to a total of 18 doses;

(vi) one dose of 10 mg/kg, or 750 mg, e.g., administered intravenously, every 12 weeks, up to a total of 8 doses; or

(vii) one dose of 30 mg/kg, e.g., administered intravenously, every 24 weeks, up to a total of 4 doses.

Kits that include the reparative agent, e.g., the LINGO-1 antagonist (e.g., the anti-LINGO-1 antibody molecule) for use in dosage regimen(s) that results in a selected cumulative exposure of the reparative agent, e.g., the LINGO-1 antagonist (e.g., the anti-LINGO-1 antibody molecule), in the subject, are also provided. The kits may, optionally, include instructions for administration of the reparative agent, e.g., differential administration of the reparative agent, e.g., discontinued use or intermittent use. In some embodiments, the selected cumulative exposure of the reparative agent is less than 65,000 μg*day/mL, less than 32,000 μg*day/mL, less than 18,000 μg*day/mL, less than 12,000 μg*day/mL, or less than 8,000 μg*day/mL. In embodiments, the selected cumulative exposure of the reparative agent is not less than 8,000 μg*day/mL or 4,000 μg*day/mL. In embodiments, the antibody molecule is instructed to be administered at one or more of the following dosage regimens:

(i) a single dose of 100 mg/kg, or 7000 mg, e.g., administered intravenously;

(ii) one dose of 30 mg/kg, or 2000 mg, e.g., administered intravenously, every four weeks, to a total of 3 doses;

(iii) one dose of 10 mg/kg, 700 mg, or 750 mg, e.g., administered intravenously, every four weeks, to a total of 8 doses;

(iv) one dose of 3 mg/kg, or 200 mg, e.g., administered intravenously, every four weeks, to a total of 18 doses;

(v) one dose of 3-5 mg/kg, or 200-350 mg, e.g., administered subcutaneouly, every four weeks, to a total of 18 doses;

(vi) one dose of 10 mg/kg, or 750 mg, e.g., administered intravenously, every 12 weeks, up to a total of 8 doses; or

(vii) one dose of 30 mg/kg, e.g., administered intravenously, every 24 weeks, up to a total of 4 doses.

Compositions comprising a reparative agent, e.g., the anti-LINGO antibody molecule described herein, for use in treating in a subject a CNS demyelinating disease, e.g., MS, are provided. In embodiments, said reparative agent is administered in at least one dosage regimen that results in a selected cumulative exposure of the reparative agent in the subject. In some embodiments, the selected cumulative exposure of the reparative agent is less than 65,000 μg*day/mL, less than 32,000 μg*day/mL, less than 18,000 μg*day/mL, less than 12,000 μg*day/mL, or less than 8,000 μg*day/mL. In embodiments, the selected cumulative exposure of the reparative agent is not less than 8,000 μg*day/mL or 4,000 μg*day/mL. In embodiments, the antibody molecule is instructed to be administered at one or more of the following dosage regimens:

(i) a single dose of 100 mg/kg, or 7000 mg, e.g., administered intravenously;

(ii) one dose of 30 mg/kg, or 2000 mg, e.g., administered intravenously, every four weeks, to a total of 3 doses;

(iii) one dose of 10 mg/kg, 700 mg, or 750 mg, e.g., administered intravenously, every four weeks, to a total of 8 doses;

(iv) one dose of 3 mg/kg, or 200 mg, e.g., administered intravenously, every four weeks, to a total of 18 doses;

(v) one dose of 3-5 mg/kg, or 200-350 mg, e.g., administered subcutaneouly, every four weeks, to a total of 18 doses;

(vi) one dose of 10 mg/kg, or 750 mg, e.g., administered intravenously, every 12 weeks, up to a total of 8 doses; or

(vii) one dose of 30 mg/kg, e.g., administered intravenously, every 24 weeks, up to a total of 4 doses.

In some embodiments, the kits, dosage regimens and compositions further comprises a second agent, e.g., a second agent as described herein (e.g., an IFN-β 1 molecule) to be administered in combination with the LINGO-1 antagonist.

Additional embodiments, features or improvements of any of the methods, compositions, dosage regimens and kits disclosed herein include one or more of the following:

### CNS Disorders and CNS Demyelinating Diseases

The CNS disorder (e.g., the CNS demyelinating disease) can be any condition, disease, disorder or injury associated with one or more of: demyelination, dysmyelination, axonal injury, loss of axonal area or axial diffusivity, or loss of neuronal synapsis/connectivity, and/or dysfunction or death of an oligodendrocyte or a neuronal cell. In certain embodiments, the CNS disorder affects the nervous system by causing damage to the myelin sheath of axons. In other embodiments, the CNS disorder includes Nogo receptor-1 (NgR1-) mediated inhibition of axonal extension or neurite extension, e.g., in the brain and spinal cord. In other embodiments, the CNS disorder has one or more inflammatory components. Exemplary CNS disorders include, but are not limited to, CNS demyelinating diseases, CNS injury, Amyotrophic lateral sclerosis (ALS), Huntington's disease, Alzheimer's disease, Parkinson's disease, diabetic neuropathy, idiopathic inflammatory demyelinating disease, multiple sclerosis (MS), optic neuritis (e.g., acute optic neuritis), transverse myelitis, neuromyelitis optica (NMO), vitamin B12 deficiency, progressive multifocal leukoencephalopathy (PML), encephalomyelitis (EPL), acute disseminated encephalomyelitis (ADEM), central pontine myelolysis (CPM), Wallerian Degeneration, adrenoleukodystrophy, Alexander's disease, Pelizaeus Merzbacher disease (PMZ), leukodystrophies, traumatic glaucoma, periventricular leukomalatia (PVL), essential tremor, white matter stroke, stroke, or radiation or toxic induced white matter injury. A CNS demyelinating disease can be chosen from one or more of the aforesaid disorders. In one embodiment, the CNS demyelinating disease is multiple sclerosis. In other embodiments, the CNS demyelinating disease is optic neuritis, e.g., acute optic neuritis.

### Subjects

For any of the methods, compositions, dosage regimens, and kits disclosed herein, the subject treated, is a subject (e.g., a human) having, or at risk of having, a CNS disorder or a CNS demyelinating disease, e.g., as described herein.

In certain embodiments, the subject is a human, e.g., a human adult. In one embodiment, the subject is a human about 40 years old or older, e.g., at least 40, 45, 50, 55, 60 years old or older. In another embodiment, the subject is a human about 40 years old or less, e.g., less than 40, 35, 30, 25, 21 years old or younger.

In one embodiment, the subject (e.g., the human) has, or is at risk of having, MS. In one embodiment, the human subject has one or more symptoms associated with MS (“an MS symptom”). The subject with MS can be at any stage of treatment. In certain embodiments, the subject with MS is chosen from a human having one or more of: Benign MS, RRMS (e.g., quiescent RRMS, active RRMS), primary progressive MS (PPMS), or secondary progressive MS (SPMS), active SPMS, clinically isolated syndrome (CIS), or clinically defined MS (CDMS). In one embodiment, the subject has a relapsing form of MS. In one embodiment, the subject has RRMS or SPMS. In one embodiment, the subject with MS has SPMS. In one embodiment, the subject with MS has RRMS. In other embodiments, the subject has one or more MS-like symptoms, such as those having clinically isolated syndrome (CIS) or clinically defined MS (CDMS). In other embodiments, the subject has one or more MS relapses (e.g. acute optic neuritis, transverse myelitis, brainstem syndrome, internuclear ophthalmoplegia). In some embodiments, the subject has internuclear ophthalmoplegia.

In other embodiments, the subject does not yet have a symptom associated with MS, but is at risk for developing the disease. In one embodiment, the subject is asymptomatic at the time of treatment, e.g., initial treatment. In some embodiments, the subject is not diagnosed with MS, or is diagnosed with MS but does not suffer from a relapse.

In one embodiment, the subject has a relapsing form of MS (e.g., RRMS or relapsing SPMS). In one embodiment, the subject has RRMS and has one or more ongoing clinical exacerbations and/or subclinical activity, e.g., as shown by gadolinium (Gd) enhancement or development of new and/or enlarged T2/FLAIR lesions on magnetic resonance imaging (e.g., brain or spinal cord MRI). In another embodiment, the subject has SPMS and has one or more ongoing clinical exacerbations and/or subclinical activity, e.g., as shown by gadolinium (Gd) enhancement or development of new and/or enlarged T2/FLAIR lesions on magnetic resonance imaging (e.g., brain or spinal cord MRI). In one embodiment, the subject has an active form of MS, e.g., an active RRMS. In other embodiments, the MS subject has at least one newly developed lesion. In other embodiment, the MS subject has at least one pre-existing lesion. In one embodiment, the subject has RRMS, and has one or more newly developed or pre-existing lesions, or a combination thereof. In other embodiments, the subject has a baseline EDSS score of 1.5 to 7.

In one embodiment, the subject is an MS patient (e.g., a patient with RRMS or SPMS) prior to administration of an MS therapy (a monotherapy or a combination therapy of the agents described herein). In one embodiment, the subject is a newly diagnosed or an undiagnosed RRMS or SPMS patient. In another embodiment, a subject has a radiologically isolated syndrome or clinically isolated syndrome. In yet another embodiment, a subject has an asymptomatic finding (e.g., does not present with MS symptoms, but has an MS associated finding noted on testing). In another embodiment, the subject is an MS patient (e.g., an RRMS patient) after administration of an MS therapy described herein (a monotherapy or a combination therapy of the agents described herein). In other embodiments, the subject is an MS patient after administration of the MS therapy for one, two weeks, one month, two months, three months, four months, six months, one year or more.

In some embodiments, the subject has, or is identified as having, 1, 2, 3, 4 or 5 of the following:

(i) A baseline Magnetization Transfer Ratio (MTR) in T2 lesion of less than or equal to about −0.25 to about −0.35, e.g., less than or equal to about −0.31 nMTRu;

(ii) A normalized MTR in T2 lesion of less than or equal to 0 nMTRu;

(iii) A baseline diffuse tensor imaging-radial diffusivity (DTI-RD) in T2 lesion of less than or equal to about 0.9×10⁻³ mm²/s to 1.0×10⁻³ mm²/s, e.g, less than or equal to about 0.95×10⁻³ mm²/s;

(iv) A DTI-RD in a value of less than or equal to 1.2×10⁻³ mm²/s; or

(v) A disease duration of less than or equal to about 15 years to about 35 years; e.g., less than or equal to about 20 years.

In some embodiments, the subject has, or is identified as having, (i) of the above. In other embodiments, the subject has, or is identified as having, (ii) of the above. In some embodiments, the subject has, or is identified as having, (iii) of the above. In some embodiments, the subject has, or is identified as having, (iv) of the above. In some embodiments, the subject has, or is identified as having, (v) of the above. In embodiments, the subject has, or is identified as having, (i) and (iii) of the above. In other embodiments, the subject has, or is identified as having, (i) and (iv) of the above. In other embodiments, the subject has, or is identified as having, (i) and (v) of the above. In yet other embodiments, the subject has, or is identified as having, (ii) and (iii) of the above. In yet other embodiments, the subject has, or is identified as having, (ii) and (iv) of the above. In other embodiments, the subject has, or is identified as having, (ii) and (v) of the above. In other embodiments, the subject has, or is identified as having, (iii) and (v) of the above. In other embodiments, the subject has, or is identified as having, (iv) and (v) of the above. In embodiments, the subject has, or is identified as having, (i), (iii) and (v) of the above. In other embodiments, the subject has, or is identified as having, (ii), (iii) and (v) of the above. In other embodiments, the subject has, or is identified as having, (i), (iv) and (v) of the above. In yet other embodiments, the subject has, or is identified as having, (ii), (iv) and (v) of the above. In yet other embodiments, the subject has, or is identified as having, (i)-(v) of the above.

In some embodiments, the subject has, or is identified as having, all of the following:

(i) A baseline Magnetization Transfer Ratio (MTR) in T2 lesion of less than or equal to about −0.31 normalized MTR units (nMTRu) or a normalized MTR in T2 lesion of less than or equal to 0 nMTRu;

(ii) A baseline diffuse tensor imaging-radial diffusivity (DTI-RD) in T2 lesion of less than or equal to about 0.95×10⁻³ mm²/s or a DTI-RD in a value of less than or equal to 1.2×10⁻³ mm²/s; and

(iii) A disease duration of less than or equal to about 20 years.

In some embodiments, the subject has, or is identified as having, one or both of the following:

(i) A baseline MTR in T2 lesion of less than or equal to about −0.25 to −0.35, e.g., less than or equal to about −0.31 nMTRu; or

(ii) A disease duration of less than or equal to about 10 years to about 15 years; e.g., less than or equal to about 12 years.

In some embodiments, the subject has, or is identified as having, (i) of the above. In other embodiments, the subject has, or is identified as having, (ii) of the above. In embodiments, the subject has, or is identified as having, (i) and (ii) of the above.

In some embodiments, the subject has, or is identified as having, one or both of the following:

(i) A baseline MTR in T2 lesion of less than or equal to about −0.15 to about −0.20, e.g., less than or equal to about −0.17 normalized MTR units (nMTRu); or

(ii) A baseline DTI-RD in T2 lesion of less than or equal to about 0.9×10⁻³ mm²/s to 1.0×10⁻³ mm²/s, e.g, less than or equal to about 0.98×10⁻³ mm²/s.

In some embodiments, the subject has, or is identified as having, (i) of the above. In other embodiments, the subject has, or is identified as having, (ii) of the above. In embodiments, the subject has, or is identified as having, (i) and (ii) of the above.

In some embodiments, the subject has, or is identified as having, both of the following:

(i) A baseline MTR in T2 lesion of less than or equal to about −0.17 normalized MTR units (nMTRu); and

(ii) A baseline DTI-RD in T2 lesion of less than or equal to about 0.98×10⁻³ mm²/s.

In some embodiments, the subject has, or is identified as having, a DTI-RD in T2 lesion of less than about 0.7×10⁻³ mm²/s to about 0.8×10⁻³ mm²/s, e.g, less than about 0.732×10⁻³ mm²/s at baseline, e.g., prior to treatment.

In some embodiments, the subject has, or is identified as having: RMS 18-58 years old; EDSS 2-6; greater than or equal to 1 relapse, 1 Gd+ lesion, or 1 new T2 lesion within the past 24 months; has been treated with one disease modifying therapy (DMT) continuously for greater than or equal to 6 months (e.g., no relapse for 6 months); disease duration less than or equal to 20 years; MTR in T2 lesions less than or equal to −0.17; and DTI RD in T2 lesions less than or equal to 0.98.

### Reparative Agents

In certain embodiments, the reparative agent causes one or more of: enhances myelination or re-myelination, enhances neuroaxonal protection, increases axonal extension, increases neuronal sprouting, and/or increases differentiated oligodendrocyte numbers (e.g., by increasing one or more of: survival or differentiation of oligodendrocytes), e.g., in a subject (e.g., a subject in need thereof). The method includes administering to the subject a reparative agent (e.g., a LINGO-1 antagonist), as a monotherapy or in combination with an immunomodulatory agent, in an amount sufficient to enhance one or more of: myelination, re-myelination, oligodendrocyte numbers, or neuroaxonal protection.

In one embodiment, the reparative agent is an antagonist of LRR and Ig domain-containing, Nogo receptor-interacting protein (“LINGO,” e.g., LINGO-1). LINGO-1, previously called Sp35, is a cell surface glycoprotein that is selectively expressed in the adult CNS in neurons and oligodendrocytes, where it is believed to function as a negative regulator of oligodendrocyte differentiation, myelination, and remyelination. Thus, antagonism of LINGO-1 can enhance myelination or re-myelination of axons, e.g., by oligodendrocytes, and enhance neuroaxonal protection in the CNS. LINGO-1 has been described in International Applications PCT/US2006/026271, filed Jul. 7, 2006, PCT/US2004/008323, filed Mar. 17, 2004, PCT/US2005/022881, filed Jun. 24, 2005 and PCT/US2008/000316, filed Jan. 9, 2008, each of which is incorporated by reference in its entirety herein.

In one embodiment, the reparative agent, e.g., the LINGO-1 antagonist, inhibits or reduces the expression or activity of LINGO-1, e.g., human LINGO-1.

In one embodiment, the reparative agent, e.g., the LINGO-1 antagonist, inhibits or reduces the formation and/or activity of a complex (e.g., a functional signaling complex) of the NgR1, p′75, and LINGO-1; and/or NgR1, TAJ (TROY), and LINGO-1. In another embodiment, the reparative agent, e.g., the LINGO-1 antagonist, inhibits or reduces LINGO-1 binding to NgR1.

In one embodiment, the reparative agent, e.g., the antagonist of LINGO-1, is an antibody molecule. In one embodiment, the antibody molecule reduces the formation and/or activity of a complex (e.g., a functional signaling complex) of the NgR1, p′75, and LINGO-1; and/or NgR1, TAJ (TROY), and LINGO-1. In one embodiment, the antibody molecule binds to at least one of the components of the complex (e.g., at least one of NgR1, p′75, and LINGO-1; and/or NgR1, TAJ (TROY), and LINGO-1), and inhibits or reduces the functional signaling.

In one embodiment, the antibody molecule binds to LINGO, e.g., human LINGO. In another embodiment, the antibody molecule binds to LINGO-1, e.g., human LINGO-1. The antibody molecule can be a monoclonal or single specificity antibody, or an antigen-binding fragment thereof (e.g., an Fab, F(ab′)₂, Fv, a single chain Fv fragment, a single domain antibody, a diabody (dAb), a bivalent or bispecific antibody or fragment thereof, a single domain variant thereof) that binds to LINGO-1, e.g., a mammalian (e.g., human LINGO-1 (or a functional variant thereof)). In one embodiment, the antibody molecule is a monoclonal antibody against LINGO-1, e.g., human LINGO-1. Typically, the antibody molecule is a human, a humanized, a CDR-grafted, a chimeric, a camelid, or an in vitro generated antibody to human LINGO-1 (or functional fragment thereof, e.g., an antibody fragment as described herein). Typically, the antibody inhibits, reduces or neutralizes one or more activities of LINGO-1 (e.g., one or more biological activities of LINGO-1 as described herein).

The antibody molecule can be full-length (e.g., can include at least one, and typically two, complete heavy chains, and at least one, and typically two, complete light chains) or can include an antigen-binding fragment (e.g., a Fab, an F(ab′)₂, an Fv, a single chain Fv fragment, or a single domain antibody or fragment thereof). In yet other embodiments, the antibody molecule has a heavy chain constant region chosen from, e.g., the heavy chain constant region of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The framework region or constant region of the antibody molecule can be altered, e.g., mutated, to modify the properties of the antibody (e.g., to increase or decrease one or more of: Fc receptor binding, antibody glycosylation, the number of cysteine residues, effector cell function, and/or complement function). In one embodiment, the framework or constant region of the antibody molecule is altered, e.g., mutated, to decrease one or more of: Fc receptor binding, antibody glycosylation, the number of cysteine residues, effector cell function, and/or complement function. In one embodiment, the framework region of the antibody molecule is modified to reduce antibody glycosylation, effector cell and/or complement function. In one embodiment, the antibody molecule includes an aglycosyl framework.

In another embodiment, the antibody molecule binds to LINGO-1, e.g., human LINGO-1, and is an immunoglobulin G subclass 1 (IgG1). In certain embodiments, the antibody molecule is modified to reduce effector cell and complement function compared to wild-type IgG1. In one embodiment, the antibody molecule includes an aglycosyl (IgG1) framework.

In certain embodiments, the antibody molecule specifically binds to the same, or substantially the same, LINGO-1 epitope as the reference monoclonal antibody Li62 or Li81, described in U.S. Pat. Nos. 8,058,406 and 8,128,926, both of which are incorporated by reference in their entirety herein. In an embodiment, the antibody molecule comprises, consists essentially of, or consists of, an immunoglobulin heavy chain variable region (VH) wherein the CDR1, CDR2 and CDR3 regions are selected from the amino acid sequences shown in Table 3, or an amino acid sequence substantially identical thereto (e.g., an amino acid sequence at least 80%, 85%, 90% or 95% identical to the amino acid sequences shown in Table 3; or at least 80%, 85%, 90, 95% or 100% identical to the VH CDR1, CDR2 and CDR3 regions of the immunoglobulin heavy chain of Li62 or Li81).

In some embodiments, the antibody molecule includes a VH that comprises, consists essentially of, or consists of, the amino acid sequence of SEQ ID NO: 4 or SEQ ID NO:8 or any one of SEQ ID NOs: 17 to 49, or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise, consist essentially of, or consist of, the amino acids of SEQ ID NOs: 6, 7, and 8, respectively, or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise, consist essentially of, or consist of, the amino acids of SEQ ID NOs: 2, 3, and 30, respectively, or an amino acid sequence substantially identical thereto (e.g., an amino acid sequence at least 80%, 85%, 90% or 95% identical thereto).

In other embodiments, the antibody molecule includes an immunoglobulin light chain variable region (VL) wherein the CDR1, CDR2 and CDR3 regions are selected from the polypeptide sequences shown in Table 4, or an amino acid sequence substantially identical thereto (e.g., an amino acid sequence at least 80%, 85%, 90% or 95% identical to the amino acid sequences shown in Table 4; or at least 80%, 85%, 90%, 95% or 100% identical to the VL CDR1, CDR2 and CDR3 regions of the immunoglobulin light chain of Li62 or Li81).

In one embodiment, the antibody molecule includes a VL wherein the VL CDR1, CDR2, and CDR3 comprise, consist essentially of, or consist of, the amino acids of SEQ ID NOs: 14, 15, and 16, respectively, or an amino acid sequence substantially identical thereto (e.g., an amino acid sequence at least 80%, 85%, 90% or 95% identical thereto).

In one embodiment, the antibody molecule includes a VL wherein the VL CDR1, CDR2, and CDR3 comprise, consist essentially of, or consist of, the amino acids of SEQ ID NOs: 10, 11, and 12, respectively, or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise, consist essentially of, or consist of, the amino acids of SEQ ID NOs: 6, 7, and 8, respectively; and a VL wherein the VL CDR1, CDR2, and CDR3 comprise, consist essentially of, or consist of, the amino acids of SEQ ID NOs: 14, 15, and 16, respectively; or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise, consist essentially of, or consist of, the amino acids of SEQ ID NOs: 2, 3, and 30, respectively; and a VL wherein the VL CDR1, CDR2, and CDR3 comprise, consist essentially of, or consist of, the amino acids of SEQ ID NOs: 10, 11, and 12, respectively; or an amino acid sequence substantially identical thereto (e.g., an amino acid sequence at least 80%, 85%, 90% or 95% identical thereto).

In other embodiments, the antibody molecule includes a VH selected from the group consisting of SEQ ID NOs: 1, 5, and 53-85, or an amino acid sequence substantially identical thereto (e.g., an amino acid sequence at least 80%, 85%, 90% or 95% identical to said SEQ ID NOs: 1, 5 and 53-85).

In one embodiment, the antibody molecule includes a VH that comprises, consists essentially of, or consists of, the amino acid sequence of SEQ ID NO: 5 or an amino acid sequence substantially identical thereto (e.g., an amino acid sequence at least 80%, 85%, 90% or 95% identical to said SEQ ID NO: 5).

In one embodiment, the antibody molecule includes a VH that comprises, consists essentially of, or consists of, the amino acid sequence of SEQ ID NO:66, or an amino acid sequence substantially identical thereto (e.g., an amino acid sequence at least 80%, 85%, 90% or 95% identical to said SEQ ID NO: 66).

In yet other embodiments, the antibody molecule includes a VL selected from the group consisting of SEQ ID NOs: 9 and 13, as shown in Table 4, or an amino acid sequence substantially identical thereto (e.g., an amino acid sequence at least 80%, 85%, 90% or 95% identical to said SEQ ID NOs: 9 and 13, as shown in Table 4).

In one embodiment, the antibody molecule includes a VL that comprises, consists essentially of, or consists of, the amino acid sequence of SEQ ID NO:13, or an amino acid sequence substantially identical thereto (e.g., an amino acid sequence at least 80%, 85%, 90% or 95% identical to said SEQ ID NO: 13).

In one embodiment, the antibody molecule includes a VL that comprises, consists essentially of, or consists of, the amino acid sequence of SEQ ID NO:9, or an amino acid sequence substantially identical thereto (e.g., an amino acid sequence at least 80%, 85%, 90% or 95% identical to said SEQ ID NO: 9).

In one embodiment, the antibody molecule includes a VH that comprises, consists essentially of, or consists of, the amino acid sequence of SEQ ID NO:5, or an amino acid sequence substantially identical thereto (e.g., an amino acid sequence at least 80%, 85%, 90% or 95% identical to said SEQ ID NO: 5); and a VL that comprises, consists essentially of, or consists of, the amino acid sequence of SEQ ID NO: 13, or an amino acid sequence substantially identical thereto (e.g., an amino acid sequence at least 80%, 85%, 90% or 95% identical to said SEQ ID NO: 13).

In one embodiment, the antibody molecule includes a VH that comprises, consists essentially of, or consists of, the amino acid sequence of SEQ ID NO:66, or an amino acid sequence substantially identical thereto (e.g., an amino acid sequence at least 80%, 85%, 90% or 95% identical to said SEQ ID NO: 66); and a VL that comprises, consists essentially of, or consists of, the amino acid sequence of SEQ ID NO: 9, or an amino acid sequence substantially identical thereto (e.g., an amino acid sequence at least 80%, 85%, 90% or 95% identical to said SEQ ID NO: 9).

In another embodiment, the antibody molecule includes a heavy chain as shown below, that comprises, consists essentially of, or consists of, the amino acid sequence of SEQ ID NO: 275, or a sequence substantially identical thereto (e.g., an amino acid sequence at least 80%, 85%, 90% or 95% identical thereto), as follows:

(SEQ ID NO: 275)
EVQLLESGGG LVQPGGSLRL SCAASGFTFS AYEMKWVRQA PGKGLEWVSV
IGPSGGFTFY ADSVKGRFTI SRDNSKNTLY LQMNSLRAED TAVYYCATEG
DNDAFDIWGQ GTTVTVSSAS TKGPSVFPLA PSSKSTSGGT AALGCLVKDY
FPEPVTVSWN SGALTSGVHT FPAVLQSSGL YSLSSVVTVP SSSLGTQTYI
CNVNHKPSNT KVDKKVEPKS CDKTHTCPPC PAPELLGGPS VFLFPPKPKD
TLMISRTPEV TCVVVDVSHE DPEVKFNWYV DGVEVHNAKT KPREEQYNSA
YRVVSVLTVL HQDWLNGKEY KCKVSNKALP APIEKTISKA KGQPREPQVY
TLPPSRDELT KNQVSLTCLV KGFYPSDIAV EWESNGQPEN NYKTTPPVLD
SDGSFFLYSK LTVDKSRWQQ GNVFSCSVMH EALHNHYTQK SLSLSPG.

In other embodiments, the antibody molecule comprises, consists essentially of, or consists of, a light chain as shown below, comprising the amino acid sequence of SEQ ID NO: 276, or a sequence substantially identical thereto (e.g., an amino acid sequence at least 80%, 85%, 90% or 95% identical thereto), as follows:

(SEQ ID NO: 276)
DIQMTQSPAT LSLSPGERAT LSCRASQSVS SYLAWYQQKP GQAPRLLIYD
ASNRATGIPA RFSGSGSGTD FTLTISSLEP EDFAVYYCQQ RSNWPMYTFG
QGTKLEIKRT VAAPSVFIFP PSDEQLKSGT ASVVCLLNNF YPREAKVQWK
VDNALQSGNS QESVTEQDSK DSTYSLSSTL TLSKADYEKH KVYACEVTHQ
GLSSPVTKSF NRGEC.

In one embodiment, the antibody molecule is the anti-LINGO-1 antibody, opicinumab (also referred to herein as “BIIB033”). In one embodiment, the anti-LINGO-1 antibody molecule comprises (i) a heavy chain comprising, consisting essentially of, or consisting of, the amino acid sequence of SEQ ID NO: 275, and (ii) a light chain comprising, consisting essentially of, or consisting of, the amino acid sequence of SEQ ID NO: 276.

In another embodiment, the reparative agent, e.g., the antagonist of LINGO-1, is a soluble LINGO molecule, e.g., a LINGO-1 molecule (e.g., a fragment of LINGO-1), or a soluble form of a component of the LINGO-1 complex (e.g., a soluble form of NgR1, p′75, or TAJ (TROY)).

A soluble form of LINGO or a complex component can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A, an MBP polypeptide sequence, or an antibody (e.g., a bispecific or a multispecific antibody). The fusion proteins may additionally include a linker sequence joining the first moiety, e.g., the soluble form of LINGO-1 or the complex component, to the second moiety. In other embodiments, additional amino acid sequences can be added to the N- or C-terminus of the fusion protein to facilitate expression, steric flexibility, detection and/or isolation or purification. For example, a soluble form of LINGO-1 or a complex component can be fused to a heavy chain constant region of the various isotypes, including: IgG1, IgG2, IgG3, IgG4, IgM, IgA1, IgA2, IgD, and IgE. Typically, the fusion protein can include the extracellular domain of LINGO or the complex component (or a sequence homologous thereto), and, e.g., fused to, a human immunoglobulin Fc chain, e.g., a human IgG (e.g., a human IgG1 or a human IgG2, or a mutated form thereof). The Fc sequence can be mutated at one or more amino acids to reduce effector cell function, Fc receptor binding and/or complement activity.

In another embodiment, one or more reparative agents are added in combination. For example, a LINGO-1 antagonist can be added in combination with another remyelinating agent.

### Immunomodulatory Agents

The methods, kits and compositions described herein can include one or more immunomodulatory agents. In certain embodiments, the immunomodulatory agent is chosen from one or more of:

an IFN-β 1 molecule;

a polymer of glutamic acid, lysine, alanine and tyrosine, e.g., glatiramer (e.g., Copaxone®);

an antibody or fragment thereof against alpha-4 integrin, e.g., natalizumab (e.g., Tysabri®);

an anthracenedione molecule, e.g., mitoxantrone (e.g., Novantrone®);

a fingolimod, e.g., FTY720 (e.g., Gilenya®);

a dimethyl fumarate, e.g., an oral dimethyl fumarate (e.g., Tecfidera®);

an antibody to the alpha subunit of the IL-2 receptor of T cells (CD25), e.g., daclizumab;

an antibody against CD52, e.g., alemtuzumab (e.g., CAMPATH);

an inhibitor of a dihydroorotate dehydrogenase, e.g., leflunomide or an active metabolite thereof, e.g., teriflunomide (e.g., AUBAGIO);

an antibody to CD20, e.g., rituximab, or ocrelizumab;

a Sphingosine 1-phosphate (S1P) modulating agent, e.g., as described in WO 2012/109108; or

a corticosteroid.

In one embodiment, the immunomodulatory agent is an IFN-β 1 molecule. The IFN-β 1 molecule can be chosen from one or more of an IFN-β1a or IFN-β 1b polypeptide, a variant, a homologue, a fragment or a pegylated variant thereof.

In one embodiment, the IFN-β 1 molecule includes an IFNβ agent chosen from an IFN-β1a molecule, an IFN-β1b molecule, or a pegylated variant of an IFN-β1a molecule or an IFN-β 1b molecule.

In one embodiment, the IFNβ1 molecule is an IFN-β1a agent (e.g., Avonex®, Rebif®). In another embodiment, the IFNβ1 molecule is an INF-β1b agent (e.g., Betaseron®, Betaferon® or Extavia®).

In one embodiment, the immunomodulatory agent is a polymer of glutamic acid, lysine, alanine and tyrosine, e.g., glatiramer (e.g., Copaxone®).

In one embodiment, the immunomodulatory agent is an antibody or fragment thereof against alpha-4 integrin (e.g., natalizumab (e.g., Tysabri®)).

In yet other embodiments, the immunomodulatory agent is an anthracenedione molecule (e.g., mitoxantrone (e.g., Novantrone®)).

In yet another embodiment, the immunomodulatory agent is a fingolimod (e.g., FTY720; e.g., Gilenya®).

In one embodiment, the immunomodulatory agent is a dimethyl fumarate (e.g., an oral dimethyl fumarate (e.g., BG-12)).

In other embodiments, the immunomodulatory agent is an antibody to the alpha subunit of the IL-2 receptor of T cells (CD25) (e.g., Daclizumab).

In other embodiments, the immunomodulatory agent is an antibody to CD20, e.g., ocrelizumab.

In other embodiments, the immunomodulatory agent is a corticosteroid, e.g., methylprednisolone (e.g., high dose corticosteroid, e.g., methylprednisolone).

In certain embodiments, the method further includes the use of one or more symptom management therapies, such as antidepressants, analgesics, anti-tremor agents, among others.

Any combination of the reparative agent (e.g., one or more reparative agents described herein, e.g., a LINGO-1 antagonist) and an immunomodulatory agent (e.g., one or more immunomodulatory agents described herein) can be used in the methods, kits and compositions described herein. For example, the reparative agent can be combined with a polymer of glutamic acid, lysine, alanine and tyrosine, e.g., glatiramer. In other embodiments, the reparative agent can be combined with an antibody or fragment thereof against alpha-4 integrin, e.g., natalizumab. In yet another embodiment, the reparative agent can be combined with an anthracenedione molecule, e.g., mitoxantrone. In yet another embodiment, the reparative agent can be combined with a fingolimod, e.g., FTY720. In yet another embodiment, the reparative agent can be combined with a dimethyl fumarate, e.g., an oral dimethyl fumarate. In other embodiments, the reparative agent can be combined with an antibody to the alpha subunit of the IL-2 receptor of T cells (CD25), e.g., daclizumab. In yet another embodiment, the reparative agent can be combined with an antibody against CD52, e.g., alemtuzumab. In yet another embodiment, the reparative agent can be combined with an inhibitor of a dihydroorotate dehydrogenase, e.g., teriflunomide. In another embodiment, the reparative agent can be combined with an antibody to CD20, e.g., ocrelizumab. In another embodiment, the reparative agent can be combined with a corticosteroid, e.g., methylprednisolone. In one embodiment, the reparative agent can be combined with a S1P modulating agent.

In other embodiment, the reparative agent is combined with two, three, four or more immunomodulatory agents, e.g., two, three, four or more of the immunomodulatory agents described herein. In one exemplary embodiment, a combination of a LINGO antagonist, an IFN-β 1 molecule and a corticosteroid is used. In other embodiments, a combination of a LINGO antagonist, an IFN-β 1 molecule and a polymer of glutamic acid, lysine, alanine and tyrosine, e.g., glatiramer, is used. In yet other embodiments, a combination of a LINGO antagonist, an IFN-β 1 molecule and an antibody or fragment thereof against alpha-4 integrin, e.g., natalizumab, is used.

In certain embodiment of the methods, kits and compositions described herein, the reparative agent is an antibody molecule against LINGO-1, e.g., an anti-LINGO antibody as described herein, and the immunomodulator agent is an IFN-β 1 molecule, e.g., an IFN-β 1 molecule as described herein.

### Monotherapy and Combination Therapy; Timing of Administration

The reparative agent can be administered as a monotherapy or a combination therapy. The combinations of reparative agent (e.g., LINGO-1 antagonist) and the immunomodulatory agent described herein can be administered in any order, e.g., concurrently or sequentially as described herein. In one embodiment, the reparative agent and the immunomodulatory agent are administered concurrently. In another embodiment, the reparative agent and the immunomodulatory agent are administered sequentially. For example, the administration of the reparative agent and the immunomodulatory agent can overlap, at least in part or completely, with each other.

In certain embodiments, initiation of the administration of the immunomodulatory agent and the reparative agent occurs at the same time. In other embodiments, the immunomodulatory agent is administered before initiating treatment with the reparative agent. In yet other embodiments, the reparative agent is administered before initiating treatment with the immunomodulatory agent. In another embodiment, the administration of the immunomodulatory agent continues after cessation of administration of the reparative agent. In other embodiments, administration of the reparative agent continues after cessation of administration of the immunomodulatory agent. In other embodiments, administration of the reparative agent continues intermittently, e.g., is discontinued once a subject achieves a selected cumulative exposure and is reinitiated if symptoms reappear or after a fixed period of time, e.g., is intermittent between dosage regimens. In one embodiment, the immunomodulatory agent is given continuously. In other embodiments, administration of the immunomodulatory agent continues intermittently (e.g., for 2 or 3 months every 3 or 6 or 12 months, or for 3-6 months every 1-2 years), while the reparative agent is given continuously, e.g., as background therapy.

In certain embodiments, the reparative agent is an antibody molecule against LINGO-1 and is administered, as a monotherapy or a combination therapy, intravenously, subcutaneously or intramuscularly. In one embodiment, the antibody molecule is administered intravenously. In such embodiments, the antibody molecule is administered, as a monotherapy or a combination therapy, at about 0.3 mg/kg, about 1 mg/kg, about 3 mg/kg, about 10 mg/kg, about 30 mg/kg, about 60 mg/kg, or about 100 mg/kg. For example, between about 1 to 150 mg/kg, e.g., 3 to 100 mg/kg (typically, at 3 mg/kg, 10 mg/kg, 30 mg/kg, about 50 mg/kg or 100 mg/kg). In other embodiments, the anti-LINGO-1 antibody is administered as a fixed dose, e.g., about 150 mg to about 7500 mg, about 100 mg to about 7000 mg, about 200 mg to about 3500 mg, about 250 mg to about 3000 mg, about 300 mg to about 2000 mg, about 350 mg to about 1500 mg, about 500 mg to about 1000 mg, about 700 mg to about 900 mg, about 200 mg to about 350 mg, or about 7000 mg, about 5000 mg, about 2000 mg, about 700 mg, about 500 mg, about 350 mg, or about 200 mg. In some embodiments, the anti-LINGO-1 antibody molecule is administered at a fixed dose of about 700 mg to about 850 mg, e.g., about 750 mg to about 800 mg every four weeks. In some embodiments, the anti-LINGO-1 antibody molecule is administered at a fixed dose of about 700 mg every four weeks, about 725 mg every four weeks, about 750 mg every four weeks, about 775 mg every four weeks, about 800 mg every four weeks, about 825 mg every four weeks, or about 850 mg every four weeks. In some embodiments, the antibody molecule is administered once every one, two, three, four or five weeks by IV infusion.

In one embodiment, the anti-LINGO-1 antibody molecule is administered at about 100 mg/kg, or 7000 mg, via IV infusion or SC injection, e.g., as a single dose.

In another embodiment, the anti-LINGO-1 antibody molecule is administered as one dose of about 30 mg/kg, or 2000 mg, e.g., administered intravenously, every four weeks, to a total of 3 doses.

In another embodiment, the anti-LINGO-1 antibody molecule is administered as one dose of about 10 mg/kg, 700 mg, or 750 mg, e.g., administered intravenously, every four weeks, to a total of 8 doses;

In another embodiment, the anti-LINGO-1 antibody molecule is administered as one dose of 3 mg/kg, or 200 mg, e.g., administered intravenously, every four weeks, to a total of 18 doses; or

In another embodiment, the anti-LINGO-1 antibody molecule is administered as one dose of 3-5 mg/kg, or 200-350 mg, e.g., administered subcutaneouly, every four weeks, to a total of 18 doses.

In another embodiment, the anti-LINGO-1 antibody molecule is administered as one dose of 10 mg/kg or 750 mg, e.g., intravenously, every 12 weeks, up to a total of 8 doses.

In another embodiment, the anti-LINGO-1 antibody molecule is administered as one dose of 30 mg/kg, e.g., intravenously, every 24 weeks, up to a total of 4 doses.

In certain embodiments, the immunomodulatory agent is an IFN-β 1 molecule is administered intravenously, subcutaneously or intramuscularly. For example, the IFN-β 1 molecule can be administered at one or more of:

(i) at 20-45 microgram (e.g., 30 microgram), e.g., once a week via intramuscular injection;

(ii) at 20-30 microgram (e.g., 22 microgram), e.g., three times a week, or at 40-50 micrograms (e.g., 44 micrograms), e.g., once a week, via subcutaneous injection; or

(iii) in an amount of between 10 and 50 μg intramuscularly, e.g., three times a week, or every five to ten days, e.g., once a week; or
(iv) in an amount between 200 and 600 μg (e.g., between 250 and 500 μg), e.g., every other day, via subcutaneous injection. In one embodiment, the IFN-β 1 molecule is an interferon β-1b (Betaseron®/Betaferon®, or Extavia®).

In one embodiment, the IFN-β 1 molecule is administered at 30 microgram, e.g., once a week, via subcutaneous injection.

Alternatively, the IFN-β 1 molecule is pegylated IFN-β 1 and is administered at 50 μg to 200 μg, e.g., 50 μg to 60 μg (e.g., 63 μg), 90 μg to 100 μg (e.g., 94 μg) or 120 μg to 130 μg (e.g., 125 μg) once every other week subcutaneously.

In other embodiments, the reparative agent is an antibody molecule against LINGO-1 and is administered once every four weeks by IV infusion or SC injection dosed at about 3 mg/kg, about 10 mg/kg, about 30 mg/kg, 50 mg/kg or about 100 mg/kg, or as a fixed dose, e.g., as described herein; and the immunomodulatory agent the IFN-β 1 is administered at one or more of:

(i) at 20-45 microgram (e.g., 30 microgram), e.g., once a week via intramuscular injection;

(ii) at 20-30 microgram (e.g., 22 microgram), e.g., three times a week, or at 40-50 micrograms (e.g., 44 micrograms), e.g., once a week, via subcutaneous injection; or

(iii) in an amount of between 10 and 50 μg intramuscularly, e.g., three times a week, or every five to ten days, e.g., once a week.

Alternatively, the IFN-β 1 molecule is pegylated IFN-β 1 and is administered at 50 μg to 200μg, e.g., 50 μg to 60 μg (e.g., 63 μg), 90 μg to 100 μg (e.g., 94 μg) or 120 μg to 130 μg (e.g., 125 μg) once every other week subcutaneously.

### Subject Monitoring

Alternatively, or in combination, with the methods disclosed herein, a method of evaluating, diagnosing, and/or monitoring the progression of, a CNS disorder or a CNS demyelinating disease is disclosed. The method includes evaluating a subject (e.g., a patient, a patient group or a patient population), having the CNS disorder or CNS demyelinating disease, or at risk of developing the disorder.

In one embodiment, the subject is evaluated based on the cumulative exposure of the reparative agent, e.g., the anti-LINGO-1 antibody molecule disclosed herein (e.g., a selected cumulative exposure as described herein). In some embodiments, depending on the cumulative exposure level of the reparative agent, administration of the reparative agent is discontinued, e.g., such that a selected cumulative exposure (e.g., a selected cumulative exposure as described herein) of the reparative agent is achieved.

In other embodiments, the subject is evaluated using one, two, three or more of:

(i) neurological assessment, e.g., an assessment chosen from one, two, three or more of: diffuse tensor imaging-radial diffusivity (DTI-RD, e.g., whole brain or abnormal T2 lesions DTI); diffuse tensor imaging-fractional anisotropy (DTI-FA, e.g., whole brain or abnormal T2 lesions DTI); Magnetization Transfer Ratio (nMTRu); brain volume assessment (e.g., whole, cerebral, cortical and/or thalamic brain volume); or gadolinium (Gd) lesion enhancement. In embodiments, the DTI-RD and/or nMTRu are evaluated in T2 lesion or in whole brain. In embodiments, the assessment comprises a change in one or more of the aforesaid assessments, e.g., a change from a baseline value;

(ii) a measure of disability (e.g., EDSS, wherein a reduced score is indicative of improvement, and an increased score is indicative of worsening);

(iii) a measure of physical function. For example, an assessment of physical function can include an assessment of ambulatory function (e.g., short distance and/or longer distance ambulatory function), alone or in combination with an assessment of upper and/or lower extremity function. For example, a measure of upper extremity function, e.g., using a 9 Hole Peg Test (9HPT dominant and non-dominant hands, can be used, wherein reduced time is indicative of improvement, and increased time is indicative of worsening), a measure of short distance ambulatory function, e.g., using a Timed Walk of 25 Feet (T25FW, wherein reduced time is indicative of improvement, and increased time is indicative of worsening), or a measure of long distance ambulatory function, e.g., using a 6 minute walk test (6 MW, wherein reduced time is indicative of improvement, and increased time is indicative of worsening); or

(iv) a measure of cognitive function, e.g., PASAT-3, wherein an increased score is indicative of improvement, and a decreased score is indicative of worsening

In certain embodiments, the subject is evaluated by one or more of:

performing a neurological examination;

acquiring the subject's status on the Expanded Disability Status Scale (EDSS);

acquiring the subject's status on the Multiple Sclerosis Functional Composite (MSFC);

acquiring the subject's status on the Overall Response Score (ORS);

detecting the subject's lesion status, e.g., as assessed using an MRI;

acquiring a measure of upper and/or lower extremity function;

acquiring a measure of ambulatory function (e.g., short distance ambulatory function) (e.g., Timed Walk of 25 Feet (T25FW)); or long distance ambulatory function (e.g. the 6 minute walk test (6 MW);

acquiring a measure of cognitive function (e.g., an MS-COG or BICAMS or SDMT); or

acquiring an assessment of visual function (e.g., evaluating versional dysconjugacy index (VDI)).

In one embodiment, the measure of upper extremity function is acquired using a 9 Hole Peg Test (9HP).

In other embodiments, the measure of short distance ambulatory function is acquired using a Timed Walk of 25 Feet (T25FW).

In other embodiments, the measure of long distance ambulatory function is acquired using a 6 minute walk test (6 MW).

In certain embodiments, an increase by at least 10%, 15%, 20%, 25% or higher in a measure of extremity and/or ambulatory function is indicative of disease progression, e.g., a steady worsening of symptoms and/or disability, in the subject; and a decrease of at least 10%, 15%, 20%, 25% or more in a measure of extremity and/or ambulatory function as described above is indicative of an improved outcome (e.g., a decrease in disease progression or an improved condition) in the subject.

In certain embodiments, the subject is evaluated using a neurological examination, e.g., EDSS. In some embodiments, the EDSS includes an assessment of neurological function, an assessment of ambulatory function, or both. In one embodiment, an EDSS score is calculated based on a combination of one or more scores for the EDSS functional systems (FS) (e.g., one, two, three, four, five, six, or all seven individual scores for the EDSS FS chosen from visual, brainstem, cerebellar, motor, sensory, bladder/bowel or cognitive systems). In other embodiments, the EDSS includes a score for ambulation. In one embodiment, the EDSS includes a determination of a subject's ambulation that includes an assessment of one or more (or all) of: Unrestricted ambulation, e.g., without aid or rest for a predetermined distance (e.g., a distance greater or equal to 500, 300, 200, or 100 meters, or less than 200 or 100 meters); unilateral assistance; bilateral assistance; essentially or fully restricted to a wheelchair; or essentially or fully restricted to a bed.

In one embodiment, the subject is evaluated using an Overall Response Score (ORS). In one embodiment, the ORS comprises the sum of scores obtained at each clinical visit every time that each of 4 clinical measures (EDSS, 9HPT dominant hand, 9HPT non-dominant hand, and T25FW) improved or worsened.

In one embodiment, the assessment of visual function is acquired by one or more of: e.g., visual acuity (e.g., low-contrast letter acuity (LCLA) or high contrast visual acuity), Visual Function Questionnaire (VFQ), a 10-Item Neuro-Ophthalmic Supplement (NOS-10), Functional Acuity Contrast Testing (FACT), VEPs, such as FF-VEP or mfVEP (described e.g., in MacKay, AM (2008) Invest Ophthalmol Vis Sci. 49(1):438-41), optical coherence tomography (OCT), some of which are described in, e.g., Balcer et al. (2010) Neurology 74 Suppl 3:S16-23; Bock, M. et al. (2012) Br J Ophthalmol. 96(1):62-7).

In yet other embodiments, the measure of cognitive function comprises an evaluation of a learning test, a memory test and/or an attention/processing speed test. For example, the measure of cognitive function can include an evaluation of one or more of auditory memory, verbal learning and/or remembering visual information (e.g., Selective Reminding Test (SRT)); tests for evaluating auditory/verbal memory (e.g., California Verbal Learning Test Second Edition (CVLT2)), the Rey Auditory Verbal Learning Test (RAVLT); tests for evaluating visual/spatial memory (e.g., Brief Visuospatial Memory Test Revised (BVMTR)); processing speed cognitive tests, e.g., Paced Auditory Serial Addition Test (PASAT), Symbol Digit Modalities Test (SDMT); MSNQ-information, MSNQ-subject, and/or SF-36. In one embodiment, the measure of cognitive function is performed using a composite of MS cognitive endpoint that comprises SDMT, PASAT-3 and -3, SRT-Total Learned (SRT-TL), SRT Delayed Recall (SRT-DR), and BVMTR Delayed Recall (BVMTR-DR) (e.g., MS-COG as described in Cadavid et al., 29^{th} Congress European Committee for Treatment and Research in MS (ECTRIMS), 2-5 Oct. 2013).

In certain embodiments, the subject's lesion status is evaluated using magnetic resonance imaging. In one embodiment, the magnetic resonance imaging comprises magnetization transfer ration and/or diffusion tensor imaging.

In certain embodiments, an improvement in the subject is defined by one or more of:

a. ≥1.0 point decrease in EDSS from a baseline score of ≤6.0;

b. ≥15% improvement from baseline in T25FW;

c. ≥15% improvement from baseline in SHPT; or

d. ≥10% (e.g., 10%, 12%, 20%, 30%) improvement from baseline in PASAT or SDMT.

In some embodiments, the subject has, or is identified as having, 1, 2, or 3 of the following:

(i) A baseline Magnetization Transfer Ratio (MTR) in T2 lesion of less than or equal to about −0.25 to about −0.35, e.g., less than or equal to about −0.31 normalized MTR units (nMTRu);

(ii) A baseline diffuse tensor imaging-radial diffusivity (DTI-RD) in T2 lesion of less than or equal to about 0.9×10⁻³ mm²/s to 1.0×10⁻³ mm²/s, e.g, less than or equal to about 0.95×10⁻³ mm²/s; or

(iii) A disease duration of less than or equal to about 15 years to about 35 years; e.g., less than or equal to about 20 years.

In some embodiments, the subject has, or is identified as having, (i) of the above. In other embodiments, the subject has, or is identified as having, (ii) of the above. In embodiments, the subject has, or is identified as having, (i) and (ii) of the above. In other embodiments, the subject has, or is identified as having, (i) and (iii) of the above. In yet other embodiments, the subject has, or is identified as having, (ii) and (iii) of the above. In yet other embodiments, the subject has, or is identified as having, (i)-(iii) of the above.

In some embodiments, the method of evaluating further includes administering a dosage regimen as described herein if the subject is identified as having 1, 2, or 3 of the above.

In some embodiments, the method includes administering a dosage regimen as described herein if the subject is identified as having (i) of the above. In embodiments, the method further comprises administering the dosage regimen if the subject is identified as having (ii) of the above, thus the subject is identified as having (i) and (ii) of the above. In embodiments, the method further comprises administering the dosage regimen if the subject is identified as having (iii) of the above, thus the subject is identified as having (i) and (iii) of the above. In embodiments, the method further comprises administering the dosage regimen if the subject is identified as having (ii)-(iii) of the above, thus the subject is identified as having (i)-(iii) of the above.

In yet other embodiments, the method includes administering a dosage regimen as described herein if the subject is identified as having (ii) of the above. In embodiments, the method further comprises administering the dosage regimen if the subject is identified as having (iii) of the above, thus the subject is identified as having (ii) and (iii) of the above.

In some embodiments, the subject has, or is identified as having, all of the following:

(i) A baseline Magnetization Transfer Ratio (MTR) in T2 lesion of less than or equal to about −0.31 normalized MTR units (nMTRu);

(ii) A baseline diffuse tensor imaging-radial diffusivity (DTI-RD) in T2 lesion of less than or equal to about 0.95×10⁻³ mm²/s; and

(iii) A disease duration of less than or equal to about 20 years.

In some embodiments, the method of evaluating includes administering an anti-LINGO-1 antibody molecule at a dose of about 10 mg/kg if the subject is identified as having (i)-(iii) of the above. In other embodiments, the method of evaluating includes administering the anti-LINGO-1 antibody molecule at a fixed dose of about 750 mg if the subject is identified as having (i)-(iii) of the above.

In some embodiments, the subject has, or is identified as having, one or both of the following:

(i) A baseline MTR in T2 lesion of less than or equal to about −0.25 to −0.35, e.g., less than or equal to about −0.31 nMTRu; or

(ii) A disease duration of less than or equal to about 10 years to about 15 years; e.g., less than or equal to about 12 years.

In some embodiments, the subject has, or is identified as having, (i) of the above. In other embodiments, the subject has, or is identified as having, (ii) of the above. In embodiments, the subject has, or is identified as having, (i) and (ii) of the above.

In some embodiments, the method of evaluating further includes administering a dosage regimen as described herein if the subject is identified as having 1 or 2 of the above.

In some embodiments, the method includes administering a dosage regimen as described herein if the subject is identified as having (i) of the above. In embodiments, the method further comprises administering the dosage regimen if the subject is identified as having (ii) of the above, thus the subject is identified as having (i) and (ii) of the above.

In other embodiments, the method includes administering a dosage regimen as described herein if the subject is identified as having (ii) of the above.

In some embodiments, the subject has, or is identified as having, one or both of the following:

(i) A baseline MTR in T2 lesion of less than or equal to about −0.15 to about −0.20, e.g., less than or equal to about −0.17 normalized MTR units (nMTRu); or

(ii) A baseline DTI-RD in T2 lesion of less than or equal to about 0.9×10⁻³ mm²/s to 1.0×10⁻³ mm²/s, e.g, less than or equal to about 0.98×10⁻³ mm²/s.

In some embodiments, the subject has, or is identified as having, (i) of the above. In other embodiments, the subject has, or is identified as having, (ii) of the above. In embodiments, the subject has, or is identified as having, (i) and (ii) of the above.

In some embodiments, the method of evaluating further includes administering a dosage regimen as described herein if the subject is identified as having 1 or 2 of the above.

In some embodiments, the method includes administering a dosage regimen as described herein if the subject is identified as having (i) of the above. In embodiments, the method further comprises administering the dosage regimen if the subject is identified as having (ii) of the above, thus the subject is identified as having (i) and (ii) of the above.

In other embodiments, the method includes administering a dosage regimen as described herein if the subject is identified as having (ii) of the above.

In some embodiments, the subject has, or is identified as having, both of the following:

(i) A baseline MTR in T2 lesion of less than or equal to about −0.17 normalized MTR units (nMTRu); and

(ii) A baseline DTI-RD in T2 lesion of less than or equal to about 0.98×10⁻³ mm²/s.

In some embodiments, the method for evaluating includes administering an anti-LINGO-1 antibody molecule at a dose of about 10 mg/kg if the subject is identified as having (i) and (ii) of the above. In other embodiments, the method for evaluating includes administering the anti-LINGO-1 antibody molecule at a fixed dose of about 750 mg if the subject is identified as having (i) and (ii) of the above.

In some embodiments, the subject has, or is identified as having, a DTI-RD in T2 lesion of less than 0.7×10⁻³ mm²/s to 0.8×10⁻³ mm²/s, e.g, less than about 0.732×10⁻³ mm²/s at baseline, e.g., prior to treatment.

In some embodiments, the method of evaluating further includes administering a dosage regimen as described herein if the subject is identified as having a DTI-RD in T2 lesion of less than about 0.7×10⁻³ mm²/s to about 0.8×10⁻³ mm²/s, e.g, less than about 0.732×10⁻³ mm²/s at baseline, e.g., prior to treatment.

In other embodiments, the method of evaluating further includes one or more of the following:

(i) identifying the subject as being in need of a therapy, e.g., a therapy as described herein;

(ii) identifying the subject as having an increased or a decreased response to a therapy, e.g., a therapy as described herein. In some embodiments, the therapy is discontinued or modified based on the subject's response and/or the cumulative exposure of the reparative agent;

(iii) identifying the subject as being stable, as showing an improvement in function or abilities (e.g., as being a disease non-progressor), or showing a decline in function or abilities (e.g., as being a disease progressor);

(iv) diagnosing, and/or prognosing the subject.

The steps in the methods described herein (e.g., administration of the reparative agent and immunomodulatory agent (“administration step”), and subject monitoring and/or evaluating (“evaluating step”) can be performed in any order. In one embodiment, the administration step occurs prior to the evaluating step. In another embodiment, the evaluating step occurs prior to the administration step.

Methods disclosed herein can further include the step of evaluating, e.g., diagnosing, a subject at risk of developing, a CNS demyelinating disorder (e.g., multiple sclerosis or optic neuritis, or both). The method includes acquiring a measure (e.g., detecting or measuring), one or both of optic nerve damage or optic nerve conductance for one or both eyes of the subject, wherein the presence of optic nerve damage and/or a delay in optic nerve conductance in one or both eyes indicates that the subject is at risk for developing the CNS demyelinating disorder. In some embodiments, the methods disclosed herein can include the step of evaluating versional dysconjugacy index (VDI) to evaluate, e.g., diagnose or monitor, a subject at risk of developing, or having, a CNS demyelinating disorder (e.g., multiple sclerosis, internuclear ophthalmoparesis (INO), and/or optic neuritis) in a patient.

In one embodiment, the subject has not been diagnosed with multiple sclerosis according to one or more of:

performing a neurological examination;

acquiring the subject's status on the Expanded Disability Status Scale (EDSS);

acquiring the subject's status on the Multiple Sclerosis Functional Composite (MSFC);

acquiring the subject's status on the Overall Response Score (ORS);

detecting the subject's lesion status;

acquiring a measure of upper and/or lower extremity function;

acquiring a measure of short distance ambulatory function;

acquiring a measure of long distance ambulatory function; or acquiring a measure of cognitive function.

In certain embodiments, the step of acquiring the measure of optic nerve damage comprises measuring visual evoked potential (VEP) amplitude, e.g., full field VEP (FF-VEP) amplitude and/or multi-field VEP (mfVEP) amplitude. In one embodiment, an mfVEP amplitude that is (i) at least 40 nanovolts lower than a control amplitude, (ii) at least 20% lower than a control amplitude, or (iii) less than or equal to 180 nanovolts, indicates the presence of optic nerve damage in the eye(s) of the subject. The control amplitude can be the average VEP amplitude, e.g., FF-VEP amplitude and/or mfVEP amplitude, of a normal eye, e.g., an eye of a subject not having an optic nerve disorder or condition, e.g., acute optic neuritis.

In other embodiments, the step of acquiring the measure of optic nerve conductance comprises measuring VEP latency, e.g., FF-VEP latency or mfVEP latency. In some embodiments, a VEP latency (i) that is at least 3 milliseconds higher than a control latency, or (ii) that is at least 3% higher than a control latency; or (iii) an FF-VEP latency that is 110 milliseconds or higher, or (iv) an mfVEP latency that is 155 milliseconds or higher, indicates a delay in optic nerve conductance in the eye. In yet other embodiments, the control latency is the average VEP latency, e.g., FF-VEP latency or mfVEP latency, of a normal eye, e.g., an eye of a subject not having an optic nerve disorder or condition, e.g., acute optic neuritis.

In some embodiments, the step of evaluating versional dysconjugacy index (VDI) comprises methods known in the art, for example, as described in Frohman et al. J Neurol Neurosurg Psychiatry. 2002 July; 73(1):51-5; Frohman et al. J Neurol Sci. 2003 Jun. 15; 210(1-2):65-71; and Jozefowicz-Korczynska et al. J Neurol. 2008 July; 255(7):1006-11. doi: 10.1007/s00415-008-0819-5, hereby incorporated by reference.

### Kits and Compositions

In another aspect, the invention features a kit that includes a reparative agent (e.g., a LINGO-1 antagonist, e.g., an anti-LINGO-1 antibody molecule as described herein). Optionally, the kit is labeled and/or contains instructions for use in treating or preventing a CNS disorder, e.g., a CNS demyelinating disease as described herein. In embodiments, the kits include instructions for administration of the reparative agent, e.g., differential administration of the reparative agent, e.g., discontinued use or intermittent use. In some embodiments, the selected cumulative exposure of the reparative agent is 12,000 μg*day/mL to about 106,000 μg*day/mL, between about 20,000 μg*day/mL to about 72,000 μg*day/mL; between about 20,000 μg*day/mL to about 55,000 μg*day/mL, between about 20,000 μg*day/mL to about 35,000 μg*day/mL, or between about 35,000 μg*day/mL to about 55,000 μg*day/mL.

In yet another aspect, the invention features a composition (e.g., a packaged composition) that includes a reparative agent (e.g., a LINGO-1 antagonist, e.g., an anti-LINGO-1 antibody molecule as described herein). Optionally, the composition is labeled and/or contains instructions for use of the reparative agent in treating or preventing a CNS disorder, e.g., a CNS demyelinating disease. In embodiments, the compositions include instructions for administration of the reparative agent, e.g., differential administration of the reparative agent, e.g., discontinued use or intermittent use. In some embodiments, the selected cumulative exposure of the reparative agent is 12,000 μg*day/mL to about 106,000 μg*day/mL, between about 20,000 μg*day/mL to about 72,000 μg*day/mL; between about 20,000 μg*day/mL to about 55,000 μg*day/mL, between about 20,000 μg*day/mL to about 35,000 μg*day/mL, or between about 35,000 μg*day/mL to about 55,000 μg*day/mL.

In one embodiment, the kits and compositions further comprises a second agent, e.g., a second agent as described herein (e.g., an IFN-β 1 molecule) to be administered in combination with the LINGO-1 antagonist.

The LINGO-1 antagonist and/or the immunomodulatory agent of the compositions, kits and packaged compositions described herein can be in a form suitable for any route of administration, e.g., peripheral administration (e.g., intravenous, subcutaneous, intramuscular, intravitreal, intrathecal, or oral administration). The route of administration can be the same or different depending on the composition used. In one embodiment, the packaged pharmaceutical composition includes a LINGO-1 antagonist (e.g., an antibody against LINGO-1) in a form or preparation suitable for intravenous administration. In another embodiment, the packaged pharmaceutical composition includes an immunomodulatory agent (e.g., an interferon) in a form or preparation suitable for intramuscular administration. One or more agents can be included in the packaged pharmaceutical composition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE FIGURES

The patent or application file contains at least one drawing executed in color. Copies of this patent or patent application publication with color drawing(s) will be provided by the Office upon request and payment of the necessary fee. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic showing a clinical trial design (RENEW trial ClinicalTrials.gov Identifier: NCT01721161).

FIG. 2 is a diagram depticting the nine sectors (corresponding to the early treatment diabetic retinopathy grid) used to calculate average retinal ganglion cell layer thickness.

FIG. 3 is a chart showing rates of withdrawal and treatment discontinuation in the clinical trial.

FIG. 4 is a bar graph showing the adjusted mean change in optic nerve conduction latency (measured by FF-VEP) in the affected eye compared with the unaffected fellow eye at baseline in the PP and ITT populations at week 32 (by MMRM) in the RENEW trial. The left bar of each set of bars refers to the placebo group, and the right bar of each set of bars refers to the anti-LINGO-1 group.

FIG. 5 is a bar graph showing the mean RGCL/IPL thickness at each visit (measured by SD-OCT) in the affected eye in the ITT population up to week 32 in the RENEW trial. The left bar of each set of bars refers to the placebo group, and the right bar of each set of bars refers to the anti-LINGO-1 group.

FIG. 6 is a bar graph showing the mean RGCL/IPL thickness at each visit (measured by SD-OCT) in the affected eye in the PP population up to week 32 in the RENEW trial. The left bar of each set of bars refers to the placebo group, and the right bar of each set of bars refers to the anti-LINGO-1 group.

FIGS. 7A and 7B are bar graphs showing the adjusted mean change in RGCL/IPL thickness in the affected eye in the PP population at 4 weeks (A) and 24 weeks (B). The left bar of each set of bars refers to the group with FF-VEP latency recovery, and the right bar of each set of bars refers to the group without FF-VEP latency recovery.

FIGS. 8A and 8B are bar graphs showing the adjusted mean change in optic nerve conduction latency (measured by FF-VEP) in the affected eye compared with the unaffected fellow eye at baseline in the PP populations in the RENEW trial in subjects <33 years old (A) and in subjects ≥33 years old (B). The left bar of each set of bars refers to the placebo group, and the right bar of each set of bars refers to the anti-LINGO-1 group.

FIG. 9 is a diagram depicting exemplary individual segments assessed using multifocal visual evoked potentials (mfVEP).

FIGS. 10A and 10B are bar graphs showing the adjusted mean change in mfVEP latency compared with FF-VEP latency at week 24 in the affected eye compared with the unaffected fellow eye at baseline (by ANCOVA). CI=confidence interval; FF-VEP=full-field visual evoked potentials; ITT=intent-to-treat; mfVEP=multifocal visual evoked potentials; PP=per-protocol in the RENEW trial. The left bar of each set of bars refers to the placebo group, and the right bar of each set of bars refers to the anti-LINGO-1 group. mfVEP latency is shown in 10A and FF-VEP latency is shown in 10B.

FIGS. 11A and 11B are bar graphs showing the adjusted mean differences in mfVEP latency and amplitude at week 24 in subjects classified as having latency recovery using the primary endpoint measure, FF-VEP*. CI=confidence interval; FF-VEP=full-field visual evoked potentials; mfVEP=multifocal visual evoked potentials; *FF-VEP latency recovery was defined as affected eye FF-VEP latency ≤10% worse than the fellow eye; FF-VEP latency was the primary endpoint in the RENEW trial. The adjusted mean difference in mfVEP latency is shown in 11A and the adjusted mean difference in mfVEP amplitude is shown in 11B.

FIG. 12 is a bar graph showing mfVEP data for the unaffected fellow eye (mfVEP fellow eye average amplitude by treatment group-ITT analysis), demonstrating preservation of amplitude with anti-LINGO-1 treatment. The left bar of each set of bars refers to the placebo group, and the right bar of each set of bars refers to the anti-LINGO-1 group.

FIG. 13 is a series of heatmaps depicting the mean change in MF-VEP amplitude (nV) in the affected eye from baseline of the affected eye during treatment with anti-LINGO-1 antibody over 32 weeks.

FIG. 14 is a series of heatmaps depicting the mean change in MF-VEP amplitude (nV) in the unaffected eye from baseline of the unaffected eye during treatment with anti-LINGO-1 antibody over 32 weeks.

FIGS. 15A, 15B, and 15C are graphs showing the change from baseline in mean (A) NEI VFQ-25 composite; (B)NOS-10; and (C) combined NEI VFQ-25 and NOS-10 composite scores to week 24 as analyzed by MMRM in the RENEW trial.

FIG. 16 is a bar graph showing the percentage of patients with change in NEI VFQ-25 composite score who declined by ≥4 points, sustained ≤4 points, or improved ≥4 points from baseline at week 24 in the RENEW trial. The left bar of each set of bars refers to the placebo group, and the right bar of each set of bars refers to the anti-LINGO-1 group.

FIG. 17 is a schematic showing a clinical trial design (SYNERGY).

FIG. 18 is a schematic showing the elements making up the endpoints of the SYNERGY clinical trial.

FIG. 19 is a table showing rates of improvement of subjects in the SYNERGY clinical trial as assessed by the primary endpoint after treatment with anti-LINGO-1 according to the clinical trial design shown in FIG. 17.

FIG. 20 is a graph showing the odds ratios for each of the treatment groups (3 mg/kg, 10 mg/kg, 30 mg/kg, and 100 mg/kg anti-LINGO-1) relative to placebo.

FIG. 21A-FIG. 21D are graphs showing overall response score over 72 weeks by mixed model for repeated measures (MMRM). The 3 mg/kg treatment group is shown in FIG. 21A. The 10 mg/kg treatment group is shown in FIG. 21B. The 30 mg/kg treatment group is shown in FIG. 21C. The 100 mg/kg treatment group is shown in FIG. 21D.

FIG. 22 is a series of graphs showing the odds ratios for each of the treatment groups (3 mg/kg, 10 mg/kg, 30 mg/kg, and 100 mg/kg anti-LINGO-1) relative to placebo, with the data stratified according to age, disease subtype, and disease duration.

FIG. 23 is a series of graphs showing the odds ratios for each of the treatment groups (3 mg/kg, 10 mg/kg, 30 mg/kg, and 100 mg/kg anti-LINGO-1) relative to placebo, with the data stratified according to whole brain DTI (RD).

FIG. 24 is a table showing rates of improvement of subjects in the SYNERGY clinical trial as assessed by the Functional Disability Endpoint (FDE) with anti-LINGO-1 according to the clinical trial design shown in in FIG. 17.

FIG. 25 is a table showing rates of improvement of RRMS subjects in the SYNERGY clinical trial as assessed by the Functional Disability Endpoint (FDE) with anti-LINGO-1 according to the clinical trial design shown in in FIG. 17.

FIG. 26 is a graph showing the mean overall responsiveness score according to the cumulative area under the curve (AUC) for exposure to anti-LINGO-1 for subjects in the SYNERGY study. Each vertical line represents a level of exposure to anti-LINGO-1 with the first line to the left being the placebo.

FIG. 27 is a table characterizing the data in bin 3. Shown are the number of subjects (Subjects (int)), the number of datapoints (n(int)), the dosage (DOSE(db1)), the number of doses (N_DOSE(dbl), and whether the subset is RRMS or SPMS (MSTYPE(chr)).

FIGS. 28A and 28B are graphs showing the mean overall responsiveness score according to the cumulative area under the curve (AUC) for exposure to anti-LINGO-1 for subjects in the SYNERGY study with RRMS (FIG. 28A) and with SPMS (FIG. 28B).

FIGS. 29A and 29B are graphs showing the mean overall responsiveness score according to the cumulative area under the curve (AUC) for exposure to anti-LINGO-1 for subjects in the SYNERGY study who are under 40 years of age (FIG. 29A) or who are age 40 years or older (FIG. 29B).

FIGS. 30A and 30B are graphs showing the mean change of 9HPT according to the cumulative area under the curve (AUC) for exposure to anti-LINGO-1 for subjects in the SYNERGY study in the dominant hand (FIG. 30A) or non-dominant hand (FIG. 30B).

FIG. 31 is a graph showing the mean change of T25FW according to the cumulative area under the curve (AUC) for exposure to anti-LINGO-1 for subjects in the SYNERGY study.

FIGS. 32A and 32B shows serum concentration (FIG. 32A) and AUC (FIG. 32B) of anti-LINGO-1 as a function of dosage at steady state.

FIGS. 33A and 33B are graphs showing change from baseline over 48 weeks by treatment arm in the intent to treat population for MTR (nMTRu) (FIG. 33A) and DTI-RD (10⁻³ mm²/s) (FIG. 33B) in pre-existing non-enhancing T2 lesions using mixed-effect model repeated measure.

FIG. 34A-34C are graphs showing the net change in brain volume from baseline at 48 weeks and 72 weeks in the whole brain (FIG. 34A), cerebral cortical brain (FIG. 34B) and thalamic brain (FIG. 34C).

FIG. 35 is a graph showing the odds ratio for likelihood of Gd+ lesion conversion to chronic black holes in treatment groups versus placebo at 0-48 weeks and at 49-72 weeks.

FIGS. 36A and 36B are graphs showing the treatment effect (anti-LINGO-1 by dose) vs. placebo for change in DTI-RD (10⁻³ mm²/s) (FIG. 36A) and DTI-FA (FIG. 36B) in pre-existing non-enhancing T2 lesions in the half of the SYNERGY population whose baseline whole brain DTI-RD was <0.732×10⁻³ mm²/s.

FIG. 37 is a graph showing the treatment effect (anti-LINGO-1 by dose) vs. placebo for change in DTI-RD (10⁻³ mm²/s) in pre-existing non-enhancing T2 lesions subjects with a disease duration of <8 years.

FIGS. 38A and 38B are graphs showing change from baseline using mixed-effect model repeated measure for MTR (nMTRu) (FIG. 38A) and DTI-RD (10⁻³ mm²/s) (FIG. 38B) in pre-existing non-enhancing T2 lesions by clinical improvement responders (without worsening) vs. clinical non-responders (worsening without improvement), based on the SYNERGY primary and secondary endpoints.

### DETAILED DESCRIPTION OF THE INVENTION

Inflammatory demyelinating CNS diseases, such as MS, are a common cause of non-traumatic neurological disability in young adults. Currently approved therapies for MS are primarily immunomodulatory, and do not have detectable direct effects on CNS repair. For example, the current standard of care for patients with relapsing MS includes the use of immunomodulatory drugs to reduce the frequency and severity of relapses and the accumulation of relapse-related physical disability, and to provide various symptomatic treatment as needed such as for depression, bladder dysfunction, or walking impairment. Several immunomodulatory drugs are currently available for relapsing MS, including, but not limited to, different preparations of interferon β (interferon β-1a given intramuscularly [IM] [Avonex] or subcutaneously [SC] [Rebif®], interferon β-1b [Betaseron/Betaferon®/Extavia®]), glatiramer acetate (Copaxone®), natalizumab (Tysabri®), and fingolimod (Gilenya®). Short courses of corticosteroids are occasionally given with mixed success. Chemotherapeutic agents, such as mitoxantrone and cyclophosphamide, are occasionally used in cases of severe relapsing MS. Although some degree of axonal remyelination by oligodendrocytes takes place early during the course of MS, the ability to endogenously repair the CNS often fails, leading to irreversible tissue injury and an increase in disease-related disability.

Several preclinical studies have demonstrated a role for LINGO-1 antagonism in enhancing CNS remyelination and neuroaxonal protection in animal models of toxic injury (Cuprizone) (Mi et al. (2009) Ann Neurology, 65: 304-15), chemical injury (lysophosphatidylcholine [LPC]), and inflammatory demyelination (myelin oligodendrocyte glycoprotein-experimental autoimmune encephalomyelitis [MOG-EAE]) [Mi et al. (2007) Nat Med, 13: 1228-33); and of toxic (1-methyl-4-phenyl-1,2,3,6-tetrahydropyridine [MPTP]) neuronal injury (Inoue et al. (2007) Proc Natl Acad Sci, 104: 14430-5), traumatic/hypertensive optic nerve injury (Fu et al. (2008) Invest Opthalmol Vis Sci, 49: 975-85) and spinal cord injury (Ji et al. (2006) Mol Cell Neurosci, 33: 311-20; Ji et al. (2008) Mol Cell Neurosci, 39: 258-67; Lv et al. (2010) Neuroimmunomodulat, 17: 270-8). Thus, antagonizing LINGO-1 with an anti-LINGO-1 antibody can enhance remyelination and neuroaxonal protection in the CNS. An anti-LINGO-1 antibody can reach the CNS in sufficient concentrations to block LINGO-1 in both axons and oligodendroyctes after peripheral administration. This in turn, can enhance remyelination via differentiation of oligodendrocyte precursor cells (OPC) normally present in the brain of MS patients.

Binding of an anti-LINGO-1 antibody to LINGO-1 in axons and neurons can also provide neuroaxonal protection via blockade of signaling by myelin debris on the Nogo66 receptor-1(NgR1)/p75/LINGO-1 receptor complex in the CNS. It has been proposed that the failure of axonal repair/neurite regeneration in MS can be due, at least in part, to signaling of myelin debris on the NgR1/p75/LINGO-1 complex and the NgR1/TROY/LINGO-1 complex in damaged axons (Mi et al. (2004) Nat Neurosci, 7: 221-8). Signaling on the NgR1 receptor complex may interfere not only with axonal regeneration (Yamashita et al. (2005) Mol Neurobiol, 32: 105-11), but also with neuronal survival following neuroaxonal injury (Mi et al. (2004) Nat Neurosci, 7: 221-8; Fu et al. (2008) Invest Opthalmol Vis Sci, 49: 975-85; Zhao et al. (2008) Cell Mol Neurobiol, 28: 727-35).

Without wishing to be bound by theory, it is believed that newly developed lesions may be easier to repair and remyelinate due, at least in part, to the greater preservation of axons and lesser interference from glial scar (Jasmin and Ohara (2002) Neuroscientists 8(3):198-203; Vick et al. (1992) J. Neurotrauma 9 Suppl 1:S93-103). However, reparative effects of LINGO-1 antagonists on pre-existing lesions can also occur. For example, the efficacy of an anti-LINGO-1 antibody treatment in pre-existing lesions is supported by (1) the finding that OPCs are found in chronically demyelinated MS lesions, (2) animal studies that show the ability of chronically demyelinated brain lesions to be remyelinated, and (3) studies showing the enhancement of remyelination by LINGO-1 blockade in established demyelinated lesions (e.g., in the Cuprizone model).

Thus, antagonism of LINGO-1 with an anti-LINGO-1 antibody can enhance remyelination and neuroaxonal protection (thus, preventing axonal degeneration) in CNS demyelinating diseases, such as MS and acute optic neuritis, leading to improved CNS repair with corresponding beneficial effects on neurological function and disability. In certain embodiments, the reparative agent (e.g., a LINGO-1 antagonist) can be used to treat multiple sclerosis (MS), e.g., a progressive form of MS.

Accordingly, methods and compositions for treating or preventing CNS disorders, e.g., CNS demyelinating disorders, using a reparative agent (e.g., a LINGO-1 antagonist, e.g., an anti-LINGO-1 antibody molecule) are disclosed. In certain embodiments, the methods and compositions described herein include a reparative agent (e.g., a LINGO-1 antagonist) as a monotherapy, or in combination with an immunomodulatory agent. Since an anti-LINGO-1 antibody does not have detectable immunomodulatory effects on the inflammatory component of MS pathogenesis, concurrent administration with an immunomodulatory agent is desirable. Therefore, combination treatments of an immunomodulatory agent, e.g., IFN-β agent, e.g., Avonex®; with a reparative agent, e.g., anti-LINGO-1 antibody, are disclosed at least in part.

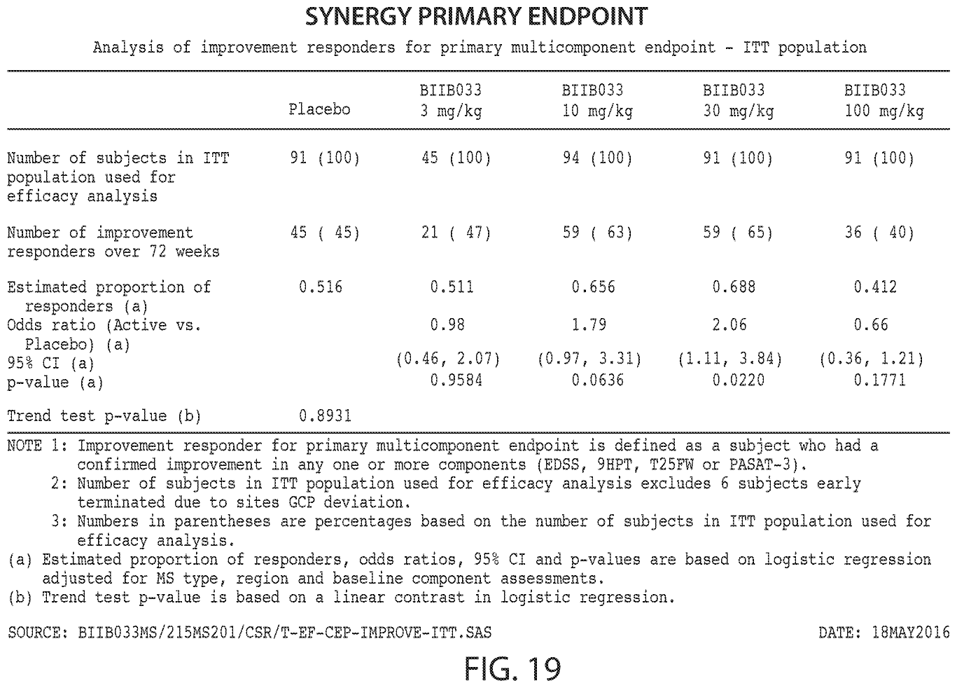

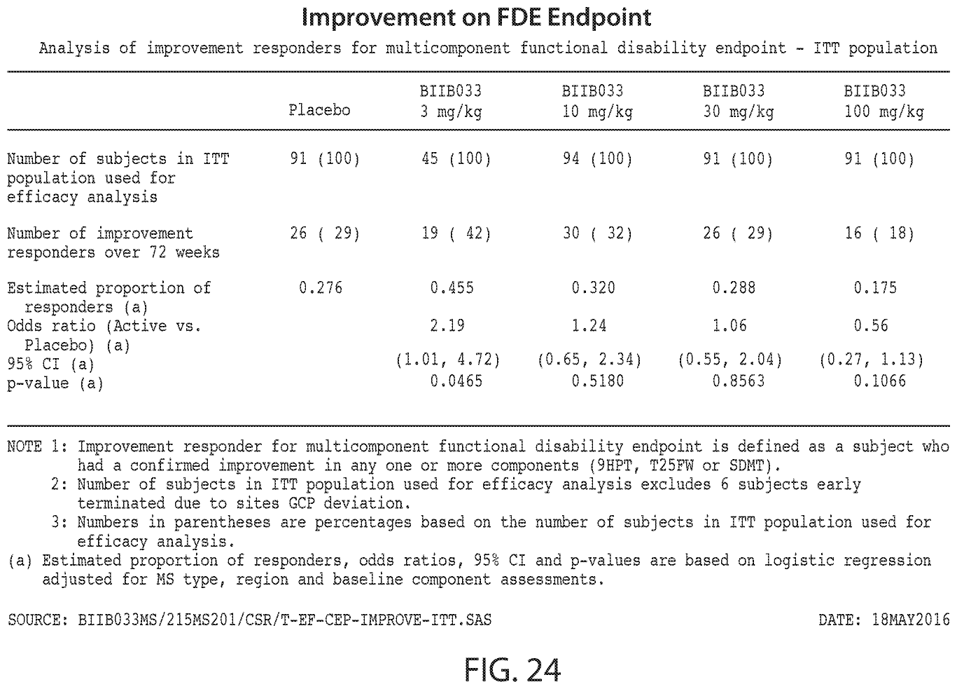

In one embodiment, Applicants have identified an unexpected dose response relationship after administration of increasing doses of an anti-LINGO-1 antibody molecule, in the presence of an immunomodulatory agent, e.g., an IFN-β 1 molecule, to a subject with MS, e.g., a subject with a relapsing form of MS (e.g., RRMS or SPMS). For example, the dose response identified showed a non-monotonic or biphasic dose-effect relationship, showing an increased improvement response at lower cumulative drug exposure, followed by less improvement at higher cumulative drug levels, e.g., as shown in FIG. 19, and FIGS. 24-31.

In some embodiments, MS subjects with less brain damage, e.g., less advanced forms of MS, showed a more pronounced improvement after treatment compared to subjects with more brain damage, e.g., more advanced form of MS, e.g., as shown in Table 35. Thus, disclosed herein are dosage regimens that result in a selected total cumulative exposure to an anti-LINGO-1 antibody therapy; said dosage regimens result in improved therapeutic outcomes in MS subjects. Said dosage regimens can be tailored according to several parameters, including disease status, improvement response, subject's age, and diagnostic test used to evaluate treatment outcome. Accordingly, improved dosage regimens for treatment of CNS demyelinating diseases, such as MS, are disclosed. Thus, methods, compositions and kits described herein can be useful for treating a CNS disorder, e.g., a CNS demyelinating disease.

In other embodiments, the reparative agent (e.g., a LINGO-1 antagonist) can be used to treat an inflammatory condition of the optic nerve, e.g., optic neuritis (e.g., acute optic neuritis (AON). Thus methods and compositions comprising a reparative agent for treating an inflammatory condition of the optic nerve, e.g., optic neuritis (e.g., AON) are also disclosed.

The term “reparative agent” as used herein includes any agent that causes one or more of: enhances myelination, re-myelination, enhances neuroaxonal protection, increases axonal extension, increases neuronal sprouting, and/or promotes oligodendrocyte numbers (e.g., by increasing one or more of: survival or differentiation of oligodendrocytes), without having a substantial (e.g., a detectable) immunomodulatory effect. In one embodiment, the reparative agent is a LINGO-1 antagonist, e.g., a LINGO-1 antagonist as described herein. In one embodiment, the reparative agent is an anti-LINGO-1 antibody molecule described herein, e.g., opicinumab (also referred to herein as “BIIB033”).

Various aspects of the invention are described in further detail in the following subsections.

### Definitions

As used herein, each of the following terms has the meaning associated with it in this section.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of, determining, or evaluating, a desired result, e.g., a value, e.g., a numerical value, by “directly acquiring” or “indirectly acquiring” the result. “Directly acquiring” means performing a process (e.g., performing a test, e.g., a measure of upper and/or lower extremity function, and/or ambulatory function) to obtain the result, e.g., the value. “Indirectly acquiring” refers to receiving the result, e.g., the value, from another party or source (e.g., a third party clinician or health professional that directly acquired the value).

“Area under the curve” or “AUC” as the terms are used herein, refer to area under the drug concentration-time curve. Typically, the pharmacokinetic plot is a plot of concentration of drug in serum against time. The area under this concentration-time curve is AUC. A “cumulative AUC” refers to the AUC value of total drug exposure for all dosage regimens received at a preselected time.

A “CNS disorder” (e.g., a “CNS demyelinating disease”) can be any disease, disorder or injury associated with one or more of: demyelination, dysmyelination, axonal injury, and/or dysfunction or death of an oligodendrocyte or a neuronal cell, or loss of neuronal synapsis/connectivity. In certain embodiments, the CNS disorder affects the nervous system by causing damage to the myelin sheath of axons. In other embodiments, the CNS disorder includes Nogo receptor-1 (NgR1-) mediated inhibition of axonal extension or neurite extension, e.g., in the brain and spinal cord. In other embodiments, the CNS disorder has one or more inflammatory components. In one embodiment, the CNS disorder (e.g., the CNS demyelinating disease) is multiple sclerosis. In one embodiment, the CNS disorder (e.g., the CNS demyelinating disease) is an optic nerve condition or disorder, e.g., optic neuritis, e.g., acute optic neuritis.

The CNS disorder (e.g., the CNS demyelinating disease) is “treated,” “inhibited” or “reduced,” if at least one symptom of the disease or disorder is reduced, alleviated, terminated, slowed, or prevented. Treatment or prevention need not be 100%, and in some embodiments a reduction or delay in at least one symptom of the disease or disorder by at least 50%, 60%, 70%, 80%, 90%, 95%, or 99% is sufficient to be considered within these terms.

In embodiments, the CNS disorder is “prevented” if at least one symptom of the disease or disorder is delayed, e.g., by about 4 weeks, 8 weeks, 12 weeks, 24 weeks, 36 weeks, 48 weeks, 1 year, 2 years, 3 years, 4 years, 5 years, 6 years, 7 years, 8 years, 9 years, 10 years, or more. In embodiments, the CNS disorder is prevented if initial onset (e.g., first occurrence of a symptom) of the disorder is delayed, e.g., by about 4 weeks, 8 weeks, 12 weeks, 24 weeks, 36 weeks, 48 weeks, 1 year, 2 years, 3 years, 4 years, 5 years, 6 years, 7 years, 8 years, 9 years, 10 years, or more.

As used herein, in some embodiments, an optic nerve condition or disorder, e.g., optic neuritis, e.g., acute optic neuritis, is “prevented” if at least one symptom of the optic nerve condition or disorder is delayed in one or both eyes, e.g., by about 4 weeks, 8 weeks, 12 weeks, 24 weeks, 36 weeks, 48 weeks, 1 year, 2 years, 3 years, 4 years, 5 years, 6 years, 7 years, 8 years, 9 years, 10 years, or more. In embodiments, an optic nerve condition or disorder, e.g., optic neuritis, e.g., acute optic neuritis, is “prevented” if initial onset (e.g., first occurrence of a symptom) of the optic nerve condition or disorder is delayed in one or both eyes, e.g., by about 4 weeks, 8 weeks, 12 weeks, 24 weeks, 36 weeks, 48 weeks, 1 year, 2 years, 3 years, 4 years, 5 years, 6 years, 7 years, 8 years, 9 years, 10 years, or more, i.e., if function is preserved for a period of time. In an example, optic neuritis is prevented in a normal fellow eye that does not show symptoms of optic neuritis if at least one symptom of the optic neuritis is delayed in the normal fellow eye, e.g., by about 4 weeks, 8 weeks, 12 weeks, 24 weeks, 36 weeks, 48 weeks, 1 year, 2 years, 3 years, 4 years, 5 years, 6 years, 7 years, 8 years, 9 years, 10 years, or more. In an example, optic neuritis is prevented in a normal fellow eye that does not show symptoms of optic neuritis if initial onset (e.g., first occurrence of a symptom) of the optic neuritis is delayed in the normal fellow eye, e.g., by about 4 weeks, 8 weeks, 12 weeks, 24 weeks, 36 weeks, 48 weeks, 1 year, 2 years, 3 years, 4 years, 5 years, 6 years, 7 years, 8 years, 9 years, 10 years, or more.

As used herein, an optic nerve condition or disorder, e.g., optic neuritis, e.g., acute optic neuritis, is “treated,” “inhibited,” or “reduced,” if recurrence or relapse of the disease is reduced, retarded, slowed, delayed, or prevented. Exemplary clinical symptoms of acute optic neuritis that can be used to aid in determining disease status in a subject can include, e.g., visual loss, edema, inflammation, damage or demyelination of the myelin sheath covering the optic nerve and axons, loss of retinal fiber layer, loss of retinal ganglion cell layer, visual field defect, color desaturation, decreased color vision, ocular pain, decreased visual acuity, Uhthoff's symptom, swollen optic disc, or relative afferent papillary defect. In embodiments, clinical outcomes can be used to aid in determining disease status in a subject, e.g., optic nerve damage (e.g., as measured by full field visual evoked potential amplitude or multi-focal visual evoked potential), optic nerve latency (e.g., as measured by full field visual evoked potential or multi-focal visual evoked potential), thickness of retinal layers such as retinal nerve fiber layer or retinal ganglion cell layer (e.g., as measured by spectral domain optical coherence tomography), visual function (e.g., as measured by visual acuity, e.g., low contrast or high contrast letter acuity), or visual quality of life (e.g., as measured by a patient reported outcome test, e.g., a NIH-NEI visual functional questionnaire or a neuro-ophthalmic supplement, NOS-10).

As used herein, “normal” eye (e.g., a “normal fellow eye”) is an eye in a subject that does not show one or more symptoms of an optic nerve condition or disorder, e.g., optic neuritis, e.g., acute optic neuritis.

As used herein, multiple sclerosis is “treated,” “inhibited,” or “reduced,” if recurrence or relapse of the disease is reduced, slowed, delayed, or prevented. Exemplary clinical symptoms of multiple sclerosis that can be used to aid in determining the disease status in a subject can include e.g., tingling, numbness, muscle weakness, loss of balance, blurred or double vision, slurred speech, sudden onset paralysis, lack of coordination, cognitive difficulties, fatigue, heat sensitivity, spasticity, dizziness, tremors, gait abnormalities, speech/swallowing difficulties, and extent of lesions assessed by imaging techniques, e.g., MRI. Treatment can include, but is not limited to, inhibiting or reducing one or more symptoms such as numbness, tingling, muscle weakness and/or other symptoms described herein for optic neuritis; reducing relapse rate or severity, reducing size or number of sclerotic lesions; inhibiting or retarding the development of new lesions; prolonging survival, or prolonging progression-free survival, and/or enhanced quality of life. Clinical signs of MS are routinely classified and standardized, e.g., using an EDSS rating system based on neurological examination and long distance ambulation. For the lower end of the scale (1-5.5) a decrease of one full step indicates an effective MS treatment (Kurtzke, Ann. Neurol. 36:573-79, 1994), while an increase of one full step will indicate the progression or worsening of the disease (e.g., exacerbation). For the higher end of the scale (5-7), a half a point typically indicates improvement (a reduction) or worsening (an increase).

A dosage regimen, e.g., a therapeutic dosage regimen, can include one or more treatment cycles. The dosage regimen can result in at least one beneficial or desired clinical result including, but not limited to, alleviation of a symptom, diminishment of extent of disease, reduction of pre-existing disability, improvement of pre-existing neurological function impairment, stabilized (i.e., not worsening) state of disease, delay or slowing of disease progression, amelioration or palliation of the disease state, whether detectable or undetectable. In embodiments, a dosage regimen includes intermittent administration of one or more doses of a reparative agent in combination with, a second therapeutic agent, e.g., one or more doses of immunomodulatory agent as described herein. In embodiments, a dosage regimen can have one or more periods of no administration of the therapeutic agent, e.g., drug holidays, in between treatment cycles.

As used herein, a “treatment cycle” refers to a course of administration of a therapeutic agent that can be repeated, e.g., on a regular schedule. For example, a treatment cycles can include one or more doses of a reparative agent administered, alone or in combination with (prior, concurrently or after), administration of a second therapeutic agent, e.g., one or more doses of immunomodulatory agent as described herein.

As used herein, a “drug holiday” refers to an interruption in a treatment, e.g., a treatment cycle of a reparative agent and/or immunomodulatory agent as described herein. in embodiments, the subject, e.g., an MS patient, stops taking a medication(s), e.g., a reparative agent as described herein, for a period of time. In some embodiments, the medication(s) is discontinued for one or more days, to months or years. As used herein, the “Expanded Disability Status Scale” or “EDSS” is intended to have its customary meaning in the medical practice. EDSS is a rating system that is frequently used for classifying and standardizing MS. The accepted scores range from 0 (normal) to 10 (death due to MS). Typically patients having an EDSS score of about 4-6 will have moderate disability (e.g., limited ability to walk), whereas patients having an EDSS score of about 7 or 8 will have severe disability (e.g., will require a wheelchair). More specifically, EDSS scores in the range of 1-3 refer to an MS patient who is fully ambulatory, but has some signs in one or more functional systems; EDSS scores in the range higher than 3 to 4.5 show moderate to relatively severe disability; an EDSS score of 5 to 5.5 refers to a disability impairing or precluding full daily activities; EDSS scores of 6 to 6.5 refer to an MS patient requiring intermittent to constant, or unilateral to bilateral constant assistance (cane, crutch or brace) to walk; EDSS scores of 7 to 7.5 means that the MS patient is unable to walk beyond five meters even with aid, and is essentially restricted to a wheelchair; EDSS scores of 8 to 8.5 refer to patients that are restricted to bed; and EDSS scores of 9 to 10 mean that the MS patient is confined to bed, and progressively is unable to communicate effectively or eat and swallow, until death due to MS.

As used herein, a “disease progression” includes a measure (e.g., one or more measures) of a worsening of one or more symptoms and/or disability in a subject. In certain embodiments, disease progression is evaluated as a steady worsening of one or more symptoms and/or disability over time, as opposed to a relapse, which is relatively short in duration. In certain embodiments, the disease progression is evaluated in a subject with a relapsing form of MS (e.g., RRMS) or a progressive form of MS (e.g., a subject with primary or secondary progressive multiple sclerosis (PPMS or SPMS, respectively), or a subject with progressive-relapsing MS (PRMS)).

In certain embodiments, the disease progression is evaluated in a subject with an optic nerve condition or disorder, e.g., optic neuritis, e.g., acute optic neuritis, e.g., in one or both eyes. In some embodiments, the evaluation of disease progression includes a measure of a clinical symptom or outcome of acute optic neuritis described herein.

In other embodiments, the evaluation of disease progression includes a measure of upper extremity function (e.g., a 9HPT assessment). Alternatively or in combination, disease progression includes a measure of lower extremity function. Alternatively or in combination, disease progression includes a measure of ambulatory function, e.g., short distance ambulatory function (e.g., T25FW). Alternatively or in combination, disease progression includes a measure of ambulatory function, e.g., longer distance ambulatory function (e.g., a 6-minute walk test). In one embodiment, the disease progression includes a measure of ambulatory function other than EDSS ambulatory function. In one embodiment, disease progression includes a measure of upper extremity function (e.g., a 9HPT assessment) and a measure of ambulatory function, e.g., short distance ambulatory function (e.g., T25FW). In one embodiment, disease progression includes a measure of upper extremity function (e.g., a 9HPT assessment) and a measure of lower extremity function. In one embodiment, disease progression includes a measure of upper extremity function (e.g., a 9HPT assessment), a measure of lower extremity function, and a measure of ambulatory function, e.g., short distance ambulatory function (e.g., T25FW) and/or longer distance ambulatory function (e.g., a timed (e.g., 6-minute) walk test (e.g., 6MWT)). In one embodiment, one, two or the combination of the T25FW, 6MWT and 9HPT assessments can be used to acquire a disease progression value. The measure of ambulatory function (e.g., short distance ambulatory function (e.g., T25FW) or longer distance ambulatory function (e.g., a timed (e.g., 6-minute) walk test (e.g., 6MWT)) and/or measure of upper extremity function (e.g., a SHPT assessment) can further be used in combination with the EDSS to evaluate MS, e.g., progressive forms of MS.

In one embodiment, a progressor is a subject who possesses a disease progression value reflecting at least one, two or all of the following criteria:

a. confirmed progression in T25FW: Time taken for 25-foot walk increased by at least 15% or 20% of the baseline walk, confirmed at a second time point at least 3, 4, 5, or 6 months apart;

b. confirmed progression in a timed (e.g., 6-minute) walk test (e.g., 6MWT): Time taken for walk increased by at least 10%, 15% or 20% of the baseline walk, confirmed at a second time point at least 3, 4, 5, or 6 months apart;

c. confirmed progression in 9HP: Time taken for 9-hole peg increased by at least 15% or 20% of the time taken at baseline, confirmed at a second time point at least 3, 4, 5, or 6 months apart. The progression in 9HP can occur on either hand, but will have to be confirmed on the same hand; and/or

d. confirmed progression in EDSS:

(i) EDSS total score increase from baseline by at least 1 point, if the change in EDSS total score is determined (or primarily determined) by evaluating a change in neurological function (e.g., one or more changes in neurological systems); and/or

(ii) EDSS total score increased from baseline by at least 0.5 point if the change in EDSS total score is determined (or primarily determined) by a change in ambulatory function, if either or both of (i) or (ii) is/are confirmed on a second examination at least 3, 4, 5 or 6 months apart (typically, at least 6 months apart).

Baseline values for the aforementioned tests (e.g., T25FW, 6MWT, EDSS, or 9HPT) can be determined using the best baseline value or the average baseline value.

“Baseline,” as used herein, refers to a value or measurement prior to administration of a therapy, e.g., a therapy described herein. In embodiments, “baseline” with respect to a “fellow eye” refers to a value or measurement of the eye of a subject other than the affected eye (e.g., affected by an optic nerve condition or disorder, e.g., optic neuritis), prior to administration of a therapy, e.g., a therapy described herein.

“Responsiveness,” to “respond” to a treatment, and other forms of this term, as used herein, refer to the reaction of a subject to treatment with a therapy as described. As an example, an MS subject responds to therapy if at least one symptom of multiple sclerosis (e.g., disease worsening) in the subject is reduced or retarded by about 10%, 20%, 30%, 40%, 50%, 60%, 70%, 80%, 90% or more. In another example, an MS subject responds to a therapy, if at least one symptom of multiple sclerosis in the subject is reduced by about 5%, 10%, 20%, 30%, 40%, 50% or more as determined by any appropriate measure, e.g., one or more of: a measure of upper or lower extremity function, a measure of ambulatory function, or an assessment of Expanded Disability Status Scale (EDSS). In another example, an MS subject responds to treatment with a therapy, if the subject has an increased time to progression. Several methods can be used to determine if a patient responds to a treatment including the assessments described herein, as set forth herein.

In certain embodiments, an improvement in the subject is defined by one or more of:

a. ≥1.0 point decrease in EDSS from a baseline score of ≤6.0;

b. ≥15% improvement from baseline in T25FW;

c. ≥15% improvement from baseline in 9HPT; or

d. ≥10% (e.g., 10%, 12%, 20%, 30%) improvement from baseline in PASAT or SDMT.

As an example, a subject with optic neuritis (e.g., acute optic neuritis) responds to treatment with a therapy if at least one symptom of optic neuritis in the subject is reduced or retarded by about 10%, 20%, 30%, 40%, 50%, 60%, 70%, 80%, 90% or more. In an example, a subject with acute optic neuritis responds to a therapy if at least one symptom of acute optic neuritis in the subject is reduced by about 5%, 10%, 20%, 30%, 40%, 50% or more as determined by any appropriate measure, e.g., one or more of: a measure of optic nerve damage, a measure of optic nerve latency, a measure of thickness of retinal layer, a measure of visual function, or a measure of visual quality of life.

A “non-responder” or “progressor” refers to a subject, e.g., an MS patient or optic neuritis (e.g., acute optic neuritis) patient, if in response to a therapy (e.g., a therapy described herein), at least one symptom or disability of e.g., multiple sclerosis or optic neuritis (e.g., acute optic neuritis), in the subject is reduced by less than about 5%, as determined by any appropriate measure, e.g., one or more of: a measure of upper or lower extremity function, a measure of ambulatory function, a measure of cognitive function, an assessment of Expanded Disability Status Scale (EDSS), a measure of optic nerve damage, a measure of optic nerve latency, a measure of thickness of retinal layer, a measure of visual function, or a measure of visual quality of life.

The methods, compositions and kits disclosed herein encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a sequence described herein are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%. 91%, 92%, 93%, 94%, 95%, 96%, 97%, 98% or 99% identity to a sequence described herein are termed substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to BMP-10/BMP-10 receptor nucleic acid (SEQ ID NO:1) molecules of the invention. BLAST protein searches can be performed with the XBLAST program, score=50, wordlength=3 to obtain amino acid sequences homologous to BMP-10/BMP-10 receptor (SEQ ID NO:1) protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Also included are fragments, derivatives, analogs, or variants of the polypeptides, and any combination thereof. The terms “fragment,” “variant,” “derivative” and “analog” include any polypeptides which retain at least some of the properties of the corresponding native polypeptide. Fragments of polypeptides include proteolytic fragments, as well as deletion fragments. Variants of polypeptides include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Derivatives of polypeptides are polypeptides which have been altered so as to exhibit additional features not found on the native polypeptide. Examples include fusion proteins.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Various aspects of the invention are described in further detail below. Additional definitions are set out throughout the specification.

### Reparative Agents

Methods, composition and kits described herein include a combination of a reparative agent (e.g., a LINGO-1 antagonist) and an immunomodulatory agent. In one embodiment, the reparative agent is an antagonist of LRR and Ig domain-containing, Nogo receptor-interacting protein (“LINGO,” e.g., LINGO-1). For example, the LINGO-1 antagonist can inhibit or reduce the expression or activity of LINGO-1, e.g., human LINGO-1. In one embodiment, the LINGO-1 antagonist inhibits or reduces the formation and/or activity of a complex (e.g., a functional signaling complex) of the NgR1, p′75, and LINGO-1; and/or NgR1, TAJ (TROY), and LINGO-1. In another embodiment, the LINGO-1 antagonist inhibits or reduces LINGO-1 binding to NgR1.

### LINGO-1 and LINGO-1 Antagonists

LINGO-1, previously called Sp35, is a cell surface glycoprotein that is selectively expressed in the adult CNS in neurons and oligodendrocytes. LINGO-1 is a member of a protein family comprising 3 other paralogs: LINGO-2 (GI: 12309630, 61% protein identity), LINGO-3 (GI: 23342615, 56% identity) and LINGO-4 (GI: 21211752, 44% identity). LINGO-1 is highly conserved evolutionarily with human and mouse orthologues sharing 99.5% identity. By Northern blot analysis, LINGO-1 was found to be highly expressed in human brain and was not detectable in non-neural tissues (Barrette et al. (2007) Mol Cell Neurosci, 34: 519-38; Carim-Todd et al. (2003) Eur Journal Neurosci, 18: 3167-82; Llorens et al. (2008) Dev Neurobiol, 68: 521-41; Mi et al. (2004) Nat Neurosci, 7: 221-8; Okafuji et al. (2005) Gene Expr Patterns, 6: 57-62; Park et al. (2006) Neurosci Lett, 404: 61-6; Shao et al. (2005) Neuron, 45: 353-9). LINGO-1 has also been described in detail in International Applications PCT/US2006/026271, filed Jul. 7, 2006, PCT/US2004/008323, filed Mar. 17, 2004, PCT/US2005/022881, filed Jun. 24, 2005 and PCT/US2008/000316, filed Jan. 9, 2008, each of which is incorporated by reference in its entirety herein.

LINGO-1 is selectively expressed in both oligodendrocyte precursor cells (OPCs) and neurons. LINGO-1 functions as a negative regulator of oligodendrocyte differentiation myelination, and remyelination; preventing myelination of axons by oligodendrocytes (Lee et al. (2007) J Neurosci, 27: 220-5; Mi et al. (2005) Nat Neurosci, 8: 745-51; Mi et al. (2008) Int Journal Biochem Cell Biol 40(10):1971-8; Mi et al. (2009) Ann Neurology, 65: 304-15). Axonal and neuronal expression of LINGO-1 increases after injury (Ji et al. (2006) Mol Cell Neurosci, 33: 311-20). LINGO-1 expression prevents myelination of axons by oligodendrocytes. Several preclinical studies have demonstrated the potential for LINGO-1 antagonism to enhance CNS remyelination and neuroaxonal protection in animal models of toxic (Cuprizone) (Mi et al. (2009) Ann Neurology, 65: 304-15), chemical injury (lysophosphatidylcholine [LPC]), and inflammatory (myelin oligodendrocyte glycoprotein-experimental autoimmune encephalomyelitis [MOG-EAE]) [Mi et al. (2007) Nat Med, 13: 1228-33) demyelination; and of toxic (1-methyl-4-phenyl-1,2,3,6-tetrahydropyridine [MPTP]) neuronal injury (Inoue et al. (2007) Proc Natl Acad Sci, 104: 14430-5), traumatic/hypertensive optic nerve injury (Fu et al. (2008) Invest Opthalmol Vis Sci, 49: 975-85) and spinal cord injury (Ji et al. (2006) Mol Cell Neurosci, 33: 311-20; Ji et al. (2008) Mol Cell Neurosci, 39: 258-67; Lv et al. (2010) Neuroimmunomodulat, 17: 270-8). Remyelination and neuroaxonal protection can be provided via blockade of signaling by myelin debris and/or sulfated proteoglycans on the NgR1 receptor complex in the CNS caused by the inhibition of LINGO-1 in axons and oligodendroyctes. This in turn may enhance remyelination via differentiation of oligodendrocyte precursor cells (OPCs) normally present in the brain of MS patients. Thus, antagonism of LINGO-1 can enhance myelination or re-myelination of axons, e.g., by oligodendrocytes, and enhance neuroaxonal protection in the CNS, and for example, in CNS demyelinating diseases such as multiple sclerosis (MS) and acute optic neuritis, leading to improved CNS repair.

LINGO-1 is also known in the art by the names LRRN6, LRRN6A, FLJ14594, LERN1, MGC17422 and UNQ201. The human, full-length wild-type LINGO-1 polypeptide contains an LRR domain consisting of 14 leucine-rich repeats (including N- and C-terminal caps), an Ig domain, a transmembrane region, and a cytoplasmic domain. The cytoplasmic domain contains a canonical tyrosine phosphorylation site. In addition, the naturally occurring LINGO-1 protein contains a signal sequence, a short basic region between the LRR-C-terminal domain (LRRCT) and Ig domain, and a transmembrane region between the Ig domain and the cytoplasmic domain. The human LINGO-1 gene (SEQ ID NO:52) contains alternative translation start codons, so that six additional amino acids, i.e., MQVSKR (SEQ ID NO:87) may or may not be present at the N-terminus of the LINGO-1 signal sequence. Table 2 lists the LINGO-1 domains and other regions, according to amino acid residue number, based on the LINGO-1 amino acid sequence presented herein as SEQ ID NO: 51. The LINGO-1 polypeptide is characterized in more detail in PCT Publication No. WO 2004/085648, which is incorporated herein by reference in its entirety.

TABLE 2
LINGO-1 Domains

| Domain or Region | Beginning Residue | Ending Residue |
|---|---|---|
| Signal Sequence | 1 | 33 or 35 |
| LRRNT | 34 or 36 | 64 |
| LRR | 66 | 89 |
| LRR | 90 | 113 |
| LRR | 114 | 137 |
| LRR | 138 | 161 |
| LRR | 162 | 185 |
| LRR | 186 | 209 |
| LRR | 210 | 233 |
| LRR | 234 | 257 |
| LRR | 258 | 281 |
| LRR | 282 | 305 |
| LRR | 306 | 329 |
| LRR | 330 | 353 |
| LRRCT | 363 | 414 or 416 |
| Basic | 415 or 417 | 424 |
| Ig | 419 | 493 |
| Connecting sequence | 494 | 551 |
| Transmembrane | 552 | 576 |
| Cytoplasmic | 577 | 614 |

Tissue distribution and developmental expression of LINGO-1 has been studied in humans and rats. LINGO-1 biology has been studied in an experimental animal (rat) model. Expression of rat LINGO-1 is localized to neurons and oligodendrocytes, as determined by northern blot and immuno-histochemical staining. Rat LINGO-1 mRNA expression level is regulated developmentally, peaking shortly after birth, i.e., ca. postnatal day one. In a rat spinal cord transection injury model, LINGO-1 is up-regulated at the injury site, as determined by RT-PCR. See Mi et al. Nature Neurosci. 7:221-228 (2004).

In the context of the amino acids comprising the various structural and functional domains of a LINGO-1 polypeptide, the term “about” includes the particularly recited value and values larger or smaller by several (e.g., 10, 9, 8, 7, 6, 5, 4, 3, 2, or 1) amino acids. Since the location of these domains as listed in Table 2 have been predicted by computer graphics, one of ordinary skill would appreciate that the amino acid residues constituting the domains may vary slightly (e.g., by about 1 to 15 residues) depending on the criteria used to define the domain.

Full-length, wild-type LINGO-1 binds to NgR1. See PCT Publication No. WO 2004/085648. LINGO-1 is expressed in oligodendrocytes and that the LINGO-1 protein is involved in the regulation of oligodendrocyte-mediated myelination of axons. See U.S Patent Publication No. 2006/0009388 A1, which is incorporated herein by reference in its entirety.

The nucleotide sequence for the full-length LINGO-1 molecule is as follows:

(SEQ ID NO: 52)
ATGCTGGCGGGGGGCGTGAGGAGCATGCCCAGCCCCCTCCTGGCCTGCTG
GCAGCCCATCCTCCTGCTGGTGCTGGGCTCAGTGCTGTCAGGCTCGGCCA
CGGGCTGCCCGCCCCGCTGCGAGTGCTCCGCCCAGGACCGCGCTGTGCTG
TGCCACCGCAAGCGCTTTGTGGCAGTCCCCGAGGGCATCCCCACCGAGAC
GCGCCTGCTGGACCTAGGCAAGAACCGCATCAAAACGCTCAACCAGGACG
AGTTCGCCAGCTTCCCGCACCTGGAGGAGCTGGAGCTCAACGAGAACATC
GTGAGCGCCGTGGAGCCCGGCGCCTTCAACAACCTCTTCAACCTCCGGAC
GCTGGGTCTCCGCAGCAACCGCCTGAAGCTCATCCCGCTAGGCGTCTTCA
CTGGCCTCAGCAACCTGACCAAGCTGGACATCAGCGAGAACAAGATTGTT
ATCCTGCTGGACTACATGTTTCAGGACCTGTACAACCTCAAGTCACTGGA
GGTTGGCGACAATGACCTCGTCTACATCTCTCACCGCGCCTTCAGCGGCC
TCAACAGCCTGGAGCAGCTGACGCTGGAGAAATGCAACCTGACCTCCATC
CCCACCGAGGCGCTGTCCCACCTGCACGGCCTCATCGTCCTGAGGCTCCG
GCACCTCAACATCAATGCCATCCGGGACTACTCCTTCAAGAGGCTCTACC
GACTCAAGGTCTTGGAGATCTCCCACTGGCCCTACTTGGACACCATGACA
CCCAACTGCCTCTACGGCCTCAACCTGACGTCCCTGTCCATCACACACTG
CAATCTGACCGCTGTGCCCTACCTGGCCGTCCGCCACCTAGTCTATCTCC
GCTTCCTCAACCTCTCCTACAACCCCATCAGCACCATTGAGGGCTCCATG
TTGCATGAGCTGCTCCGGCTGCAGGAGATCCAGCTGGTGGGCGGGCAGCT
GGCCGTGGTGGAGCCCTATGCCTTCCGCGGCCTCAACTACCTGCGCGTGC
TCAATGTCTCTGGCAACCAGCTGACCACACTGGAGGAATCAGTCTTCCAC
TCGGTGGGCAACCTGGAGACACTCATCCTGGACTCCAACCCGCTGGCCTG
CGACTGTCGGCTCCTGTGGGTGTTCCGGCGCCGCTGGCGGCTCAACTTCA
ACCGGCAGCAGCCCACGTGCGCCACGCCCGAGTTTGTCCAGGGCAAGGAG
TTCAAGGACTTCCCTGATGTGCTACTGCCCAACTACTTCACCTGCCGCCG
CGCCCGCATCCGGGACCGCAAGGCCCAGCAGGTGTTTGTGGACGAGGGCC
ACACGGTGCAGTTTGTGTGCCGGGCCGATGGCGACCCGCCGCCCGCCATC
CTCTGGCTCTCACCCCGAAAGCACCTGGTCTCAGCCAAGAGCAATGGGCG
GCTCACAGTCTTCCCTGATGGCACGCTGGAGGTGCGCTACGCCCAGGTAC
AGGACAACGGCACGTACCTGTGCATCGCGGCCAACGCGGGCGGCAACGAC
TCCATGCCCGCCCACCTGCATGTGCGCAGCTACTCGCCCGACTGGCCCCA
TCAGCCCAACAAGACCTTCGCTTTCATCTCCAACCAGCCGGGCGAGGGAG
AGGCCAACAGCACCCGCGCCACTGTGCCTTTCCCCTTCGACATCAAGACC
CTCATCATCGCCACCACCATGGGCTTCATCTCTTTCCTGGGCGTCGTCCT
CTTCTGCCTGGTGCTGCTGTTTCTCTGGAGCCGGGGCAAGGGCAACACAA
AGCACAACATCGAGATCGAGTATGTGCCCCGAAAGTCGGACGCAGGCATC
AGCTCCGCCGACGCGCCCCGCAAGTTCAACATGAAGATGATATGA.

The polypeptide sequence for the full-length LINGO-1 polypeptide is as follows:

(SEQ ID NO: 51)
MLAGGVRSMPSPLLACWQPILLLVLGSVLSGSATGCPPRCECSAQDRAVL
CHRKRFVAVPEGIPTETRLLDLGKNRIKTLNQDEFASFPHLEELELNENI
VSAVEPGAFNNLFNLRTLGLRSNRLKLIPLGVFTGLSNLTKLDISENKIV
ILLDYMFQDLYNLKSLEVGDNDLVYISHRAFSGLNSLEQLTLEKCNLTSI
PTEALSHLHGLIVLRLRHLNINAIRDYSFKRLYRLKVLEISHWPYLDTMT
PNCLYGLNLTSLSITHCNLTAVPYLAVRHLVYLRFLNLSYNPISTIEGSM
LHELLRLQEIQLVGGQLAVVEPYAFRGLNYLRVLNVSGNQLTTLEESVFH
SVGNLETLILDSNPLACDCRLLWVFRRRWRLNFNRQQPTCATPEFVQGKE
FKDFPDVLLPNYFTCRRARIRDRKAQQVFVDEGHTVQFVCRADGDPPPAI
LWLSPRKHLVSAKSNGRLTVFPDGTLEVRYAQVQDNGTYLCIAANAGGND
SMPAHLHVRSYSPDWPHQPNKTFAFISNQPGEGEANSTRATVPFPFDIKT
LIIATTMGFISFLGVVLFCLVLLFLWSRGKGNTKHNIEIEYVPRKSDAGI
SSADAPRKFNMKMI.

### Anti-LINGO-1 Antibody Molecules

In certain embodiments the antibody molecule binds to LINGO, e.g., human LINGO. In another embodiment, the antibody molecule binds to LINGO-1, e.g., human LINGO-1. In one embodiment, the antibody molecule is isolated, purified or recombinant. By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

As used herein, the term “antibody molecule” refers to a protein comprising at least one immunoglobulin variable domain sequence. The term antibody molecule includes, for example, full-length antibodies, mature antibodies, fragments, e.g., antigen-binding fragments of an antibody, derivatives, analogs, or variants of the antibodies disclosed herein, and any combination thereof.

The terms “fragment,” “variant,” “derivative” and “analog” when referring to LINGO-1 antibody molecules or antibody polypeptides include any polypeptides which retain at least some of the antigen-binding properties of the corresponding native antibody or polypeptide. Fragments of polypeptides include proteolytic fragments, as well as deletion fragments, in addition to specific antibody fragments discussed elsewhere herein. Variants of LINGO-1 antibody and antibody polypeptides include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of LINGO-1 antibody molecules and antibody polypeptides are polypeptides which have been altered so as to exhibit additional features not found on the native polypeptide. Examples include fusion proteins.

As used herein a “derivative” of a LINGO-1 antibody molecule or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the invention, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to LINGO-1, or an epitope thereof. With respect to proteins (or protein mimetics), the antigen-binding site typically includes one or more loops (of at least four amino acids or amino acid mimics) that form an interface that binds to LINGO-1. Typically, the antigen-binding site of an antibody molecule includes at least one or two CDRs, or more typically at least three, four, five or six CDRs.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

In certain embodiments, the antibody molecule can be a monoclonal or single specificity antibody, or an antigen-binding fragment thereof (e.g., an Fab, F(ab′)₂, Fv, a single chain Fv fragment, a single domain antibody, a diabody (dAb), a bivalent or bispecific antibody or fragment thereof, a single domain variant thereof) that binds to LINGO-1, e.g., a mammalian (e.g., human LINGO-1 (or a functional variant thereof)). In one embodiment, the antibody molecule is a monoclonal antibody against LINGO-1, e.g., human LINGO-1. Typically, the antibody molecule is a human, humanized, a CDR-grafted, chimeric, camelid, or in vitro generated antibody to human LINGO-1 (or functional fragment thereof, e.g., an antibody fragment as described herein). Typically, the antibody inhibits, reduces or neutralizes one or more activities of LINGO-1 (e.g., one or more biological activities of LINGO-1 as described herein).

In certain embodiments, the antibody molecule specifically binds to the same, or substantially the same, LINGO-1 epitope as the reference monoclonal antibody Li62 or Li81, described in U.S. Pat. No. 8,058,406, incorporated by reference in its entirety herein. Exemplary anti-LINGO-1 antibody molecules are described in U.S. Pat. No. 8,058,406. In one embodiment, antibody molecule includes at least the antigen-binding domains of Li62, Li81. As used herein, the term “antigen binding domain” includes a site that specifically binds an epitope on an antigen (e.g., an epitope of LINGO-1). The antigen binding domain of an antibody typically includes at least a portion of an immunoglobulin heavy chain variable region and at least a portion of an immunoglobulin light chain variable region. The binding site formed by these variable regions determines the specificity of the antibody.

In other embodiments, the anti-LINGO-1 antibody molecule competitively inhibits Li62 or Li81 from binding to LINGO-1.

In certain embodiments, the anti-LINGO-1 antibody molecule specifically or preferentially binds to a particular LINGO-1 polypeptide fragment or domain. Such LINGO-1 polypeptide fragments include, but are not limited to, a LINGO-1 polypeptide comprising, consisting essentially of, or consisting of amino acids 34 to 532; 34 to 417; 34 to 425; 34 to 493; 66 to 532; 66 to 417; 66 to 426; 66 to 493; 66 to 532; 417 to 532; 417 to 425 (the LINGO-1 basic region); 417 to 493; 417 to 532; 419 to 493 (the LINGO-11 g region); or 425 to 532 of SEQ ID NO:51; or a LINGO-1 variant polypeptide at least 70%, 75%, 80%, 85%, 90%, or 95% identical to amino acids 34 to 532; 34 to 417; 34 to 425; 34 to 493; 66 to 532; 66 to 417; 66 to 426; 66 to 493; 66 to 532; 417 to 532; 417 to 425 (the LINGO-1 basic region); 417 to 493; 417 to 532; 419 to 493 (the LINGO-11 g region); or 425 to 532 of SEQ ID NO:51.

In certain embodiments, the anti-LINGO-1 antibody molecule specifically or preferentially binds to a LINGO-1 peptide fragment comprising, consisting essentially of, or consisting of one or more leucine-rich-repeats (LRR) of LINGO-1. Such fragments, include, for example, fragments comprising, consisting essentially of, or consisting of amino acids 66 to 89; 66 to 113; 66 to 137; 90 to 113; 114 to 137; 138 to 161; 162 to 185; 186 to 209; 210 to 233; 234 to 257; 258 to 281; 282 to 305; 306 to 329; or 330 to 353 of SEQ ID NO:51. Corresponding fragments of a variant LINGO-1 polypeptide at least 70%, 75%, 80%, 85%, 90%, or 95% identical to amino acids 66 to 89; 66 to 113; 90 to 113; 114 to 137; 138 to 161; 162 to 185; 186 to 209; 210 to 233; 234 to 257; 258 to 281; 282 to 305; 306 to 329; or 330 to 353 of SEQ ID NO:51 are also contemplated.

In certain embodiments, the anti-LINGO-1 antibody molecule specifically or preferentially binds to a fragment comprising, consisting essentially of, or consisting of one or more cysteine rich regions flanking the LRR of LINGO-1. Such fragments, include, for example, a fragment comprising, consisting essentially of, or consisting of amino acids 34 to 64 of SEQ ID NO:51 (the N-terminal LRR flanking region (LRRNT)), or a fragment comprising, consisting essentially of, or consisting of amino acids 363 to 416 of SEQ ID NO:51 (the C-terminal LRR flanking region (LRRCT)), amino acids Corresponding fragments of a variant LINGO-1 polypeptide at least 70%, 75%, 80%, 85%, 90%, or 95% identical to amino acids 34 to 64 and 363 to 416 of SEQ ID NO:51 are also contemplated. In certain embodiments, the anti-LINGO-1 antibody molecule specifically or preferentially binds to a fragment comprising, consisting essentially of, or consisting of amino acids 41 to 525 of SEQ ID NO:51; 40 to 526 of SEQ ID NO:51; 39 to 527 of SEQ ID NO:51; 38 to 528 of SEQ ID NO:51; 37 to 529 of SEQ ID NO:51; 36 to 530 of SEQ ID NO:51; 35 to 531 of SEQ ID NO:51; 34 to 531 of SEQ ID NO:51; 46 to 520 of SEQ ID NO:51; 45 to 521 of SEQ ID NO:51; 44 to 522 of SEQ ID NO:51; 43 to 523 of SEQ ID NO:51; and 42 to 524 of SEQ ID NO:51.

In certain embodiments, the anti-LINGO-1 antibody molecule specifically or preferentially binds to a fragment comprising, consisting essentially of, or consisting of amino acids 1 to 33 of SEQ ID NO:51; 1 to 35 of SEQ ID NO:51; 34 to 64 of SEQ ID NO:51; 36 to 64 of SEQ ID NO:51; 66 to 89 of SEQ ID NO:51; 90 to 113 of SEQ ID NO:51; 114 to 137 of SEQ ID NO:51; 138 to 161 of SEQ ID NO:51; 162 to 185 of SEQ ID NO:51; 186 to 209 of SEQ ID NO:51; 210 to 233 of SEQ ID NO:51; 234 to 257 of SEQ ID NO:51; 258 to 281 of SEQ ID NO:51; 282 to 305 of SEQ ID NO:51; 306 to 329 of SEQ ID NO:51; 330 to 353 of SEQ ID NO:51; 363 to 416 of SEQ ID NO:51; 417 to 424 of SEQ ID NO:51; 419 to 493 of SEQ ID NO:51; and 494 to 551 of SEQ ID NO:51.

In certain embodiments, the anti-LINGO-1 antibody molecule specifically or preferentially binds to a fragment comprising, consisting essentially of, or consisting of amino acids 1 to 33 of SEQ ID NO:51; 1 to 35 of SEQ ID NO:51; 1 to 64 of SEQ ID NO:51; 1 to 89 of SEQ ID NO:51; 1 to 113 of SEQ ID NO:51; 1 to 137 of SEQ ID NO:51; 1 to 161 of SEQ ID NO:51; 1 to 185 of SEQ ID NO:51; 1 to 209 of SEQ ID NO:51; 1 to 233 of SEQ ID NO:51; 1 to 257 of SEQ ID NO:51; 1 to 281 of SEQ ID NO:51; 1 to 305 of SEQ ID NO:51; 1 to 329 of SEQ ID NO:51; 1 to 353 of SEQ ID NO:51; 1 to 416 of SEQ ID NO:51; 1 to 424 of SEQ ID NO:51; 1 to 493 of SEQ ID NO:51; 1 to 551 of SEQ ID NO:51; 1 to 531 of SEQ ID NO:51 and 1 to 532 of SEQ ID NO:51.

In certain embodiments, the anti-LINGO-1 antibody molecule specifically or preferentially binds to a fragment comprising, consisting essentially of, or consisting of amino acids 34 to 64 of SEQ ID NO:51; 34 to 89 of SEQ ID NO:51; 34 to 113 of SEQ ID NO:51; 34 to 137 of SEQ ID NO:51; 34 to 161 of SEQ ID NO:51; 34 to 185 of SEQ ID NO:51; 34 to 209 of SEQ ID NO:51; 34 to 233 of SEQ ID NO:51; 34 to 257 of SEQ ID NO:51; 34 to 281 of SEQ ID NO:51; 34 to 305 of SEQ ID NO:51; 34 to 329 of SEQ ID NO:51; 34 to 353 of SEQ ID NO:51; 34 to 416 of SEQ ID NO:51; 34 to 424 of SEQ ID NO:51; 34 to 493 of SEQ ID NO:51; and 34 to 551 of SEQ ID NO:51.

In certain embodiments, the anti-LINGO-1 antibody molecule specifically or preferentially binds to a fragment comprising, consisting essentially of, or consisting of amino acids 34 to 530 of SEQ ID NO:51; 34 to 531 of SEQ ID NO:51; 34 to 532 of SEQ ID NO:51; 34 to 533 of SEQ ID NO:51; 34 to 534 of SEQ ID NO:51; 34 to 535 of SEQ ID NO:51; 34 to 536 of SEQ ID NO:51; 34 to 537 of SEQ ID NO:51; 34 to 538 of SEQ ID NO:51; 34 to 539 of SEQ ID NO:51; 30 to 532 of SEQ ID NO:51; 31 to 532 of SEQ ID NO:51; 32 to 532 of SEQ ID NO:51; 33 to 532 of SEQ ID NO:51; 34 to 532 of SEQ ID NO:51; 35 to 532 of SEQ ID NO:51; 36 to 532 of SEQ ID NO:51; 30 to 531 of SEQ ID NO:51; 31 to 531 of SEQ ID NO:51; 32 to 531 of SEQ ID NO:51; 33 to 531 of SEQ ID NO:51; 34 to 531 of SEQ ID NO:51; 35 to 531 of SEQ ID NO:51; and 36 to 531 of SEQ ID NO:51.

In certain embodiments, the anti-LINGO-1 antibody molecule specifically or preferentially binds to a fragment comprising, consisting essentially of, or consisting of amino acids 36 to 64 of SEQ ID NO:51; 36 to 89 of SEQ ID NO:51; 36 to 113 of SEQ ID NO:51; 36 to 137 of SEQ ID NO:51; 36 to 161 of SEQ ID NO:51; 36 to 185 of SEQ ID NO:51; 36 to 209 of SEQ ID NO:51; 36 to 233 of SEQ ID NO:51; 36 to 257 of SEQ ID NO:51; 36 to 281 of SEQ ID NO:51; 36 to 305 of SEQ ID NO:51; 36 to 329 of SEQ ID NO:51; 36 to 353 of SEQ ID NO:51; 36 to 416 of SEQ ID NO:51; 36 to 424 of SEQ ID NO:51; 36 to 493 of SEQ ID NO:51; and 36 to 551 of SEQ ID NO:51.

In certain embodiments, the anti-LINGO-1 antibody molecule specifically or preferentially binds to a fragments comprising, consisting essentially of, or consisting of amino acids 36 to 530 of SEQ ID NO:51; 36 to 531 of SEQ ID NO:51; 36 to 532 of SEQ ID NO:51; 36 to 533 of SEQ ID NO:51; 36 to 534 of SEQ ID NO:51; 36 to 535 of SEQ ID NO:51; 36 to 536 of SEQ ID NO:51; 36 to 537 of SEQ ID NO:51; 36 to 538 of SEQ ID NO:51; and 36 to 539 of SEQ ID NO:51.

In certain embodiments, the anti-LINGO-1 antibody molecule specifically or preferentially binds to a fragment comprising, consisting essentially of, or consisting of amino acids 417 to 493 of SEQ ID NO:51; 417 to 494 of SEQ ID NO:51; 417 to 495 of SEQ ID NO:51; 417 to 496 of SEQ ID NO:51; 417 to 497 of SEQ ID NO:51; 417 to 498 of SEQ ID NO:51; 417 to 499 of SEQ ID NO:51; 417 to 500 of SEQ ID NO:51; 417 to 492 of SEQ ID NO:51; 417 to 491 of SEQ ID NO:51; 412 to 493 of SEQ ID NO:51; 413 to 493 of SEQ ID NO:51; 414 to 493 of SEQ ID NO:51; 415 to 493 of SEQ ID NO:51; 416 to 493 of SEQ ID NO:51; 411 to 493 of SEQ ID NO:51; 410 to 493 of SEQ ID NO:51; 410 to 494 of SEQ ID NO:51; 411 to 494 of SEQ ID NO:51; 412 to 494 of SEQ ID NO:51; 413 to 494 of SEQ ID NO:51; 414 to 494 of SEQ ID NO:51; 415 to 494 of SEQ ID NO:51; 416 to 494 of SEQ ID NO:51; 417 to 494 of SEQ ID NO:51; and 418 to 494 of SEQ ID NO:51.

In certain embodiments, the anti-LINGO-1 antibody molecule specifically or preferentially binds to a LINGO-1 polypeptide comprising, consisting essentially of, or consisting of peptides of the Ig domain of LINGO-1 or fragments, variants, or derivatives of such polypeptides. Specifically, polypeptides comprising, consisting essentially of, or consisting of the following polypeptide sequences: ITX₁X₂X₃ (SEQ ID NO:88), ACX₁X₂X₃ (SEQ ID NO:89), VCX₁X₂X₃ (SEQ ID NO:90) and SPX₁X₂X₃ (SEQ ID NO:91) where X₁ is lysine, arginine, histidine, glutamine, or asparagine, X₂ is lysine, arginine, histidine, glutamine, or asparagine and X₃ is lysine, arginine, histidine, glutamine, or asparagine. For example, LINGO-1 peptide fragments to which certain antibody molecules can bind include, those fragments comprising, consisting essentially of, or consisting of the following polypeptide sequences: SPRKH (SEQ ID NO:92), SPRKK (SEQ ID NO:93), SPRKR (SEQ ID NO:94), SPKKH (SEQ ID NO:95), SPHKH (SEQ ID NO:96), SPRRH (SEQ ID NO:97), SPRHH (SEQ ID NO:98), SPRRR (SEQ ID NO:99), SPHHH (SEQ ID NO:100) SPKKK (SEQ ID NO:101), LSPRKH (SEQ ID NO:102), LSPRKK (SEQ ID NO:103), LSPRKR (SEQ ID NO:104), LSPKKH (SEQ ID NO:105), LSPHKH (SEQ ID NO:106), LSPRRH (SEQ ID NO:107), LSPRHH (SEQ ID NO:108), LSPRRR (SEQ ID NO:109), LSPHHH (SEQ ID NO:110) LSPKKK (SEQ ID NO:111), WLSPRKH (SEQ ID NO:112), WLSPRKK (SEQ ID NO:113), WLSPRKR (SEQ ID NO:114), WLSPKKH (SEQ ID NO:115), WLSPHKH (SEQ ID NO:116), WLSPRRH (SEQ ID NO:117), WLSPRHH (SEQ ID NO:118), WLSPRRR (SEQ ID NO:119), WLSPHHH (SEQ ID NO:120) WLSPKKK (SEQ ID NO:121). These LINGO-1 polypeptides include the basic “RKH loop” (Arginine-Lysine-Histidine amino acids 456-458) in the Ig domain of LINGO-1. Additional LINGO-1 peptides which include a basic tripeptide are ITPKRR (SEQ ID NO:122), ACHHK (SEQ ID NO:123) and VCHHK (SEQ ID NO:124).

In certain embodiments, the anti-LINGO-1 antibody molecule specifically or preferentially binds to a LINGO-1 polypeptide comprising, consisting essentially of, or consisting of peptides of the Ig domain of LINGO-1 or fragments, variants, or derivatives of such polypeptides. Specifically, peptides comprising, consisting essentially of, or consisting of the following polypeptide sequences: X₄X₅RKH (SEQ ID NO:125), X₄X₅RRR (SEQ ID NO:126), X₄X₅KKK (SEQ ID NO:127), X₄X₅HHH (SEQ ID NO:128), X₄X₅RKK (SEQ ID NO:129), X₄X₅RKR (SEQ ID NO:130), X₄X₅KKH (SEQ ID NO:131), X₄X₅HKH (SEQ ID NO:132), X₄X₅RRH (SEQ ID NO:133) and X₄X₅RHH (SEQ ID NO:134) where X₄ is any amino acid and X₅ is any amino acid.

In certain embodiments, the anti-LINGO-1 antibody molecule specifically or preferentially binds to a LINGO-1 polypeptide comprising, consisting essentially of, or consisting of peptides of the Ig domain of LINGO-1 or fragments, variants, or derivatives of such polypeptides. Specifically, polypeptides comprising, consisting essentially of, or consisting of the following polypeptide sequences: ITX₆X₇X₈ (SEQ ID NO:135), ACX₆X₇X₈ (SEQ ID NO:136), VCX₆X₇X₈ (SEQ ID NO:137) and SPX₆X₇X₈ (SEQ ID NO:138) where X₆ is lysine, arginine, histidine, glutamine, or asparagine, X₇ is any amino acid and X₈ is lysine, arginine, histidine, glutamine, or asparagine. For example, a polypeptide comprising, consisting essentially of, or consisting of the following polypeptide sequence: SPRLH (SEQ ID NO:139).

In certain embodiments, the anti-LINGO-1 antibody molecule specifically or preferentially binds to a LINGO-1 polypeptide comprising, consisting essentially of, or consisting of peptides which contain amino acids 452-458 in the Ig domain of LINGO-1, or derivatives thereof, wherein amino acid 452 is a tryptophan or phenylalanine residue.

In certain embodiments, the anti-LINGO-1 antibody molecule specifically or preferentially binds to a LINGO-1 polypeptide comprising, consisting essentially of, or consisting of peptides of the basic domain of LINGO-1. Specifically, peptides comprising, consisting essentially of, or consisting of the following polypeptide sequences: RRARIRDRK (SEQ ID NO:140), KKVKVKEKR (SEQ ID NO:141), RRLRLRDRK (SEQ ID NO:142), RRGRGRDRK (SEQ ID NO:143) and RRIRARDRK (SEQ ID NO:144).

Additional exemplary soluble LINGO-1 polypeptides and methods and materials for obtaining these molecules for producing antibodies or antibody fragments of the present invention may be found, e.g., in International Patent Application No. PCT/US2004/008323, incorporated herein by reference in its entirety.

Methods of making antibodies are known in the art and described herein. Once antibodies to various fragments of, or to the full-length LINGO-1 without the signal sequence, have been produced, determining which amino acids, or epitope, of LINGO-1 to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v.2, John Wiley & Sons, Inc. (1996)). Additional epitope mapping protocols may be found in Morris, G. Epitope Mapping Protocols, N.J.: Humana Press (1996), which are both incorporated herein by reference in their entireties. Epitope mapping can also be performed by commercially available means (i.e. ProtoPROBE, Inc. (Milwaukee, Wis.)).

Additionally, antibodies produced which bind to any portion of LINGO-1 can then be screened for their ability to act as an antagonist of LINGO-1 and thus promote neurite outgrowth, neuronal and oligodendrocyte survival, proliferation and differentiation, as well as enhance myelination. Antibodies can be screened for oligodendrocyte/neuronal survival for example by using the methods described herein such as in Examples 11 or 12 or as described in PCT/US2008/000316, filed Jan. 9, 2008, and PCT/US2006/026271, filed Jul. 7, 2006, which are incorporated herein by reference in their entireties. Additionally, antibodies can be screened for example by their ability to enhance myelination by using the methods described herein such as in Examples 2, 6, 9, 10, 11 or 13 or as described in PCT/US2008/000316 and/or PCT/US2006/026271. Finally, antibodies can be screened for their ability to promote oligodendrocyte proliferation and differentiation, as well as neurite outgrowth for example by using the methods described herein such as in Examples 4 or 5 or as described in PCT/US2008/000316 and/or PCT/US2006/026271. Other antagonist functions of antibodies of the present invention can be tested using other assays as described in the Examples of U.S. Pat. No. 8,058,406, incorporated by reference herein.

In certain embodiments, the anti-LINGO-1 antibody molecule specifically or preferentially binds to at least one epitope of LINGO-1, where the epitope comprises, consists essentially of, or consists of at least about four to five amino acids of SEQ ID NO:5, at least seven, at least nine, or between at least about 15 to about 30 amino acids of SEQ ID NO:5. The amino acids of a given epitope of SEQ ID NO:51 as described may be, but need not be contiguous or linear. In certain embodiments, the at least one epitope of LINGO-1 comprises, consists essentially of, or consists of a non-linear epitope formed by the extracellular domain of LINGO-1 as expressed on the surface of a cell or as a soluble fragment, e.g., fused to an IgG Fc region. Thus, in certain embodiments the at least one epitope of LINGO-1 comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:51, where the non-contiguous amino acids form an epitope through protein folding.

In other embodiments, the anti-LINGO-1 antibody molecule specifically or preferentially binds to at least one epitope of LINGO-1, where the epitope comprises, consists essentially of, or consists of, in addition to one, two, three, four, five, six or more contiguous or non-contiguous amino acids of SEQ ID NO:51 as described above, and an additional moiety which modifies the protein, e.g., a carbohydrate moiety may be included such that the LINGO-1 antibody binds with higher affinity to modified target protein than it does to an unmodified version of the protein. Alternatively, the LINGO-1 antibody does not bind the unmodified version of the target protein at all.

In certain embodiments, the anti-LINGO-1 antibody molecule specifically or preferentially binds to a LINGO-1 polypeptide or fragment thereof, or a LINGO-1 variant polypeptide, with an affinity characterized by a dissociation constant (K_{D}) which is less than the K_{D} for said reference monoclonal antibody.

In certain embodiments, the anti-LINGO-1 antibody molecule specifically or preferentially binds to at least one epitope of LINGO-1 or fragment or variant described above, i.e., binds to such an epitope more readily than it would bind to an unrelated, or random epitope; binds preferentially to at least one epitope of LINGO-1 or fragment or variant described above, i.e., binds to such an epitope more readily than it would bind to a related, similar, homologous, or analogous epitope; competitively inhibits binding of a reference antibody which itself binds specifically or preferentially to a certain epitope of LINGO-1 or fragment or variant described above; or binds to at least one epitope of LINGO-1 or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹° M, about 10⁻¹⁰¹ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, about 10⁻¹⁵ M. In a particular aspect, the antibody or fragment thereof preferentially binds to a human LINGO-1 polypeptide or fragment thereof, relative to a murine LINGO-1 polypeptide or fragment thereof.

In other embodiments, the anti-LINGO-1 antibody molecule binds LINGO-1 polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Alternatively, an antibody, or antigen-binding fragment, variant, or derivative thereof of the invention binds LINGO-1 polypeptides or fragments or variants thereof with an off rate (k(off)) of less than or equal to 5×10⁻⁴ sec¹, 10⁻⁴ sec¹, 5×10⁻⁵ sec¹, or 10⁻⁵ sec¹, 5×10⁻⁶ sec¹, 10⁻⁶ sec¹, 5×10⁻⁷ sec¹ or 10⁻⁷ sec¹.

In other embodiments, the anti-LINGO-1 antibody molecule binds LINGO-1 polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹. Alternatively, the antibody molecule binds LINGO-1 polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, 5×10⁶ M⁻¹ sec⁻¹, or 10⁷ M⁻¹ sec⁻¹, 5×10⁷ M⁻¹ sec⁻¹.

In other embodiments, the LINGO-1 antibody molecule is an antagonist of LINGO-1 activity. In certain embodiments, for example, binding of an antagonist LINGO-1 antibody to LINGO-1, as expressed on neurons, blocks myelin-associated neurite outgrowth inhibition or neuronal cell death. In other embodiments, binding of the LINGO-1 antibody to LINGO-1, as expressed on oligodendrocytes, blocks inhibition of oligodendrocyte growth or differentiation, or blocks demyelination or dysmyelination of CNS neurons.

Modified forms of LINGO-1 antibody molecules can be made from whole precursor or parent antibodies using techniques known in the art. Exemplary techniques are discussed in more detail herein.

In certain embodiments, the antibody molecule can be recombinantly produced, e.g., produced by phage display or by combinatorial methods. Phage display and combinatorial methods for generating anti-LINGO-1 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-LINGO-1 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. The non-human antibody can be a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-LINGO-1 antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to LINGO-1 or a fragment thereof.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference. Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Humanized antibodies can have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The anti-LINGO-1 antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target LINGO-1 protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

LINGO-1 antibody molecules can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (also referred to herein as antibody-dependent cell-mediated cytotoxicity, or ADCC), release of inflammatory mediators, placental transfer and control of immunoglobulin production.

In certain embodiments, the anti-LINGO-1 antibody molecule, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain LINGO-1 antibody molecules, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

### Exemplary Anti-LINGO-1 Antibody Molecules

In certain embodiments, the anti-LINGO-1 antibody molecules comprise, consist essentially of, or consist of an immunoglobulin heavy chain variable region (VH), where at least one of the CDRs of the heavy chain variable region, or at least two the CDRs of the heavy chain variable region are at least 80%, 85%, 90% or 95% identical to reference heavy chain CDR1, CDR2, or CDR3 amino acid sequences of Li62 or Li81 or variants thereof as described in Table 3. Alternatively, the CDR1, CDR2, and CDR3 regions of the VH are at least 80%, 85%, 90% or 95% identical to reference heavy chain CDR1, CDR2, and CDR3 amino acid sequences of Li62 or Li81 or variants thereof as described in Table 3. Thus, according to this embodiment a heavy chain variable region of the invention has CDR1, CDR2, or CDR3 polypeptide sequences related to the polypeptide sequences shown in Table 3. In certain embodiment, the anti-LINGO-1 antibody molecules comprise, consist essentially of, or consist of the VH polypeptide or a fragment thereof as described in Table 3, or an amino acid sequence at least 80%, 85%, 90% or 95% identical thereto.

TABLE 3
LINGO-1 Antibody VH Sequences

| Anti- | | VH | | VH |
|---|---|---|---|---|
| body | VH SEQUENCE | CDR1 | VH CDR2 | CDR3 |
| Li62 | EVQLLESGGGLVQPGGSLRLSCAASGFTFSIYPMFWV | IYPMF | WIGPSG | EGHND |
| | RQAPGKGLEWVSWIGPSGGITKYADSVKGRFTISRD | (SEQ | GITKYA | WYFDL |
| | NSKNTLYLQMNSLRAEDTATYYCAREGHNDWYFDL | ID | DSVKG | (SEQ ID |
| | WGRGTLVTVSS (SEQ ID NO: 1) | NO: 2) | (SEQ ID | NO: 4) |
| | | | NO: 3) | |
| Li62 | EVQLLESGGGLVQPGGSLRLSCAASGFTFSIYPMFWV | IYPMF | WIGPSG | EGYYD |
| variant | RQAPGKGLEWVSWIGPSGGITKYADSVKGRFTISRD | (SEQ | GITKYA | WYFDQ |
| B06 | NSKNTLYLQMNSLRAEDTATYYCAREGYYDWYFD | ID | DSVKG | (SEQ ID |
| | QWGRGTLVTVSS (SEQ ID NO: 53) | NO: 2) | (SEQ ID | NO: 17) |
| | | | NO: 3) | |
| Li62 | EVQLLESGGGLVQPGGSLRLSCAASGFTFSIYPMFWV | IYPMF | WIGPSG | EGQYD |
| variant | RQAPGKGLEWVSWIGPSGGITKYADSVKGRFTISRD | (SEQ | GITKYA | WYFDV |
| B12 | NSKNTLYLQMNSLRAEDTATYYCAREGQYDWYFD | ID | DSVKG | (SEQ ID |
| | VWGRGTLVTVSS (SEQ ID NO: 54) | NO: 2) | (SEQ ID | NO: 18) |
| | | | NO: 3) | |
| Li62 | EVQLLESGGGLVQPGGSLRLSCAASGFTFSIYPMFWV | IYPMF | WIGPSG | EGDYD |
| variant | RQAPGKGLEWVSWIGPSGGITKYADSVKGRFTISRD | (SEQ | GITKYA | WYFDL |
| F06 | NSKNTLYLQMNSLRAEDTATYYCAREGDYDWYFDL | ID | DSVKG | (SEQ ID |
| | WGRGTLVTVSS (SEQ ID NO: 55) | NO: 2) | (SEQ ID | NO: 19) |
| | | | NO: 3) | |
| Li62 | EVQLLESGGGLVQPGGSLRLSCAASGFTFSIYPMFWV | IYPMF | WIGPSG | EGQYD |
| variant | RQAPGKGLEWVSWIGPSGGITKYADSVKGRFTISRD | (SEQ | GITKYA | WYFEL |
| B01 | NSKNTLYLQMNSLRAEDTATYYCAREGQYDWYFEL | ID | DSVKG | (SEQ ID |
| | WGRGTLVTVSS (SEQ ID NO: 56) | NO: 2) | (SEQ ID | NO: 20) |
| | | | NO: 3) | |
| Li62 | EVQLLESGGGLVQPGGSLRLSCAASGFTFSIYPMFWV | IYPMF | WIGPSG | EADID |
| variant | RQAPGKGLEWVSWIGPSGGITKYADSVKGRFTISRD | (SEQ | GITKYA | WIThDL |
| D09 | NSKNTLYLQMNSLRAEDTATYYCAREADIDWFFDL | ID | DSVKG | (SEQ ID |
| | WGRGTLVTVSS (SEQ ID NO: 57) | NO: 2) | (SEQ ID | NO: 21) |
| | | | NO: 3) | |
| Li62 | EVQLLESGGGLVQPGGSLRLSCAASGFTFSIYPMFWV | IYPMF | WIGPSG | EGHYD |
| variant | RQAPGKGLEWVSWIGPSGGITKYADSVKGRFTISRD | (SEQ | GITKYA | WYFDL |
| D12 | NSKNTLYLQMNSLRAEDTATYYCAREGHYDWYFDL | ID | DSVKG | (SEQ ID |
| | WGRGTLVTVSS (SEQ ID NO: 58) | NO: 2) | (SEQ ID | NO: 22) |
| | | | NO: 3) | |
| Li62 | EVQLLESGGGLVQPGGSLRLSCAASGFTFSIYPMFWV | IYPMF | WIGPSG | EGRYD |
| variant | RQAPGKGLEWVSWIGPSGGITKYADSVKGRFTISRD | (SEQ | GITKYA | WYFDP |
| F01 | NSKNTLYLQMNSLRAEDTATYYCAREGRYDWYFDP | ID | DSVKG | (SEQ ID |
| | WGRGTLVTVSS (SEQ ID NO: 59) | NO: 2) | (SEQ ID | NO: 23) |
| | | | NO: 3) | |
| Li62 | EVQLLESGGGLVQPGGSLRLSCAASGFTFSIYPMFWV | IYPMF | WIGPSG | EGDYD |
| variant | RQAPGKGLEWVSWIGPSGGITKYADSVKGRFTISRD | (SEQ | GITKYA | WYFGL |
| F02 | NSKNTLYLQMNSLRAEDTATYYCAREGDYDWYFGL | ID | DSVKG | (SEQ ID |
| | WGRGTLVTVSS (SEQ ID NO: 60) | NO: 2) | (SEQ ID | NO: 24) |
| | | | NO: 3) | |
| Li62 | EVQLLESGGGLVQPGGSLRLSCAASGFTFSIYPMFWV | IYPMF | WIGPSG | EGRYD |
| variant | RQAPGKGLEWVSWIGPSGGITKYADSVKGRFTISRD | (SEQ | GITKYA | WYFDL |
| F06 | NSKNTLYLQMNSLRAEDTATYYCAREGRYDWYFDL | ID | DSVKG | (SEQ ID |
| | WGRGTLVTVSS (SEQ ID NO: 61) | NO: 2) | (SEQ ID | NO: 25) |
| | | | NO: 3) | |
| Li62 | EVQLLESGGGLVQPGGSLRLSCAASGFTFSIYPMFWV | IYPMF | WIGPSG | ESHIDR |
| variant | RQAPGKGLEWVSWIGPSGGITKYADSVKGRFTISRD | (SEQ | GITKYA | YFDL |
| F10 | NSKNTLYLQMNSLRAEDTATYYCARESHIDRYFDL | ID | DSVKG | (SEQ ID |
| | WGRGTLVTVSS (SEQ ID NO: 62) | NO: 2) | (SEQ ID | NO: 26) |
| | | | NO: 3) | |
| Li62 | EVQLLESGGGLVQPGGSLRLSCAASGFTFSIYPMFWV | IYPMF | WIGPSG | EGQYD |
| variant | RQAPGKGLEWVSWIGPSGGITKYADSVKGRFTISRD | (SEQ | GITKYA | WYFDV |
| G08 | NSKNTLYLQMNSLRAEDTATYYCAREGQYDWYFD | ID | DSVKG | (SEQ ID |
| | VWGRGTLVTVSS (SEQ ID NO: 63) | NO: 2) | (SEQ ID | NO: 27) |
| | | | NO: 3) | |
| Li62 | EVQLLESGGGLVQPGGSLRLSCAASGFTFSIYPMFWV | IYPMF | WIGPSG | EGHYN |
| variant | RQAPGKGLEWVSWIGPSGGITKYADSVKGRFTISRD | (SEQ | GITKYA | GYFDL |
| H08 | NSKNTLYLQMNSLRAEDTATYYCAREGHYNGYFDL | ID | DSVKG | (SEQ ID |
| | WGRGTLVTVSS (SEQ ID NO: 64) | NO: 2) | (SEQ ID | NO: 28) |
| | | | NO: 3) | |
| Li62 | EVQLLESGGGLVQPGGSLRLSCAASGFTFSIYPMFWV | IYPMF | WIGPSG | EGYYD |
| variant | RQAPGKGLEWVSWIGPSGGITKYADSVKGRFTISRD | (SEQ | GITKYA | WYFDL |
| C10 | NSKNTLYLQMNSLRAEDTATYYCAREGYYDWYFDL | ID | DSVKG | (SEQ ID |
| | WGRGTLVTVSS (SEQ ID NO: 65) | NO: 2) | (SEQ ID | NO: 29) |
| | | | NO: 3) | |
| Li62 | EVQLLESGGGLVQPGGSLRLSCAASGFTFSIYPMFWV | IYPMF | WIGPSG | EGTYD |
| variant | RQAPGKGLEWVSWIGPSGGITKYADSVKGRFTISRD | (SEQ | GITKYA | WYLDL |
| C02 | NSKNTLYLQMNSLRAEDTATYYCAREGTYDWYLDL | ID | DSVKG | (SEQ ID |
| | WGRGTLVTVSS (SEQ ID NO: 66) | NO: 2) | (SEQ ID | NO: 30) |
| | | | NO: 3) | |
| Li62 | EVQLLESGGGLVQPGGSLRLSCAASGFTFSIYPMFWV | IYPMF | WIGPSG | EGYYD |
| variant | RQAPGKGLEWVSWIGPSGGITKYADSVKGRFTISRD | (SEQ | GITKYA | WYFEL |
| D05 | NSKNTLYLQMNSLRAEDTATYYCAREGYYDWYFEL | ID | DSVKG | (SEQ ID |
| | WGRGTLVTVSS (SEQ ID NO: 67) | NO: 2) | (SEQ ID | NO: 31) |
| | | | NO: 3) | |
| Li62 | EVQLLESGGGLVQPGGSLRLSCAASGFTFSIYPMFWV | IYPMF | WIGPSG | EGLID |
| variant | RQAPGKGLEWVSWIGPSGGITKYADSVKGRFTISRD | (SEQ | GITKYA | WFFDQ |
| F02 | NSKNTLYLQMNSLRAEDTATYYCAREGLIDWFFDQ | ID | DSVKG | (SEQ ID |
| | WGRGTLVTVSS (SEQ ID NO: 68) | NO: 2) | (SEQ ID | NO: 32) |
| | | | NO: 3) | |
| Li62 | EVQLLESGGGLVQPGGSLRLSCAASGFTFSIYPMFWV | IYPMF | WIGPSG | EGQFD |
| variant | RQAPGKGLEWVSWIGPSGGITKYADSVKGRFTISRD | (SEQ | GITKYA | WYFDL |
| C10 | NSKNTLYLQMNSLRAEDTATYYCAREGQFDWYFDL | ID | DSVKG | (SEQ ID |
| | WGRGTLVTVSS (SEQ ID NO: 69) | NO: 2) | (SEQ ID | NO: 33) |
| | | | NO: 3) | |
| Li62 | EVQLLESGGGLVQPGGSLRLSCAASGFTFSIYPMFWV | IYPMF | WIGPSG | EGTYD |
| variant | RQAPGKGLEWVSWIGPSGGITKYADSVKGRFTISRD | (SEQ | GITKYA | WYFDL |
| H08 | NSKNTLYLQMNSLRAEDTATYYCAREGTYDWYFDL | ID | DSVKG | (SEQ ID |
| | WGRGTLVTVSS (SEQ ID NO: 70) | NO: 2) | (SEQ ID | NO: 34) |
| | | | NO: 3) | |
| Li81 | EVQLLESGGGLVQPGGSLRLSCAASGFTFSAYEMKW | AYEM | VIGPSG | EGDND |
| | VRQAPGKGLEWVSVIGPSGGFTFYADSVKGRFTISRD | K (SEQ | GFTFYA | AFDI |
| | NSKNTLYLQMNSLRAEDTAVYYCATEGDNDAFDIW | ID | DSVKG | (SEQ ID |
| | GQGTTVTVSS (SEQ ID NO: 5) | NO: 6) | (SEQ ID | NO: 8) |
| | | | NO: 7) | |
| Li81 | EVQLLESGGGLVQPGGSLRLSCAASGFTFSAYEMKW | AYEM | VIGPSG | EGEND |
| variant | VRQAPGKGLEWVSVIGPSGGFTFYADSVKGRFTISRD | K (SEQ | GFTFYA | AFDV |
| F09 | NSKNTLYLQMNSLRAEDTAVYYCATEGENDAFDVW | ID | DSVKG | (SEQ ID |
| | GQGTTVTVSS (SEQ ID NO: 71) | NO: 6) | (SEQ ID | NO: 35) |
| | | | NO: 7) | |
| Li81 | EVQLLESGGGLVQPGGSLRLSCAASGFTFSAYEMKW | AYEM | VIGPSG | EGDND |
| variant | VRQAPGKGLEWVSVIGPSGGFTFYADSVKGRFTISRD | K (SEQ | GFTFYA | AYDT |
| G02 | NSKNTLYLQMNSLRAEDTAVYYCATEGDNDAYDT | ID | DSVKG | (SEQ ID |
| | WGQGTTVTVSS (SEQ ID NO: 72) | NO: 6) | (SEQ ID | NO: 36) |
| | | | NO: 7) | |
| Li81 | EVQLLESGGGLVQPGGSLRLSCAASGFTFSAYEMKW | AYEM | VIGPSG | EGTND |
| variant | VRQAPGKGLEWVSVIGPSGGFTFYADSVKGRFTISRD | K (SEQ | GFTFYA | AFDI |
| H03 | NSKNTLYLQMNSLRAEDTAVYYCATEGTNDAFDIW | ID | DSVKG | (SEQ ID |
| | GQGTTVTVSS (SEQ ID NO: 73) | NO: 6) | (SEQ ID | NO: 37) |
| | | | NO: 7) | |
| Li81 | EVQLLESGGGLVQPGGSLRLSCAASGFTFSAYEMKW | AYEM | VIGPSG | EGDND |
| variant | VRQAPGKGLEWVSVIGPSGGFTFYADSVKGRFTISRD | K (SEQ | GFTFYA | AFDS |
| A12 | NSKNTLYLQMNSLRAEDTAVYYCATEGDNDAFDSW | ID | DSVKG | (SEQ ID |
| | GQGTTVTVSS (SEQ ID NO: 74) | NO: 6) | (SEQ ID | NO: 38) |
| | | | NO: 7) | |
| Li81 | EVQLLESGGGLVQPGGSLRLSCAASGFTFSAYEMKW | AYEM | VIGPSG | EGDND |
| variant | VRQAPGKGLEWVSVIGPSGGFTFYADSVKGRFTISRD | K (SEQ | GFTFYA | AFDT(S |
| C02 | NSKNTLYLQMNSLRAEDTAVYYCATEGDNDAFDTW | ID | DSVKG | EQ ID |
| | GQGTTVTVSS (SEQ ID NO: 75) | NO: 6) | (SEQ ID | NO: 39) |
| | | | NO: 7) | |
| Li81 | EVQLLESGGGLVQPGGSLRLSCAASGFTFSAYEMKW | AYEM | VIGPSG | EGDND |
| variant | VRQAPGKGLEWVSVIGPSGGFTFYADSVKGRFTISRD | K (SEQ | GFTFYA | AYDR |
| C11 | NSKNTLYLQMNSLRAEDTAVYYCATEGDNDAYDR | ID | DSVKG | (SEQ ID |
| | WGQGTTVTVSS (SEQ ID NO: 76) | NO: 6) | (SEQ ID | NO: 40) |
| | | | NO: 7) | |
| Li81 | EVQLLESGGGLVQPGGSLRLSCAASGFTFSAYEMKW | AYEM | VIGPSG | EGDND |
| variant | VRQAPGKGLEWVSVIGPSGGFTFYADSVKGRFTISRD | K (SEQ | GFTFYA | VFDS |
| D11 | NSKNTLYLQMNSLRAEDTAVYYCATEGDNDVFDSW | ID | DSVKG | (SEQ ID |
| | GQGTTVTVSS (SEQ ID NO: 77) | NO: 6) | (SEQ ID | NO: 41) |
| | | | NO: 7) | |
| Li81 | EVQLLESGGGLVQPGGSLRLSCAASGFTFSAYEMKW | AYEM | VIGPSG | EGDDD |
| variant | VRQAPGKGLEWVSVIGPSGGFTFYADSVKGRFTISRD | K (SEQ | GFTFYA | VFDM |
| E05 | NSKNTLYLQMNSLRAEDTAVYYCATEGDDDVFDM | ID | DSVKG | (SEQ ID |
| | WGQGTTVTVSS (SEQ ID NO: 78) | NO: 6) | (SEQ ID | NO: 42) |
| | | | NO: 7) | |
| Li81 | EVQLLESGGGLVQPGGSLRLSCAASGFTFSAYEMKW | AYEM | VIGPSG | EGYND |
| variant | VRQAPGKGLEWVSVIGPSGGFTFYADSVKGRFTISRD | K (SEQ | GFTFYA | AFDF |
| H04 | NSKNTLYLQMNSLRAEDTAVYYCATEGYNDAFDFW | ID | DSVKG | (SEQ ID |
| | GQGTTVTVSS (SEQ ID NO: 79) | NO: 6) | (SEQ ID | NO: 43) |
| | | | NO: 7) | |
| Li81 | EVQLLESGGGLVQPGGSLRLSCAASGFTFSAYEMKW | AYEM | VIGPSG | EGDDD |
| variant | VRQAPGKGLEWVSVIGPSGGFTFYADSVKGRFTISRD | K (SEQ | GFTFYA | AYDM |
| B04 | NSKNTLYLQMNSLRAEDTAVYYCATEGDDDAYDM | ID | DSVKG | (SEQ ID |
| | WGQGTTVTVSS (SEQ ID NO: 80) | NO: 6) | (SEQ ID | NO: 44) |
| | | | NO: 7) | |
| Li81 | EVQLLESGGGLVQPGGSLRLSCAASGFTFSAYEMKW | AYEM | VIGPSG | EQDYD |
| variant | VRQAPGKGLEWVSVIGPSGGFTFYADSVKGRFTISRD | K (SEQ | GFTFYA | TYDL |
| A02 | NSKNTLYLQMNSLRAEDTAVYYCATEQDYDTYDLW | ID | DSVKG | (SEQ ID |
| | GQGTTVTVSS (SEQ ID NO: 81) | NO: 6) | (SEQ ID | NO: 45) |
| | | | NO: 7) | |
| Li81 | EVQLLESGGGLVQPGGSLRLSCAASGFTFSAYEMKW | AYEM | VIGPSG | EGDDD |
| variant | VRQAPGKGLEWVSVIGPSGGFTFYADSVKGRFTISRD | K (SEQ | GFTFYA | AFDT |
| B12 | NSKNTLYLQMNSLRAEDTAVYYCATEGDDDAFDTW | ID | DSVKG | (SEQ ID |
| | GQGTTVTVSS (SEQ ID NO: 82) | NO: 6) | (SEQ ID | NO: 46) |
| | | | NO: 7) | |
| Li81 | EVQLLESGGGLVQPGGSLRLSCAASGFTFSAYEMKW | AYEM | VIGPSG | EADDD |
| variant | VRQAPGKGLEWVSVIGPSGGFTFYADSVKGRFTISRD | K (SEQ | GFTFYA | AFDI |
| H06 | NSKNTLYLQMNSLRAEDTAVYYCATEADDDAFDIW | ID | DSVKG | (SEQ ID |
| | GQGTTVTVSS (SEQ ID NO: 83) | NO: 6) | (SEQ ID | NO: 47) |
| | | | NO: 7) | |
| Li81 | EVQLLESGGGLVQPGGSLRLSCAASGFTFSAYEMKW | AYEM | VIGPSG | EGEND |
| variant | VRQAPGKGLEWVSVIGPSGGFTFYADSVKGRFTISRD | K (SEQ | GFTFYA | AFDM |
| H08 | NSKNTLYLQMNSLRAEDTAVYYCATEGENDAFDM | ID | DSVKG | (SEQ ID |
| | WGQGTTVTVSS (SEQ ID NO: 84) | NO: 6) | (SEQ ID | NO: 48) |
| | | | NO: 7) | |
| Li81 | EVQLLESGGGLVQPGGSLRLSCAASGFTFSAYEMKW | AYEM | VIGPSG | EGEYD |
| variant | VRQAPGKGLEWVSVIGPSGGFTFYADSVKGRFTISRD | K (SEQ | GFTFYA | TYDI |
| E07 | NSKNTLYLQMNSLRAEDTAVYYCATEGEYDTYDIW | ID | DSVKG | (SEQ ID |
| | GQGTTVTVSS (SEQ ID NO: 85) | NO: 6) | (SEQ ID | NO: 49) |
| | | | NO: 7) | |

In another embodiment, the anti-LINGO-1 antibody molecule includes a polypeptide comprising, consisting essentially of, or consisting of an immunoglobulin heavy chain variable region (VH), wherein at least the CDR3 region is at least 80%, 85%, 90% or 95% identical to a reference CDR3 sequence selected from the group consisting of SEQ ID NOs: 4, 8 and 17-49. In further embodiments, the CDR3 region is identical to a reference CDR3 sequence selected from the group consisting of SEQ ID NOs: 4, 8 and 17-49. In still further embodiments, the anti-LINGO-1 antibody molecule includes a polypeptide comprising, consisting essentially of, or consisting of an immunoglobulin heavy chain variable region (VH), wherein, the CDR1 and CDR2 regions are at least 80%, 85%, 90%, 95% or 100% identical to the CDR1 and CDR2 amino acid sequences of SEQ ID NOs: 2 and 3, respectively, and the CDR3 region is at least 80%, 85%, 90%, 95% or 100% identical to a CDR3 amino acid sequence selected from the group consisting of SEQ ID NOs: 4 and 17-34. In other embodiments, the anti-LINGO-1 antibody molecule includes a polypeptide comprising, consisting essentially of, or consisting of an immunoglobulin heavy chain variable region (VH), wherein the CDR1 and CDR2 regions are at least 80%, 85%, 90%, 95% or 100% identical to the CDR1 and CDR2 amino acid sequences of SEQ ID NOs: 6 and 7, respectively, and the CDR3 region is at least 80%, 85%, 90%, 95% or 100% identical to a CDR3 amino acid sequence selected from the group consisting of SEQ ID NOs: 8 and 35-49.

In another embodiment, the anti-LINGO-1 antibody molecule includes a polypeptide comprising, consisting essentially of, or consisting of an immunoglobulin heavy chain variable region (VH) in which the CDR1, CDR2, and CDR3 regions have polypeptide sequences which are identical to the CDR1, CDR2, and CDR3 groups shown in Table 3. In certain embodiments, the anti-LINGO-1 antibody molecule includes the VH polypeptide specifically or preferentially binds to LINGO-1.

In a further embodiment, the anti-LINGO-1 antibody molecule includes a polypeptide comprising, consisting essentially of, or consisting of a VH polypeptide at least 80%, 85%, 90% 95% or 100% identical to a reference VH polypeptide sequence selected from SEQ ID NOs: 1, 5 and 53-85. In one particular embodiment, the VH polypeptide comprises a CDR3 amino acid sequence selected from the group consisting of SEQ ID NOs: 4, 8 and 17-49.

In certain embodiments, the anti-LINGO-1 antibody molecule includes a polypeptide comprising, consisting essentially of, or consisting of a VH polypeptide selected from the group consisting of SEQ ID NOs: 1, 5 and 53-85. In certain embodiments, an antibody or antigen-binding fragment comprising the VH polypeptide specifically or preferentially binds to LINGO-1.

In another aspect, the anti-LINGO-1 antibody molecule includes a VH comprising the amino acids of SEQ ID NO: 1 or SEQ ID NO: 5. In certain embodiments, an antibody or antigen-binding fragment comprising the VH that specifically or preferentially binds to LINGO-1. In certain embodiments, an antibody or antigen-binding fragment thereof comprising, consisting essentially of, or consisting of a VH that specifically or preferentially binds to the same epitope as Li62, Li81 or a variant thereof as described in Table 3 or will competitively inhibit such a monoclonal antibody from binding to LINGO-1.

In certain embodiments, the anti-LINGO-1 antibody molecule includes a polypeptide, comprising, consisting essentially of, or consisting of an immunoglobulin heavy chain which is identical to the polypeptide of SEQ ID NO:146 except for a replacement of one or more of the following amino acids: W50, P53, 157 and/or W104. In some embodiments, W50 is replaced with an H, F, L, M, G, I, or D residue. In some embodiments, P53 is replaced with an L, S, T, W, or G residue. In some embodiments, 157 is replaced with a G, M, N, H, L, F, W, Y, S, P, V or T residue. In some embodiments, W104 is replaced with a V, H, S, Q, M, L, T, or I residue.

In certain embodiments, the anti-LINGO-1 antibody molecule includes a polypeptide, comprising, consisting essentially of, or consisting of an immunoglobulin heavy chain variable region which is identical to the polypeptide of SEQ ID NO:5 except for a replacement of amino acid P53. In some embodiments, P53 is replaced with an L, S, T, W, or G residue.

In certain embodiments, the anti-LINGO-1 antibody molecule includes a polypeptide, comprising, consisting essentially of, or consisting of an immunoglobulin heavy chain variable region which is identical to the polypeptide of SEQ ID NO:1 except for a replacement of one or more of the following amino acids: W50, P53, 157 and/or W104. In some embodiments, W50 is replaced with an H, F, L, M, G, I, or D residue. In some embodiments, P53 is replaced with an L, S, T, W, or G residue. In some embodiments, 157 is replaced with a G, M, N, H, L, F, W, Y, S, P, V or T residue. In some embodiments, W104 is replaced with a V, H, S, Q, M, L, T, or I residue.

In certain embodiments, the anti-LINGO-1 antibody molecule includes a polypeptide, comprising, consisting essentially of, or consisting of an immunoglobulin heavy chain variable region which is identical to the polypeptide of SEQ ID NO:66 except for a replacement of one or more of the following amino acids: W50, P53, 157 and/or W104. In some embodiments, W50 is replaced with an H, F, L, M, G, I, or D residue. In some embodiments, P53 is replaced with an L, S, T, W, or G residue. In some embodiments, 157 is replaced with a G, M, N, H, L, F, W, Y, S, P, V or T residue. In some embodiments, W104 is replaced with a V, H, S, Q, M, L, T, or I residue.

In certain embodiments, the anti-LINGO-1 antibody molecule includes one or more of the VH polypeptides described above specifically or preferentially binds to the same epitope as Li62, Li81 or a variant thereof as described in Table 3, or can competitively inhibit such an antibody from binding to LINGO-1.

In another embodiment, the anti-LINGO-1 antibody molecule includes a polypeptide comprising, consisting essentially of, or consisting of an immunoglobulin light chain variable region (VL), where at least one of the CDRs of the light chain variable region or at least two of the CDRs of the light chain variable region are at least 80%, 85%, 90% or 95% identical to reference heavy chain CDR1, CDR2, or CDR3 amino acid sequences from monoclonal LINGO-1 antibodies disclosed herein. Alternatively, the CDR1, CDR2 and CDR3 regions of the VL are at least 80%, 85%, 90% or 95% identical to reference light chain CDR1, CDR2, and CDR3 amino acid sequences from monoclonal LINGO-1 antibodies disclosed herein. Thus, according to this embodiment a light chain variable region of the antibody molecule has CDR1, CDR2, and CDR3 polypeptide sequences related to the polypeptides shown in Table 4. In certain embodiments, the anti-LINGO-1 antibody molecule comprising the VL polypeptide specifically or preferentially binds to LINGO-1.

TABLE 4
LINGO-1 Antibody VL Sequences

| Anti- | | VL | VL | VL |
|---|---|---|---|---|
| body | VL SEQUENCE | CDR1 | CDR2 | CDR3 |
| Li62 | DIQMTQSPSFLSASVGDSVA | RASQD | DASNL | QQYDT |
| | ITCRASQDISRYLAWYQQRP | ISRYL | QT (SEQ | LHPS |
| | GKAPKLLIYDASNLQTGVPS | A (SEQ | ID | (SEQ ID |
| | RFSGSGSGTDFTFTITSLQP | ID | NO: 11) | NO: 12) |
| | EDFGTYYCQQYDTLHPSFGP | NO: 10) | | |
| | GTTVDIK (SEQ ID NO: | | | |
| | 9) | | | |
| Li81 | DIQMTQSPATLSLSPGERAT | RASQS | DASNR | QQRSN |
| | LSCRASQSVSSYLAWYQQKP | VSSYL | AT (SEQ | WPMY |
| | GQAPRLLIYDASNRATGIPA | A (SEQ | ID NO: | T (SEQ |
| | RFSGSGSGTDFTLTISSLEP | ID | 15) | ID NO: |
| | EDFAVYYCQQRSNWPMYTFG | NO: 14) | | 16) |
| | QGTKLEIK (SEQ ID | | | |
| | NO: 13) | | | |

In another embodiment, the anti-LINGO-1 antibody molecule includes a polypeptide comprising, consisting essentially of, or consisting of an immunoglobulin light chain variable region (VL) in which the CDR1, CDR2, and CDR3 regions have polypeptide sequences which are identical to the CDR1, CDR2, and CDR3 groups shown in Table 4. In certain embodiments, an antibody or antigen-binding fragment comprising the VL polypeptide specifically or preferentially binds to LINGO-1.

In a further embodiment, the anti-LINGO-1 antibody molecule includes a polypeptide comprising, consisting essentially of, or consisting of a VL polypeptide at least 80%, 85%, 90% or 95% identical to a reference VL polypeptide sequence selected from SEQ ID NO: 9 or SEQ ID NO: 13, shown in Table 4. In certain embodiments, the anti-LINGO-1 antibody molecule includes comprising the VL polypeptide specifically or preferentially binds to LINGO-1. In another aspect, the anti-LINGO-1 antibody molecule includes a polypeptide comprising, consisting essentially of, or consisting of a VL polypeptide selected from SEQ ID NO: 9 or SEQ ID NO: 13, shown in Table 4. In certain embodiments, the anti-LINGO-1 antibody molecule comprising the VL polypeptide specifically or preferentially binds to LINGO-1.

In certain embodiments, the anti-LINGO-1 antibody molecule includes a polypeptide consisting essentially of, or consisting of an immunoglobulin light chain which is identical to the polypeptide of SEQ ID NO:145 except for a replacement of amino acid W94. In some embodiments, W94 is replaced with an A, D, L, N, G, Q, V, or S residue.

In certain embodiments, the anti-LINGO-1 antibody molecule includes a polypeptide, comprising, consisting essentially of, or consisting of an immunoglobulin light chain variable region which is identical to the polypeptide of SEQ ID NO:5 except for a replacement of amino acid W94. In some embodiments, W94 is replaced with an A, D, L, N, G, Q, V, or S residue.

In certain embodiments, the anti-LINGO-1 antibody molecule includes a polypeptide comprising, consisting essentially of, one or more of the VL polypeptides described above specifically or preferentially binds to the same epitope as Li62 or Li81, or will competitively inhibit such a monoclonal antibody from binding to LINGO-1.

In other embodiments, the anti-LINGO-1 antibody molecule comprises, consists essentially of or consists of a VH polypeptide, as shown in Table 3, and a VL polypeptide, as shown in Table 4, selected from the group consisting of: i) SEQ ID NO: 1 or SEQ ID NOs: 53-70 and SEQ ID NO: 9; and iii) SEQ ID NO: 5 or SEQ ID NOs: 71-85 and SEQ ID NO: 13.

In some embodiments, the anti-LINGO-1 antibody molecule comprises, consists essentially of or consists of an antibody heavy chain as shown below in SEQ ID NO:86, or an amino acid sequence at least 80%, 85%, 90% or 95% identical thereto.

(SEQ ID NO: 86)
EVQLLESGGGLVQPGGSLRLSCAASGFTFSAYEMKWVRQAPGKGLEWVSV
IGPSGGFTFYADISVKGRFTISRDNSKNTLYLQMNSLRAEDTAVYYCATE
GDNDAFDIWGQGTTVTVSSASTKGPISVFPLAPSSKSTSGGTAALGCLVK
DYFPEPVTVSWNSGALTSGVHTFPAVLQSSGLYSLSSVVTIVPSSSLGTQ
TYICNVNHKPSNTKVDKKVEPKSCDKTHTCPPCPAPELLGGPSVFLFPPK
PKDTLIMISRTPEVTCVVVDVSHEDPEVKFNWYVDGVEVHNAKTKPREEQ
YNSTYRVVSVLTVLHQDIWLNGKEYKCKVSNKALPAPIEKTISKAKGQPR
EPQVYTLPPSRDELTKNQVSLTCLVKGFYPSIDIAVEWESNGQPENNYKT
TPPVLDSDGSFFLYSKLTVDKSRWQQGNVFSCSVMHEALHNHYTIQKSLS
LSPG

In other embodiments, the anti-LINGO-1 antibody molecule comprises, consists essentially of or consists of an aglycosylated version of an antibody heavy chain. For example, an aglycosylated version of Li81 is described in PCT/US2008/000316, filed Jan. 9, 2008 and U.S. Pat. No. 8,128,926, which are incorporated herein by reference in its entirety. An aglycosylated version of the Li81 antibody was created by changing a single amino acid (T to A) in the Li81 heavy chain sequence. The sequence of an aglycosylated version of Li81 heavy chain (SEQ ID NO:50) is shown below. The single amino acid change is marked in bold and underlined: EVQLLESGGGLVQPGGSLRLSCAASGFTFSAYEMKWVRQAPGKGLEWVSV IGPSGGFTFYADISVKGRFTISRDNSKNTLYLQMNSLRAEDTAVYYCATE GDNDAFDIWGQGTTVTVSSASTKGPISVFPLAPSSKSTSGGTAALGCLVK DYFPEPVTVSWNSGALTSGVHTFPAVLQSSGLYSLSSVVTIVPSSSLGTQ TYICNVNHKPSNTKVDKKVEPKSCDKTHTCPPCPAPELLGGPSVFLFPPK PKDTLIMISRTPEVTCVVVDVSHEDPEVKFNWYVDGVEVHNAKTKPREEQ YNSAYRVVSVLTVLHQDIWLNGKEYKCKVSNKALPAPIEKTISKAKGQPR EPQVYTLPPSRDELTKNQVSLTCLVKGFYPSIDIAVEWESNGQPENNYKT TPPVLDSDGSFFLYSKLTVDKSRWQQGNVFSCSVMHEALHNHYTIQKSLS LSPG. (SEQ ID NO: 50)

The anti-LINGO-1 antibody molecule includes a heavy chain at least 80%, 85%, 90% or 95% identical to a reference polypeptide comprising the amino acids of SEQ ID NO:50 or 86. In certain embodiments, an antibody or antigen-binding fragment comprising the heavy chain specifically or preferentially binds to LINGO-1.

In one embodiment, the anti-LINGO-1 antibody molecule is a fully human anti-LINGO-1 monoclonal antibody engineered into an aglycosyl immunoglobulin G subclass 1 (IgG1) framework to reduce effector function (also referred to herein as anti-LINGO-1 Antibody 1. Histological and functional evaluations of LINGO-1 knock-out mice have been performed, and in vivo pharmacological activity of anti-LINGO-1 Antibody 1 has been demonstrated in several animal models of demyelination. Anti-LINGO-1 Antibody 1 has been characterized in vitro and in vivo based on the evaluation of binding characteristics, biological activity, and pharmacological activity. The results of these studies indicate that anti-LINGO-1 Antibody 1 has the following characteristics described in Table 1.

TABLE 1
Characteristics of Anti-LINGO-1 Antibody 1

Binds to LINGO-1 with similar high apparent affinity across
human, monkey, rat and mouse [symbol]
Is selective for LINGO-1 and does not bind the other LINGO
family members, LINGO-2, LINGO-3, or LINGO-4.
Enhances differentiation of primary rat, monkey, and human
oligodendrocytes in vitro.
Enhances axonal myelination in an in vitro rat dorsal root ganglion/
OPC co-culture bioassay [symbol]
Has reduced Fc (γ) and complement effector functions compared
to wild-type IgG1.
Is efficacious in animal models using biochemical and functional
readouts. Remyelination activity has been demonstrated in the rat
LPC model following systemic administration from [symbol] to 100 mg/kg.
Functional recovery in the rat MOG-EAE model has been
demonstrated following weekly systemic administration of 3 and
10 mg/kg.
Is efficacious in animal models when given in the presence of
interferon β.
Is efficacious in animal models when given in the presence of high
dose corticosteroids. [symbol]
indicates data missing or illegible when filed

In one embodiment, the antibody molecule includes a VH wherein the VH CDR1, CDR2, and CDR3 comprise the amino acids of SEQ ID NOs: 6, 7, and 8, respectively, or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise the amino acids of SEQ ID NOs: 2, 3, and 30, respectively, or an amino acid sequence substantially identical thereto (e.g., an amino acid sequence at least 80%, 85%, 90% or 95% identical thereto).

In another embodiment, the antibody molecule includes a VL wherein the VL CDR1, CDR2, and CDR3 comprise the amino acids of SEQ ID NOs: 14, 15, and 16, respectively, or an amino acid sequence substantially identical thereto (e.g., an amino acid sequence at least 80%, 85%, 90% or 95% identical thereto).

In another embodiment, the antibody molecule includes a VH wherein the VL CDR1, CDR2, and CDR3 comprise the amino acids of SEQ ID NOs: 10, 11, and 12, respectively, or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise the amino acids of SEQ ID NOs: 6, 7, and 8, respectively; and a VL wherein the VL CDR1, CDR2, and CDR3 comprise the amino acids of SEQ ID NOs: 14, 15, and 16, respectively; or an amino acid sequence substantially identical thereto (e.g., an amino acid sequence at least 80%, 85%, 90% or 95% identical thereto).

In one embodiment, the antibody molecule includes a VH wherein the VH CDR1, CDR2, and CDR3 comprise the amino acids of SEQ ID NOs: 2, 3, and 30, respectively; and a VL wherein the VL CDR1, CDR2, and CDR3 comprise the amino acids of SEQ ID NOs: 10, 11, and 12, respectively; or an amino acid sequence substantially identical thereto (e.g., an amino acid sequence at least 80%, 85%, 90% or 95% identical thereto).

In one embodiment, the antibody molecule includes a VH that includes the amino acid sequence of SEQ ID NO: 5, or an amino acid sequence substantially identical thereto (e.g., an amino acid sequence at least 80%, 85%, 90% or 95% identical to said SEQ ID NO: 5).

In one embodiment, the antibody molecule includes a VH that includes the amino acid sequence of SEQ ID NO:66, or an amino acid sequence substantially identical thereto (e.g., an amino acid sequence at least 80%, 85%, 90% or 95% identical to said SEQ ID NO: 66).

In one embodiment, the antibody molecule includes a VL that includes the amino acid sequence of SEQ ID NO:13, or an amino acid sequence substantially identical thereto (e.g., an amino acid sequence at least 80%, 85%, 90% or 95% identical to said SEQ ID NO: 13).

In one embodiment, the antibody molecule includes a VL that includes the amino acid sequence of SEQ ID NO:9, or an amino acid sequence substantially identical thereto (e.g., an amino acid sequence at least 80%, 85%, 90% or 95% identical to said SEQ ID NO: 9).

In one embodiment, the antibody molecule includes a VH that includes the amino acid sequence of SEQ ID NO:5, or an amino acid sequence substantially identical thereto (e.g., an amino acid sequence at least 80%, 85%, 90% or 95% identical to said SEQ ID NO: 5); and a VL that includes the amino acid sequence of SEQ ID NO: 13, or an amino acid sequence substantially identical thereto (e.g., an amino acid sequence at least 80%, 85%, 90% or 95% identical to said SEQ ID NO: 13).

In one embodiment, the antibody molecule includes a VH that includes the amino acid sequence of SEQ ID NO:66, or an amino acid sequence substantially identical thereto (e.g., an amino acid sequence at least 80%, 85%, 90% or 95% identical to said SEQ ID NO: 66); and a VL that includes the amino acid sequence of SEQ ID NO: 9, or an amino acid sequence substantially identical thereto (e.g., an amino acid sequence at least 80%, 85%, 90% or 95% identical to said SEQ ID NO: 9).

In another embodiment, the antibody molecule includes a heavy chain as shown below, comprising the amino acid sequence of SEQ ID NO: 275, or a sequence substantially identical thereto (e.g., an amino acid sequence at least 80%, 85%, 90% or 95% identical thereto), as follows:

(SEQ ID NO: 275)
EVQLLESGGG LVQPGGSLRL SCAASGFTFS AYEMKWVRQA PGKGLEWVSV
IGPSGGFTFY ADSVKGRFTI SRDNSKNTLY LQMNSLRAED TAVYYCATEG
DNDAFDIWGQ GTTVTVSSAS TKGPSVFPLA PSSKSTSGGT AALGCLVKDY
FPEPVTVSWN SGALTSGVHT FPAVLQSSGL YSLSSVVTVP SSSLGTQTYI
CNVNHKPSNT KVDKKVEPKS CDKTHTCPPC PAPELLGGPS VFLFPPKPKD
TLMISRTPEV TCVVVDVSHE DPEVKFNWYV DGVEVHNAKT KPREEQYNSA
YRVVSVLTVL HQDWLNGKEY KCKVSNKALP APIEKTISKA KGQPREPQVY
TLPPSRDELT KNQVSLTCLV KGFYPSDIAV EWESNGQPEN NYKTTPPVLD
SDGSFFLYSK LTVDKSRWQQ GNVFSCSVMH EALHNHYTQK SLSLSPG.

In other embodiments, the antibody molecule includes a light chain as shown below comprising the amino acid sequence of SEQ ID NO: 276, or a sequence substantially identical thereto (e.g., an amino acid sequence at least 80%, 85%, 90% or 95% identical thereto), as follows:

(SEQ ID NO: 276)
DIQMTQSPAT LSLSPGERAT LSCRASQSVS SYLAWYQQKP GQAPRLLIYD
ASNRATGIPA RFSGSGSGTD FTLTISSLEP EDFAVYYCQQ RSNWPMYTFG
QGTKLEIKRT VAAPSVFIFP PSDEQLKSGT ASVVCLLNNF YPREAKVQWK
VDNALQSGNS QESVTEQDSK DSTYSLSSTL TLSKADYEKH KVYACEVTHQ
GLSSPVTKSF NRGEC.

Any of the polypeptides described above may further include additional polypeptides, e.g., a signal peptide to direct secretion of the encoded polypeptide, antibody constant regions as described herein, or other heterologous polypeptides as described herein. Additionally, polypeptides of the invention include polypeptide fragments as described elsewhere. Additionally polypeptides of the invention include fusion polypeptide, Fab fragments, and other derivatives, as described herein.

Also, as described in more detail elsewhere herein, the present invention includes compositions comprising the polypeptides described above.

It will also be understood by one of ordinary skill in the art that LINGO-1 antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

### Soluble LINGO Antagonists and Fusion Proteins

In another embodiment, the reparative agent, e.g., the antagonist of LINGO-1, is a soluble LINGO molecule, e.g., a LINGO-1 molecule (e.g., a fragment of LINGO-1), or a soluble form of a component of the LINGO-1 complex (e.g., a soluble form of NgR1, p75, or TAJ (TROY)).

In certain embodiments, a soluble LINGO molecule or a LINGO-1 antibody molecule comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

An antibody molecule, a soluble form of LINGO-1, or a complex component, as described herein, can be used alone or functionally linked (e.g., by chemical coupling, genetic or polypeptide fusion, non-covalent association or otherwise) to a second moiety, a heterologous moiety, e.g., a heterologous polypeptide. The term “heterologous” as applied to a polynucleotide or a polypeptide, means that a portion with which it is not naturally linked in nature. For example, the polynucleotide or polypeptide is derived from a distinct entity from that of the rest of the entity to which it is being compared. For instance, as used herein, a “heterologous polypeptide” to be fused to a LINGO-1 antibody molecule is derived from a non-immunoglobulin polypeptide of the same species, or an immunoglobulin or non-immunoglobulin polypeptide of a different species.

Exemplary heterologous moieties include, but are not limited to, an immunoglobulin Fc domain, serum albumin, pegylation, a GST, Lex-A and an MBP polypeptide sequence. The fusion proteins may additionally include a linker sequence joining the first moiety, e.g., the antibody molecule, the soluble form of LINGO-1 or the complex component, to the second moiety. In other embodiments, additional amino acid sequences can be added to the N- or C-terminus of the fusion protein to facilitate expression, steric flexibility, detection and/or isolation or purification. For example, a soluble form of LINGO-1 or a complex component can be fused to a heavy chain constant region of the various isotypes, including: IgG1, IgG2, IgG3, IgG4, IgM, IgA1, IgA2, IgD, and IgE.

It shall be understood that the antibody molecules and soluble or fusion proteins described herein can be functionally linked (e.g., by chemical coupling, genetic fusion, non-covalent association or otherwise) to one or more other molecular entities, such as an antibody (e.g., a bispecific or a multispecific antibody), among others.

In one embodiment, the fusion protein includes the extracellular domain of LINGO or the complex component (or a sequence homologous thereto), and, e.g., fused to, a human immunoglobulin Fc chain, e.g., human IgG (e.g., human IgG1 or human IgG2, or a mutated form thereof). The Fc sequence can be mutated at one or more amino acids to reduce effector cell function, Fc receptor binding and/or complement activity.

In certain embodiments, an anti-LINGO-1 antibody molecule can comprise, consist essentially of, or consist of, a fusion protein. Fusion proteins in this context are chimeric molecules which comprise, for example, an immunoglobulin antigen-binding domain with at least one target binding site, and at least one heterologous portion. The amino acid sequences can normally exist in separate proteins that are brought together in the fusion polypeptide or they may normally exist in the same protein, but are placed in a new arrangement in the fusion polypeptide. Fusion proteins can be created, for example, by chemical synthesis, or by creating and translating a polynucleotide in which the peptide regions are encoded in the desired relationship.

### Nucleic Acid Molecule/Recombinant Expression

Nucleic acid molecules, host cells and vectors that include a nucleotide sequence encoding any of the polypeptides, e.g., reparative agents and immunomodulators, described herein, are also encompassed by the invention.

In one exemplary embodiment, an isolated polynucleotide comprising, consisting essentially of, or consisting of a nucleic acid encoding an immunoglobulin heavy chain variable region (VH) of an anti-LINGO-1 antibody molecule, where at least one of the CDRs of the heavy chain variable region or at least two of the CDRs of the heavy chain variable region are at least 80%, 85%, 90% or 95% identical to reference heavy chain CDR1, CDR2, or CDR3 amino acid sequences of Li62 or Li81 or variants thereof as described in Table 3 is provided. Alternatively, the CDR1, CDR2, and CDR3 regions of the VH are at least 80%, 85%, 90% or 95% identical to reference heavy chain CDR1, CDR2, and CDR3 amino acid sequences of Li62 or Li81 or variants thereof as described in Table 3. Thus, according to this embodiment, a heavy chain variable region of the invention has CDR1, CDR2, or CDR3 polypeptide sequences related to the polypeptide sequences shown in Table 3.

In another exemplary embodiment, an isolated polynucleotide comprising, consisting essentially of, or consisting of a nucleic acid encoding an immunoglobulin light chain variable region (VL) of an anti-LINGO-1 antibody molecule, where at least one of the CDRs of the light chain variable region or at least two of the CDRs of the light chain variable region are at least 80%, 85%, 90% or 95% identical to reference light chain CDR1, CDR2, or CDR3 amino acid sequences from monoclonal LINGO-1 antibodies disclosed herein is provided. Alternatively, the CDR1, CDR2, and CDR3 regions of the VL are at least 80%, 85%, 90% or 95% identical to reference light chain CDR1, CDR2, and CDR3 amino acid sequences from monoclonal LINGO-1 antibodies disclosed herein. Thus, according to one embodiment, a light chain variable region of the invention has CDR1, CDR2, or CDR3 polypeptide sequences related to the polypeptide sequences shown in Table 4.

Any of the polynucleotides described above may further include additional nucleic acids, encoding, e.g., a signal peptide to direct secretion of the encoded polypeptide, antibody constant regions as described herein, or other heterologous polypeptides as described herein.

Compositions comprising the polynucleotides comprising one or more of the polynucleotides described above are also disclosed. In one embodiment, the compositions comprising a first polynucleotide and second polynucleotide wherein said first polynucleotide encodes a VH polypeptide as described herein and wherein said second polynucleotide encodes a VL polypeptide as described herein.

Also disclosed are fragments of the polynucleotides of the invention, as described elsewhere. Additionally polynucleotides which encode fusion polynucleotides, Fab fragments, and other derivatives, as described herein, are also contemplated by the invention.

The polynucleotides can be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Recombinant expression of a polypeptide described herein, e.g., an antibody molecule that binds to LINGO-1, requires construction of an expression vector containing the polynucleotide that encodes the polypeptide, e.g., the antibody molecule. Once a polynucleotide encoding the antibody molecule or a heavy or light chain of an antibody, or portion thereof (preferably containing the heavy or light chain variable domain), has been obtained, the vector for the production of the antibody molecule may be produced by recombinant DNA technology using techniques known in the art. Thus, methods for preparing a protein by expressing a polynucleotide containing a polypeptide encoding nucleotide sequence are described herein and in U.S. Pat. No. 8,058,406, the contents of which are incorporated by reference in their entirety.

Methods known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Replicable vectors comprising a nucleotide sequence encoding a polypeptide described herein (e.g., an anti-LINGO-1 antibody molecule, or a heavy or light chain thereof, or a heavy or light chain variable domain) operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

The term “vector” or “expression vector” is used herein to mean vectors used as a vehicle for introducing into and expressing a desired gene in a host cell. As known to those skilled in the art, such vectors can easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals.

In one embodiment, the cloned variable region genes are inserted into an expression vector along with the heavy and light chain constant region genes (preferably human) synthetic as discussed above. In one embodiment, this is effected using a proprietary expression vector of Biogen MA, Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed, e.g., in U.S. Pat. No. 6,413,777.

In other embodiments, the LINGO-1 antibody molecules can be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of binding polypeptides in eukaryotic host cells. Compatible IRES sequences are disclosed in U.S. Pat. No. 6,193,980 which is also incorporated herein. Those skilled in the art will appreciate that such expression systems can be used to effectively produce the full range of polypeptides disclosed in the instant application.

More generally, once the vector or DNA sequence encoding a monomeric subunit of the polypeptide, e.g., the anti-LINGO-1 antibody molecule, has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce a polypeptide for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed in U.S. Pat. No. 8,058,406, the contents of which are incorporated by reference herein in its entirety.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

A variety of host-expression vector systems may be utilized to express polypeptides, e.g., antibody molecules, for use in the methods described herein. These include, but are not limited to, microorganisms such as bacteria (e.g., E. coli, B. subtilis) transformed with recombinant bacteriophage DNA, plasmid DNA or cosmid DNA expression vectors containing polypeptide coding sequences; yeast (e.g., Saccharomyces, Pichia) transformed with recombinant yeast expression vectors containing antibody coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Typically, bacterial cells such as Escherichia coli, and more typically, eukaryotic cells, especially for the expression of whole recombinant antibody molecule, are used for the expression of a recombinant polypeptide or antibody molecule. For example, mammalian cells such as Chinese hamster ovary cells (CHO), in conjunction with a vector such as the major intermediate early gene promoter element from human cytomegalovirus is an effective expression system for polypeptides antibodies (Foecking et al., Gene 45:101 (1986); Cockett et al., Bio/Technology 8:2 (1990)).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, W138, R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

CHO cells are particularly preferred. In certain embodiment, the antibody molecules are expressed in CHO cells stably transfected with expression vectors containing the IgG₁-agly heavy light chain structural genes specific to the human LINGO-1 protein. A native human kappa light chain signal peptide and a human heavy chain signal peptide, which are post-translationally removed by endoplasmic reticulum-associated signal peptidase, can be used to direct secretion of the anti-LINGO-1 antibody molecule. The antibody molecule can be purified from the media and formulated as a liquid. The antibody molecule can consists of 2 heavy and 2 light chains connected by inter-chain disulfide bonds. In one embodiment, the mass of the intact antibody molecule is approximately 144.4 kDa.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds), Current Prolocols in Human Genetics, John Wiley & Sons, N Y (1994); Colberre-Garapin et al., J. Mol. Biol. 150:1 (1981), which are incorporated by reference herein in their entireties.

Additional methods and host systems expression, production and/or purification of the polypeptides, e.g., antibodies, are disclosed in U.S. Pat. No. 8,058,406, the contents of which are incorporated by reference herein in its entirety.

### Nucleic Acid Antagonists

In certain embodiments, the LINGO-1 antagonist inhibits the expression of nucleic acid encoding a LINGO-1. Examples of such LINGO-1 antagonists include nucleic acid molecules, for example, antisense molecules, ribozymes, RNAi double stranded molecules, triple helix molecules, microRNA molecules that hybridize to a nucleic acid encoding a LINGO-1, or a transcription regulatory region, and block or reduce mRNA expression of LINGO-1.

An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire LINGO-1 coding strand, or to only a portion thereof. In another embodiment, the antisense nucleic acid molecule is antisense to a “noncoding region” of the coding strand of a nucleotide sequence encoding LINGO-1 (e.g., the 5′ and 3′ untranslated regions). Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted. Exemplary antisense compounds include DNA or RNA sequences that specifically hybridize to the target nucleic acid, e.g., the mRNA encoding LINGO-1. The complementary region can extend for between about 8 to about 80 nucleobases. The compounds can include one or more modified nucleobases, which are known in the art. Descriptions of nucleic acid agents are available. See, e.g., U.S. Pat. Nos. 4,987,071; 5,116,742; and U.S. Pat. No. 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

In still another embodiment, the nucleic acid molecule is a ribozyme. A ribozyme having specificity for a LINGO-1-encoding nucleic acid can include one or more sequences complementary to the nucleotide sequence of a LINGO-1 mRNA, and a sequence having known catalytic sequence responsible for mRNA cleavage (see U.S. Pat. No. 5,093,246 or Haselhoff and Gerlach (1988) Nature 334:585-591; Cech et al. U.S. Pat. No. 4,987,071; and Cech et al. U.S. Pat. No. 5,116,742; Bartel, D. and Szostak, J. W. (1993) Science 261:1411-1418).

In one embodiment, the nucleic acid molecule is a microRNA molecule. A microRNA having specificity for a LINGO-1-encoding nucleic acid can include one or more sequences complementary to the nucleotide sequence of a LINGO-1 mRNA, which can result in gene silencing via translational repression or target degradation (see Bartel DP (2009) Cell 136 (2): 215-33; Kusenda B, et al. (2006) Biomed Pap Med Fac Univ Palacky Olomouc Czech Repub 150 (2): 205-15).

LINGO-1 gene expression can be inhibited by targeting nucleotide sequences complementary to the regulatory region of the LINGO-1 (e.g., the LINGO-1 promoter and/or enhancers) to form triple helical structures that prevent transcription of the LINGO-1 gene in target cells. See generally, Helene, C. (1991) Anticancer Drug Des. 6:569-84; Helene, C. (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule.

A LINGO-1 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44; Hyrup B. et al. (1996) Bioorganic & Medicinal Chemistry 4: 5-23).

### Immunomodulatory Agents

Several immunomodulatory agents are presently used to modify the course of multiple sclerosis in patients. Such agents include, but are not limited to, an IFN-β 1 molecule; a polymer of glutamic acid, lysine, alanine and tyrosine, e.g., glatiramer; an antibody or fragment thereof against alpha-4 integrin, e.g., natalizumab; an anthracenedione molecule, e.g., mitoxantrone; a fingolimod, e.g., FTY720; a dimethyl fumarate, e.g., an oral dimethyl fumarate; an antibody to the alpha subunit of the IL-2 receptor of T cells (CD25), e.g., daclizumab; an antibody against CD52, e.g., alemtuzumab; an inhibitor of a dihydroorotate dehydrogenase, e.g., teriflunomide; an antibody to CD20, e.g., ocrelizumab; and a corticosteroid. The reparative agents disclosed herein can be used in combination with any of these agents.

Exemplary immunomodulatory agents are described in more detail as follows.

### IFNβ Agents (Beta Interferons)

One known therapy for MS includes treatment with interferon beta. Interferons (IFNs) are natural proteins produced by the cells of the immune systems of most animals in response to challenges by foreign agents such as viruses, bacteria, parasites and tumor cells. Interferons belong to the large class of glycoproteins known as cytokines. Interferon beta has 165 amino acids. Interferons alpha and beta are produced by many cell types, including T-cells and B-cells, macrophages, fibroblasts, endothelial cells, osteoblasts and others, and stimulate both macrophages and NK cells. Interferon gamma is involved in the regulation of immune and inflammatory responses. It is produced by activated T-cells and Th1 cells.

Several different types of interferon are now approved for use in humans. Interferon alpha (including forms interferon alpha-2a, interferon alpha-2b, and interferon alfacon-1) was approved by the United States Food and Drug Administration (FDA) as a treatment for Hepatitis C. There are two currently FDA-approved types of interferon beta. Interferon beta 1a (Avonex®) is identical to interferon beta found naturally in humans, and interferon beta 1b (Betaseron®) differs in certain ways from interferon beta 1a found naturally in humans, including that it contains a serine residue in place of a cysteine residue at position 17. Other uses of interferon beta have included treatment of AIDS, cutaneous T-cell lymphoma, Acute Hepatitis C (non-A, non-B), Kaposi's sarcoma, malignant melanoma, hairy cell leukemia, and metastatic renal cell carcinoma.

IFNβ agents can be administered to the subject by any method known in the art, including systemically (e.g., orally, parenterally, subcutaneously, intravenously, rectally, intramuscularly, intravitreally, intraperitoneally, intranasally, transdermally, or by inhalation or intracavitary installation). Typically, the IFNβ agents are administered subcutaneously, or intramuscularly.

IFNβ agents can be used to treat those subjects determined to be “responders” using the methods described herein. In one embodiment, the IFNβ agents are used as a monotherapy (i.e., as a single “disease modifying therapy”) although the treatment regimen can further comprise the use of “symptom management therapies” such as antidepressants, analgesics, anti-tremor agents, etc. In one embodiment, the IFNβ agent is an IFNβ-1A agent (e.g., Avonex®, Rebif®). In another embodiment, the INFβ agent is an INFβ-1B agent (e.g., Betaseron®, Betaferon®, Extavia®).

Avonex®, an Interferon β-1a, is indicated for the treatment of patients with relapsing forms of MS that are determined to be responders using the methods described herein to slow the accumulation of physical disability and decrease the frequency of clinical exacerbations. Avonex® (Interferon beta-1a) is a 166 amino acid glycoprotein with a predicted molecular weight of approximately 22,500 daltons. It is produced by recombinant DNA technology using genetically engineered Chinese Hamster Ovary cells into which the human interferon beta gene has been introduced. The amino acid sequence of Avonex® is identical to that of natural human interferon beta. The recommended dosage of Avonex® (Interferon beta-1a) is 30 mcg injected intramuscularly once a week. Avonex® is commercially available as a 30 mcg lyophilized powder vial or as a 30 mcg prefilled syringe.

Interferon beta 1a (Avonex®) is identical to interferon beta found naturally in humans (AVONEX®, i.e., Interferon beta Ia (SwissProt Accession No. P01574 and gi:50593016). The sequence of interferon beta is:

(SEQ ID NO: 277)
MTNKCLLQIALLLCFSTTALSMSYNLLGFLQRSSNFQCQKLLWQLNGRLE
YCLKDRMNFDIPEEIKQLQQFQKEDAALTIYEMLQNIFAIFRQDSSSTGW
NETIVENLLANVYHQINHLKTVLEEKLEKEDFTRGKLMSSLHLKRYYGRI
LHYLKAKEYSHCAWTIVRVEILRNFYFINRLTGYLRN.

Methods for making Avonex® are known in the art.

Treatment of responders identified using the methods described herein further contemplates that compositions (e.g., IFN beta 1 a molecules) having biological activity that is substantially similar to that of AVONEX® will permit successful treatment similar to treatment with AVONEX® when administered in a similar manner. Such other compositions include, e.g., other interferons and fragments, analogues, homologues, derivatives, and natural variants thereof with substantially similar biological activity. In one embodiment, the 11\193 agent is modified to increase one or more pharmacokinetic properties. For example, the 11\193 agent can be a modified form of interferon 1a to include a pegylated moiety. PEGylated forms of interferon beta 1a are described in, e.g., Baker, D. P. et al. (2006) Bioconjug Chem 17(1):179-88; Arduini, R M et al. (2004) Protein Expr Purif 34(2):229-42; Pepinsky, R B et al. (2001) J. Pharmacol. Exp. Ther. 297(3):1059-66; Baker, D. P. et al. (2010) J Interferon Cytokine Res 30(10):777-85 (all of which are incorporated herein by reference in their entirety, and describe a human interferon beta 1a modified at its N-terminal alpha amino acid to include a PEG moiety, e.g., a 20 kDa mPEG-O-2-methylpropionaldehyde moiety). Pegylated forms of IFN beta 1a can be administered by, e.g., injectable routes of administration (e.g., subcutaneously).

Rebif® is also an Interferon β-1a agent, while Betaseron®, Betaferon®, and Extavia® are Interferon β-1b agents. Both Rebif® and Betaseron® are formulated for administration by subcutaneous injection.

Dosages of IFNβ agents to administer can be determined by one of skill in the art, and include clinically acceptable amounts to administer based on the specific interferon-beta agent used. For example, AVONEX® is typically administered at 30 microgram once a week via intramuscular injection. Other forms of interferon beta 1a, specifically REBIF®, is administered, for example, at 22 microgram three times a week or 44 micrograms once a week, via subcutaneous injection. Interferon beta-1A can be administered, e.g., intramuscularly, in an amount of between 10 and 50 μg. For example, AVONEX® can be administered every five to ten days, e.g., once a week, while Rebif® can be administered three times a week.

### Anti-VLA4 Antibody (e.g., Natalizumab (Tysabri®))

Anti-VLA4 antibodies (e.g., Natalizumab) inhibit the migration of leukocytes from the blood to the central nervous system. These antibodies bind to VLA-4 (also called α4β1) on the surface of activated T-cells and other mononuclear leukocytes. They can disrupt adhesion between the T-cell and endothelial cells, and thus prevent migration of mononuclear leukocytes across the endothelium and into the parenchyma. As a result, the levels of pro-inflammatory cytokines can also be reduced. Natalizumab can decrease the number of brain lesions and clinical relapses and accumulation of disability in patients with relapse remitting multiple sclerosis and relapsing secondary-progressive multiple sclerosis.

Natalizumab and related VLA-4 binding antibodies are described, e.g., in U.S. Pat. No. 5,840,299. Monoclonal antibodies 21.6 and HP1/2 are exemplary murine monoclonal antibodies that bind VLA-4. Natalizumab is a humanized version of murine monoclonal antibody 21.6 (see, e.g., U.S. Pat. No. 5,840,299). A humanized version of HP 1/2 has also been described (see, e.g., U.S. Pat. No. 6,602,503). Several additional VLA-4 binding monoclonal antibodies, such as HP2/1, HP2/4, L25 and P4C2, are described, e.g., in U.S. Pat. No. 6,602,503; Sanchez-Madrid et al, (1986) Eur. J. Immunol 16:1343-1349; Hemler et al, (1987) J Biol. Chem. 2:11478-11485; Issekutz et al. (1991) J Immunol 147: 109 (TA-2 mab); Pulido et al. (1991) J Biol. Chem. 266: 10241-10245; and U.S. Pat. No. 5,888,507). The contents of the aforesaid publications (including the antibody compositions, dosages, methods of administration and production) are incorporated herein by reference in their entirety.

### Dimethyl Fumarate (Tecfidera®)

Dimethyl fumarate, DMF, (Tecfidera®) is a fumaric acid ester. DMF is thought to decrease leukocyte passage through the blood brain barrier and exert neuroprotective effects by the activation of antioxidative pathways, specifically through activation of the Nrf-2 pathway (Lee et al. (2008) Int MS Journal 15: 12-18). Research also suggests that BG-12® has the potential to reduce the activity and impact of inflammatory cells on the CNS and induce direct cytoprotective responses in CNS cells. These effects may enhance the CNS cells' ability to mitigate the toxic inflammatory and oxidative stress that plays a role in MS pathophysiology.

### Glatiramer Acetate (Copaxone®)

Copaxone® (glatiramer acetate) consists of the acetate salts of synthetic polypeptides, specifically the four naturally occurring amino acids: L-glutamic acid, L-alanine, L-tyrosine, and L-lysine (Bornstein et al. (1987) N Engl J Med. 317: 408-414). Copaxone® exhibits structural similarity to myelin basic protein and is thought to function as an immune modulator by shifting the T helper cell type 1 response towards a T helper cell type 2 response (Duda et al. (2000) J Clin Invest 105: 967-976; Nicholas et al. (2011) Drug Design, Development, and Therapy 5: 255-274).

### Mitoxantrone (Novantrone®, an Anthracenedione Molecule)

Mitoxantrone is an anthracenedione molecule (1,4-dihydroxy-5,8-bis[2-(2-hydroxyethylamino) ethylamino]-anthracene-9,10-dione) and a type II topoisomerase inhibitor that disrupts DNA synthesis and repair of cells. It is used to treat cancers and MS. Mitoxantrone is used to treat several forms of advancing MS, including secondary progressive MS, progressive relapsing MS, and advanced relapsing-remitting MS. For example, mitoxantrone is effective in slowing the progression of secondary progressive MS and extending the time between relapses in relapsing-remitting MS and progressive relapsing MS (Fox E (2006) Clin Ther 28 (4): 461-74).

### Fingolimod (Gilenya®; Sphingosine 1-Phosphate Receptor Modulator)

Fingolimod is an immunomodulating drug, approved for treating MS. It has reduced the rate of relapses in relapsing-remitting multiple sclerosis by over half, but may have serious adverse effects. Fingolimod is a sphingosine 1-phosphate receptor modulator, which sequesters lymphocytes in lymph nodes, preventing them from moving to the central nervous system for autoimmune responses in MS.

### Antibodies to the Alpha Subunit of the IL-2 Receptor of T Cells (CD25) (e.g., Daclizumab Hyp; ZINBRYTA®)

An antibody to the alpha subunit of the IL-2 receptor of T cells (CD25), e.g., daclizumab HYP, can be used in the methods and compositions disclosed herein. Daclizumab HYP is a therapeutic humanized monoclonal antibody to the alpha subunit of the IL-2 receptor of T cells (CD25). Daclizumab HYP showed efficacy in reducing lesions and annualized relapse rate in patients with relapsing-remitting multiple sclerosis (Kappos et al. (2015). N. Engl. J. Med. 373 (15): 1418-28).

### Antibody Against CD52, e.g., Alemtuzumab

Antibodies against CD52, e.g., alemtuzumab (currently under further development as Lemtrada®), bind to CD52, which is a protein present on the surface of mature lymphocytes, but not on stem cells. Phase III studies reported positive results comparing alemtuzumab with Rebif® (high-dose subcutaneous interferon beta-1a) in the treatment of patients with relapsing-remitting MS (RRMS). Alemtuzumab has been approved in Europe.

### Antibody to CD20, e.g., Ocrelizumab

Antibodies against CD20, e.g., ocrelizumab, rituximab, of atumumab, target mature B lymphocytes. Phase 2 clinical studies of rituximab and ocrelizumab in relapse remitting MS have demonstrated a statistically significant reduction in disease activity measured by brain lesions (e.g., measured by MRI scans) and relapse rate compared to placebo. Phase 3 studies of ocrelizumab showed both reduction in relapse rate and disability compared to interferon beta-1a (e.g., Rebif®).

### Inhibitors of Dihydroorotate Dehydrogenase, e.g., Teriflunomide

Inhibitors of dihydroorotate dehydrogenase, e.g., teriflunomide, inhibit pyrimidine synthesis. Teriflunomide (also known as A77 1726 or) is an active metabolite of leflunomide. Teriflunomide inhibits rapidly dividing cells, including activated T cells, which are thought to drive the disease process in MS. Teriflunomide was investigated in clinical trials as a medication for treating MS. (Vollmer EMS News (May 28, 2009)).

### Steroids

Steroids, e.g., corticosteroid, and ACTH agents can be used to treat acute relapses in relapsing-remitting MS or secondary progressive MS. Such agents include, but are not limited to, Depo-Medrol®, Solu-Medrol®, Deltasone®, Delta-Cortef®, Medrol®, Decadron®, and Acthar®.

One or more of the aforesaid immunomodulatory agents can be used in combination with the reparative agents disclosed herein, as described in more detail below and exemplified by the combination of IFN-b and anti-LINGO-1 Antibody Therapy.

### Therapeutic Methods

Reparative agents, such as LINGO-1 antagonists, can relieve NgR1-mediated inhibition of axonal regeneration and dendritic arborization that normally takes place in CNS neurons. This is beneficial in situations where axonal repair or neurite sprouting is needed in the brain or spinal cord following CNS injury. Spinal cord injury, including partial or complete crush or severance, exemplifies a situation in which axonal repair is needed, but is normally inhibited through operation of the NgR1 pathway.

In addition, LINGO-1 is expressed in oligodendrocytes, and contributes to oligodendrocyte biology. Soluble derivatives of LINGO-1, polynucleotides (e.g. RNAi), as well as certain antibodies which specifically bind to LINGO-1 can act as antagonists to LINGO-1 function in oligodendrocytes, enhancing differentiation and survival of oligodendrocytes and promoting myelination of neurons in vitro and in vivo. This can be beneficial for treating or preventing disorders or conditions involving demyelination and dysmyelination.

Examples of diseases or disorders in which axonal extension and/or neurite sprouting in the brain, and/or oligodendrocyte proliferation, differentiation and survival, and/or myelination or remyelination, can be beneficial include, but are not limited to, CNS demyelinating diseases, CNS injury, stroke, multiple sclerosis (MS), optic neuritis (e.g., acute optic neuritis), idiopathic inflammatory demyelinating disease, transverse myelitis, neuromyelitis optica (NMO), vitamin B12 deficiency, progressive multifocal leukoencephalopathy (PML), encephalomyelitis (EPL), acute disseminated encephalomyelitis (ADEM), central pontine myelolysis (CPM), Wallerian Degeneration, adrenoleukodystrophy, Alexander's disease, Pelizaeus Merzbacher disease (PMZ), leukodystrophies, traumatic glaucoma, periventricular leukomalatia (PVL), essential tremor, white matter stroke, stroke, or radiation or toxic induced white matter injuryand other neurodegenerative diseases or disorders such as multiple sclerosis, adrenoleukodystrophy, periventricular leukomalatia (PVL), Globoid cell Leucodystrophy (Krabbe's disease) and Wallerian Degenerationamylotrophic lateral sclerosis (ALS), Huntington's disease, Alzheimer's disease, Parkinson's disease, spinal cord injury, traumatic brain injury, post radiation injury, neurologic complications of chemotherapy, neuropathy (e.g., diabetic neuropathy), acute ischemic optic neuropathy, isolated vitamin E deficiency syndrome, AR, Bassen-Kornzweig syndrome, Marchiafava-Bignami syndrome, metachromatic leukodystrophy, trigeminal neuralgia, Bell's palsy, spinal cord injury, traumatic glaucoma, essential tremor, osmotic hyponatremia, and neurological diseases related to neuronal cell death. A CNS demyelinating disease can be chosen from one or more of the aforesaid disorders. In one embodiment, the CNS demyelinating disease is multiple sclerosis. In other embodiments, the CNS demyelinating disease is optic neuritis, e.g., acute optic neuritis.

Accordingly, methods for treating spinal cord injury, diseases or disorders associated with inhibition of neuronal growth in the CNS, diseases or disorders associated with inhibition of oligodendrocyte growth or differentiation, and diseases involving demyelination or dysmyelination of CNS neurons in a subject suffering from such injury or disease or predisposed to contract such disease, are disclosed. The method includes administering to the subject an effective amount of a reparative agent, e.g., a LINGO-1 antagonist, alone or in combination with an immunomodulatory agent.

As used herein, and unless otherwise specified, the terms “manage,” “managing” and “management” encompass preventing the progression of disease symptoms in a subject who has already suffered from the disease, and/or lengthening the time that the subject who has suffered from the disease remains in remission. The terms encompass modulating the threshold, development and/or duration of the disease, or changing the way that a patient responds to the disease.

As used herein, and unless otherwise specified, a “therapeutically effective amount” of a compound is an amount sufficient to provide a therapeutic benefit in the treatment or management of the disease, or to delay or minimize one or more symptoms associated with the disease. A therapeutically effective amount of a compound means an amount of therapeutic agent, alone or in combination with other therapeutic agents, which provides a therapeutic benefit in the treatment or management of the disease. The term “therapeutically effective amount” can encompass an amount that improves overall therapy, reduces or avoids symptoms or causes of the disease, or enhances the therapeutic efficacy of another therapeutic agent.

As used herein, and unless otherwise specified, a “prophylactically effective amount” of a compound is an amount sufficient to prevent relapse of the disease, or one or more symptoms associated with the disease, or prevent its recurrence. A prophylactically effective amount of a compound means an amount of the compound, alone or in combination with other therapeutic agents, which provides a prophylactic benefit in the prevention of disease relapse. The term “prophylactically effective amount” can encompass an amount that improves overall prophylaxis or enhances the prophylactic efficacy of another prophylactic agent.

As used herein, the term “patient” or “subject” typically refers to a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; that will be or has been the object of treatment, observation, and/or experiment. When the term is used in conjunction with administration of a compound or drug, then the patient has been the object of treatment, observation, and/or administration of the compound or drug.

In one embodiment, treatment with a LINGO-1 antagonist, alone or in combination with an immunomodulatory agent, begins as soon as a subject is diagnosed with a disorder affecting myelination or is diagnosed as being at risk for a disorder affecting myelination. For example, in one embodiment, a subject having AON in one eye is treated to enhance myelination/prevent demyelination in the visual pathway that serves the fellow eye. In another embodiment, a subject with AON is treated with a LINGO-1 antagonist, alone or in combination with an immunomodulatory agent to slow or prevent progression to MS.

In one embodiment, a subject to be treated according to the methods herein has minor symptoms at the time of initiation of treatment. In another embodiment, a subject to be treated according to the methods herein has marked impairment at the time of initiation of treatment. For example, in one embodiment, a subject with AON may have marked visual impairment in one eye at the time of treatment.

Subject to be treated according the methods described herein can be of any age when they are affected by demyelination or dismyelination or are at risk thereof. In one embodiment, a subject selected for treatment with a LINGO-1 antagonist is at least about 25 years of age. In another embodiment, a subject selected for treatment with a LINGO-1 antagonist is at least about 30 years of age. In still another embodiment, a subject selected for treatment with a LINGO-1 antagonist is at least about 30 years of age. In yet another embodiment, a subject selected for treatment with a LINGO-1 antagonist is at least about 35 years of age. In yet another embodiment, a subject selected for treatment with a LINGO-1 antagonist is at least about 40 years of age.

### Treatment of MS

Multiple sclerosis (MS) is a central nervous system disease that is characterized by inflammation and loss of axons and myelin sheaths.

Subjects having MS can be identified by clinical criteria establishing a diagnosis of clinically definite MS as defined by Poser et al. (1983) Ann. Neurol. 13:227. Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al. (2001) Recommended diagnostic criteria for Multiple sclerosis: guidelines from the International Panel on the Diagnosis of Multiple Sclerosis, Ann Neurol 50:121-127); Polman, C H et al. (2005 December). Diagnostic criteria for multiple sclerosis: 2005 revisions to the “McDonald Criteria” Annals of Neurology 58 (6): 840-6; Polman, C. H. et al. (2011) Ann. Neurol. 69(2):292-302). The McDonald criteria include the use of MRI evidence of CNS impairment over time to be used in diagnosis of MS, in the absence of multiple clinical attacks. Further updates to the McDonald criteria (Polman et al, Annals of Neurology 2011) allow the diagnosis of MS at the time of first CNS demyelinating episode based on the finding of pre-existing characteristic MRI lesions. Effective treatment of multiple sclerosis may be evaluated in several different ways. The following parameters can be used to gauge effectiveness of treatment. Two exemplary criteria include: EDSS (extended disability status scale as determined by an examining neurologists), and appearance of new lesions with or without clinical manifestations on MRI (magnetic resonance imaging) scans.

Exacerbations are defined as the appearance of one or more new neurological symptoms that are attributable to MS and accompanied by an appropriate new neurologic abnormality on examination. In addition, the exacerbation must last at least 24 hours and be preceded by stability or improvement for at least 30 days, and should not have alternative explanations (such as infection, drug toxicity, primary psychiatric disorders). Briefly, patients are given a standard neurological examination by clinicians. Exacerbations are mild, moderate, or severe according to changes in a Neurological Rating Scale like, for example, the Scripps Neurological Rating Scale (Sipe et al. (1984) Neurology 34:1368); the EDSS; or by patient reported outcomes (e.g., MSWS-12). An annual exacerbation rate and proportion of exacerbation-free patients are determined to monitor effectiveness of anti-inflammatory treatments.

Anti-inflammatory therapy can be deemed to be effective using a clinical measure if there is a statistically significant difference in the rate or proportion of exacerbation-free or relapse-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group. An exacerbation-free or relapse-free period of greater than one year, 18 months, 20, or 24 months is particularly noteworthy. Clinical measurements include the relapse rate in one and two-year intervals, and a change in EDSS, including time to worsening from baseline of 1.0 unit on the EDSS that persists for three or six months. On a Kaplan-Meier curve, a delay in sustained progression of disability shows efficacy. Other criteria include a change in area and volume of T2 images on MRI, and the number and volume of lesions determined by gadolinium enhanced images.

MRI can be used to measure active inflammatory lesions using gadolinium-DTPA-enhanced imaging (McDonald et al., Ann. Neurol. 36:14, 1994) or the location and extent of lesions using T2-weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of MS lesions can be determined by radiologists. Areas of lesions can be outlined and summed slice by slice for total lesion area. Three analyses may be done: evidence of new lesions, rate of appearance of active lesions, and percentage change in lesion area (Paty et al., (1993) Neurology 43:665). Improvement due to therapy can be established by a statistically significant improvement in an individual patient compared to baseline or in a treated group versus a placebo group.

The effects of remyelinating and/or neuroaxonal protective therapies can be evaluated using one or more of Magnetization Transfer Ratio (MTR), Diffusion Tensor Imaging (DTI), and brain volume changes, and Magnetic Resonance Spectroscopy (MRS). Each of the aforesaid techniques is described in more detail herein.

Magnetization Transfer Ratio (MTR) is based on application of off-resonance radio-frequency pulses and observing their effects on MR images, as well as measuring the signal intensity with and without application of the pulses. MTR has been shown to detect microscopic white matter pathology in MS not detectable on a standard MRI. (Siger-Zajdel M. et al., J Neurol Neurosurg Psychiatry 2001 71:752-756).

Diffusion Tensor Imaging (DTI) is a magnetic resonance imaging technique that enables the measurement of the restricted diffusion of molecules in tissue in order to produce neural tract images. This allows, for example, for visualization of neurons and white matter, which have an internal fibrous structure. Molecules diffuse more rapidly in the direction aligned with the internal structure, and more slowly perpendicular to the preferred direction. DTI can reveal abnormalities in white matter fiber structure in MS.

MRI can be used to measure changes in brain volume. Brain volume loss has been correlated with disability progression and cognitive impairment in MS, with the loss of grey matter volume more closely correlated with clinical measures than loss of white matter volume. (De Stefano N. et al., CNS Drugs. 2014 February; 28(2):147-56)

In Magnetic Resonance Spectroscopy (MRS), relative concentrations of target metabolites are determined. In the context of MS, MRS can quantify specific neurometabolites representing specific MS-related events, such as demyelination, inflammation, and axonal/neuronal dysfunction.

Exemplary symptoms associated with multiple sclerosis, which can be treated with the methods described herein or managed using symptom management therapies, include: optic neuritis, decreased visual acuity, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, L′hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoffs symptom, gastroesophageal reflux, and sleeping disorders.

Each case of MS displays one of several patterns of presentation and subsequent course. Most commonly, MS first manifests itself as a series of attacks followed by complete or partial remissions as symptoms improve, only to return later after a period of stability. This is called relapsing-remitting MS (RRMS). Primary-progressive MS (PPMS) is characterized by a gradual clinical decline with no distinct remissions, although there may be temporary plateaus or minor relief from symptoms. Secondary-progressive MS (SPMS) begins with a relapsing-remitting course followed by a later progressive course independently of relapses. PPMS, SPMS, and PRMS are sometimes lumped together and called chronic progressive MS.

A few patients experience malignant MS, defined as a swift and relentless decline resulting in significant disability or even death shortly after disease onset. This decline may be arrested or decelerated by determining the likelihood of the patient to respond to a therapy early in the therapeutic regime and switching the patient to an agent that they have the highest likelihood of responding to.

### Optic Neuritis

Also disclosed herein is the discovery that a reparative agent (an anti-LINGO-1 antibody) is capable of increasing the remyelination of an optic nerve in patients after an onset (e.g., a first attack) of acute optic neuritis (AON), as well as preventing (e.g., delaying) the onset of new disease in the visual pathways served by both the normal (unaffected) and the affected eye. In addition, by utilizing methods such as VEP (e.g., FF-VEP and mfVEP) to measure the function of the visual pathway including the optic nerve (e.g., measure the latency and amplitude of the VEP), a beneficial effect of the reparative agent on remyelination was detected. As described in the appended examples, a protective effect of the anti-LINGO-1 treatment was seen for the amplitude of the mfVEP in both the affected and the fellow eye visual pathways over 32 weeks, which was highly statistically significant at 32 weeks for the fellow eye mfVEP amplitude.

Also, methods to measure latency and amplitude of an optic nerve, such as VEP (e.g., FF-VEP and mfVEP, and in particular, mfVEP) provide a way to diagnose patients at risk of developing MS earlier than using previously described methods. Methods such as VEP also provide a way to diagnose/identify patients at risk of developing AON, e.g., one or both eyes, earlier than using previously described methods. Further, methods such as VEP provide a way to identify subjects that would most likely respond positively (e.g., have improved neuronal function) to a reparative agent described herein, e.g., anti-LINGO-1. Thus, without being bound by theory, the methods described herein provide an early intervention to treat and/or prevent AON as well as MS, e.g., by preserving myelination or causing remyelination of damaged areas early in the disease.

Accordingly, in other aspects, the methods described herein treat and/or prevent AON and/or MS in a subject before neuronal (e.g., axonal) damage, e.g., optic nerve damage in the subject. In embodiments, the methods described herein treat and/or prevent AON and/or MS in a subject before neuronal (e.g., axonal) damage and after demyelination of one or more nerves (e.g., an optic nerve) in the subject. In yet other embodiments, the methods described herein prevent AON and/or MS in a subject before neuronal (e.g., axonal damage and before demyelination of one or more nerves (e.g., an optic nerve) in the subject, e.g., the subject has one eye affected by AON (with demyelination of the optic nerve) and one fellow normal eye that is asymptomatic (without demyelination of the optic nerve).

In embodiments, a method of treating or preventing an inflammatory condition or disorder of the optic nerve, e.g., optic neuritis (e.g., AON), in a subject (e.g., a subject in need of treatment) is disclosed. The method includes administering to the subject a reparative agent (e.g., a LINGO-1 antagonist), as a monotherapy or in combination with a second agent (e.g., an immunomodulatory agent as described herein) in an amount sufficient to reduce one or more manifestations associated with the optic nerve condition or disorder, thereby treating or preventing the optic nerve condition or disorder.

In certain embodiments, said treatment includes: reducing one or more symptoms associated with the optic nerve condition or disorder; reducing, retarding or preventing a relapse, or the worsening of the optic nerve condition or disorder; and/or inhibiting or retarding the development of a new lesion in the subject. In other embodiments, said prevention includes delaying or ameliorating one or more symptoms or severity of the optic nerve disorder or condition. In one embodiment, one or more symptoms associated with the optic nerve disorder or condition (e.g., acute optic neuritis) includes one, two, three, four, five or more of visual loss, VEP latency delay (e.g., time for a signal to travel from the retina to the visual cortex), edema, inflammation, damage or demyelination of the myelin sheath covering the optic nerve and axons, loss of retinal fiber layer, or loss of retinal ganglion cell layer. In embodiments, one or more symptoms associated with the optic nerve condition or disorder includes one, two, three, four, five, six, seven, eight, nine, ten or more (all) of visual loss, edema, inflammation, damage or demyelination of the myelin sheath covering the optic nerve and axons, loss of retinal fiber layer, loss of retinal ganglion cell layer, visual field defect, color desaturation, decreased color vision, ocular pain, decreased visual acuity (e.g., as measured by low contract letter acuity or high contrast visual acuity), Uhthoff's symptom, swollen optic disc, or relative afferent papillary defect.

In one embodiment, the LINGO-1 antagonist is administered at one, two or all of the following:

(i) prior to the onset or relapse of one or more symptoms of the CNS demyelinating disease;

(ii) within 7 days after the onset or relapse of one or more symptoms of the CNS demyelinating disease (e.g., to enhance neuroprotection); or

(iii) within 30 days after the onset or relapse of one or more symptoms of the CNS demyelinating disease (e.g., to enhance remyelination).

In one embodiment, administration of the LINGO-1 antagonist further comprises a second agent, e.g., a second agent as described herein (e.g., an IFN-β 1 molecule) to be administered in combination with the LINGO-1 antagonist.

In one embodiment, the administration of the anti-LINGO-1 antibody molecule is acute, e.g., it is administered less than 2 weeks after an acute MS lesion (e.g., any lesion in MS, a relapse or AON). In one embodiment, the acute administration is less than 2 weeks or 1 week after the acute MS lesion (e.g., less than 13, 12, 11, 10, 9, 8, 7, 6, 5, 4, 3, 2, 1 day, or hours after the acute MS lesion). In one embodiment, administration is chronic, e.g., is administered prophylactically and/or administration continues for a prolonged period of time, e.g., administration continues until the beneficial effects of the treatment (e.g., as detected by remyelination or reduction in neuronal damage) are reduces or not detectable.

In certain embodiments, the treatment or prevention results in a recovery of VEP latency delay. In one embodiment, the recovery of the visual evoked potential (VEP) latency, e.g., full-field VEP (FF-VEP) or multifocal VEP (mfVEP), is partial or complete (e.g., at least 10%, 20%, 30%, 40%, 50%, 60%, 70%, 80%, 90% or 100% that of the unaffected fellow eye or the reference normal control VEP latency).

In embodiments, one or more of the following is indicative of the minimal level of optic nerve damage in the eye:
(i) an mfVEP amplitude of not more than 40 nanovolts lower than a reference value, e.g., a control amplitude,
(ii) a VEP amplitude of not more than 20% lower than a reference value, e.g., control amplitude, or
(iii) an mfVEP amplitude of 180 nanovolts or higher.

In embodiments, the control amplitude is the average VEP amplitude, e.g., FF-VEP amplitude and/or mfVEP amplitude, of a normal eye, e.g., an eye of a subject not having an optic nerve disorder, e.g., AON.

In embodiments, the step of measuring optic nerve conductance comprises a measure of VEP latency, e.g., FF-VEP latency or mfVEP latency.

In embodiments, one or more of the following is indicative of a delay in optic nerve conductance in the eye:

(i) a VEP latency that is at least 3 milliseconds higher than a reference value, e.g., a control latency,

(ii) a VEP latency that is at least 3% higher than a reference value, e.g., a control latency, or

(iii) a FF-VEP latency that is 110 milliseconds or higher.

(iv) an mf-VEP latency that is 155 miliseconds or higher.

In embodiments, the control latency is the average VEP latency, e.g., FF-VEP latency or mfVEP latency, of a normal eye, e.g., an eye of a subject not having an inflammatory condition of the optic nerve, e.g., AON.

In certain embodiments, the treatment or prevention delays one or more symptoms of the optic nerve disorder or condition, e.g., acute optic neuritis, by at least a day, a week, a month, 3 months, 6 months, a year or longer.

In one embodiment, the treatment or prevention is initiated before the onset or relapse of one or more symptoms of the nerve disorder or condition, e.g., acute optic neuritis. In one embodiment, the status of a subject with AON is measured by determining the visual evoked potential latency and amplitude.

In embodiments, In some embodiments, provided herein are methods comprising chronic and/or prophylactic administration of the reparative agent as a monotherapy or a combination therapy that can preserve neuronal function and/or neuronal tissue and/or prevent (e.g., delay) a disability in a subject, e.g., an MS or AON subject as described herein. In certain embodiments, chronic or prophylactic administration of the reparative agent may prevent the onset or delay the progressive form of the disease, e.g., AON or MS, for example, by reducing axonal/neuronal degeneration and/or demyelination.

In one embodiment, the reparative agent, alone or in combination, reduces one or more symptoms of an inflammatory condition of the optic nerve (e.g., optic neuritis, e.g., acute optic neuritis (AON)). AON is an inflammatory disease of the optic nerve that often occurs in multiple sclerosis. AON is caused by inflammatory injury to the optic nerve and presents with visual loss due to edema, inflammation, and damage to the myelin sheath covering the optic nerve and axons. There is significant loss of the retinal nerve fiber layer and retinal ganglion cell layer as a result of AON. Current treatment of AON is limited to intravenous treatment with high dose corticosteroids which fasten the resolution of edema, but do not promote central nervous system (CNS) remyelination or provide neuroaxonal protection from CNS inflammatory demyelination. Thus the reparative agents disclosed herein can be used, alone or in combination, to treat such inflammation of the optic nerve.

In some embodiments, the subject does not have a symptom associated with AON in one or both eyes. In one embodiment, the subject is not diagnosed with the optic nerve disorder or condition, e.g., acute optic neuritis, in one or both eyes (e.g., has normal eyes).

In one embodiment, the subject is diagnosed with the optic nerve disorder or condition, e.g., acute optic neuritis, in one or both eyes, referred to herein as the “diseased eye(s).” In one embodiment, the subject has a diseased eye and does not show a detectable symptom in the other eye (referred to herein as the “fellow eye” or “normal eye”). In one embodiment, the subject is diagnosed with the optic nerve disorder, e.g., AON, in one or both eyes, but does not show an MS symptom. In certain embodiment, the subject can be treated with the reparative agent (e.g., a LINGO-1 antagonist), as a monotherapy or in combination, as a way of preventing or delaying the onset of the nerve disorder or condition in the normal eye. Without wishing to be bound by theory, treatment of a normal fellow eye after diagnosis of acute optic neuritis in a first eye may delay or prevent one or more symptoms of neuritis in the normal fellow eye or elsewhere in the brain. In certain embodiments, the treatment or prevention delays one or more symptoms of the optic nerve disorder or condition, e.g., acute optic neuritis, and/or MS by at least a day, a week, a month, a year or longer.

Thus, in one embodiment, administration of the anti-LINGO-1 antibody molecule to the subject treats the diseased eye. In related embodiments, administration of the anti-LINGO-1 antibody molecule delays or prevents the onset of MS or the optic nerve disorder, e.g., AON, in the normal fellow eye, or elsewhere in the CNS.

In other embodiment, the subject is diagnosed with the optic nerve disorder or condition, e.g., acute optic neuritis, in one or both eyes, but does not show other MS symptoms (e.g., is a subject not diagnosed with MS or a subject that has MS but does not suffer from a relapse or is not progressing). In one embodiment, treatment of said subject with the optic nerve disorder or condition, e.g., acute optic neuritis, delays or prevents the onset or relapse of an MS symptom. In certain embodiments, the treatment or prevention delays one or more MS symptoms by at least a day, a week, a month, a year or longer.

In another embodiment, the subject is diagnosed with internuclear ophthalmoplegia (INO) and may or may not show other MS symptoms (e.g., is a subject not diagnosed with MS or a subject that has MS but does not suffer from a relapse or is not progressing). In one embodiment, the subject is diagnosed with INO by evaluating versional dysconjugacy index (VDI). In one embodiment, treatment of a subject with INO with an anti-LINGO-1 antibody molecule delays or prevents the onset or relapse of an MS symptom, e.g., INO. In some embodiments, treatment, e.g., improvement, of a subject's INO symptoms is characterized by a change, e.g., a decrease, in VDI.

### Combination Therapy

The invention discloses combined administration of an immunomodulatory agent, e.g., an IFN-β agent, e.g., Avonex®; and a reparative agent, e.g., an anti-LINGO-1 antibody, for treatment of a demyelinating disorder, e.g., MS.

The agents, e.g., pharmaceutical compositions including the agents, can be administered concurrently with, prior to, or subsequent to, one or more other additional therapies or therapeutic agents. In general, each agent can be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the pharmaceutical composition with the additional therapeutically active agent and/or the desired therapeutic effect to be achieved. In general, it is expected that additional therapeutic agents utilized in combination be utilized at levels that do not exceed the levels at which they are utilized individually. In some embodiments, the levels utilized in combination will be lower than those utilized individually.

Treatment of a subject with a disease with a reparative agent can be combined with one or more immunomodulatory agents. Exemplary immunomodulatory agents are described herein and include, but are not limited to, an IFN-β 1 molecule; a polymer of glutamic acid, lysine, alanine and tyrosine, e.g., glatiramer; an antibody or fragment thereof against alpha-4 integrin, e.g., natalizumab; an anthracenedione molecule, e.g., mitoxantrone; a fingolimod, e.g., FTY720 or other S1P1 modulators, such as BAF312 or ozanimod; a dimethyl fumarate, e.g., an oral dimethyl fumarate; an antibody to the alpha subunit of the IL-2 receptor of T cells (CD25), e.g., daclizumab; an antibody against CD52, e.g., alemtuzumab; an inhibitor of a dihydroorotate dehydrogenase, e.g., teriflunomide; a corticosteroid; and an anti-CD20 antibody, e.g., ocrelizumab.

In one embodiment, a combination of Avonex® and anti-LINGO-1 antibody therapy is administered. Anti-LINGO-1 antibody treatment doses can include: IV infusions of: an amount ranging from about 0.3, 1, 3, 10, 30, 60, 100, or 150 mg/kg; or a flat dose of 20 mg, 70 mg, 200 mg, 700 mg, 750 mg, 2000 mg, 4000 mg, 7000 mg, or 1050 mg; concurrent with once-weekly Avonex® IM injections. In certain embodiments, an anti-LINGO-1 antibody can be administered once about every 4 weeks (plus or minus about 5 days) by intravenous (IV) infusion in addition to once weekly Avonex® intramuscular (IM) injections.

In one embodiment, the anti-LINGO-1 antibody molecule is administered at about 100 mg/kg, or 7000 mg, via IV infusion or SC injection, e.g., as a single dose.

In another embodiment, the anti-LINGO-1 antibody molecule is administered as one dose of 30 mg/kg, or 2000 mg, e.g., administered intravenously, every four weeks, to a total of 3 doses.

In another embodiment, the anti-LINGO-1 antibody molecule is administered as one dose of 10 mg/kg, 700 mg, or 750 mg, e.g., administered intravenously, every four weeks, to a total of 8 doses.

In another embodiment, the anti-LINGO-1 antibody molecule is administered as one dose of 3 mg/kg, or 200 mg, e.g., administered intravenously, every four weeks, to a total of 18 doses.

In yet another embodiment, the anti-LINGO-1 antibody molecule is administered as one dose of 3-5 mg/kg, or 200-350 mg, e.g., administered subcutaneouly, every four weeks, to a total of 18 doses.

In yet another embodiment, the anti-LINGO-1 antibody molecule is administered as one dose of 10 mg/kg, e.g., intravenously, every 12 weeks, to a total of 8 doses.

In yet another embodiment, the anti-LINGO-1 antibody molecule is administered as one dose of 30 mg/kg, e.g., intravenously, every 24 weeks, to a total of 4 doses.

In other embodiments, 3 mg/kg, or 200 mg, IV infusion once every 4 weeks of an anti-LINGO-1 antibody was selected. This regimen is expected to yield a mean average serum concentration similar to rat serum EC50 in the spinal cord lysolecithin model (adjusted for ˜0.1% CNS penetration). Additional dosing regimens, 10 mg/kg, or 700 mg, and 30 mg/kg, or 2000 mg, can also be administered. These 2 dosing regimens are expected to yield mean average serum concentrations approximately 1.2-fold and 3.7-fold higher than the rat serum EC90 (adjusted for ˜0.1% brain penetration), respectively.

In certain embodiments, the immunomodulatory agent is an IFN-β 1 molecule and is administered intravenously, subcutaneously or intramuscularly. For example, the IFN-β 1 molecule can be administered at one or more of:

(i) at 20-45 microgram (e.g., 30 microgram), e.g., once a week via intramuscular injection;

(ii) at 20-30 microgram (e.g., 22 microgram), e.g., three times a week, or at 40-50 micrograms (e.g., 44 micrograms), e.g., three times a week, via subcutaneous injection; or

(iii) in an amount of between 10 and 50 μg intramuscularly, e.g., three times a week, or every five to ten days, e.g., once a week.

Alternatively, the IFNb molecule is pegylated IFN-β 1 and is administered at 50 μg to 200 μg, e.g., 50 μg to 60 μg (e.g., 63 μg), 90 μg to 100 μg (e.g., 94 μg) or 120 μg to 130 μg (e.g., 125 μg) once every other week subcutaneously.

In one embodiment, Avonex® is administered at 30 microgram once a week via intramuscular injection. Following titration when applicable, Avonex® can be administered by IM injection following dosage and administration schedules known in the art.

In one embodiment, the IFN-β agent, e.g., Avonex®, is administered by an injection device, e.g., an autoinjection device or pen.

In one embodiment, the anti-LINGO-1 antibody molecule is supplied as a liquid drug product containing 50 mg/mL opicinumab (BIIB033) (also referred to herein as an antibody molecule having a VH that includes the amino acid sequence of SEQ ID NO: 275 and a VL that includes the amino acid sequence of SEQ ID NO: 276), 10 mM sodium citrate, 160 mM L-arginine hydrochloride (pH 6.5), and 0.03% (weight per volume) polysorbate 80. The anti-LINGO-1 antibody molecule can be administered by IV infusion following saline dilution.

In one embodiment, the immunomodulatory agent is Avonex®, which is is formulated as a sterile clear liquid for IM injection. Each 0.5 mL of Avonex in a prefilled glass syringe contains 30 mcg of interferon β-1a. Other ingredients include sodium acetate trihydrate, glacial acetic acid, arginine hydrochloride, and polysorbate 20 in Water for Injection at a pH of approximately 4.8. The immunomodulatory agent, e.g., Avonex®, can be administered by any suitable means, e.g., a pen or other device.

### Symptom Management

Treatment of a subject with a combination therapy described herein can be combined with one or more of the following therapies often used in symptom management of subjects having MS: Tegretol® (carbamazepine), Epitol® (carbamazepine), Atretol® (carbamazepine), Carbatrol® (carbamazepine), Neurontin® (gabapentin), Topamax® (topiramate), Zonegran® (zonisamide), Dilantin® (phenytoin), Norpramin® (desipramine), Elavil® (amitriptyline), Tofranil® (imipramine), Imavate® (imipramine), Janimine® (imipramine), Sinequan® (doxepine), Adapin® (doxepine), Triadapin® (doxepine), Zonalon® (doxepine), Vivactil® (protriptyline), Marinol® (synthetic cannabinoids), Trental® (pentoxifylline), Neurofen® (ibuprofen), aspirin, acetaminophen, Atarax® (hydroxyzine), Prozac® (fluoxetine), Zoloft® (sertraline), Lustral® (sertraline), Effexor XR® (venlafaxine), Celexa® (citalopram), Paxil®, Seroxat®, Desyrel® (trazodone), Trialodine® (trazodone), Pamelor® (nortriptyline), Aventyl® (imipramine), Prothiaden® (dothiepin), Gamanil® (lofepramine), Parnate® (tranylcypromine), Manerix® (moclobemide), Aurorix® (moclobemide), Wellbutrin SR® (bupropion), Amfebutamone® (bupropion), Serzone® (nefazodone), Remeron® (mirtazapine), Ambien® (zolpidem), Xanax® (alprazolam), Restoril® (temazepam), Valium® (diazepam), BuSpar® (buspirone), Symmetrel® (amantadine), Cylert® (pemoline), Provigil® (modafinil), Ditropan XL® (oxybutynin), DDAVP® (desmopressin, vasopressin), Detrol® (tolterodine), Urecholine® (bethane), Dibenzyline® (phenoxybenzamine), Hytrin® (terazosin), Pro-Banthine® (propantheline), Urispas® (hyoscyamine), Cystopas® (hyoscyamine), Lioresal® (baclofen), Hiprex® (methenamine), Mandelamine® (metheneamine), Macrodantin® (nitrofurantoin), Pyridium® (phenazopyridine), Cipro® (ciprofloxacin), Dulcolax® (bisacodyl), Bisacolax® (bisacodyl), Sani-Supp® (glycerin), Metamucil® (psyllium hydrophilic mucilloid), Fleet Enema® (sodium phosphate), Colace® (docusate), Therevac Plus®, Klonopin® (clonazepam), Rivotril® (clonazepam), Dantrium® (dantrolen sodium), Catapres® (clonidine), Botox® (botulinum toxin), Neurobloc® (botulinum toxin), Zanaflex® (tizanidine), Sirdalud® (tizanidine), Mysoline® (primidone), Diamox® (acetozolamide), Sinemet® (levodopa, carbidopa), Laniazid® (isoniazid), Nydrazid® (isoniazid), Antivert® (meclizine), Bonamine® (meclizine), Dramamine® (dimenhydrinate), Compazine® (prochlorperazine), Transderm® (scopolamine), Benadryl® (diphenhydramine), Antegren® (natalizumab), Campath-1H® (alemtuzumab), Fampridine® (4-aminopyridine), Gammagard® (IV immunoglobulin), Gammar-IV® (IV immunoglobulin), Gamimune N® (IV immunoglobulin), Iveegam® (IV immunoglobulin), Panglobulin® (IV immunoglobulin), Sandoglobulin® (IV immunoglobulin), Venoblogulin® (IV immunoglobulin), pregabalin, ziconotide, Baclofen and AnergiX-MS®.

### Clinical Tests/Assessments for the Evaluation of Combination Avonex® and Anti-LINGO-1 Antibody Therapy

Efficacy endpoints of the therapy in any subject can be evaluated using tests and assessments known in the art. For example, for an RRMS patient, the subject can be evaluated by acquiring the subject's status using EDSS. In other embodiments where the subject has a progressive form of MS, e.g., SPMS or PPMS, the subject can be evaluated by obtaining a measure of upper and/or lower extremity function, and/or a measure of ambulatory function, e.g., short distance ambulatory function, in addition to acquiring the subject's status using EDSS. In certain embodiments, an assessment of lower extremity ambulatory function (e.g., Timed Walk of 25 Feet (T25FW)), and/or an assessment of upper extremity function (e.g., 9 Hole Peg Test (9HP)) can be performed.

Additional exemplary efficacy endpoints that can be evaluated include one or more of the following.

### Efficacy Endpoints

### Exemplary Primary Endpoints

Subjects can be evaluated for confirmed improvement of neurophysical and/or cognitive function over treatment as measured by a composite endpoint comprising the Expanded Disability Status Scale (EDSS), Timed 25-Foot Walk (T25FW), 9-Hole Peg Test (9HPT), and (3-Second) Paced Auditory Serial Addition Test (PASAT). Improvement on neurophysical and/or cognitive function can be defined as at least 1 of the following:

a) A ≥1.0 point decrease in EDSS from a baseline score of ≤6.0 (decrease sustained for 3 months or greater);

b) A ≥15% improvement from baseline in T25FW (improvement sustained for 3 months or greater);

c) A ≥15% improvement from baseline in 9HPT (improvement sustained for 3 months or greater); and

d) A ≥10% (e.g., 10%, 12%, 20%, 30%) improvement from baseline in PASAT (improvement sustained for 3 months or greater). Alternatively, the improvement can be detected using the Symbol Digit Modalities Test (SDMT).

In one embodiment, an exemplary end point for measuring the status of a patient with AON is measurement of the recovery of latency of the VEP (e.g., time for a signal to travel from the retina to the visual cortex). Latency is a measure of how well neurons can conduct, e.g., their conduction timing. Neurons that have intact myelin can conduct better (transmit a signal faster) than neurons that have lost or damaged myelin. The amplitude as measured by VEP is a measure of the number of funtioning neurons (e.g., that contain axons capable of transmitting information) and the number of inactive (e.g., dead/damaged) neurons, with a higher amplitude indicative of a greater number of normally functioning neurons. Such measurement can be made using methods known in the art, e.g., using full field visual evoked potential (FF-VEP). Visual evoked potential (VEP) can be measured by methods known in the art, including the traditional method (referred to as full-field VEP) or with a multifocal VEP (mfVEP) that measures a larger sample of visual pathway and with better precision. These methods can be applied to increase the sensitivity of detection. For example, the fellow eye in AON can show amplitude changes (in nanovolts) with the mfVEP as the FF-VEP which measures amplitude changes in microvolts may not be sufficiently sensitive FF-VEP measures the latency and amplitude of the central visual field, e.g., sum of the latencies or amplitudes of the visual pathway representing about 5 degrees of the central vision (macular vision). mfVEP measures the latency and amplitude of up to 56 segments covering up to 60 degrees of the visual field for each individual eye. (Hood et al. Trans. Am. Ophthalmol. Soc. 104(2006):71-77). mfVEP permits the mapping of individual segements of the visual pathway, which may have different amplitudes and latencies depending on the degree of injury and repair.

In embodiments, an improvement in latency delay (e.g., reduced FF-VEP and/or mfVEP latency delay in miliseconds) is an indication of remyelination of lesions along the visual pathway, including optic nerves, optic radiations, or visual cortex. In embodiments, preserved amplitude (e.g., preserved FF-VEP or mfVEP amplitude) is an indication of neuroaxonal protection and/or repair along the visual pathway anywhere between the ganglion cell neurons in the retina and the cerebral visual cortical neurons.

### Exemplary Secondary Endpoints

Subjects can be evaluated for confirmed worsening of neurophysical and/or cognitive function and/or disability treatment as measured by a composite endpoint of the EDSS, T25FW, 9HPT, and PASAT. Progression of disability or worsening of neuro-physical and/or cognitive function is defined as at least 1 of the following:

a) A ≥1.0 point increase in EDSS from a baseline score of ≤5.5 or a ≥0.5 point increase from a baseline score equal to 6.0 (increase sustained for 3 months or greater);

b) A ≥15% worsening from baseline in T25FW (worsening sustained for 3 months or greater);

c) A ≥15% worsening from baseline in 9HPT (worsening sustained for 3 months or greater); and

d) A ≥10% (e.g., 10%, 12%, 20%, 30%) worsening from baseline in PASAT (worsening sustained for 3 months or greater). Alternatively, the worsening from baseline can be measured by SDMT.

In one embodiment, exemplary secondary endpoints to measure the status of a patient with AON include measuring the change in thickness of the retinal layers (retinal ganglion cells neurons and unmyelinated axonal segment) and/or measurement of retinal structure and function. Retinal structure can be measured using known methods, e.g., spectral domain optical coherence tomography (SD-OCT) while clinical visual function can be measured using visual acuity, e.g. low contrast letter acuity (1.25% and 2.5%), and/or high contrast visual acuity.

Exemplary techniques described herein for primary and secondary efficacy biomarker endpoint analysis can be further described as follows:

Latency recovery as measured by Full Field Visual evoked potential (FF-VEP) or Multifocal visual evoked potential (mfVEP) which measure whether remaining (live) axons can be repaired via remyelination following AON.

In embodiments, the FF-VEP and/or mfVEP amplitude is a different but equally important measure of visual pathway damage for each eye. In embodiments, an amplitude of at least 40 nanovolts (e.g., at least 40, 50, 60, 70, 80, 90, 100 nanovolts or more) lower than a control amplitude on mfVEP indicates the presence of visual pathway damage serving each of the two eye(s) of the subject. In embodiments, an amplitude that is at least 20% (e.g., at least 20%, 30%, 40%, 50%, 60%, 70%, 80%, 90%, or more) lower than a control amplitude indicates the presence of optic nerve damage in the eye(s) of the subject. In embodiments, an mfVEP amplitude that is about 180 nanovolts or lower (e.g., about 180, 170, 160, 150, 140, 130, 120, 110, 100, 90 nanovolts or lower) indicates the presence of optic nerve damage in the eye(s) of the subject. In embodiments, a control amplitude is the average VEP amplitude (e.g., FF-VEP in microvolts or mfVEP amplitude in microvolts) of a normal eye, e.g., an eye of a subject not having one or more symptoms of an optic nerve disorder or condition (e.g., acute optic neuritis); or the fellow eye of the subject where the fellow eye does not exhibit one or more symptoms of an optic nerve disorder or condition (e.g., acute optic neuritis) but the visual pathway can become affected over time as a results of new lesion development anywhere along the pathway. In embodiments, a control amplitude is the baseline VEP amplitude (e.g., FF-VEP or mfVEP amplitude) of a fellow normal eye.

In embodiments, the FF-VEP and/or mfVEP latency is a measure of optic nerve conductance. In embodiments, a latency that is at least 3 milliseconds higher (e.g., at least 3, 4, 5, 6, 7, 8, 9, 10, or more milliseconds higher) than a control latency indicates a delay in optic nerve conductance in the eye(s) of the subject. In embodiments, a latency that is at least 3% (e.g., at least 3%, 4%, 5%, 6%, 7%, 8%, 9%, 10%, 12%, 15%, 20%, 30%, 40%, 60%, 80%, or more) higher than a control latency indicates a delay in optic nerve conductance in the eye(s) of the subject. In embodiments, a FF-VEP latency that is at least about 110 milliseconds (e.g., at least about 110, 120, 130, 140, 150, 160, 170, 180 milliseconds or more) indicates a delay in optic nerve conductance in the eye(s) of the subject. In embodiments, an mfVEP latency can be at least about 155 msec (e.g., at least about 155, 165, 175, 185 milliseconds or more). In embodiments, a control latency is the average VEP latency (e.g., FF-VEP or mfVEP latency) of a normal eye, e.g., an eye of a subject not having one or more symptoms of an optic nerve disorder or condition (e.g., acute optic neuritis); or the fellow eye of the subject where the fellow eye does not exhibit one or more symptoms of an optic nerve disorder or condition (e.g., acute optic neuritis). In embodiments, a control latency is the baseline VEP latency (e.g., FF-VEP or mfVEP latency) of a fellow normal eye.

In embodiments, latency recovery is indicated by a FF-VEP and/or mfVEP latency of an affected eye after treatment that is within 15% (e.g., within 15%, 12%, 10%, 8%, 6%, 4%, or less) of the FF-VEP and/or mfVEP latency of a control latency, e.g., the baseline latency of the fellow normal eye or the unaffected eyes of healthy adults of similar age, sex, and head circumference (normative data). In embodiments, latency recovery is indicated by a FF-VEP and/or mfVEP latency of an affected eye after treatment that is within 15 milliseconds (e.g., within 15, 14, 13, 12, 11, 10, 9, 8, 7, 6, 5, 4, 3, or 2 milliseconds) of the FF-VEP and/or mfVEP latency of a control latency, e.g., the baseline latency of the fellow normal eye. In embodiments, latency recovery is indicated by a FF-VEP latency of an affected eye after treatment that is about 120 milliseconds or less (e.g., about 120, 110, 100, 90, 80 nanovolts, or less). Without being bound by theory, it is believed that the presence of latency recovery, e.g., as measured by FF-VEP and/or mfVEP, in one or both eyes after treatment is an indicator of remyelination of the optic nerve(s).

Spectral Domain Optical Coherence Tomography (SD-OCT) (secondary endpoint) is different, and measures the thickness of the retinal nerve fiber layer (RNFL—unmyelinated portion of the optic nerve within the retina) and the retinal ganglion cell layer (RGCL—neuron cell body for optic nerve axons within the retina). The reduction of thickness in RNFL and RGCL following AON are considered evidence of axonal and neuronal loss (death). In embodiments, reduced loss of RNFL and/or RGCL thickness, e.g., a less than 12% (e.g., less than 12%, 11%, 10%, 9%, 8%, 6%, 4%, or less) reduction in thickness compared to the thickness of RNFL and/or RGCL in a normal eye (e.g., normal fellow eye), is evidence of neuroaxonal protection and/or repair.

In embodiments, visual function is measured by visual acuity, e.g., low-contrast (e.g., 1.25 or 2.5%) letter acuity (LCLA) or high-contrast (e.g., 100%) visual acuity (HCVA). Low Contrast Letter Acuity (secondary endpoint) is a measure of the ability of a patient to distinguish between degrees of low contrast (faint grey letters on white background). High contrast visual acuity is a measure of the ability to distinguish between degrees of high contrast (black letters on white background). In embodiments, the visual acuity is reported in number of letters that a subject is able to correctly read. In embodiments, an increase in visual acuity (e.g., by at least 6 letters, e.g., at least 6, 7, 8, 9, 10, 12, 14, 16, 18, 20, 30, 40, or more letters), e.g., LCLA and/or HCVA, is evidence of improvement or preservation of visual function in an AON-affected eye.

In embodiments, visual quality of life is an endpoint used in accordance with the methods described herein. In embodiments, visual quality of life is measured as described herein, e.g., in Example 6. For example, visual quality of life is measured by a patient reported outcome test. Exemplary patient reported outcome tests include but are not limited to a NIH-NEI visual functional questionnaire (NEI-VFQ) (see, e.g., Mangione et al. Arch. Opthalmol. 116.11(1998):1496-1504) and a neuro-ophthalmic supplement (NOS-10) (see, e.g., Raphael et al. Am. J. Ophthalmol. 142.6(2006):1026-35.e2). In embodiments, an increase of at least 4 points (e.g., at least 4, 5, 6, 7, 8, 9, 10, 11, 12, 13, 14, 15, 16, 17, 18, 19, 20, or more points) in a patient reported outcome test described herein, e.g., NEI-VFQ and/or NOS-10, is evidence of improvement of visual related quality of life.

### Additional Clinical Efficacy Endpoints

In certain embodiments, subjects can be evaluated using additional clinical measures, including:

a) A change from baseline in cognitive function as measured by an MS cognitive composite endpoint comprising 2 tests of processing speed (the PASAT and the Symbol-Digit Modalities Test [SDMT]) and 2 tests of memory and learning (the Selective Reminding Test [SRT] for verbal memory and the Brief Visuospatial Memory Test-Revised [BVMT-R] for visual memory);
b) Severity of clinical relapses as determined by the Scripps Neurological Rating Scale (SNRS) or EDSS examination; and/or
c) A ≥10% (e.g., ≥15%, ≥20%, ≥30%) worsening from baseline in Six Minute Walk (6 MW) walking time (worsening sustained for 3 months or greater).

Exemplary MRI Efficacy Endpoints

Analysis of brain MRI focused on measures of repair at the focal and diffuse levels with both new and preexisting lesions can include one or more of:

(i) Analysis of new brain lesions:

a) Percentage Gd lesion volume with increased and decreased magnetization transfer ratio (MTR);

b) A change from onset in new Gd lesion mean MTR relative to the normal appearing white matter (NAWM) with lesions per subject as the unit of measure;

c) A change from onset in MTR signal for voxels per scan whose MTR drops below the normal value (new MTR lesions) with subject as the unit of measure;

d) A change from onset in new Gd lesion radial diffusivity with subject as the unit of measure;

e) A change from onset in radial diffusivity for voxels per scan whose MTR drops below the normal value (new MTR lesions) with subject as the unit of measure; or f) Percentage conversion from new Gd brain lesions to chronic black hole with chronic black holes defined as T1 hypointensity still visible after at least 6 months from onset.

(ii) Analysis of pre-existing brain lesions (lesions that are present at baseline scan):

a. A change in MTR from baseline for abnormal T1 volume;

b. A change in MTR from baseline for abnormal T2 volume;

c. A change in MTR from baseline for abnormal T2 volume not associated with T1 hypointensity;

d. A change in diffusion tensor imaging (DTI) from baseline for abnormal T1 volume;

e. A change in DTI from baseline for abnormal T2 volume; or

f. A change in DTI from baseline for abnormal T2 volume not associated with T1 hypointensity.

(iii) Analysis of diffuse brain MRI metrics:

a) Percentage brain volume change;

b) A change from baseline in cerebral cortical brain volume;

c) A change from baseline in thalamic volume; or

d) A change from baseline in whole brain radial diffusivity.

e) A chance from baseline in whole brain MTR.

Exemplary Patient-Reported Outcomes (PROs) Efficacy Endpoints

In certain embodiments, subjects can be evaluated by patient reported outcomes, including one or more of:

a) 12-Item Multiple Sclerosis Walking Scale (MSWS-12).

b) ABILHAND 56-Item Questionnaire.

c) 29-Item Multiple Sclerosis Impact Scale (MSIS-29).

d) The Short Form (36) Health Survey (SF-36).

e) MSNQ-informant and MSNQ-patient

### Efficacy Endpoint Analysis

### General Methods of Analysis

Summary statistics may be presented. For continuous endpoints, the summary statistics may generally include: the number of subjects randomized or dosed; or the number of subjects with data, mean, SD, median, and range. For categorical endpoints, the summary statistics may generally include: the number of subjects randomized or dosed; the number of subjects with data, or the percent of subjects with data in each category.

### Exemplary Primary Endpoint Analyses

Exemplary primary efficacy endpoints can include the percentage of subjects with confirmed clinical improvement in 1 or more of the components of the composite endpoint (EDSS, T25FW, 9HPT, or PASAT). The percentage of confirmed improvers can be presented by treatment groups, and the data analyzed by a logistic regression model. Time to confirmed improvement may be analyzed using the Cox proportional hazards model. Baseline EDSS, T25FW, 9HPT (both dominant and non-dominant hands), PASAT, and stratification factors may be included in both logistic regression and Cox models as covariates. If 2 baseline EDSS assessments are performed, the higher EDSS score can be used for analysis. MRI activity may be explored as potential covariates as well.

### Exemplary Secondary Endpoint Analyses

The secondary efficacy endpoint may include the percentage of subjects with confirmed clinical worsening in 1 or more of the components of the composite endpoint (EDSS, T25FW, 9HPT, or PASAT). The percentage of confirmed worseners can be presented by treatment groups, and the data analyzed by a logistic regression model. Time to confirmed worsening may be analyzed using the Cox proportional hazards model. Baseline EDSS, T25FW, 9HPT (both dominant and non-dominant hands), PASAT, and stratification factors may be included in both logistic regression and Cox models as covariates. If 2 baseline EDSS assessments are performed, the higher EDSS score can be used for analysis. MRI activity may be explored as potential covariates as well.

### Exploratory Endpoint Analyses

The exploratory endpoints may include clinical metrics, MRI metrics, and PRO variables. They can be summarized by presenting summary statistics for continuous variables or frequency distributions for categorical variables. The statistical methods used will depend on the nature of the variables. Binary variables can be analyzed by using a logistic regression model; continuous variables can be analyzed by using the analysis of covariance model, adjusting for the corresponding baselines and stratification factors. Time-to-event variables can be analyzed using the Cox proportional hazards regression model, by adjusting for the corresponding baselines and stratification factors. Count variables can be analyzed by a Negative Binomial regression model or a Wilcoxon rank-sum test.

### Ambulatory Assessments

### T25FW

The T25FW is a timed walk of 25 feet. The T25W is a measure of quantitative ambulatory capacity over a short distance that is responsive to deterioration mostly for subjects who are very disabled, e.g., EDSS steps 6-6.5. It can be used as quantitative measure of lower extremity function. Broadly, the patient is directed to one end of a clearly labeled 25-foot course and is instructed to walk 25 feet as quickly as possible, but safely. The task can be immediately administered again by having the patient walk back the same distance. Patients may use assistive devices when completing the T25W. A time limit of 3 minutes to complete the test is usually used. The test is discontinued if the patient cannot complete Trial 2 of the T25W after a 5 minute rest period, or if the patient cannot complete a trial in 3 minutes.

### 9HPT

The 9HPT is a 9-hole peg test. It is a quantitative measure that captures a clinically important aspect of upper extremity (e.g., arm and hand) function that is not measured by the EDSS or the T25FW. Unlike the EDSS and the T25FW, the 9HPT is responsive across a wide EDSS range. Broadly, a patient is asked to pick up 9 pegs one at a time, using their hands only, and put the pegs into the holes on a peg board as quickly as possible until all of the holes are filled. The patient must then, without pausing, remove the pegs one at a time and return them to the container as quickly as possible. Both the dominant and non-dominant hands are tested twice (two consecutive trials of the dominant hand, followed immediately by two consecutive trials of the non-dominant hand). A time limit of 5 minutes to complete the test is usually used. The test is discontinued if the patient cannot complete one trial of the 9HPT test in 5 minutes; if the patient cannot complete a trial with his or her dominant hand within 5 minutes, the patient is usually instructed to move onto the trials with the non-dominant hand.

### 6 MW

The 6 minute walking test (6 MW) is used to assess walking distance. Broadly, the patient is asked to walk the fastest speed possible without physical assistance for 6 minutes and the distance is measured. Assistive devices can be used but should be kept consistent and documented from test to test. The patient should walk continuously if possible, but the patient can slow down to stop or rest during the test.

### SNRS

The Scripps Neurological Rating Scale (SNRS) measures several parameters, including, mentation and mood; eyes and related cranial nerves, e.g., visual acuity, visual fields, eye movements, nystagmus; lower cranial nerves; motor function in each extremity, e.g., right upper, left upper, right lower, left lower; deep tendon reflexes, e.g., upper extremities, lower extremities; Babinski sign, e.g., left side, right side; sensory function in each extremity, e.g., right upper, left upper, right lower, left lower; cerebellar signs, e.g., upper extremities, lower extremities; and gait trunk balance, e.g., special category for autonomic dysfunction, e.g., bladder dysfunction, sexual dysfunction.

### EDSS

As described above, the EDSS is based on a standardized neurological examination, focusing on the symptoms that occur frequently in MS. The EDSS assess the seven functional systems: visual, brainstem, pyramidal, cerebellar, sensory, bowel/bladder and cerebral; through neurological examination. In addition the EDSS also includes an assessment of walking range. Based on the functional system scores and the walking range, an EDSS step is determined. The range of the EDSS includes 19 steps from 0 to 10, with EDSS step 0 corresponding to a completely normal examination and EDSS step 10 to death due to MS. For EDSS ratings between 0 and 4, the scale relies mainly on the scores of the individual FS. For ratings over 4, the EDSS is primarily determined by the ability and range of walking.

### Patient Reported Outcome Assessments

### MSWS-12

The Multiple Sclerosis Walking Scale-12 (MSWS-12) test is a self rated measure of walking ability. The test contains 12 questions with Likert-type responses, describing the impact of MS on walking. The questions were generated from 30 MS patient interviews, expert opinions, and literature reviews.

### ABILHAND 56-Item Questionnaire

The ABILHAND 56-Item Questionnaire is a measure of manual ability designed to measure a patient's experience of problems in performing everyday tasks such as feeding, dressing, or managing tasks. The ABILHAND contains 56 unbiased and bimanual activities, which the patients are asked to judge on a four-level scale: 0=impossible, 1=very difficult, 2=difficult, 3=easy.

### MSIS-29

The Multiple Sclerosis Impact Scale 29 (MSIS-29) is a 29 item self report rating scale which measures physical and psychological parameters of MS. Three of the items deal with limited abilities, and the remaining 26 items are related to being impacted by symptoms or consequences of disease. Twenty of the items refer to physical function. Responses use a 5 point Likert scale range from 1 to 5.

### SF-36

The short form 36 (SF-36) test measures overall health related quality of life. The SF-36 is a structured, self report questionnaire that the patient can generally complete with little to no intervention from a physician. There is no single overall score for the SF-36, instead it generates 8 subscales and two summary scores. The 8 subscales include physical functioning, role limitations due to physical problems, bodily pain, general health perceptions, vitality, social functioning, role limitations due to emotional problems, and mental health. The two summary scores include a physical component summary and a mental health component summary.

### Cognitive Test Assessments

Several cognitive test instruments can be used to determine the value of the composite parameter, as follows.

### Symbol Digit Modalities Test (SDMT)

The SDMT is a test that evaluates processing speed and working memory in which the subject is given 90 seconds to pair specific numbers with given geometric figures based on a reference key. It is modeled after the Digit Symbol or Coding Tasks tests, which have been included in the Wechsler intelligence scales for many years (e.g., Wechsler et al. (1974) Manual for the Wechsler Intelligence Scale for Children-Revised. New York: Psychological Corporation; Wechsler et al. (1981) WAIS-R Manual. New York: Psychological Corporation). Recognizing the limitations some patients have with manual dexterity, Rao and colleagues modified the SDMT to include only an oral response (Rao et al. (1991) Neurology 41: 685-691). In this oral SDMT selected in the present invention, participants are presented with an 8.5×11 inch sheet that contains the numbers and symbols to be processed. The top row of stimuli includes nine symbols, each of which is paired with a single digit in the key. The remainder of the page has a pseudo-randomized sequence of these symbols, and the participant's task is to respond orally with the digit associated with each of the symbols as quickly as possible. The score is the total number of correct matches (out of 110) made by the subject within the 90 second time frame.

Good test-retest reliability (r=0.93-0.97, p<0.001) has been established in MS subjects (Benedict et al. (2006) Journal of the International Neuropsychological Society 12: 549-558; Benedict et al. (2008) Multiple Sclerosis 14: 940-946). Good discriminative validity for distinguishing between MS patients and normal controls (d=1.0-1.5, p<0.001) (see e.g., Deloire et al. (2005) Journal of Neurology, Neurosurgery & Psychiatry 76: 519-526; Benedict et al. (2006) Journal of the International Neuropsychological Society 12: 549-558; Houtchens et al. (2007) Neurology 69: 113-123; Strober et al. (2009) Multiple Sclerosis 15: 1077-1084; Parmenter et al. (2010) J Int Neuropsychol Soc 16: 6-16) and for distinguishing between RRMS and SPMS patients (d=0.8, p<0.001) (see Benedict et al. (2006) Archives of Neurology 63: 1301-1306) has also been confirmed. In addition, correlations between performance and brain MRI have also been documented (see e.g., Benedict et al. (2007) Multiple Sclerosis 13: 722-730; Houtchens et al. (2007) Neurology 69: 113-123; Tekok-Kilic et al. (2007) Neuramage 36: 1294-1300).

### Paced Serial Addition Test (PASAT)

First developed by Gronwall et al. to assess patients recovering from concussion, the PASAT requires patients to monitor a series of 61 audiotaped digits while adding each consecutive digit to the one immediately preceding it (Gronwall et al. (1977) Perceptual and Motor Skills 44: 367-373). The PASAT requires both rapid information processing and simultaneous allocation of attention to two tasks, as well as reasonably intact calculation ability. In its original format, the PASAT was administered at four inter-stimulus intervals (2.4 seconds, 2.0 seconds, 1.6 seconds, and 1.2 seconds). The number of inter-stimulus intervals and presentation rates were subsequently modified by Rao and colleagues for use with MS patients to 3.0 seconds and 2.0 seconds (Rao et al. (1991) A Manual for the Brief, Repeatable Battery of Neuropsychological Tests in Multiple Sclerosis: National Multiple Sclerosis Society; Rao et al. (1991) Neuropsychological Screening Battery for Multiple Sclerosis: National Multiple Sclerosis Society; Rao et al. (1991) Neurology 41: 685-691; Rao et al. (1991) Neurology 41: 692-696).

This latter version of the test was selected to be a component of the MS Functional Composite (MSFC) and the MACFIMS battery (Benedict et al. (2002) Clinical Neuropsychologist 16: 381-397). Test-retest reliability in MS populations ranges from r=0.78 to 0.93 (Benedict et al. (2006) Journal of the International Neuropsychological Society 16: 228-237; Drake et al. (2010) Multiple Sclerosis 16: 228-237). Good discriminative validity for distinguishing between MS patients and normal controls (d=0.5-0.7, p<0.001 to 0.34) (Deloire et al. (2005) Journal of Neurology, Neurosurgery & Psychiatry 76: 519-526; Benedict et al. (2006) Journal of the International Neuropsychological Society 12: 549-558; Houtchens et al. (2007) Neurology 69: 113-123; Strober et al. (2009) Multiple Sclerosis 15: 1077-1084; Parmenter et al. (2010) J Int Neuropsychol Soc 16: 6-16; Drake et al. (2010) Multiple Sclerosis 16: 228-237) and for distinguishing between RRMS and SPMS patients (d=0.5, p<0.002) (Benedict et al. (2006) Archives of Neurology 63: 1301-1306) has been confirmed. The PASAT score of interest is the total number of correct responses at each presentation rate. Two alternate forms of the Rao version of the PASAT are available (PASAT 3″ and PASAT 2″) and were selected in the current invention. In the PASAT 3″, the stimulus is presented every 3 seconds, where as in the PASAT 2″, the stimulus is presented every 2 seconds.

### Selective Reminding Test (SRT)

The SRT was first developed by Buschke et al. (see Buschke et al. (1974) Neurology 24: 1019-1025) who conducted research in the area of anterograde amnesia. Rather than ask patients to recall an entire word list on each successive learning trial, the experimenter only repeated words not recalled on each successive learning trial. Subsequently, several memory investigators developed normative data for the test, and alternate forms. Note, the original versions were based on a form of the test using 15 words and 12 learning trials. Such an administration is arduous and time consuming, and therefore there has been much interest in shorter forms of the SRT. The administration procedure widely used in MS research is a six-trial form developed by Rao et al. (see e.g., Rao et al. (1991) A Manual for the Brief, Repeatable Battery of Neuropsychological Tests in Multiple Sclerosis: National Multiple Sclerosis Society; Rao et al. (1991) Neuropsychological Screening Battery for Multiple Sclerosis: National Multiple Sclerosis Society; Rao et al. (1991) Neurology 41: 685-691; Rao et al. (1991) Neurology 41: 692-696). This six-trial format is utilized in the current invention. A number of different versions of SRT word lists exist. Hannay and Levin's word lists for adults, test forms 1 and 3, are utilized in the current invention (Hannay et al. (1985) J Clin Exp Neuropsychol. 7: 251-263). Discriminative validity of the SRT has been established in several studies, with SRT discriminating between MS subjects and normal controls d=0.6 to d=1.0 (see e.g., Rao et al. (1991) A Manual for the Brief, Repeatable Battery of Neuropsychological Tests in Multiple Sclerosis: National Multiple Sclerosis Society; Deloire et al. (2005) Journal of Neurology, Neurosurgery & Psychiatry 76: 519-526; Strober et al. (2009) Multiple Sclerosis 15: 1077-1084). It has also been shown that SRT findings can be associated with ventricular enlargement as seen on brain MRI (R²=0.14; p=0.05) (Christodoulou et al. (2003) Neurology 60: 1793-1798).

### Brief Visuospatial Memory Test—Revised (BVMT-R)

The BVMT-R is based on an initial effort to develop an equivalent alternate form visual memory test along the lines of the visual reproduction subtest from the Wechsler Memory Scale (Benedict et al. (1993) Neuropsychological Rehabilitation 3: 37-51; Benedict et al. (1995) Clinical Neuropsychologist 9: 11-16; Wechsler et al. (1987) Wechsler Memory Scale-Revised Manual. New York: Psychological Corporation). Initially, the BVMT included just a single exposure to a one-page presentation of six visual designs. The revised version includes three 10-second exposures to the stimulus (Benedict et al. (1997) Brief Visuospatial Memory Test—Revised: Professional Manual. Odessa, Fla.: Psychological Assessment Resources, Inc.; Benedict et al. (1996) Psychological Assessment 8: 145-153). After each exposure, the subject is asked to reproduce the matrix using a pencil on a blank sheet of paper. There are rigid scoring criteria for accuracy and location. After a 25 minute delay, the patient is asked to reproduce the information again without another exposure. Finally a yes/no recognition task is presented. The BVMT-R has excellent reproducibility, with test-retest reliability ranging from r=0.85 to r=0.91 (Benedict et al. (1996) Psychological Assessment 8: 145-153; Benedict et al. (2005) Journal of the International Neuropsychological Society 11: 727-736); as well as good discriminative validity between MS and normal control subjects (d=0.9, p<0.) (Strober et al. (2009) Multiple Sclerosis 15: 1077-1084; Parmenter et al. (2010) J Int Neuropsychol Soc 16: 6-16) and RRMS and SPMS patients (d=0.6, p<0.001) (Benedict et al. (2006) Archives of Neurology 63: 1301-1306). Predictive validity, in the form of correlation between BVMT-R performance and brain MRI findings, has also been established. Further, there is extensive research showing that all 6 forms of the test are of equivalent difficulty. Variables of interest in the current invention are the Total Learning and Delayed Recall scores.

This invention is further illustrated by the following examples which should not be construed as limiting. The contents of all references, figures, sequence listing, patents and published patent applications cited throughout this application are hereby incorporated by reference.

### EXAMPLES

### Example 1: Efficacy of Anti-LINGO-1 Antibodies in a Clinical Study

AON damages the optic nerve, causing loss of the myelin sheath and axonal injury, and may result in loss of visual function, e.g., result in permanent structural and functional visual deficits. AON is one of the initial manifestations of MS. There are commonalities in the pathology of MS and AON lesions (e.g., demyelination, axonal loss, inflammation). Current treatment is limited to high-dose intravenous (IV) corticosteroids that decrease inflammation in the acute phase but do not affect long-term visual outcome. Hence, there is an unmet need for therapies that can support repair and protection in AON and more generally in the central nervous system (CNS) during acute injury.

AON is considered a good clinical model to measure the mechanisms of action of anti-LINGO-1: remyelination and neuroprotection. Anti-LINGO-1 is a first-in-class human monoclonal antibody directed against LINGO-1 (leucine-rich repeat and immunoglobulin domain-containing neurite outgrowth inhibitor receptor-interacting protein-1), a CNS-specific cell surface glycoprotein and inhibitor of oligodendrocyte differentiation, myelination and remyelination. Anti-LINGO-1 has shown efficacy in preclinical models of remyelination and neuroprotection, and was well tolerated in Phase 1 clinical studies. See, e.g., Mi et al. CNS Drugs 27.7(2013):493-503; and Tran et al. Neurol. Neuroimmunol. Neuroinflamm. 1.2(2014):e18.

The randomized, double-blind, placebo-controlled, parallel-group Phase 2 (RENEW) trial (ClinicalTrials.gov Identifier: NCT01721161) was aimed to determine the efficacy and safety of anti-LINGO-1, e.g., BIIB033, e.g., opicinumab, for CNS remyelination following the onset of a first episode of AON and establish proof of biology. In the RENEW trial, two distinct mechanisms of action (MOA) of anti-LINGO-1 were studied: (i) remyelination via latency recovery as measured by visual evoked potentials (VEP); and (ii) neuroprotection via reduction of retinal nerve fiber layer (RNFL) and retinal ganglion cell layer (RGCL) thinning as measured by spectral-domain optical coherence tomography (SD-OCT).

### Methods

A group of 82 patients receiving a total of 6 intravenous infusions of 100 mg/kg of anti-LINGO-1 antibody or placebo every four weeks was treated to evaluate the effect of an anti-LINGO-1 antibody in patients treated following a first episode of AON according to the RENEW) trial ClinicalTrials.gov Identifier: NCT01721161.

Eligible subjects were 18-55 years of age, had no history of multiple sclerosis (MS), and were experiencing a first unilateral AON episode. A diagnosis AON was based on the presence of at least two of the following: reduced visual acuity; afferent pupillary defect; color vision loss; visual field abnormality; and pain on eye movement. Enrollment was permitted irrespective of whether demyelinating lesions were present on brain magnetic resonance imaging. AON as onset of multiple sclerosis (MS; newly diagnosed) was acceptable. Participants were excluded if they had: a prior episode of optic neuritis/previous central nervous system demyelinating event indicative of MS; an established diagnosis of MS; refractive errors of ±6 diopters or more in either eye; loss of vision not due to AON; a history or evidence of severe disc edema or haemorrhage; an abnormal full-field visual evoked potential (FF-VEP) in the fellow eye at screening; a concomitant ophthalmologic disorder, e.g., diabetic retinopathy, macular degeneration, macular exudate, macular edema, glaucoma, severe astigmatism, ocular trauma, neuromyelitis optica, ischemic optic neuropathy, congenital nystagmus, or other ophthalmologic conditions that could confound the assessment of functional and anatomic endpoint; a history of any clinically significant cardiac, endocrinologic, hematologic, hepatic, immunologic, metabolic, urologic, pulmonary, neurologic, dermatologic, psychiatric, oncologic, renal, severe allergic or anaphylactic reactions, or other major disease; a history of HIV, hepatitis C infection, or hepatitis B infection; a history of drug or alcohol abuse in the last 2 years; been enrolled in another study within the last 3 months or participated in a previous study with anti-leucine-rich repeat and immunoglobulin domain-containing neurite outgrowth inhibitor receptor-interacting protein-1 (anti-LINGO-1); or an inability to comply with study requirements. Participants also were excluded if the investigator felt there were other reasons making participation unsuitable or, if female participants were currently pregnant, were breastfeeding or were planning to conceive during the study.

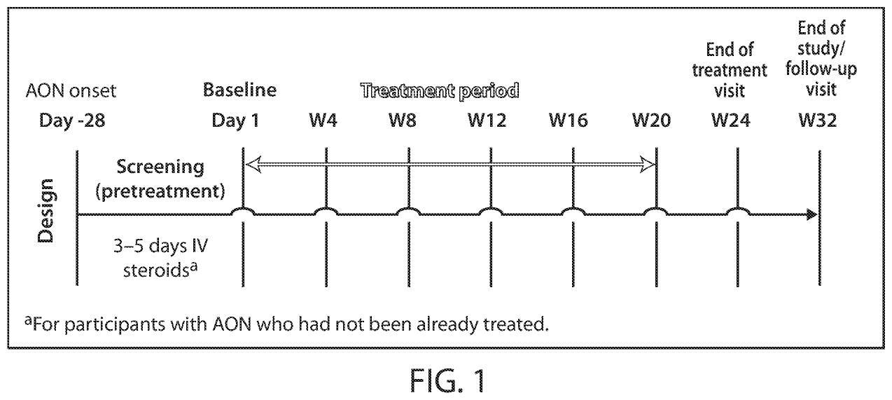

All participants received treatment with 1 g methylprednisolone/day IV for 3-5 days before randomization. Following treatment with high-dose methylprednisolone, participants were randomized 1:1 to placebo or anti-LINGO-1 100 mg/kg IV every 4 weeks from baseline to week 20 (6 treatments) and followed to week 32 (FIG. 1). Only the pharmacists preparing treatments/pharmacy monitors were unblinded. Participants were randomized by the permuted block method using a centralized interactive voice and web response system. The block size was four and the number of blocks was 50. An independent biostatistician was responsible for overseeing the randomization.

Full-field VEP (FF-VEP), spectral-domain optical coherence tomography (SD-OCT), and low-contrast letter acuity (LCLA) were assessed at screening, baseline, and every 4-8 weeks to study end (week 32) to assess efficacy. The primary endpoint was optic nerve conduction latency at week 24 in the affected eye compared with the unaffected fellow eye at baseline, as measured using full-field VEP (FF-VEP). Recovery of optic nerve conduction latency using FF-VEP compared with the unaffected fellow eye at baseline was a way to evaluate remyelination. A final latency assessment was performed at study end (week 32).

A number of additional analyses, based on FF-VEP latency, were performed, including evaluating the number of participants with FF-VEP latency recovery, defined as affected eye FF-VEP latency <10% worse than the fellow eye, at week 24 (prespecified). A post hoc sensitivity analysis around this recovery threshold was performed. The change in FF-VEP latency at week 24 in the intent-to-treat (ITT) population between treatment groups for participants who received ≥4 infusions (post hoc) was also determined.

Secondary efficacy endpoints included change in the following at week 24: (i) RNFL thickness in the affected eye compared with the unaffected eye at baseline; (ii) RGCL/inner plexiform retinal layer (IPL) thickness in the affected eye compared with the unaffected eye at baseline; (iii) change in low-contrast letter acuity (LCLA) from baseline measured using 1.25% and 2.5% Sloan letter charts; the affected eye's own baseline value was used. Thickness of the retinal nerve fiber layer and ganglion cell layer (measured using spectral-domain optical coherence tomography (SD-OCT)) and change in low-contract letter acuity (LCLA) were methods used to evaluate neuroprotection.

Between-treatment comparisons were evaluated by analysis of covariance (ANCOVA) and mixed-effect model repeated measure (MMRM) in the following subject populations: (i) per-protocol (PP; subjects who completed the study, did not miss >1 dose of treatment, and did not receive MS modifying therapy) and (ii) intent-to-treat (ITT; all randomized subjects who received ≥1 dose of study treatment).

For the efficacy endpoints, adjusted mean change was calculated and between-treatment comparisons evaluated by analysis of covariance (ANCOVA) at week 24 and mixed-effect model repeated measure (MMRM) through week 32. Week 32 data were used as a supportive analysis to check whether treatment effect was sustained between end of treatment (week 24) and end of study (week 32).

A more detailed description of these patient populations is as follows:

### Intent-to-Treat Population (ITT)

All randomized patients who received at least 1 dose of anti-LINGO-1 or placebo (regardless of their compliance with the protocol), but did not necessarily complete the study. It carries forward the last observed data point per patient who discontinued, until end of study. The ITT population received fewer doses and last observation data was carried forward, potentially impacting the treatment effect observed.

### Per-Protocol Population

The per-protocol population is defined as subjects from the ITT population who complete the study, did not miss more than one dose of anti-LINGO-1 or placebo, and did not receive MS modifying therapies during the study period. Last observation carried forward was used for imputation in the ANCOVA analyses.

The subgroups of patients with vs. without FF-VEP latency recovery, defined as affected eye FF-VEP latency ≤10% worse than the fellow eye at week 24, also were compared (post hoc analysis) between treatment groups using a chi-squared test; the sensitivity analyses surrounding the 10% cutoff for these analyses used both chi-squared and Fisher's exact tests. The baseline of the unaffected fellow eye was used as the baseline covariate in ANCOVA and MMRM.

### Measurement of FF-VEP

All centers were required to perform all FF-VEP studies using a standard protocol which complied with both the International Society for Clinical Electrophysiology of Vision and American Clinical Neurophysiology Society guidelines. Each VEP study was interpreted independently by two masked clinical electrophysiologists. If the data agreement was not within specified parameters, a third masked, independent clinical electrophysiologist arbitrated the data by reconciling the reader disagreements according to his/her best judgment and expertise, and providing the final interpretation. Each VEP was interpreted without reference to any of the participant's other VEP data. Central readers were not involved in data collection or analysis.

### Measurement of RGCL/IPL and RNFL by SD-OCT

SD-OCT scans were obtained according to a standardized study protocol. Images were obtained at each site from a Spectralis (Heidelberg Engineering, Heidelberg Germany) or Cirrus (Carl Zeiss Meditec, Dublin, Calif.) system.

For each participant for whom images were obtained on the Spectralis System, the following scan patterns were obtained on each eye: a dense 97-line preset scan covering a 20°×20° area of the macula centered on the foveal center point in high-speed mode with an ART setting of 16 was used to image the neurosensory retinal layers and vitreoretinal interface; a seven-line preset scan pattern covering a 30°×5° area centered on the foveal center point in high-resolution mode with an ART setting of 25 was used to assess the central macula; an optic nerve head 73-line preset scan covering a 15°×15° area centered on the optic nerve in high-speed mode with an ART setting of 9 was used to image the optic nerve, peripapillary area, and corresponding vitreoretinal interface; an RNFL preset optic nerve 12° diameter circle scan centered on the optic nerve was used to measure the RNFL thickness.

For each participant for whom images were obtained on the Cirrus System, the following scan patterns were obtained on each eye: a 512×128 macular cube covering a 6 mm×6 mm area of the macula centered on the foveal center point was used to image the neurosensory retinal layers and vitreoretinal interface; a five-line raster (HD) preset raster scan pattern centered on the foveal center point was used to assess the central macula; a 200×200 optic nerve cube preset scan centered on the optic nerve was used to image the optic nerve, peripapillary area, and corresponding vitreoretinal interface, and to measure RNFL thickness.

Two certified readers in a masked and independent manner determined macular and RNFL thickness and made morphological assessments on coded scans for each participant. A data specialist entered all concordant values from the two readers into the trial database and flagged discrepant values. A third certified senior reader arbitrated the discrepant values. The senior reader reconciled all reader disagreements according to his/her best judgment and expertise, recording his/her decision as the final arbitrated value that the data specialist entered into the trial database. Each SD-OCT categorical variable was graded as present, absent, or unreadable (due to poor quality or centration of the scan), according to either the agreed decision between masked readers or the arbitrated decision when the readers disagreed. For ganglion cell complex measurement, two independent readers assessed segmentation line placement, and the two readers adjudicated discrepancies. After finishing arbitration, adjudication, and data entry, another masked data specialist verified the accuracy of all values entered into the trial database.

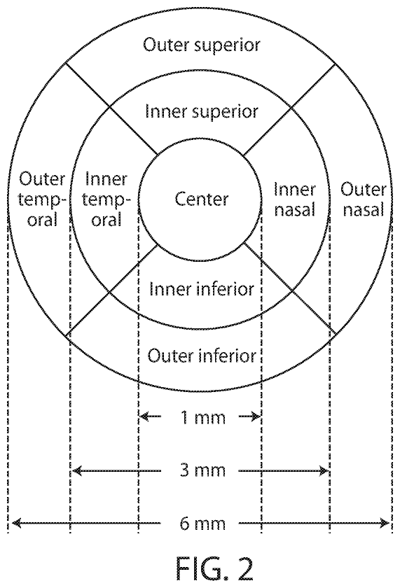

Central subfield and RNFL thickness were measured on macular and RNFL Spectralis and Cirrus scans using Heyex software (Heidelberg Engineering), and Cirrus software (Zeiss Meditech), respectively. Spectralis macular and RNFL segmentation line artifacts were corrected manually. Cirrus macular segmentation line artifacts were also corrected manually. It was not possible to correct Cirrus RNFL segmentation artifacts manually because of software limitations. Ganglion cell complex that comprised the RGCL/IPL thickness was measured using customized semiautomated software (DOCTRAP). The average RGCL thickness was calculated in each of nine sectors corresponding to the Early Treatment Diabetic Retinopathy grid as shown in FIG. 2. In addition, a global average thickness across all nine sectors was determined.

### Results

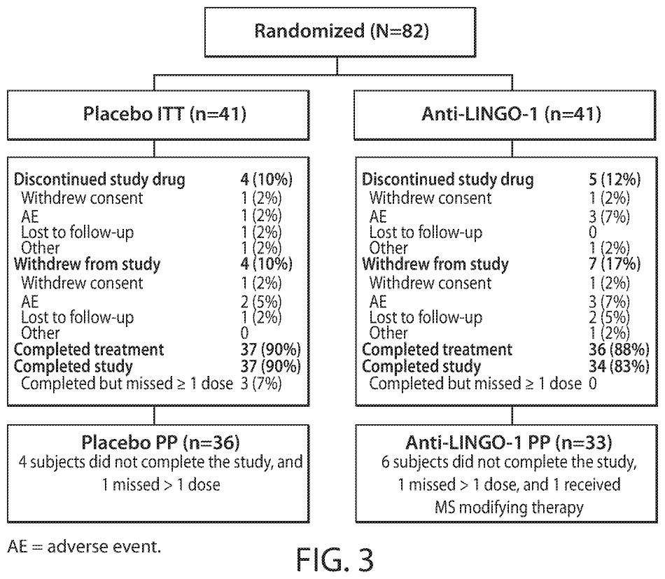

Eighty-two subjects were randomized to placebo or anti-LINGO-1 at 33 sites across Europe, Australia, and Canada. All subjects were included in the ITT analyses (n=41 in each group). The PP population comprised 36 subjects in the placebo group and 33 treated with anti-LINGO-1 (FIG. 3). The groups were similar in baseline demographics (Table 5) and rates of study withdrawal and treatment discontinuation (FIG. 3).

TABLE 5
Subject demographic characteristics at baseline

| | ITT | | | PP | | |
|---|---|---|---|---|---|---|
| | | Anti- | All | | Anti- | All |
| | Placebo | LINGO-1 | subjects | Placebo | LINGO-1 | subjects |
| Characteristic | n = 41 | n = 41 | N = 82 | n = 36 | n = 33 | N = 69 |
| Female, % | 76 | 66 | 71 | 75 | 64 | 70 |
| White, % | 95 | 98 | 96 | 97 | 97 | 97 |
| Mean ± SD | 32.4 ± 8.85 | 31.8 ± 7.17 | 32.1 ± 8.01 | 32.2 ± 8.80 | 31.2 ± 7.12 | 31.7 ± 8.00 |
| age, y | | | | | | |
| Median | 75 | 71.2 | 72.2 | 73.8 | 72.2 | 72.5 |
| (range) | (47-119) | (46-106) | (46-119) | (50-119) | (46-106) | (46-119) |
| weight, kg | | | | | | |
| Median | 169.0a | 170.0a | 170.0a | 169.5b | 170.0b | 170.0b |
| (range) | (155-194) | (158-188) | (155-194) | (155-194) | (158-188) | (155-194) |
| height, cm | | | | | | |

an = 39 in each group; total N = 78.
bPlacebo, n = 34; anti-LINGO-1, n = 31; total N = 65.

However, more severe cases of AON were randomized more frequently to anti-LINGO-1 than placebo as shown by the prevalence of conduction block (lack of any FF-VEP response; 2:1 for the anti-LINGO-1 vs. placebo groups) and severity of AON signs and symptoms post steroid treatment (Table 6).

TABLE 6
Subject clinical characteristics

| | ITT | | | PP | | |
|---|---|---|---|---|---|---|
| | | Anti- | All | | Anti- | |
| | Placebo | LINGO-1 | subjects | Placebo | LINGO-1 | All subjects |
| Characteristic | n = 41 | n = 41 | N = 82 | n = 36 | n = 33 | N = 69 |
| Mean ± SD days | 24.6 ± 3.44 | 23.6 ± 3.98 | 24.1 ± 3.73 | 24.3 ± 3.49 | 24.0 ± 3.75 | 24.2 ± 3.59 |
| from first AON | | | | | | |
| symptom to | | | | | | |
| first dose^{a} | | | | | | |
| Mean ± SD days | 19.2 ± 4.85 | 18.7 ± 4.73 | 19.0 ± 4.77 | 19.1 ± 4.97 | 19.2 ± 4.55 | 19.1 ± 4.74 |
| from confirmed | | | | | | |
| AON diagnosis | | | | | | |
| to first dose | | | | | | |
| No. with affected | 19 (46) | 25 (61) | 44 (54) | 16 (44) | 20 (61) | 36 (52) |
| right eye (%) | | | | | | |
| Criteria for AON | | | | | | |
| diagnosis, n (%) | | | | | | |
| Decreased visual | 36 (88) | 41 (100) | 77 (94) | 31 (86) | 33 (100) | 64 (93) |
| acuity | | | | | | |
| Decreased color | 30 (73) | 33 (80) | 63 (77) | 27 (75) | 26 (79) | 53 (77) |
| vision | | | | | | |
| Relative afferent | 34 (83) | 31 (76) | 65 (79) | 29 (81) | 23 (70) | 52 (75) |
| pupillary defect | | | | | | |
| Visual field defect | 32 (78) | 37 (90) | 69 (84) | 27 (75) | 30 (91) | 57 (83) |
| Ocular pain | 31 (76) | 36 (88) | 67 (82) | 27 (75) | 28 (85) | 55 (80) |
| Other | 5 (12) | 4 (10) | 9 (11) | 5 (14) | 4 (12) | 9 (13) |
| AON signs and | | | | | | |
| symptoms^{b} at | | | | | | |
| Screening or | | | | | | |
| Baseline, n (%) | | | | | | |
| Visual field defect | 29 (71) | 34 (83) | 63 (77) | 25 (69) | 27 (82) | 52 (75) |
| Color desaturation | 33 (80) | 32 (78) | 65 (79) | 28 (78) | 25 (76) | 53 (77) |
| Uhthoff's symptom | 8 (20) | 18 (44) | 26 (32) | 6 (17) | 16 (48) | 22 (32) |
| Swollen optic disc | 8 (20) | 12 (29) | 20 (24) | 6 (17) | 11 (33) | 17 (25) |
| Relative afferent | 33 (80) | 30 (73) | 63 (77) | 28 (78) | 24 (73) | 52 (75) |
| pupillary defect | | | | | | |
| FF-VEP conduction | 5 (12) | 10 (24) | 15 (18) | 5 (14) | 6 (18) | 11 (16) |
| block in the affected | | | | | | |
| eye at Baseline, | | | | | | |
| n (%) | | | | | | |
| Mean ± SD FF-VEP | 101.66 ± 5.248 | 102.66 ± 6.353 | 102.16 ± 5.812 | 101.00 ± 4.905 | 102.62 ± 6.074 | 101.78 ± 5.51 [symbol] |
| latency in the fellow | | | | | | |
| eye at Baseline, ms | | | | | | |
| Mean ± SD brain | 0.5 ± 1.61 | 0.2 ± 1.02 | 0.4 ± 1.35 | 0.2 ± 0.65 | 0.1 ± 0.35 | 0.1 ± 0.53 |
| Gd+ lesions before | | | | | | |
| first dose^{c} | | | | | | |
| Mean ± SD | 66.0 ± 6.9 | 63.8 ± 7.4 | 64.8 ± 7.2 | 65.9 ± 7.2 | 63.6 ± 8.1 | 64.8 ± 7.7 |
| RGCL/IPL | | | | | | |
| thickness in the | | | | | | |
| affected eye at | | | | | | |
| baseline - μm§ | | | | | | |
| Mean ± SD | 1.0895 ± 1.31543 | 1.0900 ± 1.90443 | 1.0898 ± 1.62570 | 0.9461 ± 1.03425 | 0.8419 ± 1.54836 | 0.8948 ± 1.30383 |
| volume of brain T2 | | | | | | |
| lesions before | | | | | | |
| first dose,^{a} mL | | | | | | |

Gd+ = gadolinium-enhancing.
^{a}First dose given on average 2 weeks after completion of high-dose IV methylprednisolone treatment (1 g daily for 3-5 days).
^{b}Symptoms were not uniformly tested in accordance with a predefined protocol.
^{c}ITT population: n = 38 in each group; total N = 76. § n = 38 in the placebo group and n = 40 in the opicinumab group, total = 78 for the ITT population; n = 34 in the placebo group and n = 32 in the opicinumab group, total = 66 for the per-protocol (PP) population.
PP population: placebo, n = 34; anti-LINGO-1, n = 33; total N = 67. [symbol]
indicates data missing or illegible when filed

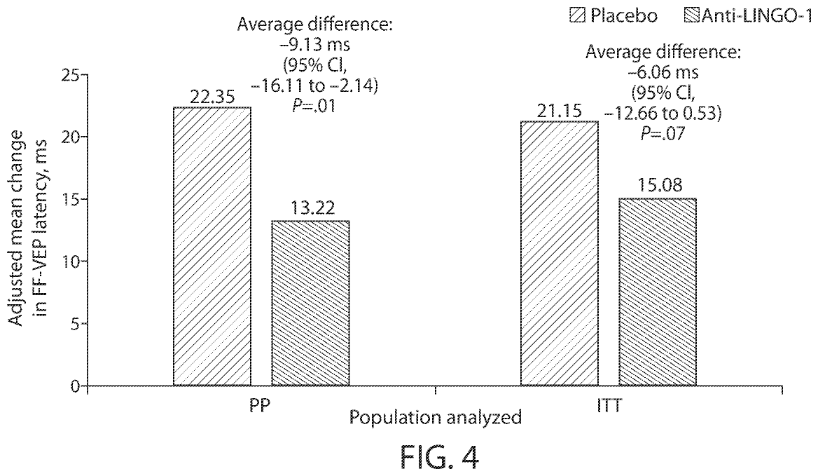

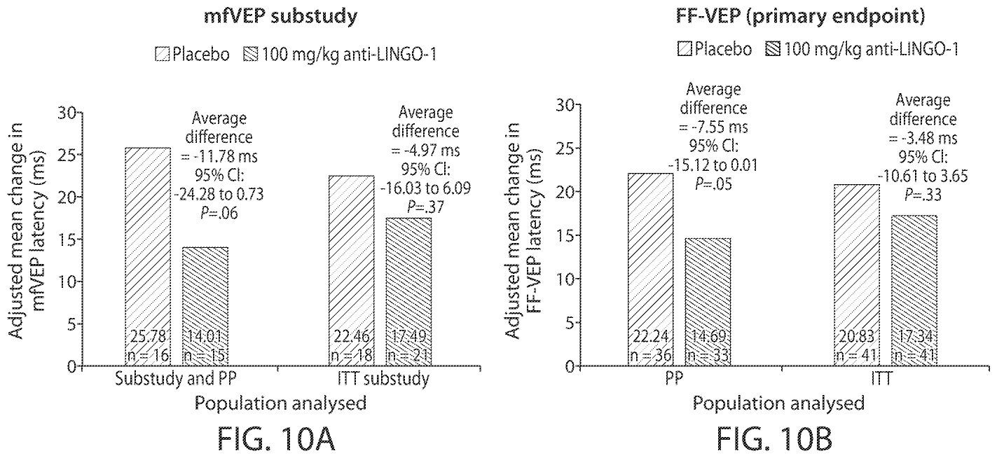

The average time on treatment was 23.0±4.12 weeks in the placebo group and 22.5±5.01 weeks in the anti-LINGO-1 group. In the PP and ITT populations, the anti-LINGO-1 group showed improved optic nerve conduction latency vs. placebo at week 24 (Table 7) and week 32. In the PP population, anti-LINGO-1-treated patients showed a significantly improved average difference in latency recovery vs placebo: 7.55 msec at 24 wks (95% CI, −15.1 to 0.0, ANCOVA p=0.05) (FIG. 4) and 9.13 msec (95% CI, −16.1 to −2.1, MMRM p=0.01) at 32 wks (FIG. 10B). Corresponding differences in the ITT population were 3.48 msec (95% CI, −10.6 to 3.7, p=0.33) at 24 wks (FIG. 4) and 6.06 msec (95% CI, −12.7 to 0.5, p=0.07) at 32 wks (FIG. 10B). In participants from the ITT population who received ≥4 infusions, improvement in FF-VEP latency at week 24 was similar to the PP population analysis: adjusted mean change of 22.0 (placebo; n=38) versus 15.8 (anti-LINGO-1; n=37), a treatment difference versus placebo of −6.1 ms (95% CI, −13.3 to 1.1; P=0.10) using ANCOVA.

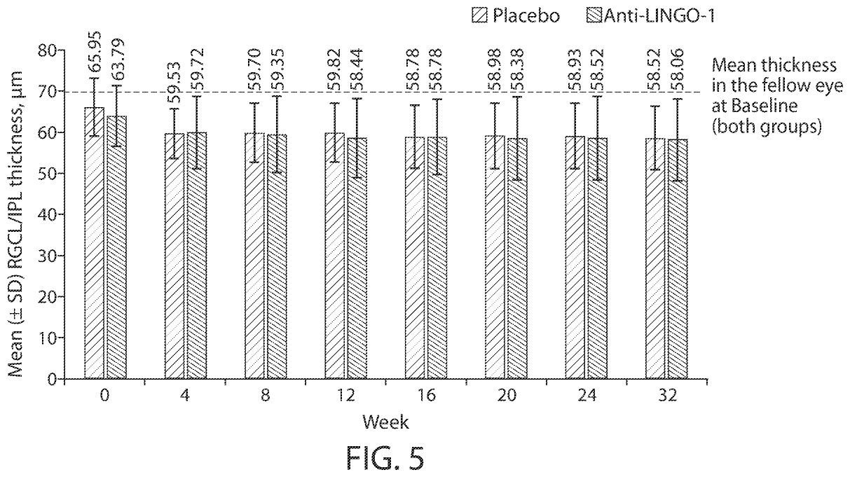

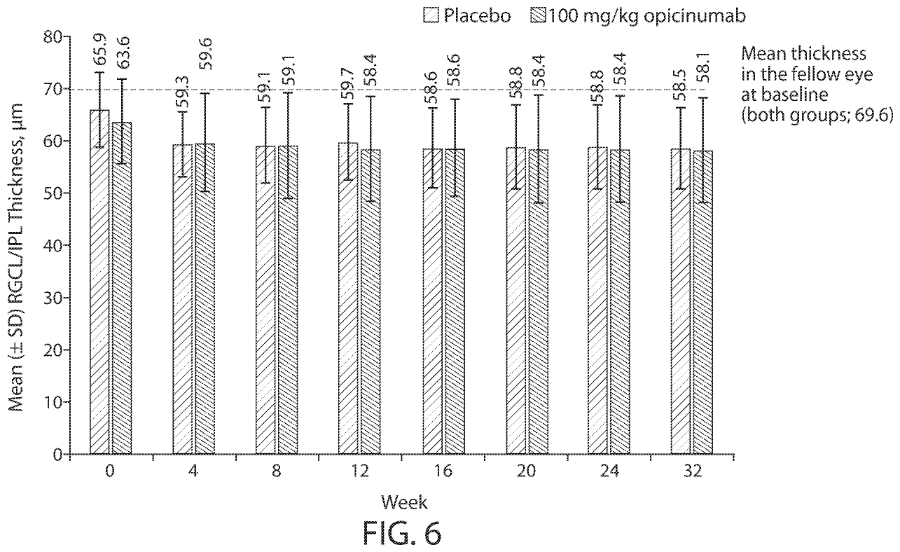

During the 32 week period, no differences were observed in secondary efficacy endpoints, SD-OCT and LCLA. (Table 7 and FIGS. 5 and 6). Overall incidence and severity of adverse events (AEs) were comparable across treatment arms. Treatment-related serious AEs were infusion-related hypersensitivity reactions (N=2) and asymptomatic transient elevation in liver transaminases (N=1).

TABLE 7
Summary of primary and secondary efficacy outcomes at week 24

| | ITT | | PP | |
|---|---|---|---|---|
| | Change for | | Change for | |
| | placebo and anti- | | placebo and anti- | |
| | LINGO-1, plus | | LINGO-1, plus | |
| | difference vs. | | difference vs. | |
| | placebo | | placebo | |
| Endpoint | (95% CI) | P value | (95% CI) | P value |
| Mean change | 20.83 ms; | .33 | 22.24 ms; | .05 |
| in optic nerve | 17.34 ms | | 14.69 ms | |
| latency (FF- | −3.48 ms | | −7.55 ms | |
| VEP) | (−10.61 to 3.65) | | (−15.12 to 0.01) | |
| Mean | −11.77%; | .19 | −12.22%; | .15 |
| percentage | −15.66% | | −16.98% | |
| change in | −3.89% | | −4.76% | |
| RNFL | (−9.70 to 1.92) | | (−11.26 to 1.74) | |
| thickness | | | | |
| (SD-OCT) | | | | |
| Mean change | −9.90 μm; | .50 | −10.17 μm; | .35 |
| in RGCL/IPL | −11.05 μm | | −11.93 μm | |
| thickness | −1.15 μm | | −1.76 μm | |
| | (−4.51 to 2.21) | | (−5.50 to 1.98) | |
| Change in | 8.1; 6.5 | .54 | 7.2; 6.0 | .66 |
| LCLA; | −1.6 | | −1.2 | |
| affected eye, | (−6.9 to 3.6) | | (−6.6 to 4.3) | |
| 1.25% Sloan | | | | |
| letter chart | | | | |
| Change in | 11.9; 11.0 | .77 | 11.6; 10.8 | .80 |
| LCLA; | −0.8 | | −0.8 | |
| affected eye, | (−6.5 to 4.9) | | (−6.7 to 5.2) | |
| 2.5% Sloan | | | | |
| letter chart | | | | |

CI = confidence interval.

Among participants in the ITT population whose FF-VEP latency was impaired in the affected eye at baseline (defined as >3% worse than fellow eye or with conduction block), 53% of patients (16/30) in the anti-LINGO-1 group had FF-VEP latency recovery at week 24 (defined as affected eye FF-VEP latency ≤10% worse than the fellow eye) compared with 26% (9/34) of the placebo group (P=0.0279). At week 12, 29% (10/35) of the anti-LINGO-1 group and 12% (4/33) of the placebo group had normal/mildly prolonged FF-VEP latency (P=0.09). Similar results were observed in the PP population with 54% (15/28) of the anti-LINGO-1 and 27% (9/33) of the placebo groups having normal/mildly prolonged FF-VEP latency at week 24 (P=0.04) and 30% (9/30) of the anti-LINGO-1 and 13% (4/31) of the placebo groups at week 12 (P=0.10). Post hoc sensitivity analyses performed in the PP population demonstrated that 10% was an appropriate cutoff to indicate latency recovery (see Table 8).

TABLE 8
Full-Field Visual Evoked Potential Latency Recovery Sensitivity Analyses in the
Per-Protocol Population.

| | Baseline | | Week 12 | | Week 24 | |
|---|---|---|---|---|---|---|
| | | Anti- | | Anti- | | Anti- |
| | Placebo | LINGO-1 | Placebo | LINGO-1 | Placebo | LINGO-1 |
| No. with >3% | 34 | 30 | | | | |
| prolonged latency or | (n = 36) | (n = 33) | | | | |
| conduction failure | | | | | | |
| ≤5% worse than | 1/34 (3) | 2/30 (7) | 0/31 | 4/30 (13) | 5/33 (15) | 9/28 (32) |
| fellow eye | | | | | | |
| >5% worse than | 33/34 (97) | 28/30 (93) | 31/31 (100) | 26/30 (87) | 28/33 (85) | 19/28 (68) |
| fellow eye or | | | | | | |
| conduction block - | | | | | | |
| no. (%) | | | | | | |
| Difference* | 4 | | 13 | | 17 | |
| Chi-squared P | 0.48 | | 0.04 | | 0.12 | |
| value | | | | | | |
| Fisher exact P | 0.60 | | 0.05 | | 0.14 | |
| value | | | | | | |
| ≤8% worse than | 1/34 (3) | 2/30 (7) | 2/31 (6) | 7/30 (23) | 8/33 (24) | 14/28 (50) |
| fellow eye | | | | | | |
| >8% worse than | 33/34 (97) | 28/30 (93) | 29/31 (94) | 23/30 (77) | 25/33 (76) | 14/28 (50) |
| fellow eye or | | | | | | |
| conduction block - | | | | | | |
| no. (%) | | | | | | |
| Difference* | 4 | | 17 | | 26 | |
| Chi-squared P | 0.48 | | 0.06 | | 0.04 | |
| value | | | | | | |
| Fisher exact P | 0.60 | | 0.08 | | 0.06 | |
| value | | | | | | |
| ≤10% worse than | 1/34 (3) | 2/30 (7) | 4/31(13) | 9/30 (30) | 9/33 (27) | 15/28 (54) |
| fellow eye | | | | | | |
| >10% worse than | 33/34 (97) | 28/30 (93) | 27/31 (87) | 21/30 (70) | 24/33 (73) | 13/28 (46) |
| fellow eye or | | | | | | |
| conduction block - | | | | | | |
| no. (%) | | | | | | |
| Difference* | 4 | | 17 | | 27 | |
| Chi-squared P | 0.48 | | 0.10 | | 0.04 | |
| value | | | | | | |
| Fisher exact P | 0.60 | | 0.13 | | 0.06 | |
| value | | | | | | |
| ≤12% worse than | 3/34 (9) | 4/30 (13) | 7/31 (23) | 13/30 (43) | 9/33 (27) | 15/28 (54) |
| fellow eye - no. (%) | | | | | | |
| >12% worse than | 31/34 (91) | 26/30 (87) | 24/31 (77) | 17/30 (57) | 24/33 (73) | 13/28 (46) |
| fellow eye or | | | | | | |
| conduction block - | | | | | | |
| no. (%) | | | | | | |
| Difference* | 4 | | 20 | | 27 | |
| Chi-squared P | 0.56 | | 0.08 | | 0.04 | |
| value | | | | | | |
| Fisher exact P | 0.70 | | 0.11 | | 0.06 | |
| value | | | | | | |
| ≤15% worse than | 3/34 (9) | 10/30 (33) | 11/31 (35) | 16/30 (53) | 11/33 (33) | 16/28 (57) |
| fellow eye - no. (%) | | | | | | |
| >15% worse than | 31/34 (91) | 20/30 (67) | 20/31 (65) | 14/30 (47) | 22/33 (67) | 12/28 (43) |
| fellow eye or | | | | | | |
| conduction block - | | | | | | |
| no. (%) | | | | | | |

| | | | |
|---|---|---|---|
| Difference* | 24 | 18 | 24 |
| Chi-squared P | 0.02 | 0.16 | 0.06 |
| value | | | |
| Fisher exact P | 0.03 | 0.20 | 0.08 |
| value | | | |

*Difference was calculated as percentage of anti-LINGO-1 minus percentage of placebo.

No treatment effect was observed for the secondary efficacy endpoints of RNFL and RGCL/IPL thickness by SD-OCT or change in LCLA for placebo versus anti-LINGO-1 at week 24 (see Table 9 for PP population). However, the majority of RGCL/IPL thinning in the PP population occurred before the first study dose administration and all had occurred before the second dose was given on week 4 (Table 6, FIG. 6; corresponding fellow eye data given in Table 10). The results for the secondary endpoints were similar for the ITT population (Table 11, FIG. 5).

TABLE 9
Secondary Efficacy Outcomes at Week 24 (Analysis of
Covariance) for the Per-Protocol Population.

| | Placebo | Opicinumab |
|---|---|---|
| Endpoint | (N = 36) | (N = 33) |
| Mean percentage change in RNFL | −12.2 | −17.0 |
| thickness—SD-OCT*; affected eye | | |
| at week 24 vs. baseline fellow eye | | |
| Treatment difference at week 24 vs. | −4.8 (−11.3 to 1.7) | |
| placebo (95% CI)* | | |
| P value | 0.15 | |
| Mean change in RGCL/IPL | −10.2 | −11.9 |
| thickness—μm*; affected eye at | | |
| week 24 vs. baseline fellow eye | | |
| Treatment difference at week 24 vs. | −1.8 (−5.5 to 2.0) | |
| placebo (95% CI)* | | |
| P value | 0.35 | |
| Change in LCLA—1.25% Sloan chart^{†}; | 7.2 | 6.0 |
| affected eye at week 24 vs. baseline | | |
| Treatment difference at week 24 vs. | −1.2 (−6.6 to 4.3) | |
| placebo (95% CI)^{†} | | |
| P value | 0.66 | |
| Change in LCLA—2.5% Sloan chart^{†}; | 11.6 | 10.8 |

| | |
|---|---|
| affected eye at week 24 vs. baseline | |
| Treatment difference at week 24 vs. | −0.8 (−6.7 to 5.2) |
| placebo (95% CI)^{†} | |
| P value | 0.80 |

CI denotes confidence interval, RGCL/IPL retinal ganglion cell layer/inner plexiform retinal layer, RNFL retinal nerve fiber layer, and SD-OCT spectral-domain optical coherence tomography.
*A decrease (negative value) represents loss of retinal layers; the difference vs. placebo represents the more severe acute optic neuritis evident pretreatment initiation in the opicinumab group (Table 6).
^{†}An increase in low-contrast letter acuity (LCLA) from baseline represents recovery; a negative difference vs. placebo indicates a lack of treatment effect.

TABLE 10
Mean Retinal Ganglion Cell Layer/Inner Plexiform
Layer (RGCL/IPL) Thickness (Measured by Spectral-Domain
Optical Coherence Tomography) in the Fellow Eye in the
Intent-to-Treat (ITT) and Per-Protocol (PP) Populations
over the Course of the Study.

| Mean ± SD | ITT | | PP | |
|---|---|---|---|---|
| GCL/IPL | Placebo | Anti-LINGO-1 | Placebo | Anti-LINGO-1 |
| thickness—μm | (N = 41) | (N = 41) | (N = 36) | (N = 33) |
| Baseline | 69.2 ± 5.7 | 69.8 ± 5.5 | 68.9 ± 5.8 | 70.5 ± 5.7 |
| Week 12 | 69.0 ± 6.1 | 69.8 ± 5.9 | 68.7 ± 5.9 | 70.4 ± 6.0 |
| Week 24 | 69.1 ± 5.8 | 69.9 ± 6.5 | 68.8 ± 5.7 | 70.2 ± 6.5 |
| Week 32 | 68.7 ± 5.7 | 69.9 ± 6.5 | 68.7 ± 5.7 | 69.9 ± 6.4 |

TABLE 11
Secondary Efficacy Outcomes at Week 24 (Analysis of
Covariance) for the Intent-to-Treat Population.

| | Placebo | Anti-LINGO-1 |
|---|---|---|
| Endpoint | (N = 41) | (N = 41) |
| Mean percentage change in RNFL | −11.8 | −15.7 |
| thickness—SD-OCT*; affected eye | | |
| at week 24 vs. baseline fellow eye | | |
| Treatment difference at week 24 vs. | −3.9 (−9.7 to 1.9) | |
| placebo (95% CI)* | | |
| P value | 0.19 | |
| Mean change in RGCL/IPL | −9.9 | −11.1 |
| thickness—μm*; affected eye at | | |
| week 24 vs. baseline fellow eye | | |
| Treatment difference at week 24 vs. | −1.2 (−4.5 to 2.2) | |
| placebo (95% CI)* | | |
| P value | 0.50 | |
| Change in LCLA—1.25% Sloan chart^{†}; | 8.1 | 6.5 |
| affected eye at week 24 vs. baseline | | |
| Treatment difference at week 24 vs. | −1.6 (−6.9 to 3.6) | |
| placebo (95% CI)^{†} | | |
| P value | 0.54 | |
| Change in LCLA—2.5% Sloan chart^{†}; | 11.9 | 11.0 |

| | |
|---|---|
| affected eye at week 24 vs. baseline | |
| Treatment difference at week 24 vs. | −0.8 (−6.5 to 4.9) |
| placebo (95% CI)^{†} | |
| P value | 0.77 |

CI denotes confidence interval, RGCL/IPL retinal ganglion cell layer/inner plexiform retinal layer, RNFL retinal nerve fiber layer, and SD-OCT spectral-domain optical coherence tomography.
*A decrease (negative value) represents loss of retinal layers; as retinal thinning occurred very rapidly (≥half before the first dose and all by the second dose), the difference versus placebo represents the more severe acute optic neuritis evident in the anti-LINGO-1 treatment group.
^{†}An increase in low-contrast letter acuity (LCLA) from baseline represents recovery; a negative difference versus placebo indicates a lack of treatment effect.

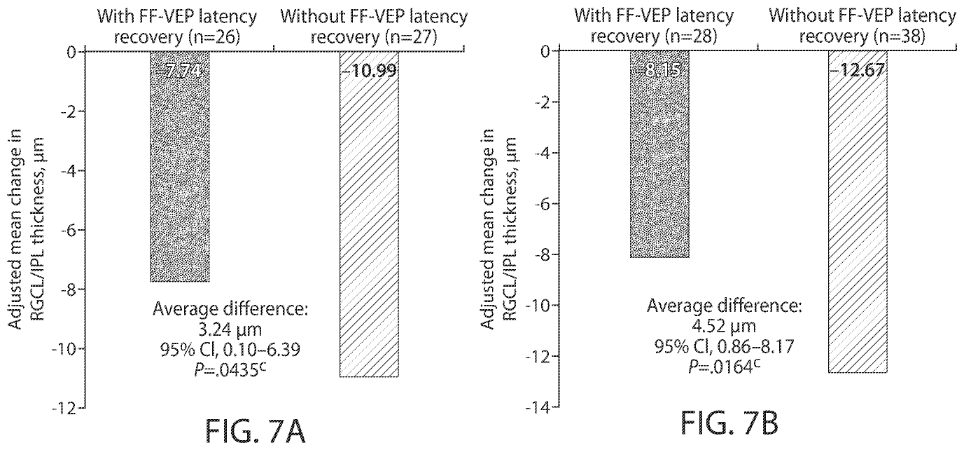

Mean RGCL thinning (as measured by SD-OCT; post hoc analysis) at week 24 was significantly lower in subjects in the ITT population with FF-VEP latency recovery than without. Average difference was 4.92 μm (95% CI, 1.39-8.46; P=0.0071). Adjusted mean change in RGCL/IPL thickness was −7.83 μm in 29 subjects with FF-VEP latency recovery (placebo, n=10; anti-LINGO-1, n=19) and −12.75 μm in 40 subjects without (placebo, n=26; anti-LINGO-1, n=14). In the PP population, at week 4, the average difference was 3.24 μm (95% CI, 0.10-6.39 P=0.0435 by ANCOVA) in subjects having FF-VEP latency recovery relative to those without FF-VEP latency recovery. At week 24, the average difference was 4.52 μm (95% CI, 0.86-8.17 P=0.0164 by ANCOVA). The results for the PP population are shown in FIG. 7.

Change in FF-VEP latency was stratified in subgroups by baseline characteristics (post hoc). The results are shown in Table 12. The median value was used as the cutoff for each characteristic (except for brain T2 lesion volume).

TABLE 12
Change in FF-VEP latency stratified in subgroups.

| | | FF-VEP | |
|---|---|---|---|
| | | treatment | |
| | | difference at | |
| | | Week 24 vs. | P |
| Characteristic | | placebo, ms^{a} | value^{a} |
| Age | <33 years | −0.89 | .87 |
| | ≥33 years | −14.17 | .01 |
| Treated | <25 days from AON | −9.01 | .12 |
| | onset | | |
| | ≥25 days from AON | −6.68 | .19 |
| | onset | | |
| Treated | <15 days after steroids | −8.21 | .14 |
| | ≥15 days after steroids | −7.16 | .20 |
| Low-Contrast Letter | 0, 2.5% Sloan chart | −6.46 | .27 |
| Acuity (LCLA) score | >0, 2.5% Sloan chart | −3.79 | .48 |
| High-Contrast Visual | <49 | −10.92 | .08 |
| Acuity (HCVA) score | ≥49 | −4.14 | .41 |
| Brain T2 lesion volume= | 0^{b} | −10.48 | .25 |
| | >0^{c} | −5.13 | .23 |

^{a}Based on ANCOVA.
^{b}n = 5, placebo; n = 8, anti-LINGO-1.
^{c}n = 29, placebo; n = 25, anti-LINGO-1

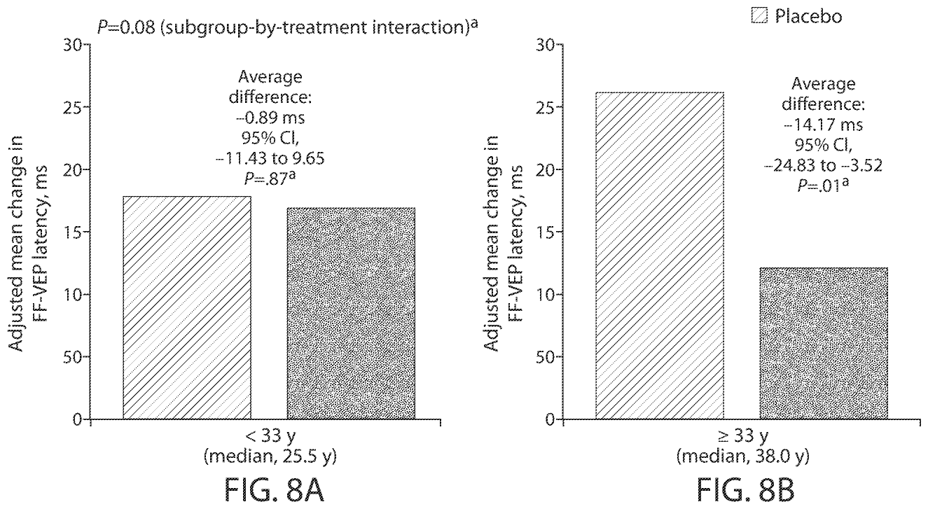

Treatment effects of anti-LINGO-1 were age-related in the PP population. Younger subjects had a smaller effect of anti-LINGO-1 on FF-VEP latency than older subjects, as shown in Table 13. The change in FF-VEP latency at week 24, according to age is shown in FIG. 8. Subjects ≤33 years of age in the treated group showed a 16.93 millisecond delay, amounting to a difference of −0.89 msec relative to untreated subjects (95% CI −11.43 to 9.65; P=0.87 based on ANCOVA). However, subjects >33 years of age showed a 12.15 millisecond delay, amounting to a difference of −14.17 msec relative to untreated subjects (95% CI=95% CI −24.83 to −3.52, P=0.01 based on ANCOVA).

TABLE 13

| | Placebo latency | Anti-LINGO-1 | |
|---|---|---|---|
| Age | in ms | latency in ms | p-value |
| <33 | 116.9 (10.8)* | 118.8 (15.9) | 0.86 |
| ≥33 | 127.9 (18.1) | 116.9 (17.4) | 0.0099** |

*Data are mean (SD) msec
**p-value for the treatment by age interaction = 0.081

The effects of anti-LINGO-1 were affected by time of first dose in the PP population. For patients in which the first dose was administered <25 days from AON onset, the placebo-treated group showed a 20.2 msec delay in latency and the anti-LINGO-1 treated group showed a 11.19 msec delay in latency, amounting to a difference of −9.01 msec [95% CI −20.44 to 2.42; p=0.12]. For patients in which the first dose was administered ≥25 days after AON onset, the placebo-treated group showed a 23.91 msec delay in latency and the anti-LINGO-1 treated group showed a 17.23 msec delay in latency, amounting to a difference of −6.68 msec [95% CI −16.75 to 3.39; p=0.19]. Thus, administration of anti-LINGO-1 shortly after AON onset (e.g., less than 25 days after AON onset) led to a greater reduction in latency delay when compared to placebo.

Additionally, the treatment effect of anti-LINGO-1 was more pronounced in patients with more severe baseline visual acuity impairment as determined by high contrast visual acuity (HCVA). In particular, subjects having a HCVA of less than 49 letters at baseline (more severely visually impaired at baseline) showed a latency recovery of −10.92 milliseconds [95% CI −23 to 1.2; p=0.076]. Subjects having a HCVA of 49 or more letters at baseline (less severely visually impaired at baseline) showed a latency recovery of −4.14 milliseconds [−14.1 to 5.86; p=0.41].

A summary of the efficacy of anti-LINGO-1 on FF-VEP latency is shown in Table 14.

TABLE 14

| | Wk 24 | | |
|---|---|---|---|
| | ANCOVA | Wk 24 | Wk 32 |
| | (LOCF) | MMRM | MMRM |
| ITT | −3.48 | −4.11 | −6.06 |
| Δ diff (p-value) | (0.3337) | (0.2546) | (0.0711) |
| Improvement | 16.8% | 19.8% | 28.7% |
| over placebo | | | |
| Per-Protocol | −7.55 | −7.67 | −9.13 |
| Δ diff (p-value) | (0.0504) | (0.0514) | (0.0112) |
| Improvement | 33.9% | 34.6% | 40.9% |
| over placebo | | | |

Baseline fellow eye reading: around 100 msec (normal average latency)
Wk 24 placebo affected eye reading: around 123 msec (~20% worse than normal)

There were three categories of FF-VEP responders based on impairments at baseline and week 24. The three categories were:

(1) subjects with non-recordable baseline latency for the affected eye (indicating a severe initial latency delay) and a first recordable latency >3% worse than the baseline of the unaffected fellow eye, in whom the week 24 latency for the affected eye returned to within 10% of the week 24 latency of the unaffected eye (indicating a mild improvement);

(2) subjects with measurable latency of the affected eye that was ≥10% worse than the baseline latency for the unaffected fellow eye, in whom (a) the week 24 latency for the affected eye returned to within 10% of the week 24 latency of the unaffected fellow eye (indicating a mild improvement), or (b) the week 24 latency for the affected eye was ≥15% improved from baseline (indicating a substantial improvement); and

(3) subjects with abnormal measurable baseline latency for the affected eye that was within 10% of the baseline latency for the unaffected fellow eye (indicating mild or moderate latency impairment), in whom the week 24 latency for the affected eye returned to normal (within 3% of the week 24 latency of the unaffected eye).

Also, MRI was used to measure the burden of disease by detecting lesions (e.g., GD-enhancing or T2 lesions) in the anti-LINGO-1 or placebo groups. Table 10 shows the size of T2 lesions in the brain of subjects, the placebo latency, and the anti-LINGO-1 treated latency. Lower anti-LINGO-1 latency appeared to correlate with smaller T2 lesions.

TABLE 15

| | | | | Anti | |
|---|---|---|---|---|---|
| T2 burden of | | Placebo | Anti | LINGO | Treatment |
| disease | Placebo | latency | LINGO | latency | difference |
| (BOD) in ml | subjects | (msec) | subjects | (msec) | (p value) |
| 0 | 4 | 17.72 | 8 | 7.35 | −10.37 (0.24) |
| >0 but <0.78 | 14 | 20.13 | 15 | 13.08 | −7.05 (0.207) |
| ≥0.78 | 13 | 24.43 | 9 | 24.31 | 0.1 (0.59) |

### Example 2: Efficacy by Multifocal Visual Evoked Potentials in Subjects Treated with the Anti-LINGO-1 Monoclonal Antibody BIIB033 in AON

Increased latency of cortical responses to monocular stimulation, indicative of demyelinating events in the optic nerve, is a hallmark of AON and has been demonstrated using full-field (FF) visual evoked potentials (VEP) and multifocal VEP (mfVEP). With mfVEP, visual stimuli are provided simultaneously to multiple regions of the visual field to provide stimulation to a wider visual field and more precise analysis. See Klistorner A, et al. Invest Ophthalmol Vis Sci. 2010; 51(5):2770-2777.

As described in Example 1, the RENEW trial was aimed at determining the efficacy and safety of anti-LINGO-1 antibody for CNS remyelination following the onset of a first episode of AON. In the RENEW trial described in Example 1, a mfVEP substudy was also conducted to explore the use of mfVEP as a potentially improved measure of treatment efficacy in AON trials than traditional FF-VEP (the pre-specified primary endpoint for the study). The mfVEP substudy is described in this example.

### Methods

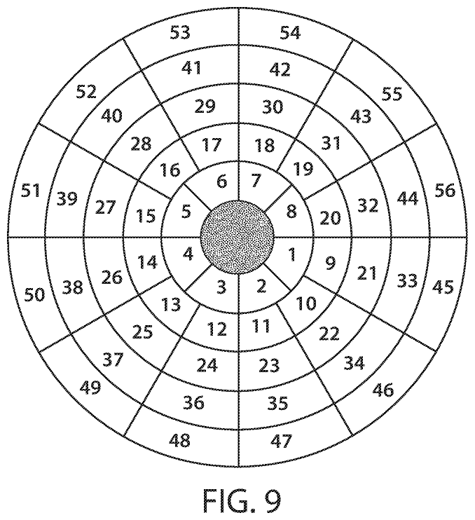

The protocols for the RENEW trial are described in detail in Example 1. In the mfVEP substudy, visual evoked potential latency measured by mfVEP was included as an exploratory endpoint at selected study sites. The individual segments assessed using mfVEP are shown in FIG. 9. mfVEP was performed at 4-week intervals from randomization to week 24 (primary efficacy analysis) and week 32 (end-of-study follow-up). Change in mfVEP latency from its own baseline in the fellow eye and affected eyes to weeks 24 and 32 were determined using progression analysis by a blind examiner. A central reading center was used for training, qualification, quality control, and data analysis of the mfVEP substudy (Duke Reading Center in cooperation with Vision Search, Sydney Australia).

Between-treatment comparisons were evaluated by ANCOVA and MMRM. Correlations with FF-VEP and retinal ganglion cell layer/inner plexiform layer thickness were assessed using pairwise correlation and the Pearson correlation coefficient. Subject groups analyzed (ITT, PP, those with or without FF-VEP latency recovery) are described in detail in Example 1.

### Results

The mfVEP substudy comprised 48% of the subjects participating in the RENEW trial (N=39/82); 18 were treated with placebo and 21 with anti-LINGO-1. The groups were similar in baseline demographics (Table 16). Sixteen participants treated with placebo and 15 treated with anti-LINGO-1 were part of the PP population in the RENEW trial.

TABLE 16
Demographic characteristics of the
subjects in the mfVEP substudy at baseline

| | | | 100 mg/kg |
|---|---|---|---|
| | All | | anti- |
| | patients | Placebo | LINGO-1 |
| Characteristic | (n = 39) | (n = 18) | (n = 21) |
| Sex, % female | 72 | 78 | 67 |
| Race, % white | 97 | 94 | 100 |
| Mean age | 32.3 ± 8.78 | 31.8 ± 9.93 | 32.7 ± 7.90 |
| (years ± SD) | | | |
| Weight, kg | 75.0 | 75.0 | 72.2 |
| (median, range) | (47-119) | (47-119) | (57-106) |
| Height, cm | 170.0 | 170.0 | 171.0 |
| (median, range) | (155-194)* | (155-194) | (158-185)* |
| Mean ± SD latency | | 144.4 ± 6.2 | 147.7 ± 5.3 |
| of the fellow eye, ms | | | |
| Mean ± SD | | 87.2 ± 48.6 | 78.4 ± 57.6 |
| amplitude of the | | | |
| affected eye, nV | | | |
| Mean ± SD | | 156.8 ± 57.3 | 167.4 ± 34.6 |
| amplitude of the | | | |
| fellow eye, nV | | | |
| Subjects with <60% | 13 (34) | 4 (22) | 9 (45) |
| measurable | | | |
| segments, n (%) | | | |

mfVEP = multifocal visual evoked potentials;
SD = standard deviation
*n = 20;
total = 38.

In subjects from the mfVEP substudy as a whole (ITT) and included in the PP population, the anti-LINGO-1-treated group showed improved VEP latency vs placebo at week 24 (difference of −4.97 ms [P=0.37] in the ITT substudy population and −11.78 ms [P=0.06] in the substudy PP population; FIG. 10). An improvement vs placebo of −3.82 ms (P=0.50) was seen at week 32 in the ITT substudy and of −9.38 ms (P=0.15) in the PP population substudy (by MMRM).

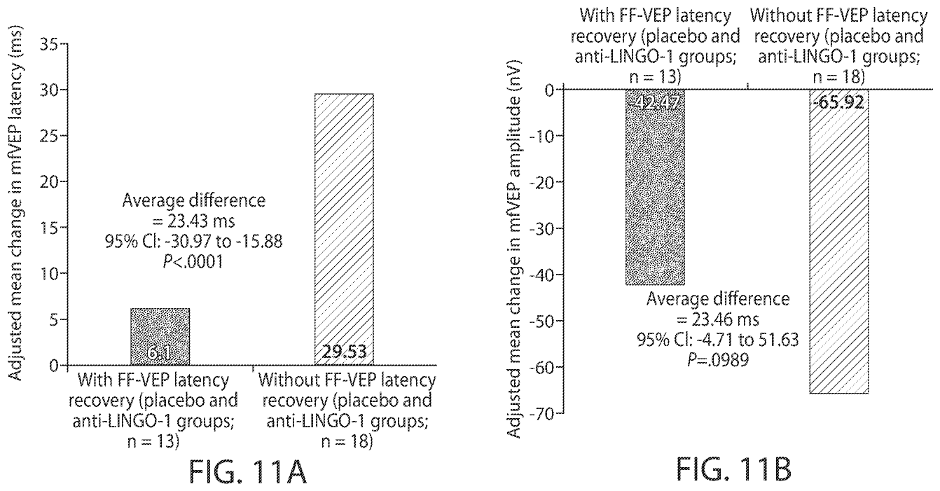

A total of 13 subjects (4 assigned to placebo and 9 to anti-LINGO-1) participating in the substudy had been identified as having latency recovery using FF-VEP (latency of the affected eye at 24 weeks returning to within 10% of the fellow eye baseline latency), compared with 18 without (11 placebo; 7 anti-LINGO-1). The extent of latency recovery by mfVEP was compared in these subjects. There was significantly less mfVEP latency delay at week 24 in those with FF-VEP latency recovery (FIG. 11). There was also a trend toward a significant difference in recovery of mfVEP amplitude (FIG. 11) in those with FF-VEP latency recovery that was not apparent using FF-VEP.

A summary of the efficacy of anti-LINGO-1 on mfVEP latency is shown in Table 17.

TABLE 17

| | Wk 24 | | |
|---|---|---|---|
| | ANCOVA | Wk 24 | Wk 32 |
| | (LOCF) | MMRM | MMRM |
| ITT population | −4.970 | −5.636 | −3.592 |
| Δ diff (p-value) | (0.3680) | (0.3127) | (0.5543) |
| Improvement | 22.1% | 25% | 18.5% |
| over placebo | | | |
| Per-Protocol | −11.78 | −10.720 | −9.379 |
| Δ diff (p-value) | (0.0638) | (0.0962) | (0.1456) |
| Improvement | 47.5% | 44.9% | 42.2% |
| over placebo | | | |

RENEW baseline fellow eye average = 146 msec
RENEW placebo affected eye wk24 average = 169 msec

Comparing the substudy mfVEP latency or amplitude changes with other efficacy endpoints in the RENEW trial PP and ITT populations revealed several correlations (Table 18).

TABLE 18
High correlations identified for change at week 24 for the
affected eye from baseline of the unaffected fellow eye*

| | Substudy and PP (n =31) | | | Total substudy (n =39) | | |
|---|---|---|---|---|---|---|
| | | 100 | | | 100 | |
| | | mg/kg | | | mg/kg | |
| | | anti- | | | anti- | |
| | | LINGO- | | | LINGO- | |
| | Placebo | 1 | | Placebo | 1 | |
| | n = 16 | n = 15 | Total | n = 18 | n = 21 | Total |
| Mean mfVEP | r = 0.98 | r = 0.93 | r = | r = 0.98 | r = 0.91 | r = |
| latency and | | | 0.96 | | | 0.95 |
| FF-VEP | | | | | | |
| latency | | | | | | |
| Mean mfVEP | | r = −0.50 | | | r = −0.50 | |
| latency and | | | | | | |
| mean | | | | | | |
| RGCL/IPL | | | | | | |
| thickness | | | | | | |
| Mean mfVEP | r = −0.54 | | | r = −0.54 | | |
| latency and | | | | | | |
| mean mfVEP | | | | | | |
| amplitude | | | | | | |
| Mean mfVEP | | r = −0.52 | | | r = −0.58 | |
| amplitude | | | | | | |
| and FF-VEP | | | | | | |
| latency | | | | | | |
| Mean mfVEP | r = 0.63 | | | r = 0.63 | | |
| amplitude | | | | | | |
| and FF-VEP | | | | | | |
| amplitude | | | | | | |
| Mean mfVEP | | r = 0.51 | | | r = 0.51 | |
| amplitude | | | | | | |
| and mean | | | | | | |
| RGCL/IPL | | | | | | |
| thickness | | | | | | |

FF-VEP = full-field visual evoked potentials;
ITT = intent-to-treat;
mfVEP = multifocal visual evoked potentials;
PP = per-protocol;
RGCL/IPL = retinal ganglion cell layer/inner plexiform layer
*Subgroup mfVEP latency or amplitude results were compared with other efficacy endpoints in the RENEW trial PP and ITT populations.

Additionally, the condition of the fellow eye was observed over time during the study by mfVEP latency and amplitude. The mfVEP amplitude of the fellow eye over time is shown in Table 19.

TABLE 19

| | Placebo | Anti-LINGO-1 | |
|---|---|---|---|
| | (N = 18) | (N =21) | |
| Visit | nanovolts | nanovolts | p-value |
| baseline | 180.927(72.75) | 196.237(55.89) | |
| Week 4 | 161.802(54.83) | 183.7(52.55) | 0.236 |
| Week 8 | 165.581(58.5) | 179.5(52.36) | 0.78 |
| Week 12 | 159.456(57.28) | 182.16(45.28) | 0.18 |
| Week 16 | 165.748(58.34) | 186.14(49.15) | 0.18 |
| Week 20 | 153.78(55.74) | 191.54(49.4) | 0.0019 |
| Week 24 | 162.726(58.81) | 182.875(61.89) | 0.048 |
| Week 32 | 146.142(51.14) | 186.218(55.57) | 0.0006 |

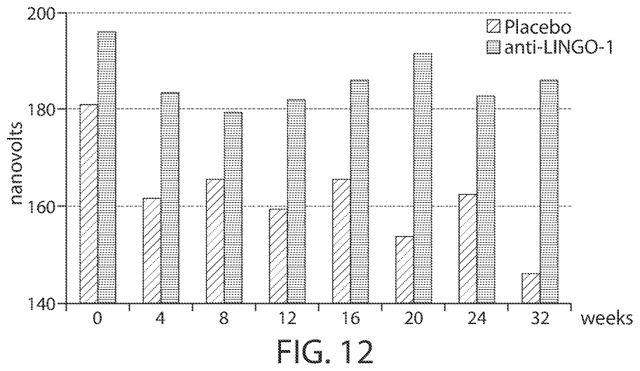

A strong treatment effect was observed by mfVEP on preventing amplitude loss and preserving amplitude in the fellow eye. See FIG. 12. As shown in FIG. 12, the mfVEP amplitude dropped in fellow eye in the placebo treated group as early as week 20, which indicates the presence of damage to the fellow eye. In groups treated with anti-LINGO-1, the mfVEP amplitude was preserved through the 32 week study. Thus, anti-LINGO-1 prevented damage from becoming established in the fellow eye of subjects following a first episode of AON.

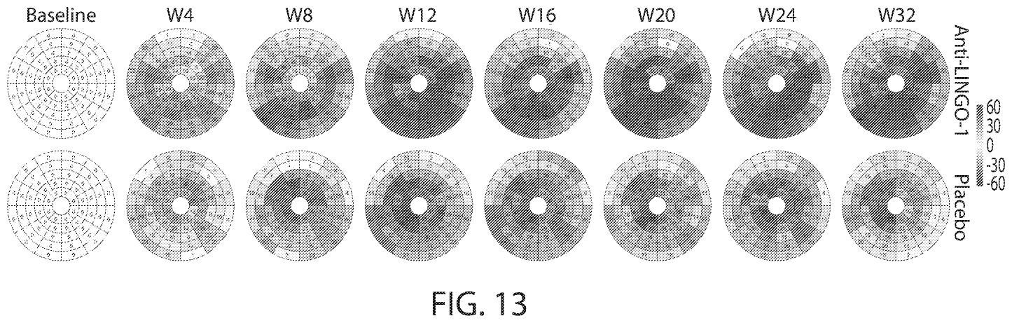

For both the affected and unaffected eye, the change in MF-VEP Amplitude from baseline in the same eye over 32 weeks was determined. The mean change in MF-VEP amplitude by treatment in the affected eye from baseline in the affected eye (nV) is shown in Table 20 and FIG. 13.

TABLE 20
Mean change in MF-VEP amplitude by treatment in the
affected eye from baseline in the affected eye over 32 weeks.

| | Average | | | | |
|---|---|---|---|---|---|
| | amplitude, nV; | | | | |
| | 56 segments | | Difference vs. placebo | | |
| Affected | | Anti- | Estimated | | |
| eye^{a} | Placebo | LINGO-1 | difference | 95% CI | P value |
| Change at | 23.548 | 25.210 | 1.662 | −18.462 to 21.786 | .8678 |
| Week 4 | | | | | |
| Change at | 30.743 | 30.335 | −0.408 | −21.083 to 20.267 | .9683 |
| Week 8 | | | | | |
| Change at | 29.920 | 41.556 | 11.636 | −13.257 to 36.528 | .3491 |
| Week 12 | | | | | |
| Change at | 34.443 | 43.119 | 8.677 | −15.225 to 32.578 | .4662 |
| Week 16 | | | | | |
| Change at | 29.547 | 50.178 | 20.631 | −3.621 to 44.883 | .0930 |
| Week 20 | | | | | |
| Change at | 29.864 | 43.516 | 13.652 | −11.658 to 38.962 | .2808 |
| Week 24 | | | | | |
| Change at | 25.882 | 48.198 | 22.316 | −1.261 to 45.893 | .0628 |
| Week 32 | | | | | |

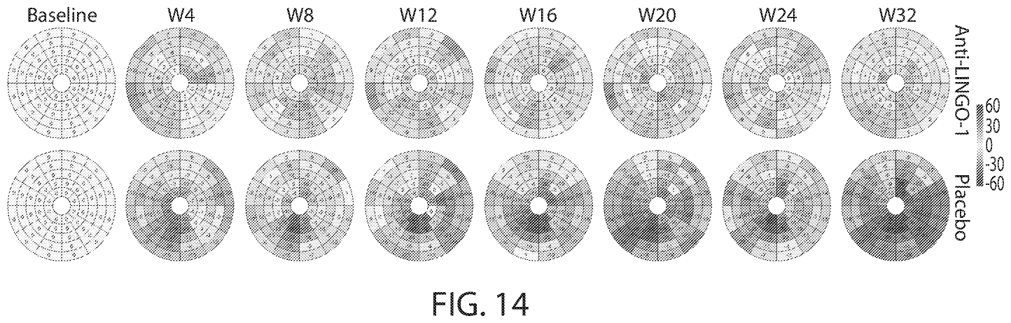

The mean change in MF-VEP Amplitude by Treatment in the unaffected eye from baseline in the unaffected eye over 32 weeks (nV) is shown in Table 21 and FIG. 14.

TABLE 21
Mean change in MF-VEP amplitude by treatment in the
unaffected eye from baseline in the unaffected eye over 32 weeks.

| | Average | | | | |
|---|---|---|---|---|---|
| | amplitude, nV; | | | | |
| | 56 segments | | Difference vs. placebo | | |
| | | Anti- | Estimated | | |
| Fellow eye | Placebo | LINGO-1 | difference | 95% CI | P value |
| Change at | −14.017 | −6.126 | 7.891 | −5.926 to 21.708 | .2543 |
| Week 4 | | | | | |
| Change at | −12.361 | −4.814 | 7.547 | −9.399 to 24.493 | .3723 |
| Week 8 | | | | | |
| Change at | −18.452 | −2.628 | 15.824 | −0.608 to 32.256 | .0585 |
| Week 12 | | | | | |
| Change at | −17.522 | −2.845 | 14.677 | −1.456 to 30.809 | .0731 |
| Week 16 | | | | | |
| Change at | −24.336 | 1.120 | 25.456 | 8.150 to 42.762 | .0053 |
| Week 20 | | | | | |
| Change at | −17.565 | −0.588 | 16.977 | −0.225 to 34.178 | .0529 |
| Week 24 | | | | | |
| Change at | −31.407 | 1.926 | 33.333 | 16.408 to 50.258 | .0004 |
| Week 32 | | | | | |

Also, the change in latency (msec) of the fellow eye was measured by mfVEP over time during the study. A MMRM-ITT analysis was performed using all timepoints and shown in Table 22. There was no significant difference in latency measured by mfVEP over time in the fellow eye when treated with placebo versus anti-LINGO-1.

TABLE 22

| | Placebo | Anti-LINGO-1 | | |
|---|---|---|---|---|
| Visit | msec | msec | Difference | p-value |
| baseline | 144.45(6.2) | 147.68(5.33) | | |
| Week 4 | 145.35(6.8) | 148.68(4.78) | 0.623 | 0.43 |
| Week 8 | 145.405(6.4) | 148.37(4.8) | −0.442 | 0.63 |
| Week 12 | 146.65(5.8) | 149.110(4.7) | −0.772 | 0.41 |
| Week 16 | 145.83(6.18) | 149.56(4.3) | −0.487 | 0.59 |
| Week 20 | 146.55(6.5) | 148.7(5.1) | −1.885 | 0.09 |
| Week 24 | 146.95(5.1) | 148.18(5.28) | −1.749 | 0.11 |
| Week 32 | 145.94(5.29) | 149.14(4.68) | −0.771 | 0.39 |

A comparison of the efficacy outcome measures in FF-VEP latency recoverers versus non-recoverers is shown in Table 23 (the numbers indicate the change at 24 weeks versus baseline (ITT)).

TABLE 23

| | Recoverer on | Non-recoverer on | |
|---|---|---|---|
| | anti-LINGO-1 or | anti-LINGO-1 or | |
| Efficacy measures | placebo | placebo | P value |
| FF-VEP latency | 4.51 (N = 29) | 29.33 (N = 37) | <0.0001 |
| (msec)* | | | |
| FF-VEP amplitude | −3.22 (N = 29) | −2.64 (N = 37) | 0.587 |
| (uV)* | | | |
| mfVEP latency (msec)* | 6.10 (N = 13) | 29.53 (N = 16) | <0.0001 |
| mfVEP amplitude | −42.47 (N = 13) | −65.92 (N = 16) | 0.098 |
| (nV)* | | | |
| RGCL thickness | −7.83 (N = 29) | −12.75 (N = 40) | 0.0071 |
| (microns)* | | | |
| 1.25% LCLA (# of | 9.8 (N = 27) | 6.09 (40) | 0.206 |
| letters)** | | | |
| 2.5% LCLA (# of | 13.47 (N = 27) | 11.44 (N = 40) | 0.512 |
| letters)** | | | |
| HCVA (# of letters)** | 10.78 (N = 28) | 12.40 (N = 40) | 0.528 |
| 39 items VFQ | 14.87 (N = 28) | 15.24 (N = 39) | 0.828 |
| 10 item NOS | 17.65 (N = 27) | 16.78 (N = 38) | 0.780 |

*Adjusted mean vs baseline of fellow eye;
**Adjusted mean vs baseline of affected eye

### Conclusions for RENEW Trial

The results described in Example 1 show improved latency recovery (a greater shortening of latency delay) measured by FF-VEP in the anti-LINGO-1 group compared with placebo. Analysis in the PP population was statistically significant; analysis in the ITT population showed a positive trend (MMRM; Week 32). At the individual level, FF-VEP latency recovery analysis showed that latency in the affected eye recovered to normal or close to normal in twice as many subjects treated with anti-LINGO-1 (53%) than placebo (26%). Subjects whose FF-VEP latency recovered to normal or close to normal had experienced significantly less RGCL thinning (average, −7.83 μm) than those that did not (average, −12.75 μm). The magnitude of the treatment effect (about 40-50% improvement in conduction delay or about 8-10 msec reduction in P100 latency) is consistent with optic nerve remyelination by anti-LINGO-1 following AON.

No detectable treatment effect was observed on the secondary endpoints of SD-OCT thinning, LCLA, and high contrast visual acuity that measured the neuroprotective effects. However, the results revealed that retinal thinning occurred very rapidly, at least half before the first dose and the remainder before the second dose.

Also, the treatment effect by anti-LINGO-1 was better in the higher age group, in patients with greater baseline acuity impairment, and in patients with earlier treatment initiation.

The anti-LINGO-1 antibody, opicinumab, demonstrated an improvement in the study's primary endpoint, recovery of latency (time for a signal to travel from the retina to the visual cortex), as measured by full field visual evoked potential (FF-VEP), relative to placebo. The study showed no detectable effect on secondary endpoints, including change in thickness of the retinal layers (optic nerve neurons and axons) and visual function, as measured by spectral domain optical coherence tomography (SD-OCT) and low contrast letter acuity, respectively, as herein. The primary and secondary endpoints were measuring two different aspects of the potential drug's impact of the visual system (remyelination and neuroprotection). The lack of treatment effects in both secondary endpoints, low contrast visual acuity and OCT, are consistent. No neuroprotective effect on the retina was detected in the AON lesion in this study. However, a protective treatment effect of anti LINGO-1 treatment was seen for the amplitude of the mfVEP in both the affected and the fellow eye visual pathways over 32 weeks, which was highly statistically significant at 32 weeks for the fellow eye mfVEP amplitude.

NCT01721161 was designed to study anti-LINGO-1's ability to enable repair of an optic nerve lesion via axonal remyelination following the onset of a first episode of AON. It characterized the effects on remyelination by measuring the latency of nerve conduction between the retina and the visual cortex in the brain using FF-VEP. The primary endpoint measured FF-VEP latency for the affected eye at week 24 compared to the unaffected fellow eye at baseline. Results demonstrated a 34 percent improvement (p=0.0504) in the recovery of optic nerve latency compared to placebo in the per-protocol population at 24 weeks, and even higher at 32 weeks.

The analysis of the intent-to-treat (ITT) population, which includes patients in both arms who did not complete the study, showed a positive trend but did not reach statistical significance.

The results herein show that anti-LINGO-1 was effective in causing optic nerve remyelination in patients who experienced a first episode of AON, but who did not yet have diagnosable MS. Furthermore, it showed clearly protective effects on the amplitude of the mfVEP in both the affected and fellow eye visual pathways over 32 weeks. Thus, administering anti-LINGO-1 early on, soon after a first episode of AON and before development of diagnosable MS, may prevent the worsening of AON and/or prevent the development of MS.

RENEW is the first clinical trial demonstrating the clinical efficacy of anti-LINGO-1 with the observed shortening of FF-VEP latency consistent with optic nerve remyelination following a first episode of AON. Consistent results were observed in a substudy that measured latency using multifocal VEP (mfVEP) and are described in Example 2. Protective effects of the amplitude were seen by mfVEP for both the affected and fellow eyes, and by FF-VEP amplitude for the affected eye (data not shown). Results from the safety analyses show that anti-LINGO-1 was well tolerated, with an overall incidence and severity of AEs similar to placebo-treated subjects. Safety and tolerability analyses from the trial are described in Example 3, and patient reported outcomes from the trial are described in Example 4.

A second ongoing Phase 2 dose-range finding study (SYNERGY) that investigates anti-LINGO-1 in participants with relapsing forms of MS is described, e.g., at Clinical Trials Identifier No. ClinicalTrials.gov Identifier: NCT01864148.

The per-protocol population is defined as subjects from the ITT population who complete the study, did not miss more than one dose of anti-LINGO-1 or placebo, and did not receive MS modifying therapies during the study period.

Example 2 describes the first reported use of mfVEP in the context of a randomized clinical trial. Results from the mfVEP substudy showed improved mfVEP latency (a greater shortening of latency delay) in the anti-LINGO-1 group compared with placebo at both week 24 and week 32. Differences between the placebo and anti-LINGO-1 arms were greater when comparing substudy subjects included in the PP population rather than all substudy participants. Subjects whose FF-VEP latency (RENEW study primary endpoint) recovered to normal or close to normal experienced significantly less delay in mfVEP latency from baseline to week 24 (average 6.1 ms) and a trend towards better amplitude recovery (P<0.10) than those who did not (average 29.53 ms). mfVEP latency changes were highly correlated with FF-VEP latency changes (r≥0.91), while the correlation between amplitude changes was lower (r=0.63).

Results from the mfVEP substudy are consistent with the overall efficacy results for the primary endpoint (FF-VEP), as described in Example 1. This suggests that mfVEP could be a powerful efficacy endpoint in clinical trials investigating the efficacy of candidate CNS remyelinating and neuroprotective agents. Furthermore, by generating reliable and informative results in a multicenter international substudy, the feasibility of this technique has been established.

The treatment effects from the RENEW study (including the mfVEP substudy) occurred within several months (3-6 months) and was of a magnitude (about 8-10 msec) consistent with a CNS remyelination mechanism of action by anti-LINGO-1. Due to the significantly reduced loss of mfVEP amplitude on the fellow eye over time in the anti-LINGO-1 treated group, anti-LINGO-1 likely has neuroprotective effects as well. These results suggest that anti-LINGO-1 may be neuroprotective in MS when used prophylactically. These results also suggest that mfVEP of healthy eyes could be a way to diagnose MS early on, before the development of diagnosable MS symptoms and/or before the onset of AON. Though the reduced loss of amplitude was not observed in the FF-VEP study, this was likely due to the lack of sensitivity of the FF-VEP measurement for amplitude changes. No retinal neuroaxonal protection was observed in the RENEW study, likely because much of the thinning had already taken place by the time of the first dose. However, cerebral neuroprotection in the visual pathway was likely observed with anti LINGO-1 treatment during the longitudinal 32 week follow up of the RENEW subjects enrolled in the mfVEP substudy.

### Example 3: Safety and Tolerability of Anti-LINGO1 Antibody in AON

The safety and tolerability of anti-LINGO-1 antibody in participants of the RENEW trial (NCT01721161) described herein (e.g., in Examples 1 and 2) were assessed. The RENEW trial protocol is described in detail in the examples above. Safety and tolerability assessments included adverse events (AEs) and severe AEs, clinical laboratory results (hematology, blood chemistry, urinalysis), physical exam findings, clinically-relevant vital sign abnormalities, brain magnetic resonance imaging results, 12-lead electrocardiogram readings, measurement of anti-LINGO-1 antibody in blood, and AON signs and symptoms. All subjects who received ≥1 dose of study treatment were included in the safety population.

### Results

Eighty-two subjects received placebo (n=41) or anti-LINGO-1 (n=41) and were largely similar in baseline characteristics (Table 5). The number of subjects with an AE and the severity of the AEs were similar between the placebo and anti-LINGO-1 groups (Tables 24 and 25). The number of subjects with a serious AE and discontinuing treatment were higher in the anti-LINGO-1 group than the placebo group (Table 24).

TABLE 24
Summary of AEs

| | Placebo | Anti-LINGO-1 | All subjects |
|---|---|---|---|
| AE, n (%) | n = 41 | n = 41 | N = 82 |
| Any AE | 34 (83) | 34 (83) | 68 (83) |
| Serious AE | 2 (5) | 5 (12) | 7 (9) |
| Treatment-related serious AE | 0 | 3 (7) | 3 (4) |
| Discontinued treatment due to | 1 (2) | 3 (7) | 4 (5) |
| AE | | | |
| Withdrew from study due to | 2 (5) | 3 (7) | 5 (6) |
| AE | | | |

TABLE 25
Severity of AEs

| | | | All subjects |
|---|---|---|---|
| AE, n (%) | Placebo n = 41 | Anti-LINGO-1 n = 41 | N = 82 |
| Any AE | 34 (83) | 34 (83) | 68 (83) |
| Mild AE | 12 (29) | 13 (32) | 25 (30) |
| Moderate AE | 20 (49) | 18 (44) | 38 (46) |
| Severe AE | 2 (5) | 3 (7) | 5 (6) |

Four subjects discontinued treatment due to an AE. In the placebo group, one patient discontinued treatment (due to MS). In the anti-LINGO-1 treated group, 3 patients discontinued treatment due to treatment-related serious AEs. Two of the three patients had hypersensitivity, and in both patients, reactions occurred shortly after the start of the second infusion and fully resolved shortly after discontinuation of infusion. The third patient had an asymptomatic case of increased alanine and aspartate aminotransferases that was reported as hepatopathy, first observed after the second infusion, and resolved following treatment discontinuation.

Serious AEs occurred in 7 subjects. Two subjects in the placebo group experienced serious AEs. In addition to the subject who discontinued treatment, a second subject had viral pericarditis and tested positive for cytomegalovirus. Five subjects had serious AEs in the anti-LINGO-1 group; 3 resulted in treatment discontinuation (see above), 1 suffered an MS relapse, and 1 had optic neuritis in the fellow eye.

The incidence of AEs by System Organ Class (SOC) was generally similar between groups (Table 26). However, gastrointestinal disorders occurred more frequently in those treated with anti-LINGO-1 than placebo. The most commonly reported gastrointestinal symptoms were nausea (anti-LINGO-1, 12% vs. placebo, 7%) and dyspepsia (anti-LINGO-1, 5% vs. placebo, 2%). Six AEs occurred in ≥10% of all subjects, regardless of treatment group (Table 27). Of these, 3 occurred more frequently with anti-LINGO-1 than placebo: (i) fatigue (15% vs. 12%), (ii) nausea (12% vs. 7%), and (iii) paresthesia (10% vs. 0). The remainder occurred at a similar frequency between groups or more frequently in the placebo group (Uhthoff's phenomenon). AEs occurring ≤4 hours after the start of infusion were higher in the anti-LINGO-1 group than the placebo group (Table 28). The same event did not occur with every infusion and the events were most frequent after the second and third infusions. The most common infusion-related events were headache (7%) and nausea (7%). Both events of hypersensitivity occurred during the second infusion.

TABLE 26
Incidence of AEs by System Organ Class

| | Placebo | Anti-LINGO-1 |
|---|---|---|
| AE, n (%) | n = 41 | n = 41 |
| Infections and infestations | 22 (54) | 19 (46) |
| Nervous system disorders | 22 (54) | 22 (54) |
| General disorders and administration site | 10 (24) | 9 (22) |
| conditions | | |
| Eye disorders | 7 (17) | 8 (20) |
| Musculoskeletal and connective tissue disorders | 7 (17) | 8 (20) |
| Skin and subcutaneous tissue disorders | 7 (17) | 6 (15) |
| Gastrointestinal disorders | 5 (12) | 12 (29) |
| Respiratory, thoracic, and mediastinal disorders | 4 (10) | 4 (10) |
| Injury, poisoning, and procedural complications | 3 (7) | 2 (5) |
| Investigations | 3 (7) | 6 (15) |
| Psychiatric disorders | 3 (7) | 3 (7) |
| Renal and urinary disorders | 2 (5) | 2 (5) |
| Reproductive system and breast disorders | 2 (5) | 1 (2) |

TABLE 27
Incidence of AEs occurring in ≥ 10% of subjects

| AE, n (%) | Placebo n = 41 | Anti-LINGO-1 n = 41 |
|---|---|---|
| Nasopharyngitis | 13 (32) | 12 (29) |
| Headache | 11 (27) | 11 (27) |
| Fatigue | 5 (12) | 6 (15) |
| Nausea | 3 (7) | 5 (12) |
| Paresthesia | 0 | 4 (10) |
| Uhthoff's phenomenon | 6 (15) | 3 (7) |

TABLE 28
No. of subjects with AEs occurring ≤4 hours after infusion

| | | Dose 2 | Dose 3 | Dose 4 | Dose 5 | Dose 6 |
|---|---|---|---|---|---|---|
| | Dose 1 | (Week | (Week | (Week | (Week | (Week |
| | (Baseline) | 4) | 8) | 12) | 16) | 20) |
| Placebo | | | | | | |
| No. dosed | 41 | 39 | 40 | 37 | 37 | 37 |
| No. with AE | 1 | 0 | 0 | 0 | 0 | 0 |
| Anti- | | | | | | |
| LINGO-1 | | | | | | |
| No. dosed | 41 | 40 | 38 | 37 | 35 | 36 |
| No. with AE | 3 | 8^{a} | 6 | 4 | 5 | 2 |

^{a}Includes 2 cases of hypersensitivity leading to treatment discontinuation.

Seventeen subjects had weight gain >7% from Baseline. Placebo, n=4 (10%) vs. anti-LINGO-1, n=13 (32%). Subgroup analyses showed that subjects who gained >7% during the study had worse baseline disease (including higher frequency of conduction block, high contrast visual acuity impairment, and visual evoked potential latency delay). Three participants had weight decrease >7% (placebo, n=2; anti-LINGO-1, n=1). Other safety and tolerability investigations were similar between groups.

### Conclusions for Safety and Tolerability

The results show that anti-LINGO-1 at the dose of 100 mg/kg was generally well tolerated, and the overall incidence and severity of AEs was comparable with that of placebo-treated subjects. In the study, few treatment-related serious AEs were observed, and they all resolved on discontinuation of treatment. The incidence of serious AEs not related to treatment also was low, and the majority was MS related.

The most common AEs regardless of treatment were nasopharyngitis, headache, fatigue, Uhthoff phenomenon, and nausea. The most common AEs occurring at a higher rate on anti-LINGO-1 than placebo were fatigue, nausea, and paresthesia. No deaths occurred during the trial. The incidence of serious adverse effects (SAEs) were higher in the anti-LINGO-1 treated group (12%) than the placebo treated group (5%). Two patients treated with anti-LINGO-1 had SAEs of hypersensitivity reactions occurring around the time of infusion. One patient had a SAE of asymptomatic elevation in liver transaminases, which was resolved after drug discontinuation.

No immunogenicity was observed. Increased numbers of asymptomatic elevation in serum transaminases >3×ULN were seen in the anti-LINGO-1 treated group than in the placebo treated group (anti-LINGO-1 7% versus placebo 0). An increased incidence of post-baseline weight changes of greater than 7% (increase at any post-baseline timepoint) were seen in the anti-LINGO-1 treated subjects (anti-LINGO-1 32% versus placebo 10%).

The safety and tolerability of anti-LINGO-1 in the treatment of AON support the use and ongoing clinical development of anti-LINGO-1 for CNS demyelinating diseases.

### Example 4: Effects of Anti-LINGO-1 Antibody on Vision-Related Quality of Life in Subjects with AON

The RENEW clinical trial is described in detail in the above examples. There are currently no established patient reported outcomes (PROs) for AON or to measure remyelination. Thus, in the RENEW trial, a further exploratory endpoint was assessed. In particular, a PRO measure for vision-related quality of life (QoL), the National Eye Institute Visual Functioning Questionnaire-25 (NEI VFQ-25) (Mangione C M, et al. Arch Ophthalmol. 1998; 116(11):1496-1504), was performed to evaluate the treatment benefit of anti-LINGO-1. This analysis evaluated the self-reported visual functioning of patients with AON randomized to anti-LINGO-1 or placebo in RENEW.

The protocol of the RENEW trial is described in detail in the above examples. The NEI-VFQ-25, including 13 addendum items, and the 10-Item Neuro-Ophthalmic Supplement (NOS-10 (Raphael B A, et al. Am J Ophthalmol. 2006; 142(6):1026-1035.e2); exploratory endpoints), ranged from 0 (high impairment) to 100 (no impairment). A change of 4 points was considered clinically meaningful (Surer I J, et al. Invest Ophthalmol Vis Sci. 2009; 50(8):3629-3635). The NEI VFQ-25 was administered at baseline and at weeks 4, 8, 12, 16, 20, 24, and 32. Results of the NEI VFQ-25 and the NOS-10 are presented separately and combined.

### Statistical Analyses

The PP population was defined as subjects who completed the study, did not miss >1 dose of treatment, and did not receive multiple sclerosis modifying therapy. Data from the PP population are presented in this example. Between-treatment differences in mean change in score from baseline for each of the vision-related QoL assessments were analyzed by mixed-effect model repeated measure (MMRM) and analysis of covariance (ANCOVA). MMRM and ANCOVA models were adjusted for baseline vision-related QoL assessment value and treatment group. Last observation carried forward (LOCF) was used for imputation in ANCOVA analyses.

### Results

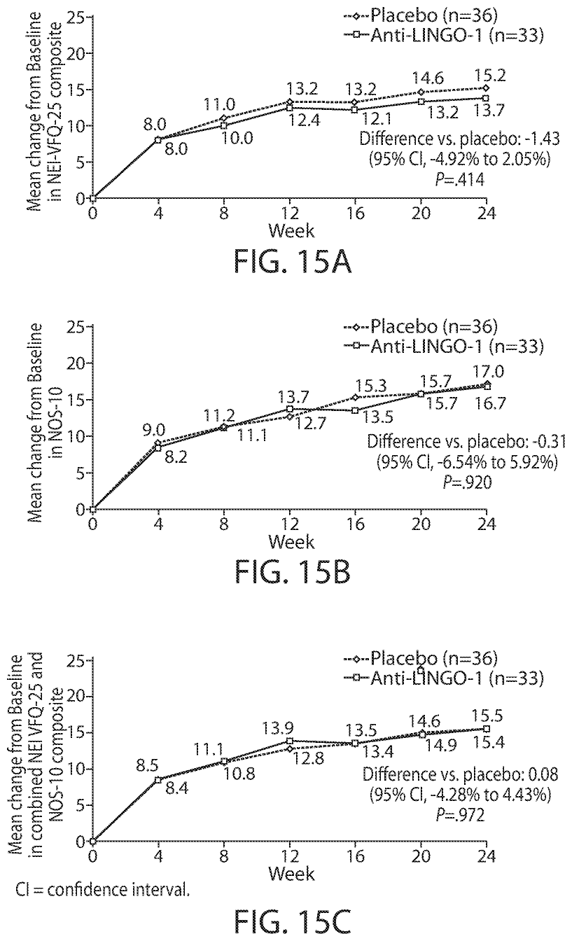

A total of 69 patients were included in the PP population of RENEW and received either placebo (n=36) or anti-LINGO-1 (n=33). Demographic characteristics were similar at baseline (Table 5). Patient scores reflected considerable impairment at baseline on the NEI VFQ-25 composite (Table 29). Patients in the placebo group had slightly higher mean scores at baseline compared with the anti-LINGO-1 group (Table 29). Both groups experienced substantial improvements from baseline in adjusted mean NEI VFQ-25 composite, NOS-10, and combined NEI VFQ-25 and NOS-10 composite scores through week 24 (FIG. 15). Between-treatment differences in mean change in score from baseline for each of the vision-related QoL assessments were not significantly different at any time point (FIG. 15).

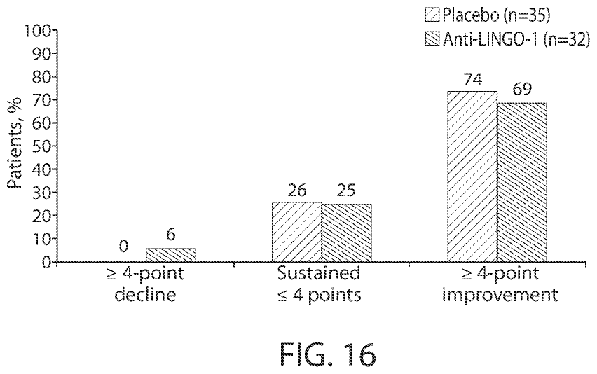

At Week 24, the majority of patients in both groups had ≥4-point improvement in NEI VFQ-25 composite score, while few patients experienced ≥4-point decline (FIG. 16). Mean change from baseline for NEI VFQ-25 composite, NOS-10, and combined NEI VFQ-25 and NOS-10 composite scores did not differ between treatment groups based on ANCOVA models (Table 30).

TABLE 29
Demographic characteristics and baseline vision-related QoL (PP population)

| | Placebo | Anti-LINGO-1 | All subjects |
|---|---|---|---|
| Parameter | n = 36 | n = 33 | N = 69 |
| Female, % | 75 | 64 | 70 |
| White, % | 97 | 97 | 97 |
| Mean ± SD age, y | 32.2 ± 8.80 | 31.2 ± 7.12 | 31.7 ± 8.00 |
| Median (range) weight, kg | 73.8 | 72.2 | 72.5 |
| | (50-119) | (46-106) | (46-119) |
| Median (range) height, cm^{a} | 169.5 | 170.0 | 170.0 |
| | (155-194) | (158-188) | (155-194) |
| Mean ± SD NEI VFQ-25 | 79.0 ± 16.6 | 75.5 ± 17.6 | 77.3 ± 17.02d |
| composite score^{b} | | | |
| Mean ± SD NOS-10 score^{a} | 69.8 ± 21.2 | 63.6 ± 19.8 | 66.9 ± 20.6 |
| Mean ± SD combined NEI | 77.2 ± 16.9 | 70.4 ± 18.3 | 74.0 ± 17.8 |
| VFQ-25 and NOS-10 | | | |
| composite score^{a} | | | |

^{a}Placebo, n = 34; anti-LINGO-1, n = 31; total N = 65.
^{b}Placebo, n = 35; anti-LINGO-1, n = 32; total N = 67.

TABLE 30
Change from Baseline in mean in NEI VFQ-25 composite,
NOS-10, and combined NEI VFQ-25 and NOS-10 composite
scores at Week 24 analyzed by ANCOVA^{a} (PP population)

| | Placebo | Anti-LINGO-1 |
|---|---|---|
| Parameter | n = 36 | n = 33 |
| NEI VFQ-25 composite score | | |
| Mean change from Baseline | 15.17 | 13.51 |
| Difference vs. placebo (95% CI) | — | −1.66 |
| | | (−5.11% to 1.78%) |
| P value | — | .3374 |
| NOS-10 score | | |
| Mean change from Baseline | 17.40 | 16.04 |
| Difference vs. placebo (95% CI) | — | −1.35 |
| | | (−7.38% to 4.67%) |
| P value | — | .6550 |
| Combined NEI VFQ-25 and | | |
| NOS-10 composite score | | |
| Mean change from Baseline | 15.83 | 14.88 |
| Difference vs. placebo (95% CI) | — | −0.95 |
| | | (−5.08% to 3.19%) |
| P value | — | .6487 |

^{a}Adjusted for baseline vision-related QoL assessment value and treatment group.

### Conclusions for Patient Reported Outcomes

The RENEW trial evaluated patient-reported visual functioning in an AON episode with an intervention. Baseline NEI VFQ-25 composite and NOS-10 scores indicated that all patients had visual functioning impairment at the start of the study. Regardless of initial injury or treatment group, patients demonstrated notable improvements in vision-related QoL scores from Baseline to week 24. Despite improvements, mean NEI VFQ-25 composite score by week 24 for each group remained below the maximum possible score of 100 (no visual impairment). The residual PRO visual functioning impairment is consistent with the permanent loss of neurons that occurred early on (see Example 3) and likely prevented treatment effect on neuroprotection and visual-related QoL assessments. It is unclear whether any of the 49 items included in the NEI VFQ-25 and NOS-10 are sensitive to demyelination or remyelination in the optic nerve. The NOS-10 may be more sensitive, as it showed more visual function impairment at baseline and was more sensitive to change over time.

### Example 5: Efficacy of Anti-LINGO-1 Antibodies in a Clinical Study for Improvement of Relapsing-Remitting MS (RRMS) and Secondary Progressive MS (SPMS)

The SYNERGY trial was a phase 2 clinical study performed to assess the efficacy of anti-LINGO-1 e.g., BIIB033, e.g., opicinumab, for improving and slowing the worsening of Relapsing-remitting MS (RRMS) and Secondary progressive MS (SPMS).

### Methods

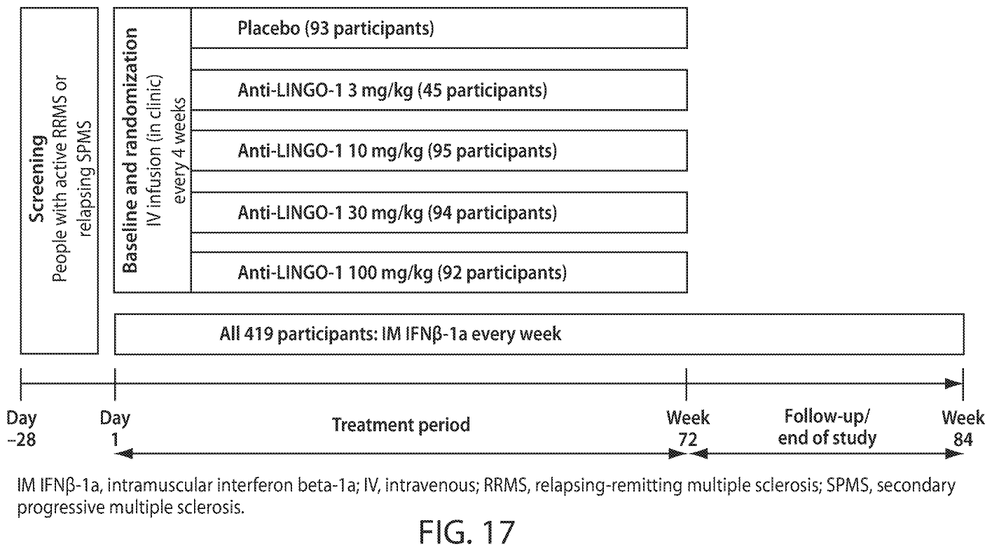

Patients received placebo or anti-LINGO-1 every 4 weeks for 72 weeks followed by a twelve week follow up period (see FIG. 17). The treatment period was preceded by a 28 day screening period. 93 patients were in the placebo group. Patients in the treatment group received one of four dosages: 3 mg/kg (45 patients); 10 mg/kg (95 patients); 30 mg/kg (94 patients); or 100 mg/kg (92 patients). Patients in both the treatment group and the placebo group were also treated with 30 micrograms of Avonex (IFN-β1) given intramuscularly once per week for the duration of the study.

Eligible subjects were aged 18 to 58 years (inclusive) and had a confirmed diagnosis of RRMS (by the McDonald criteria) or SPMS (by the Lublin and Reingold criteria), a baseline EDSS score of 2.0 to 6.0, and disease activity over the past year. For subjects with RRMS, disease activity was defined as 2 or more distinct occurrences of any of the following 3 events within 12 months prior to enrollment: a clinical relapse; a gadolinium-enhancing lesion on brain or spinal cord magnetic resonance imaging (MRI); and/or a new T2 lesion on brain or spinal cord MRI. For subjects with SPMS, disease activity was defined as 1 or more distinct occurrences of any of the following 2 events within 12 months prior to enrollment: a clinical relapse and/or a gadolinium-enhancing lesion on brain or spinal cord MRI. New T2 lesions were not included for participants with SPMS as they are not a reliable indicator in this population for qualifying individuals as having “actively relapsing” disease. 80% of subjects had RRMS and 20% of subjects had SPMS. Subjects were either naïve to disease modifying therapies (DMTs) or had previously received DMTs. Subjects were excluded if they had a history of abnormal laboratory results indicative of significant major disease. Subjects with RRMS who have previously had an inadequate response to any approved IFN beta treatment are also excluded. This is because SYNERGY participants received an IFN beta disease-modifying therapy (IM IFN beta-1a) to treat the inflammatory component of the disease. In addition, subjects with MS were also excluded if they have received treatment with: any investigational MS drug within 3 weeks or 5 half-lives (whichever is longer) prior to baseline; if they have received high-dose oral or IV steroids within 30 days prior to baseline; or if they have received fingolimod or natalizumab within 3 months prior to baseline. This 3-month washout period was used to ensure that no anti-inflammatory effect conferred by previous treatment with fingolimod or natalizumab was present at baseline. The baseline demographic characteristics of the patients are shown in Table 31.

TABLE 31
The baseline demographic characteristics of the patients
treated with anti-LINGO-1

| | Placebo | 3 mg/kg | 10 mg/kg | 30 mg/kg | 100 mg/kg | All patients |
|---|---|---|---|---|---|---|
| Characteristic | (n = 91) | (n = 45) | (n = 94) | (n = 91) | (n = 91) | (N = 412) |
| Sex, % female | 71 | 53 | 63 | 66 | 71 | 66 |
| Race, % white^{a} | 40 | 44 | 39 | 38 | 38 | 40 |
| Mean age | 39.7 ± 9.27 | 36.5 ± 9.47 | 40.5 ± 9.60 | 40.9 ± 9.60 | 39.8 ± 9.14 | 39.8 ± 9.51 |
| (years ± SD) | | | | | | |
| Weight, kg | 71.0 | 67.0 | 69.0 | 69.8 | 71.8 | 70.0 |
| (median, | (44-129) | (47-110) | (43-140) | (40-116) | (46-123) | (40-140) |
| range) | | | | | | |
| Height, cm | 168.0 | 172.0 | 167.0 | 170.5 | 168.0 | 168.0 |
| (median, | (147-193) | (152-188) | (154-195) | (151-193) | (152-197) | (147-197) |
| range) | | | | | | |

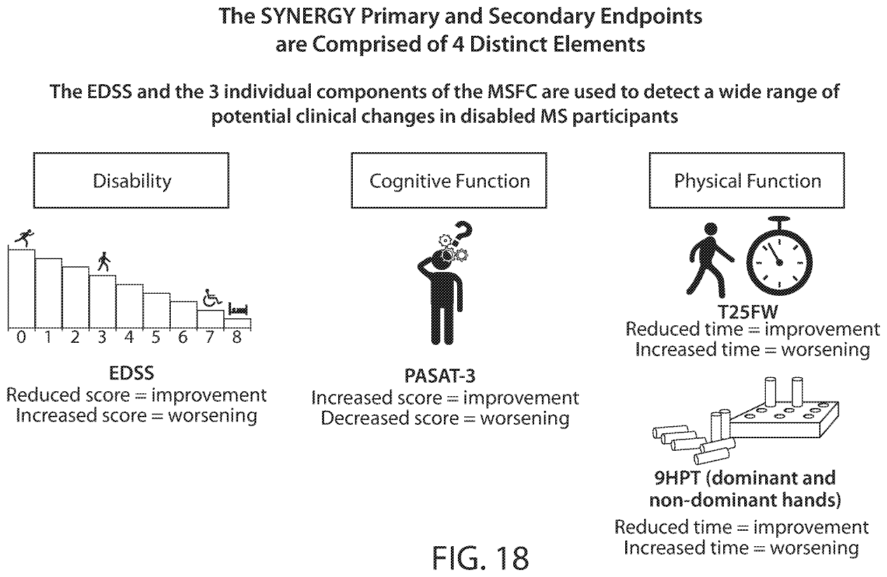

The SYNERGY trial primary and secondary endpoints comprised 4 distinct elements, as is shown in FIG. 18. The composite primary and secondary endpoints used in SYNERGY use well-established measures of disability, cognitive function, and physical function in MS: EDSS; PASAT-3; T25FW; and/or 9HPT dominant and non-dominant hands. The use of a multicomponent endpoint ensured that treatment effects were better detected in the context of this phase 2, proof-of-concept study. A change in PASAT, T25FW, and 9HPT (dominant and non-dominant hands) defined as ≥15% change from baseline confirmed 3 months or later. A Change in EDSS defined in the traditional way, confirmed 3 months or later. The baseline clinical characteristics of the patients are shown in Table 32.

TABLE 32
The baseline clinical characteristics of the patients treated with anti-LINGO-1

| | Placebo | 3 mg/kg | 10 mg/kg | 30 mg/kg | 100 mg/kg |
|---|---|---|---|---|---|
| Characteristic^{a} | (n = 91) | (n = 45) | (n = 94) | (n = 91) | (n = 91) |
| EDSS | 3.66 ± 1.45 | 3.34 ± 1.39 | 3.39 ± 1.52 | 3.54 ± 1.40 | 3.42 ± 1.48 |
| T25W | 8.18 ± 7.76 | 6.36 ± 2.42 | 7.39 ± 5.10 | 7.21 ± 4.30 | 7.56 ± 4.78 |
| 9HPT dominant | 25.12 ± 10.25 | 27.06 ± 13.12 | 27.74 ± 20.53 | 24.52 ± 9.19 | 25.17 ± 12.26 |
| 9HPT non- | 25.57 ± 8.00 | 27.87 ± 13.88 | 35.67 ± 78.50 | 24.93 ± 7.60 | 26.71 ± 13.63 |
| dominant | | | | | |
| PASAT-3 | 44.6 ± 13.05 | 49.4 ± 10.63 | 46.9 ± 10.80 | 46.3 ± 12.60 | 46.1 ± 12.99 |
| Disease | 11.2 ± 9.18 | 9.4 ± 9.23 | 9.9 ± 8.35 | 10.7 ± 8.42 | 8.8 ± 7.52 |
| duration (years) | | | | | |
| Number (%) | 72 (77) | 37 (82) | 73 (77) | 72 (77) | 72 (78) |
| with RRMS | | | | | |
| Relapses in the | 1.4 ± 0.66 | 1.5 ± 0.66 | 1.4 ± 0.71 | 1.5 ± 0.67 | 1.5 ± 0.70 |
| previous year | | | | | |
| Whole brain | 1421.9 ± 86.0 | 1442.0 ± 96.4 | 1416.4 ± 85.6 | 1416.5 ± 92.0 | 1410.2 ± 86.2 |
| volume (mL) | | | | | |
| Whole brain | 0.19 ± 0.21 | 0.20 ± 0.27 | 0.25 ± 0.24 | 0.25 ± 0.22 | 0.17 ± 0.27 |
| MTR (nMTRu) | | | | | |
| Whole brain | 0.74 ± 0.04 | 0.74 ± 0.05 | 0.74 ± 0.04 | 0.73 ± 0.04 | 0.74 ± 0.05 |
| DTI-RD | | | | | |
| (10⁻³ mm²/s) | | | | | |
| Number (%) | 41 (44) | 21 (47) | 38 (40) | 52 (56) | 42 (46) |
| with Gd⁺ lesions | | | | | |

The EDSS is used to assess neurologic impairment (including disability) in people with MS. Patients with MS are given an EDSS score based on their degree of abnormalities in 7 functional systems, plus their walking ability. The scores range from 0.0 (for a normal neurological examination) to 10.0 (death due to MS); the scores in between denote increasing disability. Therefore, a reduction in EDSS score indicates an improvement.

In the PASAT-3, subjects listen to a series of spoken numbers (1 number every 3 seconds), and must add each one to the previous number. The final score is the total number of correct additions made in the series. For this element, an increase in the final score indicates improved cognitive functioning, specifically information processing.

The T25FW and the 9HPT quantitatively measure leg function and ambulation, and arm and hand function, respectively. In the T25FW, subjects are required to complete a 25-foot walk as quickly as possible. The walking test is conducted a second time, and the final score is the mean time of the 2 completed walks. The 9HPT records the time taken by a subject to insert and remove 9 pegs from a board, with their dominant hand first and then with their non-dominant hand. The test is done twice and the final score is the mean time for both tests for each of the 2 hands. For both of these elements, a reduction in time represents an improvement.

EDSS, PASAT-9, T25FW, and 9HPT were measured every 12 weeks for 84 weeks. Changes in PASAT-9, T25FW, and 9HPT (either hand) were defined as ≥15% change from baseline confirmed 3 months or later. Change was EDSS defined in the traditional way, confirmed 3 months or later.

The use of a multicomponent endpoint ensured that treatment effects were better detected in the context of this phase 2, proof-of-concept study. Study participants exhibit a wide range of neurological impairments at baseline; as per the inclusion criteria, participants may have RRMS or SPMS with minimal-to-severe disability.

A multicomponent Functional Disability Endpoint (FDE) was also used. The FDE comprises T25FW, 9HPT and Symbol Digit Modalities Test (SDMT). The SDMT is a test that evaluates processing speed and working memory in which the subject is given 90 seconds to pair specific numbers with given geometric figures based on a reference key. For this the threshold for change was 5 or more digits.

### Results

### Primary Endpoint Analysis

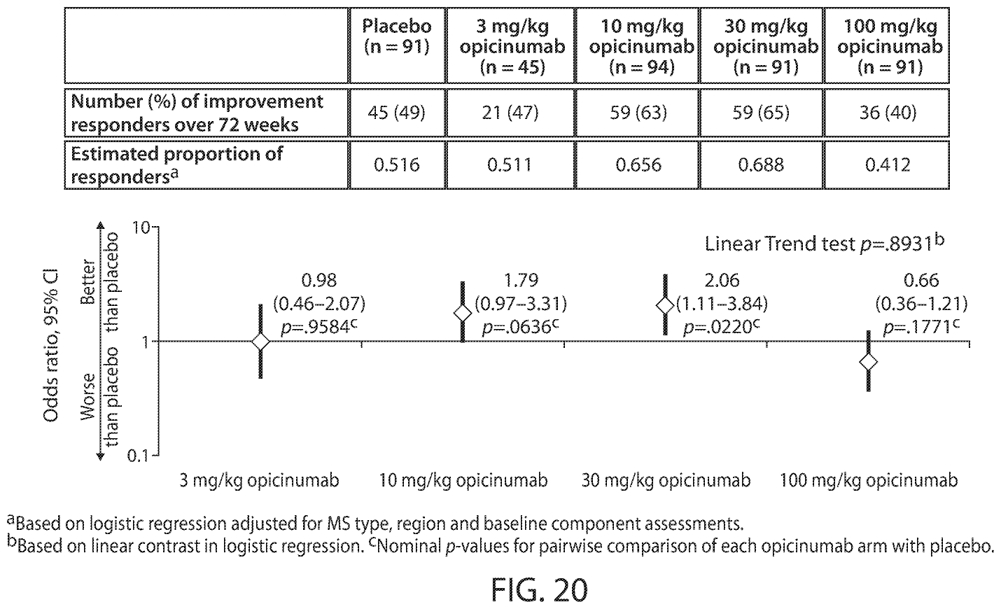

FIG. 19 shows the number of subjects in the SYNERGY trial in each treatment group that improved in the primary endpoint at the end of 72 weeks. Subjects were considered to be improved if they improved in any of EDSS, PASAT-3, T25FW, and/or 9HPT dominant and/or non-dominant hands. As is shown in FIG. 19, 51.6% of subjects in the placebo group showed improvement at 72 weeks, 51.1% of subjects in the 3 mg/kg treatment group showed improvement at 72 weeks, 65.6% of subjects in the 10 mg/kg treatment group showed improvement at 72 weeks, 68.8% of subjects in the 30 mg/kg treatment group showed improvement at 72 weeks, and 41.2% of subjects in the 100 mg/kg treatment group showed improvement at 72 weeks Subjects in the 10 mg/kg and 30 mg/kg treatment group showed improvement relative to the placebo group, having odds ratios of 1.79 (95% CI 0.97-3.31; p=0.0636) and 2.06 (95% CI 1.11-3.84; p=0.022), respectively. Subjects in the 3 mg/kg and 100 mg/kg treatment groups did not show improvement relative to the placebo, having odds ratios of 0.98 (95% CI 0.46-2.07; p=0.9584) and 0.66 (95% CI 0.36-1.21; p=0.1771), respectively. The estimated progression of responders, odds ratios, 95% confidence intervals and p-values shown in FIG. 19 are based on logistic regression adjusted for MS type, region and baseline component assessments. Trend test p-value is based on a linear contrast in logistic regression. FIG. 20 shows the odds ratio as a function of anti-LINGO-1 dosage. The results presented herein suggest that the response to anti-LINGO-1 is non-monotonic.

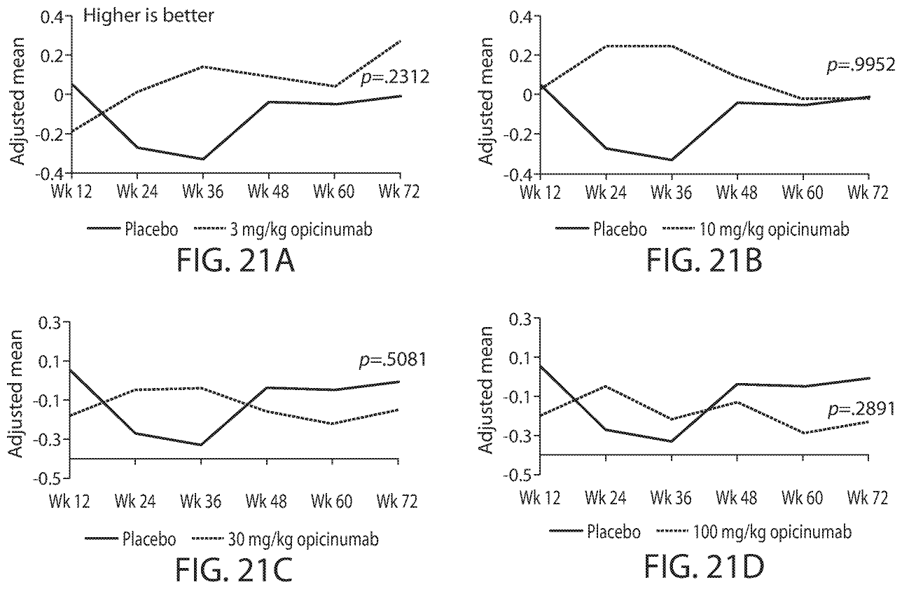

FIGS. 21A-21D shows the overall response score over 72 weeks by mixed model for repeated measures (MMRM) at each dosage. The data was adjusted for MS type, region and baseline component assessments. The overall response score incorporates increased improvement and reduced worsening and is based on all components of the primary and secondary endpoints except PASAT-3.

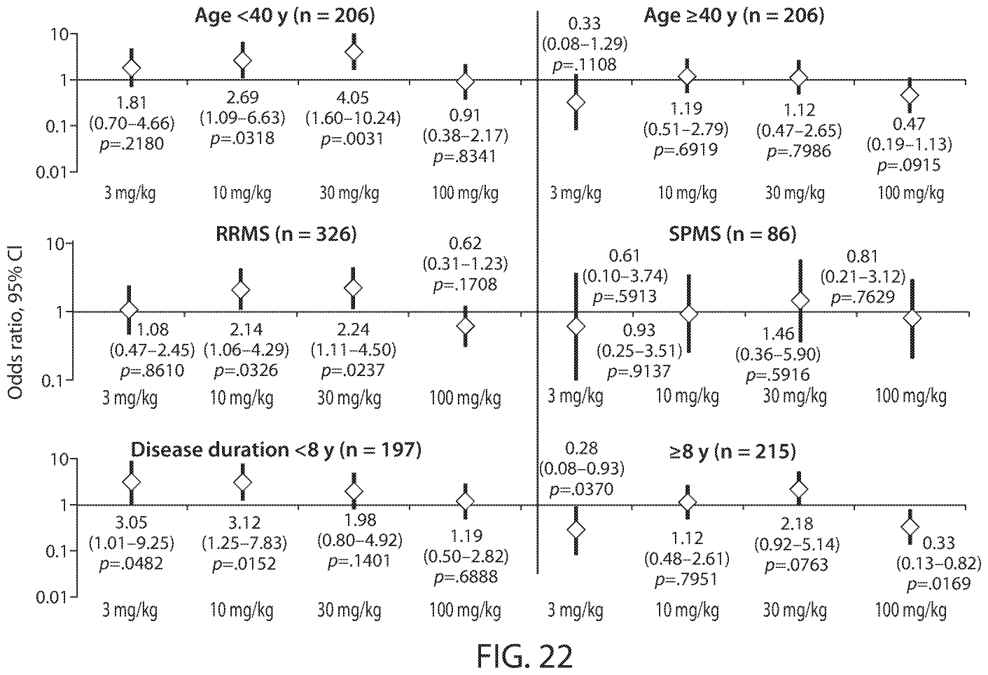

As is shown in Table 33, the primary endpoint data was stratified according to age, disease subtype, and disease duration. FIG. 22 shows the odds ratio as a function of anti-LINGO-1 dosage for each of the subgroups.

Among sunjects who were younger than 40, subjects in the 3 mg/kg, 10 mg/kg and 30 mg/kg treatment group showed improvement relative to the placebo group, having odds ratios of 1.81 (95% CI 0.70-4.66, p=0.2180), 2.69 (95% CI 1.09-6.63, p=0.0318), and 4.05 (95% CI 1.60-10.24, p=0.0031), respectively. Subjects in the 100 mg/kg treatment group did not show improvement relative to the placebo, having an odds ratio of 0.91 (95% 0.38-2.17, p=0.8341). Among sunjects who were 40 years old or older, subjects in the 10 mg/kg and 30 mg/kg treatment group showed improvement relative to the placebo group, having odds ratios of 1.19 (95% CI 0.51-2.79, p=0.6919) and 1.12 (95% CI 0.47-2.65, p=0.7986), respectively. Subjects in the 3 mg/kg and 100 mg/kg treatment group did not show improvement relative to the placebo group, having odds ratios of 0.33 (95% CI 0.08-1.29, p=0.11080) and 0.47 (95% CI 0.19-1.13, p=0.0915). The results presented herein suggest a stronger treatment effect for improvement of pre-existing disability and neurological function impairment in subjects under 40 years old.

Among subjects with RRMS, subjects in the 3 mg/kg, 10 mg/kg and 30 mg/kg treatment group showed improvement relative to the placebo group, having odds ratios of 1.08 (95% CI 0.47-2.45, p=0.8610), 2.14 (95% CI 1.06-4.29, p=0.0326), and 2.24 (95% CI 1.11-4.50, p=0.0237), respectively. Subjects in the 100 mg/kg treatment group did not show improvement relative to the placebo, having an odds ratio of 0.62 (95% CI 0.31-1.23, p=0.1708). Among subjects with SPMS, subjects in the 30 mg/kg treatment group showed improvement relative to the placebo group, having an odds ratio of 1.46 (95% CI 0.36-5.90, p=0.5916). Subjects in the 3 mg/kg, 10 mg/kg, and 100 mg/kg treatment groups did not show improvement relative to the placebo, having odds ratios of 0.61 (95% CI 0.10-3.74, p=0.5913), 0.93 (95% CI 0.25-3.51, p=0.9137), and 0.81 (95% CI 0.21-3.12, p=0.7629), respectively. The results presented herein suggest a stronger treatment effect for subjects having RRMS than for subjects having SPMS.

Among subjects with a disease duration of less than 8 years, subjects in the 3 mg/kg, 10 mg/kg, 30 mg/kg, and 100 mg/kg treatment groups showed improvement relative to the placebo, having odds ratios of 3.05 (95% CI 1.01-9.25, p=0482), 3.12 (95% CI 1.25-7.83=0.0152), 1.98 (95% CI 0.80-4.92, p=0.1401), and 1.19 (95% CI 0.50-2.82, p=0.6888), respectively. Among subjects with a disease duration of 8 years or greater, subjects in the 10 mg/kg and 30 mg/kg treatment groups showed improvement relative to the placebo, having odds ratios of 1.12 (95% CI 0.48-2.61, p=0.7951) and 2.18 (95% CI 0.92-5.14, p=0.0763), respectively. Subjects in the 3 mg/kg and 100 mg/kg treatment groups did not show improvement relative to the placebo, having odds ratios of 0.28 (95% CI 0.08-0.93, p=0.0370) and 0.33 (95% CI 0.13-0.82, p=0.0169), respectively. The results presented herein suggest that subjects with a disease duration of less than 8 years experience a greater treatment effect when treated with anti-LINGO-1.

TABLE 33
Efficacy on the primary endpoint by pre-specified baseline demographic
subgroup analyses versus placebo; based on logistic regression adjusted for MS type,
region and baseline component assessments.

| | 3 mg/kg | 10 mg/kg | 30 mg/kg | 100 mg/kg |
|---|---|---|---|---|
| Age < 40 y, n | 32 | 41 | 41 | 47 |
| Odds ratio (95% CI) | 1.81 (0.70-4.66) | 2.69 (1.09-6.63) | 4.05 (1.60-10.24) | 0.91 (0.38-2.17) |
| p-value | .2180 | .0318 | .0031 | .8341 |
| Age ≥ 40 y, n | 13 | 53 | 50 | 44 |
| Odds ratio (95% CI) | 0.33 (0.08-1.29) | 1.19 (0.51-2.79) | 1.12 (0.47-2.65) | 0.47 (0.19-1.13) |
| p-value | .1108 | .6919 | .7986 | .0915 |
| RRMS, n | 37 | 73 | 72 | 72 |
| Odds ratio (95% CI) | 1.08 (0.47-2.45) | 2.14 (1.06-4.29) | 2.24 (1.11-4.50) | 0.62 (0.31-1.23) |
| p-value | .8610 | .0326 | .0237 | .1708 |
| SPMS, n | 8 | 21 | 19 | 19 |
| Odds ratio (95% CI) | 0.61 (0.10-3.74) | 0.93 (0.25-3.51) | 1.46 (0.36-5.90) | 0.81 (0.21-3.12) |
| p-value | .5913 | .9137 | .5916 | .7629 |
| Disease duration < 8 y, n | 23 | 44 | 40 | 48 |
| Odds ratio (95% CI) | 3.05 (1.01-9.25) | 3.12 (1.25-7.83) | 1.98 (0.80-4.92) | 1.19 (0.50-2.82) |
| p-value | .0482 | .0152 | .1401 | .6888 |
| Disease duration ≥ 8 y, n | 22 | 50 | 51 | 43 |
| Odds ratio (95% CI) | 0.28 (0.08-0.93) | 1.12 (0.48-2.61) | 2.18 (0.92-5.14) | 0.33 (0.13-0.82) |
| p-value | .0370 | .7951 | .0763 | .0169 |

Measurements for the individual components of the primary endpoint are provided in Table 34. Subjects in the 3 mg/kg, 10 mg/kg, and 30 mg/kg treatment groups showed improvement in EDSS score relative to the placebo, having odds ratios of 1.08 (95% CI 0.36-3.26, p=0.8938), 1.58 (95% CI 0.67-3.72, p=0.2967), and 1.09 (95% CI 0.44-2.72, p=0.8464), respectively. Subjects in the 100 mg/kg treatment group did not show improvement in EDSS score relative to the placebo, having an odds ratio of 0.97 (95% CI 0.38-2.45, p=0.9433). Subjects in the 10 mg/kg treatment group showed improvement in 9HPT dominant hand relative to the placebo, having an odds ratio of 1.25 (95% CI 0.58-2.70, p=0.5767). Subjects in the 3 mg/kg, 30 mg/kg, and 100 mg/kg treatment groups did not show improvement in 9HPT dominant hand relative to the placebo, having odds ratios of 0.84 (95% CI 0.31-2.30, p=0.7296), 0.96 (95% CI 0.43-2.17, p=0.9293), and 0.83 (95% CI 0.36-1.91, p=0.6624), respectively. Subjects in the 10 mg/kg and 30 mg/kg treatment groups showed improvement in 9HPT non-dominant hand relative to the placebo, having odds ratios of 2.42 (95% CI 1.03-5.68, p=0.0415) and 1.36 (95% CI 0.54-3.42, p=0.5080), respectively. Subjects in the 3 mg/kg and 100 mg/kg treatment groups did not show improvement in 9HPT non-dominant hand relative to the placebo, having odds ratios of 0.88 (95% CI 0.26-3.03, p=0.8392) and 0.99 (95% CI 0.37-2.61, p=0.9767), respectively. Subjects in the 3 mg/kg and 10 mg/kg treatment groups showed improvement in T25FW relative to the placebo, having odds ratios of 1.81 (95% CI 0.69-4.76, p=0.2269) and 1.31 (95% CI 0.57-3.00, p=0.5261), respectively. Subjects in the 30 mg/kg and 100 mg/kg treatment groups did not show improvement in T25FW relative to the placebo, having odds ratios of 0.77 (95% CI 0.30-1.93, p=0.5724) and 0.94 (95% CI 0.39-2.28, p=0.8981), respectively. Subjects in the 3 mg/kg, 10 mg/kg, and 30 mg/kg treatment groups showed improvement in PASAT-3 relative to the placebo, having odds ratios of 1.21 (95% CI 0.45-3.25, p=0.7048), 1.24 (95% CI 0.58-2.68, p=0.5823), and 1.60 (95% CI 0.74-3.44, p=0.2312), respectively. Subjects in the 100 mg/kg treatment group did not show improvement in PASAT-3 relative to the placebo, having an odds ratio of 0.51 (95% CI 0.22-1.20, p=0.1213).

TABLE 34
Confirmed improvers on individual components of the primary endpoint versus
placebo; based on logistic regression adjusted for MS type, region and baseline component
assessments.

| | 3 mg/kg | 10 mg/kg | 30 mg/kg | 100 mg/kg |
|---|---|---|---|---|
| | (n = 45) | (n = 94) | (n = 91) | (n = 91) |
| EDSS | 1.08 (0.36-3.26) | 1.58 (0.67-3.72) | 1.09 (0.44-2.72) | 0.97 (0.38-2.45) |
| Odd ratio (95% CI) | .8938 | .2967 | .8464 | .9433 |
| p-value | | | | |
| 9HPT dominant hand | 0.84 (0.31-2.30) | 1.25 (0.58-2.70) | 0.96 (0.43-2.17) | 0.83 (0.36-1.91) |
| Odd ratio (95% CI) | .7296 | .5767 | .9293 | .6624 |
| p-value | | | | |
| 9HPT non-dominant hand | 0.88 (0.26-3.03) | 2.42 (1.03-5.68) | 1.36 (0.54-3.42) | 0.99 (0.37-2.61) |
| Odd ratio (95% CI) | .8392 | .0415 | .5080 | .9767 |
| p-value | | | | |
| T25FW | 1.81 (0.69-4.76) | 1.31 (0.57-3.00) | 0.77 (0.30-1.93) | 0.94 (0.39-2.28) |
| Odd ratio (95% CI) | .2269 | .5261 | .5724 | .8981 |
| p-value | | | | |
| PASAT-3 | 1.21 (0.45-3.25) | 1.24 (0.58-2.68) | 1.60 (0.74-3.44) | 0.51 (0.22-1.20) |
| Odd ratio (95% CI) | .7048 | .5823 | .2312 | .1213 |
| p-value | | | | |

### Whole Brain DTI Analysis

The primary endpoint data was stratified according to whole brain Diffusion Tensor Imaging (DTI) radial diffusivity (RD) at baseline. Increased DTI (RD) is thought to be associated with demyelination and increased MS disease severity. The primary endpoint data was stratified according to whether the patient had a baseline whole brain DTI (RD) of less than the population median (0.73×10\hat{ }−3 mm\hat{ }2/s) or greater than or equal to the population median (0.73×10\hat{ }−3 m\hat{m} 2/s).

As shown in Tables 35 and 36, in patients having a whole brain DTI (RD) of less than 0.73×10\hat{ }−3 mm\hat{ }2/s, all treatment groups (e.g., 3 mg/kg, 10 mg/kg, 30 mg/kg, and 100 mg/kg) showed improvement in the primary endpoint relative to the placebo group. The 3 mg/kg, 10 mg/kg, 30 mg/kg, and 100 mg/kg treatment groups had odds ratios of 3.55 (95% CI 1.18-10.69, p=0.0242), 6.35 (95% CI 2.43-16.62, p=0.0002), 2.90 (1.17-7.19, p=0.0217), and 1.59 (95% CI 0.64-3.98, p=0.3193), respectively. Similarly, when the primary endpoint was evaluated without PASAT-3, or when only EDSS was used as an endpoint at 72 weeks, all treatment groups (e.g., 3 mg/kg, 10 mg/kg, 30 mg/kg, and 100 mg/kg) showed improvement relative to the placebo group.

In contrast, in patients having a baseline whole brain DTI (RD) of greater than or equal to 0.73×10\hat{ }−3 mm\hat{ }2/s, only one treatment group, 30 mg/kg, showed improvement in the primary endpoint relative to the placebo, with an odds ratio of 1.75 (95% CI 0.68-4.53, p=0.2488). The 3 mg/kg, 10 mg/kg, and 100 mg/kg treatment groups did not show improvement, having odds ratios of 0.33 (95% CI 0.10-1.07, p=0.0645), 0.76 (95% CI 0.31-1.88, p=0.5581) and 0.34 (95% CI 0.14-0.85, p=0.0204), respectively.

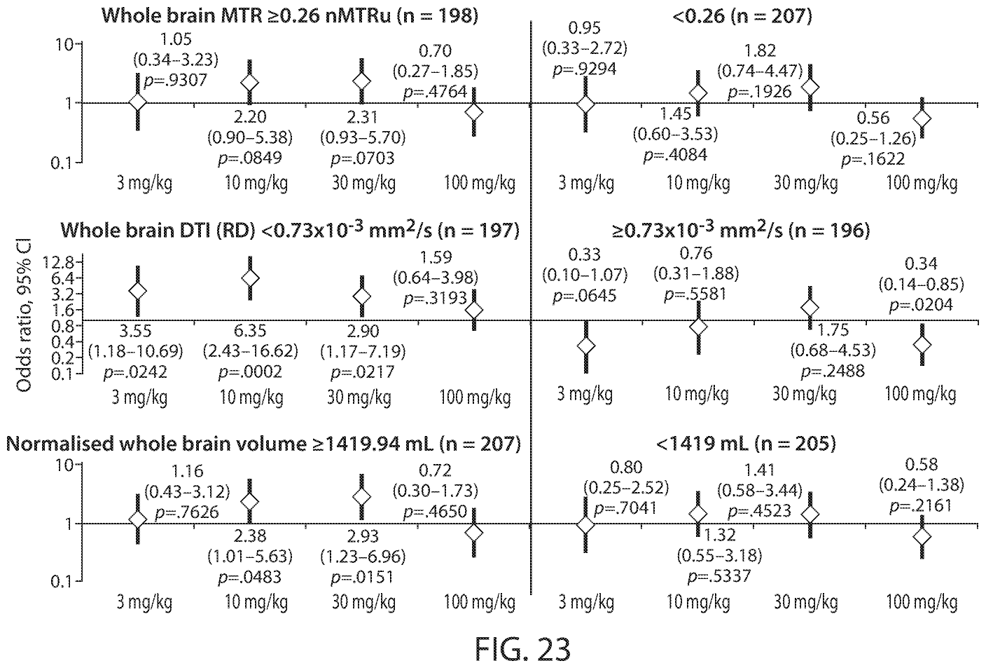

FIG. 23 shows the odds ratio as a function of anti-LINGO-1 dosage for whole brain DTI (RD) of less than 0.73×10\hat{ }−3 mm\hat{ }2/s and for whole brain DTI (RD) of greater than or equal to 0.73×10\hat{ }−3 mm\hat{ }2/s.

When the primary endpoint was evaluated without PASAT-3, the 10 mg/kg, and 30 mg/kg treatment groups showed improvement. When only EDSS was used as an endpoint at 72 weeks, none of the treatment groups showed improvement (See Table 35).

TABLE 35
Improvement by baseline whole brain DTI (RD).

| | | | | | |
|---|---|---|---|---|---|
| % | Placebo | 3 mg/kg | 10 mg/kg | 30 mg/kg | 100 mg/kg |
| Odds Ratio | n = 91 | n = 45 | n = 94 | n = 91 | n = 91 |

Baseline whole brain DTI (RD) < 0.73 × 10\hat{ } −3 mm\hat{ }2/s (median)

| | | | | | |
|---|---|---|---|---|---|
| Primary | 39.7% | 70.1% | 80.7% | 65.6% | 51.2% |
| | | 3.55 | 6.35 | 2.90 | 1.59 |
| Primary | 30.8% | 56.2% | 66.0% | 38.1% | 40.5% |
| w/o PASAT3 | | 2.89 | 4.38 | 1.69 | 1.53 |
| EDSS | 9.0% | 26.2% | 25.9% | 12.4% | 18.3% |
| | | 3.57 | 3.53 | 1.43 | 2.26 |

Baseline whole brain DTI (RD) > = 0.73 × 10\hat{ } −3 mm\hat{ }2/s (median)

| | | | | | |
|---|---|---|---|---|---|
| Primary | 60.8% | 34.2% | 54.3% | 73.1% | 34.6% |
| | | 0.33 | 0.76 | 1.75 | 0.34 |
| Primary | 47.0% | 28.1% | 48.8% | 49.8% | 29.3% |
| w/o PASAT3 | | 0.44 | 1.07 | 1.12 | 0.47 |
| EDSS | 16% | 0 | 11% | 15% | 9% |

In patients having a whole brain MTR of less than or equal to 0.26 nMTRu, the 3 mg/kg, 10 mg/kg, and 30 mg/kg treatment groups showed improvement in the primary endpoint relative to the placebo group. The 3 mg/kg, 10 mg/kg, 30 mg/kg, and 100 mg/kg treatment groups had odds ratios of 1.05 (95% CI 0.34-3.23, p=0.9307), 2.20 (95% CI 0.90-5.38, p=0.0849), 2.31 (95% CI 0.93-5.70, p=0.0703), and 0.70 (95% CI 0.27-1.85, p=0.4′764), respectively. In contrast, in patients having a baseline whole brain MTR of greater than 0.26 nMTRu, only subjects in the 10 mg/kg and the 30 mg/kg treatment groups showed improvement in the primary endpoint relative to the placebo group, having odds ratios of 1.45 (95% CI 0.60-3.53, p=0.4084) and 1.82 (95% CI 0.74-4.47, p=0.1926) respectively. Subjects in the 3 mg/kg and the 100 mg/kg treatment groups did not show improvement in the primary endpoint relative to the placebo group, having odds ratios of 0.95 (95% CI 0.33-2.72, p=0.9294) and 0.56 (95% CI 0.25-1.26, p=0.1622) respectively.

FIG. 23 shows the odds ratio as a function of anti-LINGO-1 dosage for whole brain MTR of less than or equal to 0.26 nMTRu and for whole brain MTR of greater than 0.26 nMTRu. nMTRu are normalized MTR units. In order to directly compare data acquired at different centers, MTR values are mapped onto a normalized scale where 0 is the median MTR of normal gray matter and 1 is the median MTR of normal white matter. These reference values are measured for a particular scanner using the dummy run for the site, which is performed on a healthy subject. Using the reference values from the dummy run, a linear mapping function that translates MTR values from that scanner into calibrated MTR values on the normalized scale is constructed. On the normalized scale, MTR values in brain tissue, including non-acute lesions, typically range from about −0.5 to 1.5. MTR in acute lesions can be much lower. (See Brown R A, et al. Proc Intl Soc Mag Reson Med. 2011; 19:4082).

In patients having a normalized whole brain volume of less than or equal to 1419.94 mL, the 3 mg/kg, 10 mg/kg, and 30 mg/kg treatment groups showed improvement in the primary endpoint relative to the placebo group. The 3 mg/kg, 10 mg/kg, and 30 mg/kg, treatment groups had odds ratios of 1.16 (95% CI 0.43-3.12, p=0.7626), 2.38 (95% CI 1.01-5.63, p=0.0483), and 2.93 (95% CI 1.23-6.96, p=0.0151) respectively. Subjects in the 100 mg/kg treatment groups did not show improvement in the primary endpoint relative to the placebo group, having an odds ratio of 0.72 (95% CI 0.30-1.73, p=0.4650). In patients having a baseline normalized whole brain volume of greater than 1419.94 mL, subjects in the 10 mg/kg and the 30 mg/kg treatment groups showed improvement in the primary endpoint relative to the placebo group, having odds ratios of 1.32 (95% CI 0.55-3.18, p=0.5337) and 1.41 (95% CI 0.58-3.44, p=0.4523), respectively. Subjects in the 3 mg/kg and the 100 mg/kg treatment groups did not show improvement in the primary endpoint relative to the placebo group, having odds ratios of 0.80 (95% CI 0.25-2.52, p=0.7041) and 0.58 (95% CI 0.24-1.38, p=0.2161) respectively.

FIG. 23 shows the odds ratio as a function of anti-LINGO-1 dosage for normalized whole brain volume of less than or equal to 1419.94 mL and for normalized whole brain volume of greater than 1419.94 mL.

TABLE 36
Efficacy on the primary endpoint by pre-specified baseline MRI subgroup
analyses versus placebo; based on logistic regression adjusted for MS type, region and
baseline component assessments.

| | 3 mg/kg | 10 mg/kg | 30 mg/kg | 100 mg/kg |
|---|---|---|---|---|
| Whole brain MTR ≥ 0.26 nMTRu, n | 21 | 53 | 50 | 37 |
| Odds ratio (95% CI) | 1.05 (0.34-3.23) | 2.20 (0.90-5.38) | 2.31 (0.93-5.70) | 0.70 (0.27-1.85) |
| p-value | .9307 | .0849 | .0703 | .4764 |
| Whole brain MTR < 0.26 nMTRu, n | 22 | 40 | 41 | 53 |
| Odds ratio (95% CI) | 0.95 (0.33-2.72) | 1.45 (0.60-3.53) | 1.82 (0.74-4.47) | 0.56 (0.25-1.26) |
| p-value | .9294 | .4084 | .1926 | .1622 |
| Whole brain DTI (RD) < 0.73 × 10⁻³ | | | | |
| mm²/s, n | 22 | 45 | 46 | 42 |
| Odds ratio (95% CI) | 3.55 (1.18-10.69) | 6.35 (2.43-16.62) | 2.90 (1.17-7.19) | 1.59 (0.64-3.98) |
| p-value | .0242 | .0002 | .0217 | .3193 |
| Whole brain DTI (RD) ≥ 0.73 × 10⁻³ | | | | |
| mm²/s, n | 21 | 45 | 39 | 46 |
| Odds ratio (95% CI) | 0.33 (0.10-1.07) | 0.76 (0.31-1.88) | 1.75 (0.68-4.53) | 0.34 (0.14-0.85) |
| p-value | .0645 | .5581 | .2488 | .0204 |
| Whole brain volume ≥ 1419.94 mL, n | 26 | 45 | 47 | 42 |
| Odds ratio (95% CI) | 1.16 (0.43-3.12) | 2.38 (1.01-5.63) | 2.93 (1.23-6.96) | 0.72 (0.30-1.73) |
| p-value | .7626 | .0483 | .0151 | .4650 |
| Whole brain volume < 1419.94 mL, n | 19 | 49 | 44 | 49 |
| Odds ratio (95% CI) | 0.80 (0.25-2.52) | 1.32 (0.55-3.18) | 1.41 (0.58-3.44) | 0.58 (0.24-1.38) |
| p-value | .7041 | .5337 | .4523 | .2161 |

The results presented herein demonstrate that patients with a lower whole brain DTI (RD) are more likely to show improvement in response to anti-LINGO-1 treatment at a range of opicinumab doses. Accordingly, the results presented herein suggest that subjects with a lower whole brain DTI (RD) are more likely to be responsive to anti-LINGO-1 treatment.

### FDE Endpoint Analysis

The data from the SYNERGY clinical trial was also analysed using a multicomponent Functional Disability Endpoint (FDE). The FDE comprises T25FW, 9HPT and SDMT. FIG. 24 shows the number of subjects in each treatment group that improved in the FDE at the end of 72 weeks. Subjects were considered to be improved if they improved in any of T25FW, 9HPT and SDMT. As is shown in FIG. 24, 27.6% of subjects in the placebo group showed improvement at 72 weeks, 45.5% of subjects in the 3 mg/kg treatment group showed improvement at 72 weeks, 32.0% of subjects in the 10 mg/kg treatment group showed improvement at 72 weeks, 28.8% of subjects in the 30 mg/kg treatment group showed improvement at 72 weeks, and 17.5% of subjects in the 100 mg/kg treatment group showed improvement at 72 weeks Subjects in the 3 mg/kg, 10 mg/kg and 30 mg/kg treatment group showed improvement relative to the placebo group, having odds ratios of 2.19(95% CI 1.01-4.72; p=0.0465), 1.24 (95% CI 0.65-2.34; p=0.5180) and 1.06 (95% CI 0.55-2.04; p=0.8563), respectively. Subjects in the 100 mg/kg treatment groups did not show improvement relative to the placebo, having an odds ratio of 0.56 (95% CI 0.27-1.13; p=0.1066). The estimated progression of responders, odds ratios, 95% confidence intervals and p-values shown in FIG. 24 are based on logistic regression adjusted for MS type, region and baseline component assessments. Trend test p-value is based on a linear contrast in logistic regression.

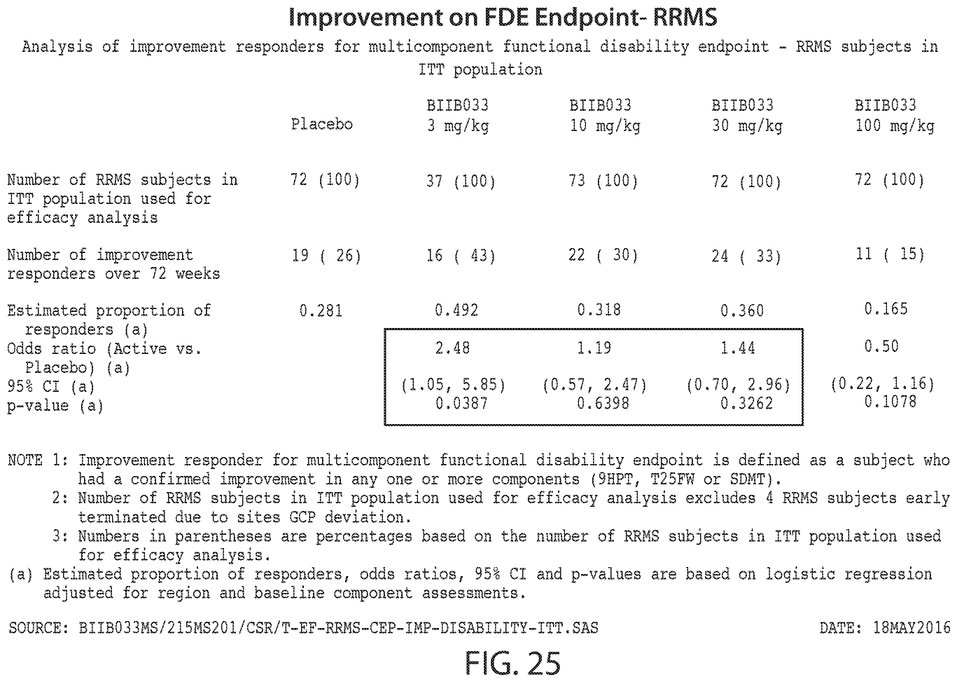

In the SYNERGY trial, 80% of subjects had RRMS and 20% of subjects had SPMS. The data from the subjects with RRMS was also analysed using a multicomponent Functional Disability Endpoint (FDE). As is shown in FIG. 25, 28.1% of subjects in the placebo group showed improvement at 72 weeks, 49.2% of subjects in the 3 mg/kg treatment group showed improvement at 72 weeks, 31.8% of subjects in the 10 mg/kg treatment group showed improvement at 72 weeks, 36.0% of subjects in the 30 mg/kg treatment group showed improvement at 72 weeks, and 16.5% of subjects in the 100 mg/kg treatment group showed improvement at 72 weeks Subjects in the 3 mg/kg, 10 mg/kg and 30 mg/kg treatment group showed improvement relative to the placebo group, having odds ratios of 2.48(95% CI 1.05-5.85; p=0.0387), 1.19 (95% CI 0.57-2.47; p=0.6398) and 1.44 (95% CI 0.70-2.96; p=0.3262), respectively. Subjects in the 100 mg/kg treatment groups did not show improvement relative to the placebo, having an odds ratio of 0.50 (95% CI 0.22-1.16; p=0.1078). The estimated progression of responders, odds ratios, 95% confidence intervals and p-values shown in FIG. 25 are based on logistic regression adjusted for MS type, region and baseline component assessments. Trend test p-value is based on a linear contrast in logistic regression.

According to the results presented herein, the FDE shows improvement at 3 mg/kg, 10 mg/kg, and 30 mg/kg in both the combined RRMS and SPMS patient population and in the RRMS patient population. The improvement in both the combined RRMS and SPMS patient population and in the RRMS patient population was greatest at 3 mg/kg. At both 3 mg/kg and 30 mg/kg, the effect is greater in the RRMS patient population than in the combined RRMS and SPMS patient population. At 3 mg/kg, the odds ratio in the RRMS and SPMS patient population is 1.06 and it is 1.44 in the RRMS patient population. At 30 mg/kg, the odds ratio in the RRMS and SPMS patient population is 2.19 and it is 2.48 in the RRMS patient population. Accordingly, the results presented herein suggest an increased responsiveness of RRMS to treatment with anti-LINGO-1 relative to SPMS.

### Total Cumulative Exposure

The data from the SYNERGY clinical trial was also analysed according to total cumulative exposure. Cumulative exposure is a measure of the total dose that a subject has received over time. At each timepoint, the total cumulative exposure can be determined by the dosage of the anti-LINGO-1 antibody being administered and the number of doses received.

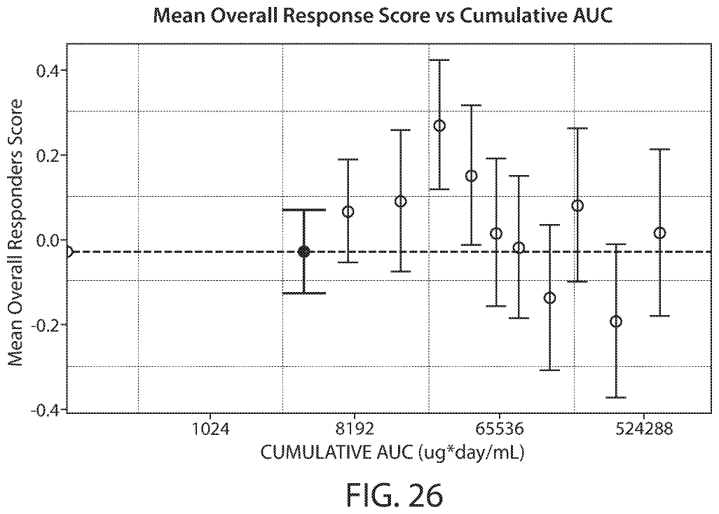

FIGS. 26-31 show cumulative exposure as accumulative area under the curve (AUC) in μg*day/ml. The accumulative AUC for treated subjects was divided into 10 bins, with each of the ten bins comprising approximately 200 data points. The placebo bin comprised 573 data points. In FIGS. 26-31, each of the 10 bins are represented by 10 vertical lines, which are the mean accumulative AUC value for each of bins 1-10. The 11^{th} line, which is left-most, represents the placebo. The accumulative AUC ranges and the mean accumulative AUC for each bin are summarized in Table 37. The number of data points comprising each bin are also shown. The first row of Table 37 shows the placebo, with an accumulative AUC of 0. Bin 1 has an accumulative AUC range of 1 to 1, 11,700 μg*day/ml. The mean accumulative AUC for Bin 1 is 7,517.426 μg*day/ml. Bin 2 has an accumulative AUC range of 11,700 to 20,800 μg*day/ml. The mean accumulative AUC for Bin 2 is 16,020.197. Bin 3 has an accumulative AUC range of 20,800 to 34,900 μg*day/ml. The mean accumulative AUC for Bin 3 is 28,025.722 μg*day/ml. Bin 4 has an accumulative AUC range of 34,900 to 54,300 μg*day/ml. The mean accumulative AUC for Bin 4 is 43,978.674 μg*day/ml. Bin 5 has an accumulative AUC range of 54,300 to 71,900 μg*day/ml. The mean accumulative AUC for Bin 5 is 63,071.467 μg*day/ml. Bin 6 has an accumulative AUC range of 71,900 to 106,000 μg*day/ml. The mean accumulative AUC for bin 6 is 86,778.400 μg*day/ml. Bin 7 has an accumulative AUC range of 106,000 to 166,000 μg*day/ml. The mean accumulative AUC for bin 7 is 134,934.113 μg*day/ml. Bin 8 has an accumulative AUC range of 166,000 to 240,000 μg*day/ml. The mean accumulative AUC of bin 8 is 200,708.540 μg*day/ml. Bin 9 has an accumulative AUC range of 240,000 to 486,000 μg*day/ml. The mean accumulative AUC of bin 9 is 350,064.450 μg*day/ml. Bin 10 has an accumulative AUC range of 486,000 to 1,370,000 μg*day/ml. The mean accumulative AUC of bin 10 is 655,217.980 μg*day/ml.

TABLE 37
AUC ranges for Bins 1-10 and placebo.

| | AUC Range | Mean AUC | |
|---|---|---|---|
| Bin | (μg*day/ml) | (μg*day/ml) | n (data points) |
| Placebo | −1, 1 | 0 | 573 |
| 1 | 1, 11,700 | 7,517.426 | 203 |
| 2 | 11,700, 20,800 | 16,020.197 | 203 |
| 3 | 20,800, 34,900 | 28,025.722 | 202 |
| 4 | 34,900, 54,300 | 43,978.674 | 203 |
| 5 | 54,300, 71,900 | 63,071.467 | 204 |
| 6 | 71,900, 106,000 | 86,778.400 | 201 |
| 7 | 106,000, 166,000 | 134,934.113 | 203 |
| 8 | 166,000, 240,000 | 200,708.540 | 202 |
| 9 | 240,000, 486,000 | 350,064.450 | 203 |
| 10 | 486,000, 1,370,000 | 655,217.980 | 203 |

FIG. 26 shows mean overall responsiveness score as a function of cumulative exposure. Mean overall response score comprises the sum of scores obtained at each clinical visit every time that each of 4 clinical measures (EDSS, 9HPT dominant hand, 9HPT non-dominant hand, and T25FW) improved or worsened. Every improvement event at every visit was scores +1, and every worsening event was scored as −1. All changes referred back to the baseline visit. The maximum score at each visit is +4 if all 4 components improve from baseline, and the worse score is −4 if each component worsened from baseline. The threshold for change were at least 1 point on the EDSS and at least 15% on the 9HPT (either hand) and the T25FW.

FIG. 26 shows that bin 3 (e.g., the third line to the right of the placebo) has the highest mean overall response score and bin 4 has the second highest overall response score. Thus, the results presented herein suggest that there is a level of total cumulative exposure that is less than the maximal total cumulative exposure produced in the study, which has the greatest therapeutic effect.

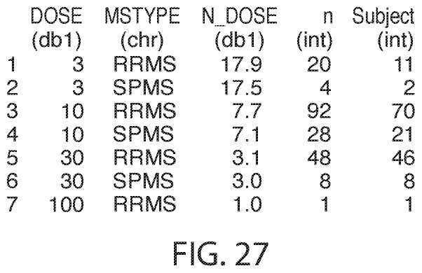

The dosages that comprise Bin 3 are shown in FIG. 27. Shown are the number of subjects (Subjects (int)), the number of datapoints (n(int)), the dosage (DOSE(db1)), the number of doses (N_DOSE(db1), and whether the subset is RRMS or SPMS (MSTYPE(chr)). As is shown in this table, among RRMS patients, bin 3 comprises subjects who have had, on average, 17.9 doses of 3 mg/kg anti-LINGO-1, 7.7 doses of 10 mg/kg anti-LINGO-1, 3.1 doses of 30 mg/kg anti-LINGO-1 and 1 dose of 100 mg/kg anti-LINGO-1.

When analysed by total cumulative exposure, the data presented herein demonstrate that certain dosing regimens that achieve a certain total cumulative exposure are associated with the greatest therapeutic effect. This total cumulative exposure can be achieved by more doses of a lower dose of anti-LINGO-1 or fewer doses of a higher dose of anti-LINGO-1.

### Total Cumulative Exposure in SPMS and RRMS

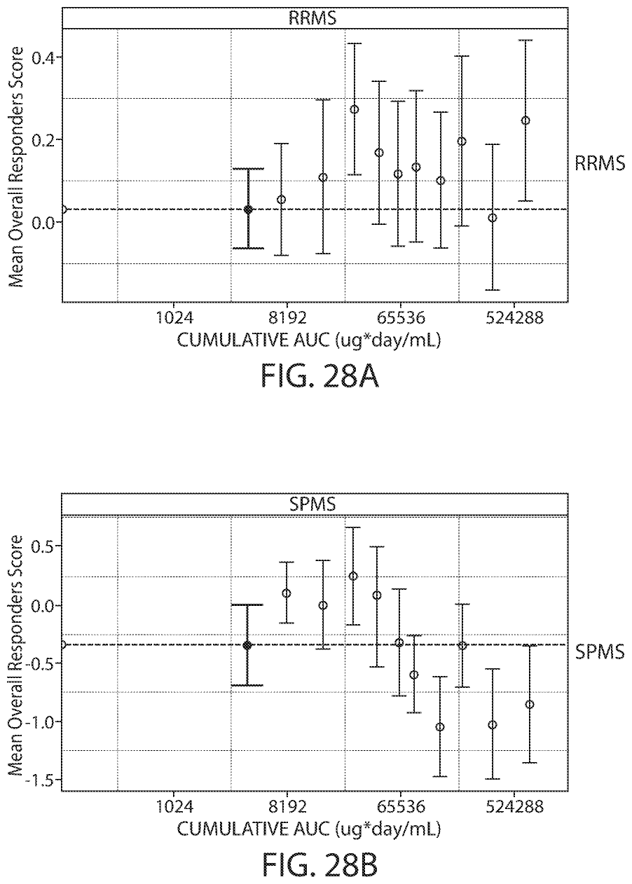

In FIGS. 28A and 28B, the SYNERGY data is stratified according to type of MS, and mean overall responsiveness score is then graphed as a function of cumulative exposure. FIG. 28A shows RRMS and FIG. 28B shows SPMS. With both RRMS and SPMS, the highest mean overall responsiveness score is seen in bin 3. One difference between the SPMS and RRMS data is that, with the RRMS data, the only bin with a lower mean overall responsiveness score than the placebo is bin 9. In contrast, for the SPMS data, bins 6, 7, 9, and 10 have mean overall responsiveness scores below the placebo. The results presented herein suggest that, for SPMS, having a higher exposure of anti-LINGO-1 can cause subjects to perform worse than those receiving a placebo, e.g., the anti-LINGO-1 can be harmful.

### Total Cumulative Exposure and Age

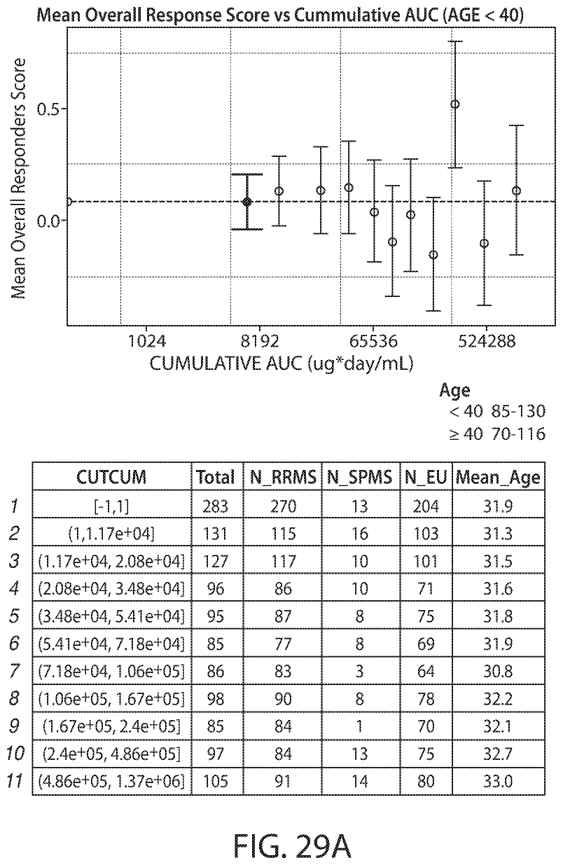

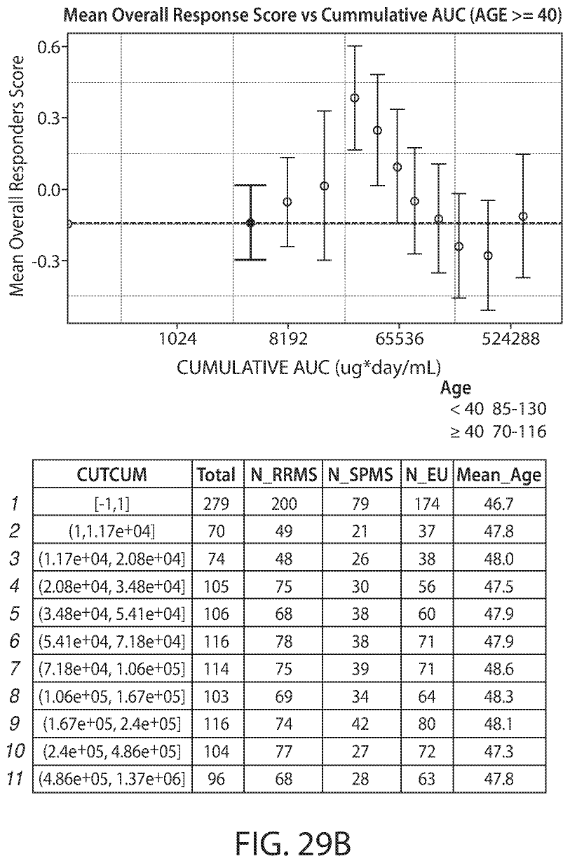

In FIGS. 29A and 29B, the SYNERGY data is stratified according to age, and mean overall responsiveness score is then graphed as a function of cumulative exposure. FIG. 29A shows subjects under forty years of age and FIG. 29B shows subjects aged forty and over. For subjects under forty, the highest mean overall responsiveness score was seen in bin 8, and bins 4-7 and 9 performed worse than placebo. There is an upward trend in bins 1-3 suggesting that bin 3 has an optimized therapeutic effect. Because bins 7 and 9 are both below the placebo, bin 8 may be an outlier. For subjects over forty years of age, the highest mean overall responsiveness score is seen with bin 3 and only bins 8 and 9 have a mean overall responsiveness score that is less than the placebo. The results presented herein suggest that subjects forty years of age and older have increased responsiveness to anti-LINGO-1 relative to subjects under forty.

### Total Cumulative Exposure and 9HPT

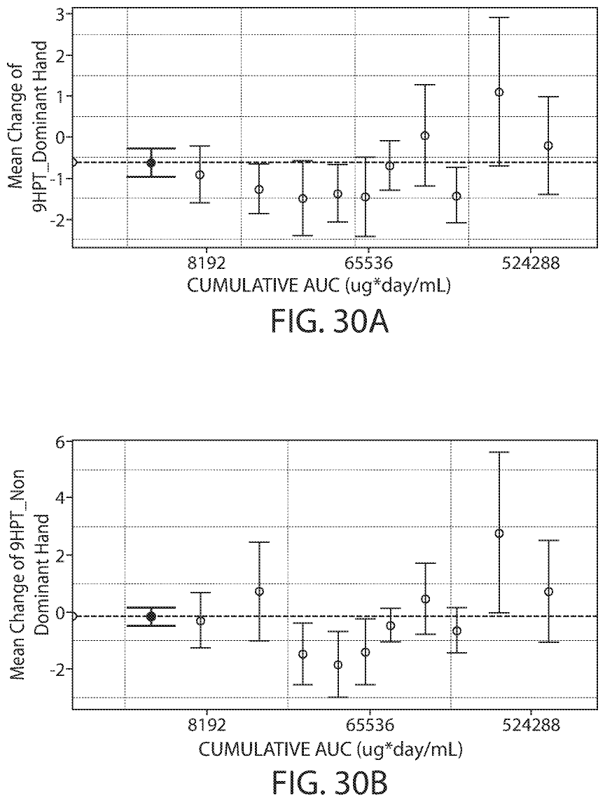

FIGS. 30A and 30B shows the 9HTP score as a function of cumulative exposure for both the dominant (FIG. 30A) and non-dominant (FIG. 30B) hands. The 9HPT records the time taken by a subject to insert and remove 9 pegs from a board. For both the dominant and non-dominant hand, the lowest 9HTP was in Bin 4.

### Total Cumulative Exposure and Change of T25FW

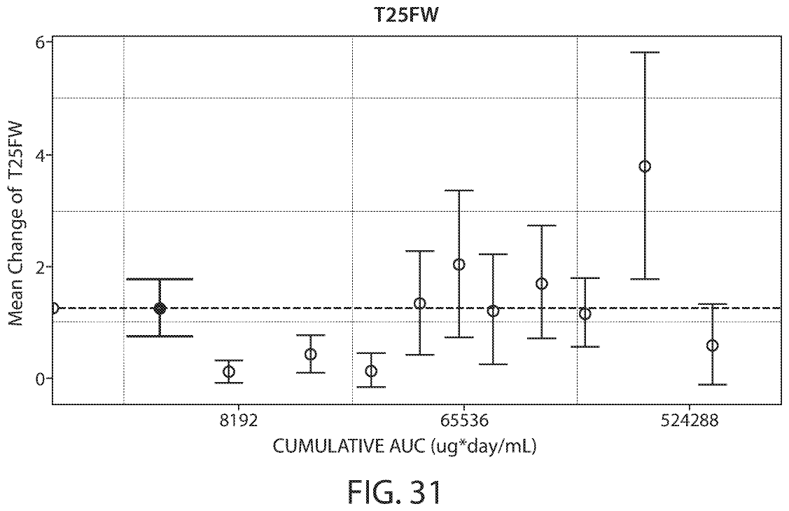

FIG. 31 shows the mean change of T25FW as a function of cumulative exposure. In bins 1-3, the mean change of T25FW is below 1, suggesting that subjects were able to walk more quickly than when treated with a placebo at low cumulative exposures of anti-LINGO-1.

### Pharmacokinetics

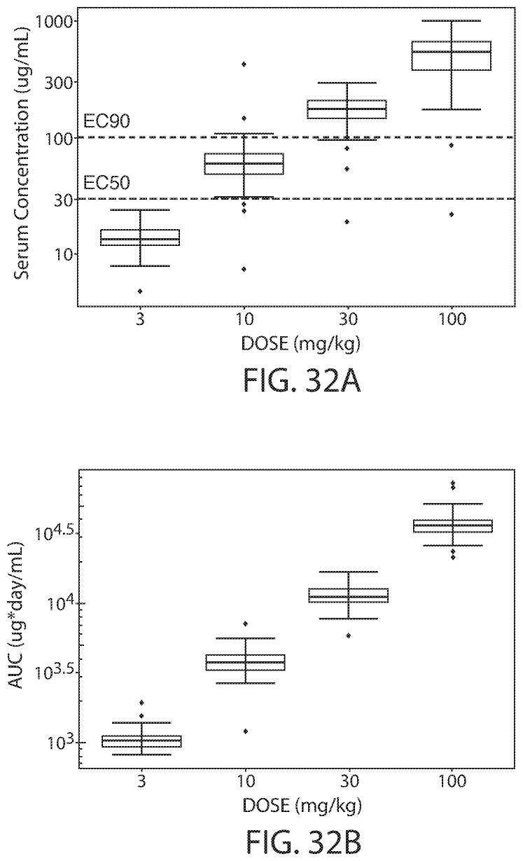

FIG. 32A shows serum concentration as a function of dosage at steady state relative to the predicted EC50 and EC90. FIG. 32B shows AUC of anti-LINGO-1 as a function of dosage at steady state. The results presented herein suggest that the pharmacokinetics are linear and that exposure increases dose-proportionally.

### Safety

Table 38 shows safety data for the SYNERGY study.

TABLE 38
Safety data for the SYNERGY study.

| | Placebo | 3 mg/kg | 10 mg/kg | 30 mg/kg | 100 mg/kg | All patients |
|---|---|---|---|---|---|---|
| | (n = 93) | (n = 45) | (n = 95) | (n = 93) | (n = 92) | (N = 418) |
| Number (%) with an | 79 (85) | 39 (87) | 84 (88) | 79 (85) | 73 (79) | 354 (85) |
| event | | | | | | |
| Number (%) with an | 8 (9) | 8 (18) | 15 (16) | 12 (13) | 16 (17) | 59 (14) |
| opicinumab or placebo | | | | | | |
| related event | | | | | | |
| Number (%) with a | 7 (8) | 2 (4) | 6 (6) | 6 (6) | 7 (8) | 28 (7) |
| severe event | | | | | | |
| Number (%) with a | 13 (14) | 4 (9) | 11 (12) | 20 (22) | 16 (17) | 64 (15) |
| serious event | | | | | | |
| Number (%) with an | 1 (1) | 0 | 0 | 1 (1) | 5 (5) | 7 (2) |
| opicinumab or placebo | | | | | | |
| related serious event | | | | | | |
| Incidence of post- | 3/75 | 1/42 | 12/84 | 13/76 (17) | 15/72 (21%) | — |
| baseline increase >7% | (4%) | (2%) | (14%) | | | |
| at week 72 | | | | | | |

### CONCLUSIONS

A pre-specified primary endpoint using a linear trend test across 5 arms was not met, although there was an increased proportion (not statistically significant) of improvement responders at 10 mg/kg and 30 mg/kg opicinumab vs placebo. This corresponds to an inverted U-shaped dose response. A higher proportion of improvement was seen with shorter disease duration, RRMS or less severe whole brain volume loss or whole brain DTI-RD impairment at baseline.

When analysed according to total cumulative exposure, bins 3 and 4 (corresponding to 20,800-54,300 μg*day/ml) showed the highest treatment effect.

### Example 6: Exploratory MRI Biomarkers of Opicinumab (Anti-LINGO-1) Repair of Pre-Existing Demyelination in Relapsing MS: Results from the Phase 2b Synergy Trial

### Introduction

There are currently no validated biomarkers for CNS remyelination. SYNERGY (Phase 2b; NCT01864148) is the first international, multicentre, randomised, double-blind, placebo-controlled trial to evaluate the effect of opicinumab (anti-LINGO-1) vs. placebo in active relapsing disabled (Expanded Disability Status Scale [EDSS] score 2-6) participants with multiple sclerosis. The primary objective of SYNERGY was to assess clinical improvement of pre-existing disability. Pre-clinical and single centre studies suggest magnetisation transfer ratio (MTR) and diffusion tensor imaging (DTI) are semi-quantitative measures of demyelination/remyelination. (Mallik S, et al. J Neurol Neurosurg. Psychiatry 2014; 85(12):1396-1404; Galetta S L, et al. Neuro Neuroimmunol Neuroinflammation. 2015; 2(4):e135). Change in MTR and/or DTI in Baseline non-enhancing T2 lesions was the lead candidate MRI biomarker of repair of pre-existing disability in SYNERGY. The objective of this study was to report initial results from exploratory analyses of SYNERGY brain magnetic resonance imaging (MRI) data, focusing on the changes in MTR and DTI in pre-existing T2 lesions.

### Methods

Brain MRI scans with nine sequences were run at each visit:
Prior to gadolinium (Gd⁺) injection:
   Sagittal T-1 global
   Proton density-weighted
   T2-weighted
   MT-OFF
   MT-ON
   DTI sequence
   T1-weighted
Immediately after Gd⁺ injection (during 10 min wait period)
   Fluid-attenuated inversion recovery
Post-Gd⁺ injection (after 10 min wait period)
   T1-weighted

Quality control (QC) and image analysis was performed by a third party (NeuroRx Research, Montreal, QC, Canada) blinded to treatment assignment/clinical status.

MT images were normalised to a calibrated scale, based on healthy volunteer data similarly acquired for each scanner. (Brown R A, et al. Proc Intl Soc Mag Reson Med. 2011; 19:4082).

Results are reported by: randomisation to opicinumab or placebo in the intention-to-treat (ITT) population; a pre-specified Baseline subgroup associated with treatment effect on the SYNERGY primary efficacy endpoint; and clinical improvement and worsening as defined in the primary and secondary endpoints.

All MRI analyses were exploratory and not powered for statistical significance.

Exploratory MRI Biomarker Analyses of Pre-existing T2 lesions in SYNERGY included change in MTR from Baseline for abnormal non-enhancing T2 lesions and change in DTI-radial diffusivity (RD) and fractional anisotropy (FA) from baseline for abnormal non-enhancing T2 lesions. A full listing of exploratory biomarkers is shown in Table 39.

TABLE 39
Exploratory biomarker analyses in SYNERGY (unit of
measure is the participant not the lesion)

| | |
|---|---|
| New brain | Percentage conversion from new Gd⁺ brain lesions |
| lesions | to CBH (defined as T1 hypointensity still visible |
| | after ≥ 6 months from onset) |
| | Percentage Gd⁺ lesion volume with significantly |
| | MTR |
| | Change from onset in new Gd⁺ lesion mean MTR |
| | relative to the NAWM^{4,5} |
| | Change from onset in MTR signal for voxels per |
| | scan whose MTR drops below the normal value |
| | (new ΔMTR lesions)^{4,5} |
| | Change from onset in new Gd⁺ lesion DTI |
| | Change from onset in DTI for voxels per scan |
| | whose MTR drops below the normal value |
| | (new ΔMTR lesions) |
| Pre-existing | Change in MTR from baseline for abnormal |
| brain lesions | non-enhancing T2 volume |
| (present at | Change in DTI-RD from baseline for abnormal |
| baseline scan) | non-enhancing T2 volume |
| | Change in DTI-FA from baseline for abnormal |
| | non-enhancing T2 volume |
| Diffuse brain | Percentage brain volume change⁶ |
| MRI metrics | Percentage change from baseline in cerebral cortical |
| | brain volume⁶ |
| | Percentage change from baseline in thalamic volume⁶ |
| | Change from baseline in whole brain MTR |
| | Change from baseline in NAWM MTR |
| | Change from baseline in whole brain DTI-RD |
| | Change from baseline in whole brain DTI-FA |

CBH, chronic black holes;
FA, functional anisotropy;
NAWM, normal appearing white matter;
RD, radial diffusivity.

### Results

Four hundred eighteen participants were randomised, and 412 analysed (6 excluded due to Good Clinical Practice [GCP] violations; Table). 4360 total scans were performed; 4325 (99.2%) passed QC, and 35 (0.8%) failed and were excluded from analysis. Baseline MRI characteristics are shown in Table 40.

TABLE 40
Baseline MRI characteristics

| Characteristic | | | | | | |
|---|---|---|---|---|---|---|
| mean ± SD | | | 10 mg/kg | 30 mg/kg | 100 mg/kg | Healthy adults |
| (median) | Placebo (n = 91) | 3 mg/kg (n = 45) | (n = 94) | (n = 91) | (n = 91) | (n = 47^{a}) |
| Whole brain | 1421.9 ± 86.0 | 1442.0 ± 96.4 | 1416.4 ± 85.6 | 1416.5 ± 92.1 | 1410.2 ± 86.2 | 1497.1 ± 75.4 |
| volume (mL) | (1421.0) | (1443.2) | (1411.5) | (1425.6) | (1418.2) | (1498.7) |
| Cerebral cortical | 535.7 ± 49.4 | 540.9 ± 55.7 | 534.5 ± 48.5 | 530.6 ± 51.3 | 527.2 ± 45.9 | 575.1 ± 44.7 |
| brain volume | (534.5) | (554.4) | (536.1) | (534.3) | (527.7) | (585.7) |
| (mL) | | | | | | |
| Thalamic volume | 15.0 ± 2.2 | 15.1 ± 2.2 | 15.1 ± 2.1 | 15.0 ± 2.0 | 15.0 ± 1.9 | 17.3 ± 1.3 |
| (mL) | (15.2) | (15.6) | (15.6) | (15.2) | (15.2) | (17.3) |
| Whole brain | 0.193 ± 0.21 | 0.200 ± 0.27 | 0.246 ± 0.24 | 0.248 ± 0.22 | 0.165 ± 0.27 | 0.310 ± 0.17 |
| median MTR | (0.202) | (0.258) | (0.287) | (0.273) | (0.215) | (0.350) |
| (nMTRu) | | | | | | |
| NAWM median | 0.764 ± 0.18 | 0.756 ± 0.24 | 0.790 ± 0.20 | 0.783 ± 0.22 | 0.725 ± 0.23 | 0.933 ± 0.05 |
| MTR (nMTRu) | (0.777) | (0.793) | (0.827) | (0.796) | (0.739) | (0.950) |
| Whole brain | 0.736 ± 0.04 | 0.741 ± 0.05 | 0.737 ± 0.04 | 0.732 ± 0.04 | 0.740 ± 0.05 | 0.705 ± 0.04 |
| DTI-RD (10⁻³ mm²/s) | (0.736) | (0.728) | (0.732) | (0.726) | (0.734) | (0.699) |
| Whole brain | 0.280 ± 0.03 | 0.281 ± 0.03 | 0.280 ± 0.02 | 0.284 ± 0.03 | 0.282 ± 0.03 | 0.288 ± 0.03 |
| DTI-FA | (0.281) | (0.279) | (0.278) | (0.287) | (0.281) | (0.288) |
| T1 lesion volume | 4.66 ± 6.4 | 4.54 ± 6.5 | 3.90 ± 6.8 | 3.62 ± 5.1 | 4.57 ± 6.6 | — |
| (mL) | (1.71) | (2.07) | (1.36) | (1.41) | (1.96) | |
| T2 lesion volume | 12.36 ± 14.76 | 12.94 ± 15.27 | 10.24 ± 12.85 | 10.54 ± 12.58 | 12.50 ± 15.1 | — |
| (mL) | (6.27) | (6.26) | (5.51) | (5.47) | (7.23) | |
| Summary | 2.0 ± 4.13 | 1.5 ± 3.62 | 1.3 ± 2.79 | 1.9 ± 4.10 | 2.1 ± 6.39 | — |
| consensus Gd⁺ | (0.0) | (0.0) | (0.0) | (0.0) | (0.0) | |
| lesion count | | | | | | |

^{a}From 47 healthy volunteers scanned at participating sites.
mL = millilitres;
nMTRu = normalised magnetization transfer units

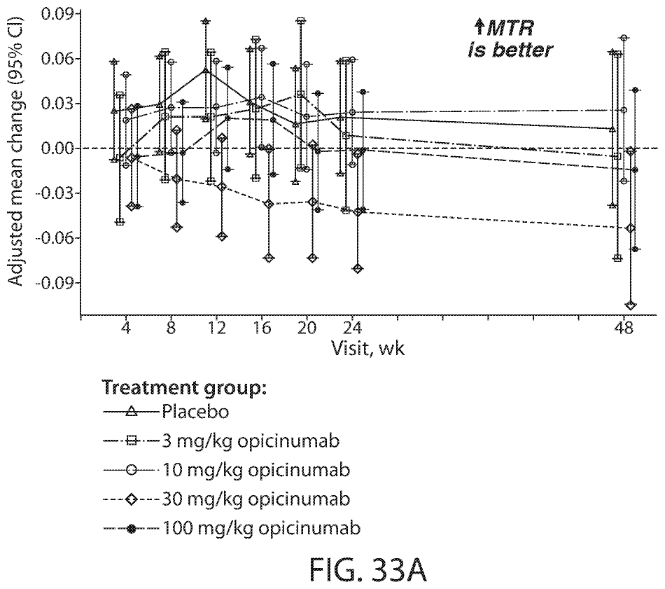

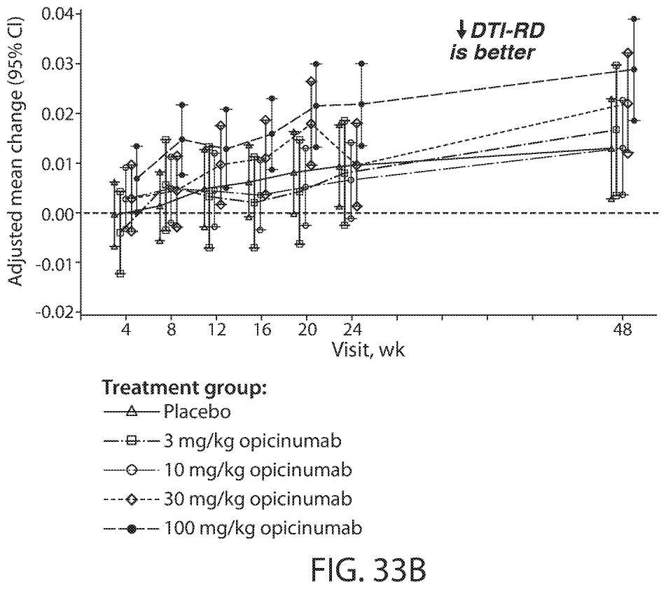

FIGS. 33A and 33B show the change in MTR and DTI, respectively, from baseline over the treatment period. No clear treatment effect was seen on change from Baseline in MTR or DTI on pre-existing non-enhancing T2 lesions between placebo and the 4 opicinumab treatment arms in the ITT population.

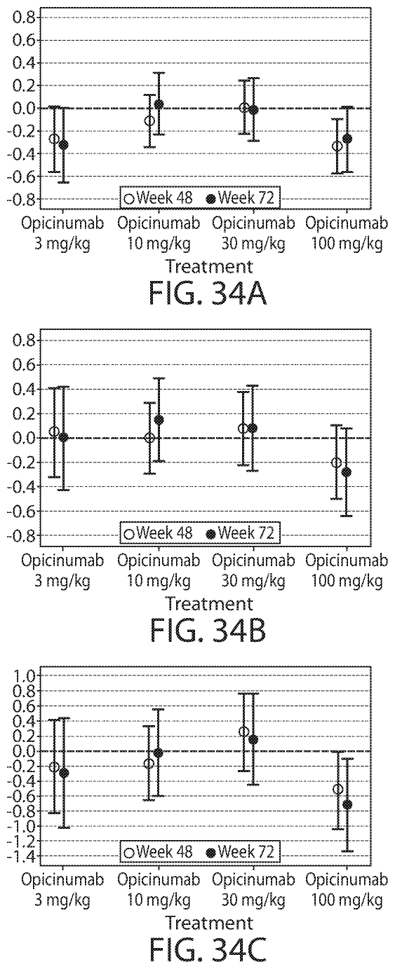

FIGS. 34A-34C shown the net change in brain volume from baseline at 48 weeks and 72 weeks in the whole brain (FIG. 34A), cerebral cortical brain (FIG. 34B) and thalamic brain (FIG. 34C). Diffuse brain MRI metrics demonstrated loss of volume across all treatment arms, with no clear differences between opicinumab and placebo.

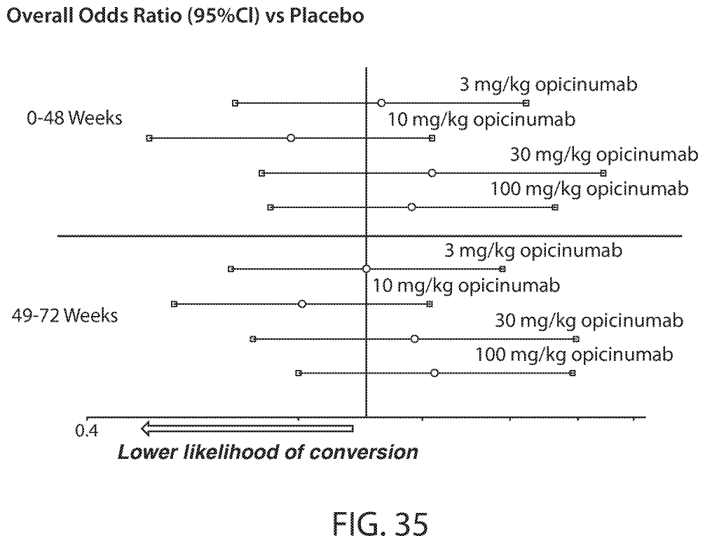

A potential benefit was observed with the 10 mg/kg treatment group showing the lowest rate of Gd+ lesion conversion to chronic black holes (FIG. 35).

Assessment of pre-specified baseline brain MRI by subgroups identified participants with greater whole brain volume, thalamic volume, MTR and lower DTI-RD are more likely to meet the clinical improvement endpoint. Treatment affect according to baseline DTI-RD, MTR, whole brain volume, and thalamic volume are shown in Table 40.

In subjects having a baseline whole brain DTI (RD) of <0.73×10⁻³ mm²/s, subjects in all four treatment groups showed improvement in the primary endpoint relative to the placebo group, having odds ratios of 3.55, 6.35, 2.90, and 1.59, respectively. In contrast, in subjects having a baseline whole brain DTI (RD) of ≥0.73×10⁻³ mm²/s, only subjects in the 30 mg/kg treatment group showed improvement in the primary endpoint relative to the placebo group, having an odds ratio of 1.75. Subjects in the 3 mg/kg, 10 mg/kg, and 100 mg/kg treatment groups did not show improvement in the primary endpoint relative to the placebo group, having odds ratios of 0.33, 0.76., and 0.34, respectively.

In patients having a whole brain MTR of less than or equal to 0.26 nMTRu, the 3 mg/kg, 10 mg/kg, and 30 mg/kg treatment groups showed improvement in the primary endpoint relative to the placebo group. The 3 mg/kg, 10 mg/kg, 30 mg/kg, and 100 mg/kg treatment groups had odds ratios of 1.05, 2.20, 2.31, and 0.70, respectively. In contrast, in patients having a baseline whole brain MTR of greater than 0.26 nMTRu, only subjects in the 10 mg/kg and the 30 mg/kg treatment groups showed improvement in the primary endpoint relative to the placebo group, having odds ratios of 1.45 and 1.82, respectively. Subjects in the 3 mg/kg and the 100 mg/kg treatment groups did not show improvement in the primary endpoint relative to the placebo group, having odds ratios of 0.95 and 0.56, respectively.

In patients having a whole brain volume of less than or equal to 1419.94 mL, the 3 mg/kg, 10 mg/kg, and 30 mg/kg treatment groups showed improvement in the primary endpoint relative to the placebo group. The 3 mg/kg, 10 mg/kg, and 30 mg/kg, treatment groups had odds ratios of 1.16, 2.38, and 2.93. respectively. Subjects in the 100 mg/kg treatment groups did not show improvement in the primary endpoint relative to the placebo group, having an odds ratio of 0.72. In patients having a baseline whole brain volume of greater than 1419.94 mL, subjects in the 10 mg/kg and the 30 mg/kg treatment groups showed improvement in the primary endpoint relative to the placebo group, having odds ratios of 1.32 and 1.41, respectively. Subjects in the 3 mg/kg and the 100 mg/kg treatment groups did not show improvement in the primary endpoint relative to the placebo group, having odds ratios of 0.80 and 0.58, respectively.

In patients having a baseline thalamic volume of ≥15.31 mL, the 3 mg/kg, 10 mg/kg, and 30 mg/kg treatment groups showed improvement in the primary endpoint relative to the placebo group. The 3 mg/kg, 10 mg/kg, and 30 mg/kg, treatment groups had odds ratios of 1.15, 2.43, and 2.65, respectively. Subjects in the 100 mg/kg treatment groups did not show improvement in the primary endpoint relative to the placebo group, having an odds ratio of 0.90. In patients having a baseline thalamic volume of <15.31 mL, subjects in the 10 mg/kg and the 30 mg/kg treatment groups showed improvement in the primary endpoint relative to the placebo group, having odds ratios of 1.24 and 1.67, respectively. Subjects in the 3 mg/kg and the 100 mg/kg treatment groups did not show improvement in the primary endpoint relative to the placebo group, having odds ratios of 0.79 and 0.52, respectively.

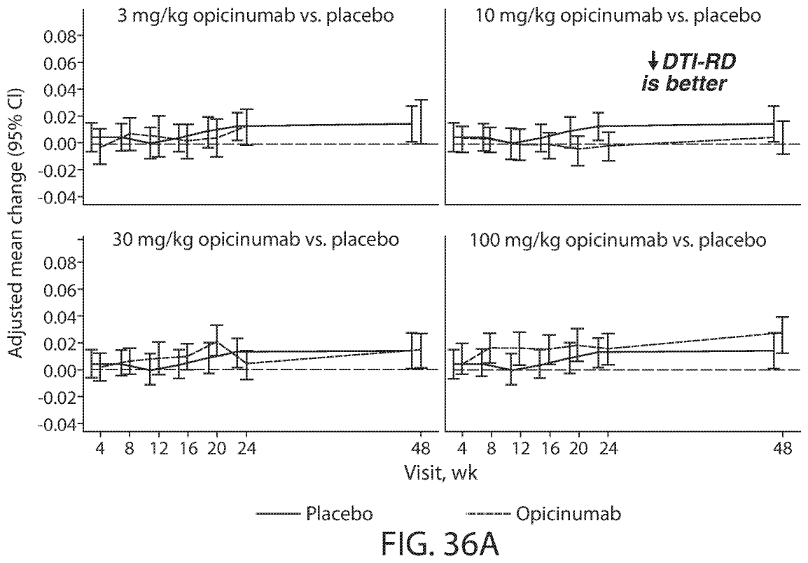

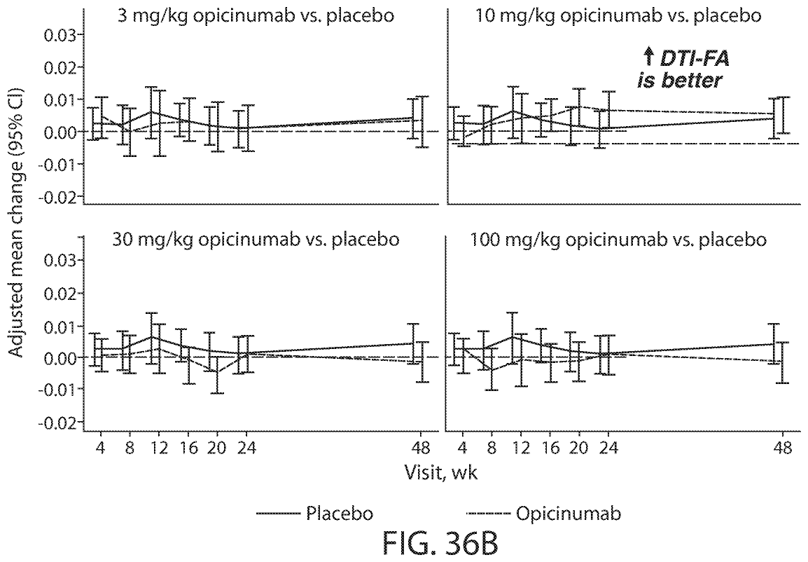

In analyses of pre-specified baseline subgroups, participants with better whole brain DTI-RD (<median, ITT population) showed the best treatment effect on the SYNERGY primary endpoint (Table 40). This is also true when looking at other endpoints, for example, DTI-RD and DTI-FA. Among subjects with a baseline whole brain DTI-RD<0.732×10⁻³ mm²/s, a treatment effect was seen for both DTI-RD and DTI-FA at the 10 mg/kg dose. This is shown for DTI-RD in FIG. 36A, and for DTI-FA in FIG. 36B.

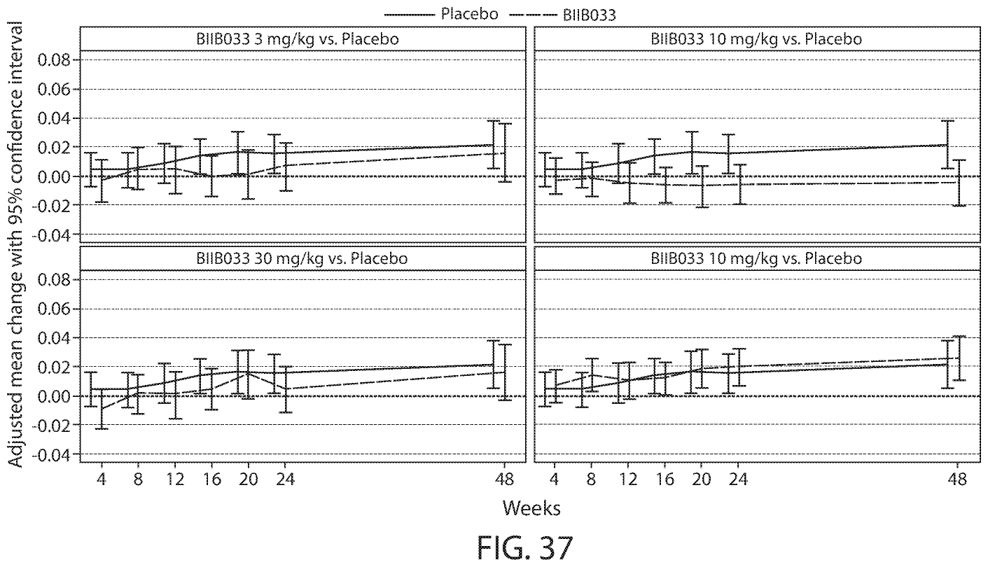

Participants with a disease duration of less than 8 years also showed a better clinical response, as shown by DTI-RD (FIG. 37). Among subjects with a disease duration of less than 8 years, a treatment effect was seen for DTI-RD at the 10 mg/kg dose.

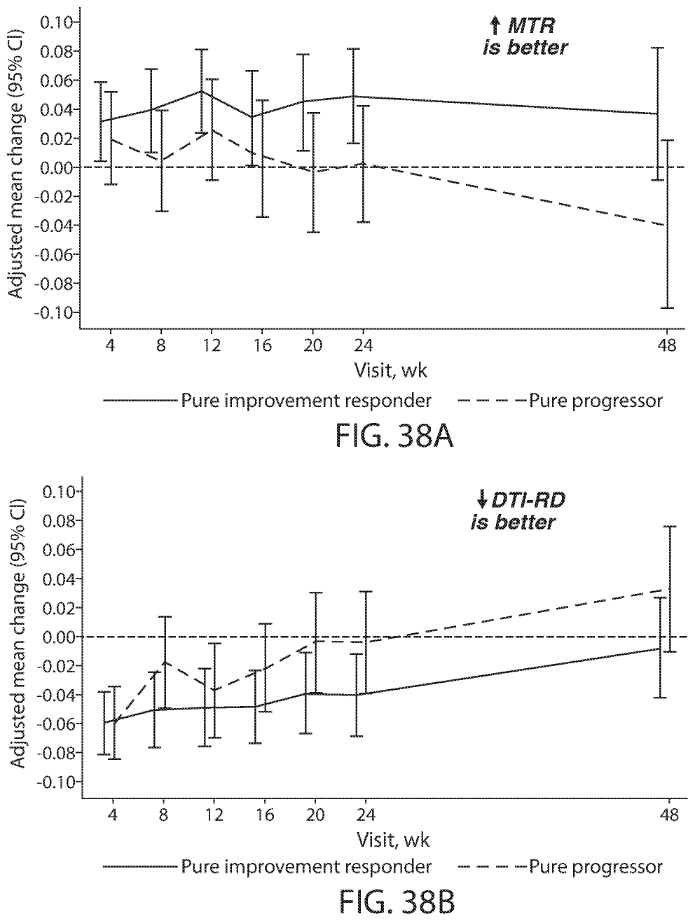

Change from baseline MTR and DTI-RD evolution in non-enhancing T2 lesion volume was observed according to clinical response (on the SYNERGY primary endpoint, regardless of treatment arm). As is shown in FIGS. 38A and 38B Clinical improvement responders (primary endpoint) demonstrated better MTR (FIG. 38A) and DTI (FIG. 38B) effects over time than non-responders (secondary endpoint). Primary endpoint was the percentage of participants experiencing confirmed improvement from baseline of disability and neurological function and/or cognitive function over 72 weeks measured using a multicomponent endpoint comprising the EDSS, Timed 25-Foot Walk, 9-Hole Peg Test and 3-second Paced Auditory Serial Addition Test; secondary endpoint was the percentage of participants experiencing confirmed worsening from Baseline of disability and neurological function and/or cognitive function over 72 weeks measured using the same components.

### Conclusions

The SYNERGY study demonstrated it is feasible to perform MTR and DTI imaging in global clinical trials. In the ITT population, no clear evidence of an anti-LINGO-1 treatment effect was observed in any of the anti-LINGO-1 treatment arms in pre-existing T2 lesions. In analyses of pre-specified Baseline subgroups, participants with lower whole brain DTI-RD (<median, ITT population) showed a treatment effect on the SYNERGY primary endpoint. In this subgroup the 10 mg/kg opicinumab dose demonstrated a favourable trend over placebo on DTI (RD and FA) in pre-existing T2 lesions. In participants who experienced clinical improvement, both MTR and DTI showed favourable changes in pre-existing T2 lesions.

### Example 7: Exploratory MRI Biomarkers of Opicinumab (Anti-LINGO-1) Show Stabilization of Pre-existing T2 Lesions in Relapsing Multiple Sclerosis: Results from the Phase 2b Synergy Trial

### Introduction

MRI markers of myelin integrity and/or repair are important to assess opicinumab treatment. The objective of this analysis was to determine the effect of treatment on MRI measures/clinical response in the SYNERGY clinical trial described in Example 5. SYNERGY (NCT01864148) evaluated opicinumab (BIIB033) vs. placebo in participants with active relapsing multiple sclerosis.

### Methods

In the SYNERGY trial described in Example 5, participants received intravenous 3, 10, 30 or 100 mg/kg opicinumab or placebo every 4 weeks (19 doses) and 30 mcg intramuscular interferon beta-1a once weekly for 72-84 weeks. Brain MRI scans were performed including conventional clinical sequences, magnetization transfer ratio (MTR) and diffusion tensor imaging (DTI), with derived measures of fractional anisotropy (FA) and radial diffusivity (RD). The putative myelin integrity markers examined were changes from baseline in mean MTR, DTI-RD and DTI-FA within pre-existing, non-enhancing T2 lesions, denoted as MTR_{T2-lesion}, DTI-RD_{T2-lesion} and DTI-FA_{T2-lesion}, respectively. Results were analyzed by: treatment group (intention-to-treat; ITT population); baseline subgroups associated with treatment effect on the SYNERGY primary endpoint (clinical improvement); and subgroups based on clinical response regardless of treatment.

### Results

418 participants were randomized and dosed; 412 were analyzed. No clear treatment effect was seen in change from baseline in MTR_{T2-lesion}, DTI-RD_{T2-lesion} and DTI-FA_{T2-lesion} in the ITT population. In participants with baseline whole brain DTI-RD<median (<0.732×10⁻³ mm²/s), favorable DTI-RD_{T2-lesion} and DTI-FA_{T2-lesion} changes were observed in participants in the 10-mg/kg treatment arm. Furthermore, change in MTR_{T2-lesion}, DTI-RD_{T2-lesion} and DTI-FA_{T2-lesion} showed more favorable changes in pure improvement responders (no worsening) vs. pure progressors (worsening; no improvement).

### Conclusions

SYNERGY demonstrated the feasibility of performing MTR and DTI imaging in global clinical trials. In participants with clinical improvement (vs. worsening), both MTR and DTI showed favorable changes in pre-existing T2 lesions. Potential biological efficacy was seen for the 10 mg/kg arm in participants with lower than population median whole brain DTI.

### INCORPORATION BY REFERENCE

The contents of all references, figures, sequence listing, patents and published patent applications cited throughout this application are hereby incorporated by reference. All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the worldwide web at tigr.org and/or the National Center for Biotechnology Information (NCBI) on the worldwide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed.

1.-4. (canceled)

5. A method of monitoring a subject having multiple sclerosis (MS) who has received, or is receiving, an anti-LINGO antibody molecule, alone or in combination with an immunomodulatory agent, comprising: acquiring a value for the level of the anti-LINGO antibody molecule in the subject at one or more time intervals, e.g., before, during, or after administration of the anti-LINGO antibody molecule, thereby monitoring the subject, wherein administration of the anti-LINGO-1 antibody molecule is continued until a cumulative exposure of the anti-LINGO-1 antibody molecule between about 20,000 μg*day/mL to about 55,000 μg*day/mL is achieved in the subject.

6. A method of treating multiple sclerosis (MS), in a subject in need thereof, comprising: administering to the subject an anti-LINGO-1 antibody molecule, alone or in combination with an immunomodulatory agent, in an amount and/or at a frequency sufficient to result in a cumulative exposure of the anti-LINGO-1 antibody molecule between about 20,000 μg*day/mL to about 55,000 μg*day/mL in the subject, wherein administration of the anti-LINGO-1 antibody molecule is continued until the cumulative exposure of the anti-LINGO-1 antibody molecule is achieved.

7. A method of treating secondary progressive MS (SPMS) in a subject, comprising: administering to the subject an anti-LINGO-1 antibody molecule, in an amount and/or at a frequency sufficient to result in a cumulative exposure of the anti-LINGO-1 antibody molecule between about 20,000 μg*day/mL to about 35,000 μg*day/mL in the subject, thereby treating the SPMS disorder, wherein administration of the anti-LINGO-1 antibody molecule is continued until the cumulative exposure of the anti-LINGO-1 antibody molecule is achieved.

8. A method of improving a disability, e.g., a walking disability, in a subject with MS, comprising: administering to the subject an anti-LINGO-1 antibody molecule, in an amount and/or at a frequency sufficient to result in a cumulative exposure of the anti-LINGO-1 antibody molecule between about 20,000 μg*day/mL to about 35,000 μg*day/mL in the subject, thereby improving the disability, wherein administration of the anti-LINGO-1 antibody molecule is continued until the cumulative exposure of the anti-LINGO-1 antibody molecule is achieved.

9.-18. (canceled)

19. The method of claim 5, wherein the anti-LINGO antibody molecule is administered in an amount ranging from about 3 to 100 mg/kg, 50 to 50 mg/kg, 10 to 30 mg/kg, 20 to 30 mg/kg, or about 3 mg/kg, about 10 mg/kg, about 30 mg/kg, or about 100 mg/kg; or a flat dose of about 150 mg to about 7500 mg, about 100 mg to about 7000 mg, about 200 mg to about 3500 mg, about 250 mg to about 3000 mg, about 300 mg to about 2000 mg, about 350 mg to about 1500 mg, about 500 mg to about 1000 mg, about 700 mg to about 900 mg, about 200 mg to about 350 mg, or about 7000 mg, about 5000 mg, about 2000 mg, about 750 mg, about 700 mg, about 500 mg, about 350 mg, or about 200 mg.

20. The method of claim 19, wherein the anti-LINGO-1 antibody molecule is administered once, or every 4 weeks, e.g., intravenously.

21. The method of claim 5, wherein the anti-LINGO-1 antibody molecule is administered at one or more of the following dosage regimens:
(i) a single dose of 100 mg/kg, or 7000 mg, e.g., administered intravenously;
(ii) one dose of 30 mg/kg, or 2000 mg, e.g., administered intravenously, every four weeks, to a total of 3 doses;
(iii) one dose of 10 mg/kg, 700 mg, or 750 mg, e.g., administered intravenously, every four weeks, to a total of 8 doses;
(iv) one dose of 3 mg/kg, or 200 mg, e.g., administered intravenously, every four weeks, to a total of 18 doses;
(v) one dose of 3-5 mg/kg, or 200-350 mg, e.g., administered subcutaneouly, every four weeks, to a total of 18 doses;
(vi) one dose of 10 mg/kg, or 750 mg, e.g., administered intravenously, every 12 weeks, up to a total of 8 doses; or
(vii) one dose of 30 mg/kg, e.g., administered intravenously, every 24 weeks, up to a total of 4 doses.

22.-82. (canceled)

83. A kit comprising an antibody molecule against human LINGO-1 with instructions for use in treating multiple sclerosis, wherein the antibody molecule is instructed to be administered at one or more of the following dosage regimens:
(i) a single dose of 100 mg/kg, or 7000 mg, e.g., administered intravenously;
(ii) one dose of 30 mg/kg, or 2000 mg, e.g., administered intravenously, every four weeks, to a total of 3 doses;
(iii) one dose of 10 mg/kg, 700 mg, or 750 mg, e.g., administered intravenously, every four weeks, to a total of 7 doses;
(iv) one dose of 3 mg/kg, or 200 mg, e.g., administered intravenously, every four weeks, to a total of 18 doses;
(v) one dose of 3-5 mg/kg, or 200-350 mg, e.g., administered subcutaneouly, every four weeks, to a total of 18 doses;
(vi) one dose of 10 mg/kg or 750 mg, e.g., administered intravenously, every 12 weeks, up to a total of 8 doses; or
(vii) one dose of 30 mg/kg, e.g., administered intravenously, every 24 weeks, up to a total of 4 doses.

84. (canceled)

85. A packaged composition comprising an antibody molecule against human LINGO-1 with instructions for use in treating multiple sclerosis, wherein the antibody molecule is instructed to be administered at a time interval chosen from one, two, or all of one or more of the following dosage regimens:
(i) a single dose of 100 mg/kg, or 7000 mg, e.g., administered intravenously;
(ii) one dose of 30 mg/kg, or 2000 mg, e.g., administered intravenously, every four weeks, to a total of 3 doses;
(iii) one dose of 10 mg/kg, 700 mg, or 750 mg, e.g., administered intravenously, every four weeks, to a total of 7 doses;
(iv) one dose of 3 mg/kg, or 200 mg, e.g., administered intravenously, every four weeks, to a total of 18 doses;
(v) one dose of 3-5 mg/kg, or 200-350 mg, e.g., administered subcutaneouly, every four weeks, to a total of 18 doses;
(vi) one dose of 10 mg/kg or 750 mg, e.g., administered intravenously, every 12 weeks, up to a total of 8 doses; or
(vii) one dose of 30 mg/kg, e.g., administered intravenously, every 24 weeks, up to a total of 4 doses.

86.-88. (canceled)

89. A method of treating a human subject having a central nervous system (CNS) demyelinating disease, comprising administering to the human subject a fixed dose of about 750 mg of an anti-LINGO-1 antibody, wherein the anti-LINGO-1 antibody comprises a heavy chain variable domain comprising heavy chain complementarity determining regions (CDRs) 1, 2 and 3 set forth in SEQ ID NO:6, 7 or 8, respectively, and a light chain variable domain comprising light chain CDRs 1, 2 and 3 set forth in SEQ ID NO:14, 15 or 16, respectively.

90. The method of claim 89, wherein the heavy chain variable domain comprises the amino acid sequence set forth in SEQ ID NO:5 and the light chain variable domain comprises the amino acid sequence set forth in SEQ ID NO:13.

91. The method of claim 89, wherein the anti-LINGO-1 antibody comprises a heavy chain comprising the amino acid sequence set forth in SEQ ID NO:275 and a light chain comprising the amino acid sequence set forth in SEQ ID NO:276.

92. The method of claim 89, wherein the anti-LINGO-1 antibody is administered intravenously.

93. The method of claim 89, wherein the anti-LINGO-1 antibody is administered every four weeks.

94. The method of claim 89, wherein the CNS demyelinating disease is multiple sclerosis.

95. The method of claim 94, wherein the human subject has a relapsing form of multiple sclerosis.

96. The method of claim 94, wherein the human subject has relapsing-remitting multiple sclerosis, primary progressive multiple sclerosis, secondary progressive multiple sclerosis, or active secondary progressive multiple sclerosis.

97. The method of claim 89, wherein the CNS demyelinating disease is optic neuritis.

98. The method of claim 89, wherein the anti-LINGO-1 antibody is administered in combination with an immunomodulatory agent.

99. The method of claim 98, wherein the immunomodulatory agent is an IFN-β1 molecule, an antibody or fragment thereof against alpha-4 integrin, or dimethyl fumarate.

100. The method of claim 89, wherein the method further comprises selecting a subject suitable for treatment with the anti-LINGO-1 antibody, wherein the subject suitable for treatment has, or is identified as having, 1, 2, 3, 4 or 5 of:
(i) a baseline Magnetization Transfer Ratio (MTR) in T2 lesion of less than or equal to about −0.25 to about −0.35 normalized MTR units (nMTRu);
(ii) a normalized MTR in T2 lesion of less than or equal to 0 nMTRu;
(iii) a disease duration of less than or equal to about 15 years to about 35 years;
(iv) a baseline diffuse tensor imaging-radial diffusivity (DTI-RD) in T2 lesion of less than or equal to about 0.9×10−3 mm²/s to 1.0×10−3 mm²/s; or
(v) a diffuse tensor imaging-radial diffusivity (DTI-RD) in a value of less than or equal to 1.2×10⁻³ mm²/s.

101. The method of claim 89, wherein the human subject is identified as having one, two, or all of the following prior to administration of the anti-LINGO-1 antibody:
(i) a baseline Magnetization Transfer Ratio (MTR) in T2 lesion of less than or equal to about −0.31 normalized MTR units (nMTRu);
(ii) a baseline diffuse tensor imaging-radial diffusivity (DTI-RD) in T2 lesion of less than or equal to about 0.95×10⁻³ mm²/s; and
(iii) a disease duration of less than or equal to about 20 years.

102. A packaged composition comprising an anti-LINGO-1 antibody with instructions for administering said antibody at a fixed dose of about 750 mg, wherein the anti-LINGO-1 antibody comprises a heavy chain variable domain comprising heavy chain complementarity determining regions (CDRs) 1, 2 and 3 set forth in SEQ ID NO:6, 7 or 8, respectively, and a light chain variable domain comprising light chain CDRs 1, 2 and 3 set forth in SEQ ID NO:14, 15 or 16, respectively.

103. A packaged composition comprising a fixed dose of about 750 mg of an anti-LINGO-1 antibody, wherein the anti-LINGO-1 antibody comprises a heavy chain variable domain comprising heavy chain complementarity determining regions (CDRs) 1, 2 and 3 set forth in SEQ ID NO:6, 7 or 8, respectively, and a light chain variable domain comprising light chain CDRs 1, 2 and 3 set forth in SEQ ID NO:14, 15 or 16, respectively.


SEQUENCE LISTING (277 sequences)

SEQ ID NO: 1 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSS

SEQ ID NO: 2 - protein, 5 aa, Artificial Sequence
  source: /note=“Description of Artificial Sequence Synthetic peptide”
IYPMF

SEQ ID NO: 3 - protein, 17 aa, Artificial Sequence
  source: /note=“Description of Artificial Sequence Synthetic peptide”
WIGPSGGITKYADSVKG

SEQ ID NO: 4 - protein, 10 aa, Artificial Sequence
  source: /note=“Description of Artificial Sequence Synthetic peptide”
EGHNDWYFDL

SEQ ID NO: 5 - protein, 118 aa, Artificial Sequence [sequence omitted]

SEQ ID NO: 6 - protein, 5 aa, Artificial Sequence
  source: /note=“Description of Artificial Sequence Synthetic peptide”
AYEMK

SEQ ID NO: 7 - protein, 17 aa, Artificial Sequence
  source: /note=“Description of Artificial Sequence Synthetic peptide”
VIGPSGGFTFYADSVKG

SEQ ID NO: 8 - protein, 9 aa, Artificial Sequence
  source: /note=“Description of Artificial Sequence Synthetic peptide”
EGDNDAFDI

SEQ ID NO: 9 - protein, 107 aa, Artificial Sequence
  source: /note=“Description of Artificial Sequence Synthetic polypeptide”
DIQMTQSPSFLSASVGDSVAITCRASQDISRYLAWYQQRPGKAPKLLIYDASNLQTGVPSRFSGSGSGTDFTFTITSLQPEDFGTYYCQQYDTLHPSFGPGTTVDIK

SEQ ID NO: 10 - protein, 11 aa, Artificial Sequence
  source: /note=“Description of Artificial Sequence Synthetic peptide”
RASQDISRYLA

SEQ ID NO: 11 - protein, 7 aa, Artificial Sequence
  source: /note=“Description of Artificial Sequence Synthetic peptide”
DASNLQT

SEQ ID NO: 12 - protein, 9 aa, Artificial Sequence
  source: /note=“Description of Artificial Sequence Synthetic peptide”
QQYDTLHPS

SEQ ID NO: 13 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIK

SEQ ID NO: 14 - protein, 11 aa, Artificial Sequence
  source: /note=“Description of Artificial Sequence Synthetic peptide”
RASQSVSSYLA

SEQ ID NO: 15 - protein, 7 aa, Artificial Sequence
  source: /note=“Description of Artificial Sequence Synthetic peptide”
DASNRAT

SEQ ID NO: 16 - protein, 10 aa, Artificial Sequence
  source: /note=“Description of Artificial Sequence Synthetic peptide”
QQRSNWPMYT

SEQ ID NO: 17 - protein, 10 aa, Artificial Sequence
  source: /note=“Description of Artificial Sequence Synthetic peptide”
EGYYDWYFDQ

SEQ ID NO: 18 - protein, 10 aa, Artificial Sequence
  source: /note=“Description of Artificial Sequence Synthetic peptide”
EGQYDWYFDV

SEQ ID NO: 19 - protein, 10 aa, Artificial Sequence
  source: /note=“Description of Artificial Sequence Synthetic peptide”
EGDYDWYFDL

SEQ ID NO: 20 - protein, 10 aa, Artificial Sequence
  source: /note=“Description of Artificial Sequence Synthetic peptide”
EGQYDWYFEL

SEQ ID NO: 21 - protein, 10 aa, Artificial Sequence
  source: /note=“Description of Artificial Sequence Synthetic peptide”
EADIDWFFDL

SEQ ID NO: 22 - protein, 10 aa, Artificial Sequence
  source: /note=“Description of Artificial Sequence Synthetic peptide”
EGHYDWYFDL

SEQ ID NO: 23 - protein, 10 aa, Artificial Sequence
  source: /note=“Description of Artificial Sequence Synthetic peptide”
EGRYDWYFDP

SEQ ID NO: 24 - protein, 10 aa, Artificial Sequence
  source: /note=“Description of Artificial Sequence Synthetic peptide”
EGDYDWYFGL

SEQ ID NO: 25 - protein, 10 aa, Artificial Sequence
  source: /note=“Description of Artificial Sequence Synthetic peptide”
EGRYDWYFDL

SEQ ID NO: 26 - protein, 10 aa, Artificial Sequence
  source: /note=“Description of Artificial Sequence Synthetic peptide”
ESHIDRYFDL

SEQ ID NO: 27 - protein, 10 aa, Artificial Sequence
  source: /note=“Description of Artificial Sequence Synthetic peptide”
EGQYDWYFDV

SEQ ID NO: 28 - protein, 10 aa, Artificial Sequence
  source: /note=“Description of Artificial Sequence Synthetic peptide”
EGHYNGYFDL

SEQ ID NO: 29 - protein, 10 aa, Artificial Sequence
  source: /note=“Description of Artificial Sequence Synthetic peptide”
EGYYDWYFDL

SEQ ID NO: 30 - protein, 10 aa, Artificial Sequence
  source: /note=“Description of Artificial Sequence Synthetic peptide”
EGTYDWYLDL

SEQ ID NO: 31 - protein, 10 aa, Artificial Sequence
  source: /note=“Description of Artificial Sequence Synthetic peptide”
EGYYDWYFEL

SEQ ID NO: 32 - protein, 10 aa, Artificial Sequence
  source: /note=“Description of Artificial Sequence Synthetic peptide”
EGLIDWFFDQ

SEQ ID NO: 33 - protein, 10 aa, Artificial Sequence
  source: /note=“Description of Artificial Sequence Synthetic peptide”
EGQFDWYFDL

SEQ ID NO: 34 - protein, 10 aa, Artificial Sequence
  source: /note=“Description of Artificial Sequence Synthetic peptide”
EGTYDWYFDL

SEQ ID NO: 35 - protein, 9 aa, Artificial Sequence
  source: /note=“Description of Artificial Sequence Synthetic peptide”
EGENDAFDV

SEQ ID NO: 36 - protein, 9 aa, Artificial Sequence
  source: /note=“Description of Artificial Sequence Synthetic peptide”
EGDNDAYDT

SEQ ID NO: 37 - protein, 9 aa, Artificial Sequence
  source: /note=“Description of Artificial Sequence Synthetic peptide”
EGTNDAFDI

SEQ ID NO: 38 - protein, 9 aa, Artificial Sequence
  source: /note=“Description of Artificial Sequence Synthetic peptide”
EGDNDAFDS

SEQ ID NO: 39 - protein, 9 aa, Artificial Sequence
  source: /note=“Description of Artificial Sequence Synthetic peptide”
EGDNDAFDT

SEQ ID NO: 40 - protein, 9 aa, Artificial Sequence
  source: /note=“Description of Artificial Sequence Synthetic peptide”
EGDNDAYDR

SEQ ID NO: 41 - protein, 9 aa, Artificial Sequence
  source: /note=“Description of Artificial Sequence Synthetic peptide”
EGDNDVFDS

SEQ ID NO: 42 - protein, 9 aa, Artificial Sequence
  source: /note=“Description of Artificial Sequence Synthetic peptide”
EGDDDVFDM

SEQ ID NO: 43 - protein, 9 aa, Artificial Sequence
  source: /note=“Description of Artificial Sequence Synthetic peptide”
EGYNDAFDF

SEQ ID NO: 44 - protein, 9 aa, Artificial Sequence
  source: /note=“Description of Artificial Sequence Synthetic peptide”
EGDDDAYDM

SEQ ID NO: 45 - protein, 9 aa, Artificial Sequence
  source: /note=“Description of Artificial Sequence Synthetic peptide”
EQDYDTYDL

SEQ ID NO: 46 - protein, 9 aa, Artificial Sequence
  source: /note=“Description of Artificial Sequence Synthetic peptide”
EGDDDAFDT

SEQ ID NO: 47 - protein, 9 aa, Artificial Sequence
  source: /note=“Description of Artificial Sequence Synthetic peptide”
EADDDAFDI

SEQ ID NO: 48 - protein, 9 aa, Artificial Sequence
  source: /note=“Description of Artificial Sequence Synthetic peptide”
EGENDAFDM

SEQ ID NO: 49 - protein, 9 aa, Artificial Sequence
  source: /note=“Description of Artificial Sequence Synthetic peptide”
EGEYDTYDI

SEQ ID NO: 50 - protein, 454 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADISVKGRFTISRDNSKNTLYLQMNSLRAEDTAVYYCATEGDNDAFDIWGQGTTVTVSSASTKGPISVFPLAPSSKSTSGGTAALGCLVKDYFPEPVTVSWNSGALTSGVHTFPAVLQSSGLYSLSSVVTIVPSSSLGTQTYICNVNHKPSNTKVDKKVEPKSCDKTHTCPPCPAPELLGGPSVFLFPPKPKDTLIMISRTPEVTCVVVDVSHEDPEVKFNWYVDGVEVHNAKTKPREEQYNSAYRVVSVLTVLHQDIWLNGKEYKCKVSNKALPAPIEKTISKAKGQPREPQVYTLPPSRDELTKNQVSLTCLVKGFYPSIDIAVEWESNGQPENNYKTTPPVLDSDGSFFLYSKLTVDKSRWQQGNVFSCSVMHEALHNHYTIQKSLSLSPG

SEQ ID NO: 51 - protein, 614 aa, Homo sapiens
MLAGGVRSMPSPLLACWQPILLLVLGSVLSGSATGCPPRCECSAQDRAVLCHRKRFVAVPEGIPTETRLLDLGKNRIKTLNQDEFASFPHLEELELNENIVSAVEPGAFNNLFNLRTLGLRSNRLKLIPLGVFTGLSNLTKLDISENKIVILLDYMFQDLYNLKSLEVGDNDLVYISHRAFSGLNSLEQLTLEKCNLTSIPTEALSHLHGLIVLRLRHLNINAIRDYSFKRLYRLKVLEISHWPYLDTMTPNCLYGLNLTSLSITHCNLTAVPYLAVRHLVYLRFLNLSYNPISTIEGSMLHELLRLQEIQLVGGQLAVVEPYAFRGLNYLRVLNVSGNQLTTLEESVFHSVGNLETLILDSNPLACDCRLLWVFRRRWRLNFNRQQPTCATPEFVQGKEFKDFPDVLLPNYFTCRRARIRDRKAQQVFVDEGHTVQFVCRADGDPPPAILWLSPRKHLVSAKSNGRLTVFPDGTLEVRYAQVQDNGTYLCIAANAGGNDSMPAHLHVRSYSPDWPHQPNKTFAFISNQPGEGEANSTRATVPFPFDIKTLIIATTMGFISFLGVVLFCLVLLFLWSRGKGNTKHNIEIEYVPRKSDAGISSADAPRKFNMKMI

SEQ ID NO: 52 - DNA, 1845 bp, Homo sapiens
atgctggcggggggcgtgaggagcatgcccagccccctcctggcctgctggcagcccatcctcctgctggtgctgggctcagtgctgtcaggctcggccacgggctgcccgccccgctgcgagtgctccgcccaggaccgcgctgtgctgtgccaccgcaagcgctttgtggcagtccccgagggcatccccaccgagacgcgcctgctggacctaggcaagaaccgcatcaaaacgctcaaccaggacgagttcgccagcttcccgcacctggaggagctggagctcaacgagaacatcgtgagcgccgtggagcccggcgccttcaacaacctcttcaacctccggacgctgggtctccgcagcaaccgcctgaagctcatcccgctaggcgtcttcactggcctcagcaacctgaccaagctggacatcagcgagaacaagattgttatcctgctggactacatgtttcaggacctgtacaacctcaagtcactggaggttggcgacaatgacctcgtctacatctctcaccgcgccttcagcggcctcaacagcctggagcagctgacgctggagaaatgcaacctgacctccatccccaccgaggcgctgtcccacctgcacggcctcatcgtcctgaggctccggcacctcaacatcaatgccatccgggactactccttcaagaggctctaccgactcaaggtcttggagatctcccactggccctacttggacaccatgacacccaactgcctctacggcctcaacctgacgtccctgtccatcacacactgcaatctgaccgctgtgccctacctggccgtccgccacctagtctatctccgcttcctcaacctctcctacaaccccatcagcaccattgagggctccatgttgcatgagctgctccggctgcaggagatccagctggtgggcgggcagctggccgtggtggagccctatgccttccgcggcctcaactacctgcgcgtgctcaatgtctctggcaaccagctgaccacactggaggaatcagtcttccactcggtgggcaacctggagacactcatcctggactccaacccgctggcctgcgactgtcggctcctgtgggtgttccggcgccgctggcggctcaacttcaaccggcagcagcccacgtgcgccacgcccgagtttgtccagggcaaggagttcaaggacttccctgatgtgctactgcccaactacttcacctgccgccgcgcccgcatccgggaccgcaaggcccagcaggtgtttgtggacgagggccacacggtgcagtttgtgtgccgggccgatggcgacccgccgcccgccatcctctggctctcaccccgaaagcacctggtctcagccaagagcaatgggcggctcacagtcttccctgatggcacgctggaggtgcgctacgcccaggtacaggacaacggcacgtacctgtgcatcgcggccaacgcgggcggcaacgactccatgcccgcccacctgcatgtgcgcagctactcgcccgactggccccatcagcccaacaagaccttcgctttcatctccaaccagccgggcgagggagaggccaacagcacccgcgccactgtgcctttccccttcgacatcaagaccctcatcatcgccaccaccatgggcttcatctctttcctgggcgtcgtcctcttctgcctggtgctgctgtttctctggagccggggcaagggcaacacaaagcacaacatcgagatcgagtatgtgccccgaaagtcggacgcaggcatcagctccgccgacgcgccccgcaagttcaacatgaagatgatatga

SEQ ID NO: 53 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGYYDWYFDQWGRGTLVTVSS

SEQ ID NO: 54 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGQYDWYFDVWGRGTLVTVSS

SEQ ID NO: 55 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGDYDWYFDLWGRGTLVTVSS

SEQ ID NO: 56 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGQYDWYFELWGRGTLVTVSS

SEQ ID NO: 57 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREADIDWFFDLWGRGTLVTVSS

SEQ ID NO: 58 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHYDWYFDLWGRGTLVTVSS

SEQ ID NO: 59 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGRYDWYFDPWGRGTLVTVSS

SEQ ID NO: 60 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGDYDWYFGLWGRGTLVTVSS

SEQ ID NO: 61 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGRYDWYFDLWGRGTLVTVSS

SEQ ID NO: 62 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCARESHIDRYFDLWGRGTLVTVSS

SEQ ID NO: 63 - protein, 119 aa, Artificial Sequence; identical to SEQ ID NO: 54
  source: /note=“Description of Artificial Sequence Synthetic polypeptide”

SEQ ID NO: 64 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHYNGYFDLWGRGTLVTVSS

SEQ ID NO: 65 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGYYDWYFDLWGRGTLVTVSS

SEQ ID NO: 66 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDWYLDLWGRGTLVTVSS

SEQ ID NO: 67 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGYYDWYFELWGRGTLVTVSS

SEQ ID NO: 68 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGLIDWFFDQWGRGTLVTVSS

SEQ ID NO: 69 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGQFDWYFDLWGRGTLVTVSS

SEQ ID NO: 70 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDWYFDLWGRGTLVTVSS

SEQ ID NO: 71 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGENDAFDVWGQGTTVTVSS

SEQ ID NO: 72 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAYDTWGQGTTVTVSS

SEQ ID NO: 73 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGTNDAFDIWGQGTTVTVSS

SEQ ID NO: 74 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDSWGQGTTVTVSS

SEQ ID NO: 75 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDTWGQGTTVTVSS

SEQ ID NO: 76 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAYDRWGQGTTVTVSS

SEQ ID NO: 77 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDVFDSWGQGTTVTVSS

SEQ ID NO: 78 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDDDVFDMWGQGTTVTVSS

SEQ ID NO: 79 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGYNDAFDFWGQGTTVTVSS

SEQ ID NO: 80 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDDDAYDMWGQGTTVTVSS

SEQ ID NO: 81 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEQDYDTYDLWGQGTTVTVSS

SEQ ID NO: 82 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDDDAFDTWGQGTTVTVSS

SEQ ID NO: 83 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEADDDAFDIWGQGTTVTVSS

SEQ ID NO: 84 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGENDAFDMWGQGTTVTVSS

SEQ ID NO: 85 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGEYDTYDIWGQGTTVTVSS

SEQ ID NO: 86 - protein, 454 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADISVKGRFTISRDNSKNTLYLQMNSLRAEDTAVYYCATEGDNDAFDIWGQGTTVTVSSASTKGPISVFPLAPSSKSTSGGTAALGCLVKDYFPEPVTVSWNSGALTSGVHTFPAVLQSSGLYSLSSVVTIVPSSSLGTQTYICNVNHKPSNTKVDKKVEPKSCDKTHTCPPCPAPELLGGPSVFLFPPKPKDTLIMISRTPEVTCVVVDVSHEDPEVKFNWYVDGVEVHNAKTKPREEQYNSTYRVVSVLTVLHQDIWLNGKEYKCKVSNKALPAPIEKTISKAKGQPREPQVYTLPPSRDELTKNQVSLTCLVKGFYPSIDIAVEWESNGQPENNYKTTPPVLDSDGSFFLYSKLTVDKSRWQQGNVFSCSVMHEALHNHYTIQKSLSLSPG

SEQ ID NO: 87 - protein, 6 aa, Artificial Sequence
  source: /note=“Description of Artificial Sequence Synthetic peptide”
MQVSKR

SEQ ID NO: 88 - protein, 5 aa, Artificial Sequence
  source: /note=“Description of Artificial Sequence Synthetic peptide”
  VARIANT (3)..(5): /replace=“Arg” or “His” or “Gln” or “Asn”
  MISC_FEATURE (1)..(5): /note=“Variant residues given in the sequence have no preference with respect to those in the annotations for variant positions”
ITKKK

SEQ ID NO: 89 - protein, 5 aa, Artificial Sequence
  source: /note=“Description of Artificial Sequence Synthetic peptide”
  VARIANT (3)..(5): /replace=“Arg” or “His” or “Gln” or “Asn”
  MISC_FEATURE (1)..(5): /note=“Variant residues given in the sequence have no preference with respect to those in the annotations for variant positions”
ACKKK

SEQ ID NO: 90 - protein, 5 aa, Artificial Sequence
  source: /note=“Description of Artificial Sequence Synthetic peptide”
  VARIANT (3)..(5): /replace=“Arg” or “His” or “Gln” or “Asn”
  MISC_FEATURE (1)..(5): /note=“Variant residues given in the sequence have no preference with respect to those in the annotations for variant positions”
VCKKK

SEQ ID NO: 91 - protein, 5 aa, Artificial Sequence
  source: /note=“Description of Artificial Sequence Synthetic peptide”
  VARIANT (3)..(5): /replace=“Arg” or “His” or “Gln” or “Asn”
  MISC_FEATURE (1)..(5): /note=“Variant residues given in the sequence have no preference with respect to those in the annotations for variant positions”
SPKKK

SEQ ID NO: 92 - protein, 5 aa, Artificial Sequence
  source: /note=“Description of Artificial Sequence Synthetic peptide”
SPRKH

SEQ ID NO: 93 - protein, 5 aa, Artificial Sequence
  source: /note=“Description of Artificial Sequence Synthetic peptide”
SPRKK

SEQ ID NO: 94 - protein, 5 aa, Artificial Sequence
  source: /note=“Description of Artificial Sequence Synthetic peptide”
SPRKR

SEQ ID NO: 95 - protein, 5 aa, Artificial Sequence
  source: /note=“Description of Artificial Sequence Synthetic peptide”
SPKKH

SEQ ID NO: 96 - protein, 5 aa, Artificial Sequence
  source: /note=“Description of Artificial Sequence Synthetic peptide”
SPHKH

SEQ ID NO: 97 - protein, 5 aa, Artificial Sequence
  source: /note=“Description of Artificial Sequence Synthetic peptide”
SPRRH

SEQ ID NO: 98 - protein, 5 aa, Artificial Sequence
  source: /note=“Description of Artificial Sequence Synthetic peptide”
SPRHH

SEQ ID NO: 99 - protein, 5 aa, Artificial Sequence
  source: /note=“Description of Artificial Sequence Synthetic peptide”
SPRRR

SEQ ID NO: 100 - protein, 5 aa, Artificial Sequence
  source: /note=“Description of Artificial Sequence Synthetic peptide”
SPHHH

SEQ ID NO: 101 - protein, 5 aa, Artificial Sequence
  source: /note=“Description of Artificial Sequence Synthetic peptide”
SPKKK

SEQ ID NO: 102 - protein, 6 aa, Artificial Sequence
  source: /note=“Description of Artificial Sequence Synthetic peptide”
LSPRKH

SEQ ID NO: 103 - protein, 6 aa, Artificial Sequence
  source: /note=“Description of Artificial Sequence Synthetic peptide”
LSPRKK

SEQ ID NO: 104 - protein, 6 aa, Artificial Sequence
  source: /note=“Description of Artificial Sequence Synthetic peptide”
LSPRKR

SEQ ID NO: 105 - protein, 6 aa, Artificial Sequence
  source: /note=“Description of Artificial Sequence Synthetic peptide”
LSPKKH

SEQ ID NO: 106 - protein, 6 aa, Artificial Sequence
  source: /note=“Description of Artificial Sequence Synthetic peptide”
LSPHKH

SEQ ID NO: 107 - protein, 6 aa, Artificial Sequence
  source: /note=“Description of Artificial Sequence Synthetic peptide”
LSPRRH

SEQ ID NO: 108 - protein, 6 aa, Artificial Sequence
  source: /note=“Description of Artificial Sequence Synthetic peptide”
LSPRHH

SEQ ID NO: 109 - protein, 6 aa, Artificial Sequence
  source: /note=“Description of Artificial Sequence Synthetic peptide”
LSPRRR

SEQ ID NO: 110 - protein, 6 aa, Artificial Sequence
  source: /note=“Description of Artificial Sequence Synthetic peptide”
LSPHHH

SEQ ID NO: 111 - protein, 6 aa, Artificial Sequence
  source: /note=“Description of Artificial Sequence Synthetic peptide”
LSPKKK

SEQ ID NO: 112 - protein, 7 aa, Artificial Sequence
  source: /note=“Description of Artificial Sequence Synthetic peptide”
WLSPRKH

SEQ ID NO: 113 - protein, 7 aa, Artificial Sequence
  source: /note=“Description of Artificial Sequence Synthetic peptide”
WLSPRKK

SEQ ID NO: 114 - protein, 7 aa, Artificial Sequence
  source: /note=“Description of Artificial Sequence Synthetic peptide”
WLSPRKR

SEQ ID NO: 115 - protein, 7 aa, Artificial Sequence
  source: /note=“Description of Artificial Sequence Synthetic peptide”
WLSPKKH

SEQ ID NO: 116 - protein, 7 aa, Artificial Sequence
  source: /note=“Description of Artificial Sequence Synthetic peptide”
WLSPHKH

SEQ ID NO: 117 - protein, 7 aa, Artificial Sequence
  source: /note=“Description of Artificial Sequence Synthetic peptide”
WLSPRRH

SEQ ID NO: 118 - protein, 7 aa, Artificial Sequence
  source: /note=“Description of Artificial Sequence Synthetic peptide”
WLSPRHH

SEQ ID NO: 119 - protein, 7 aa, Artificial Sequence
  source: /note=“Description of Artificial Sequence Synthetic peptide”
WLSPRRR

SEQ ID NO: 120 - protein, 7 aa, Artificial Sequence
  source: /note=“Description of Artificial Sequence Synthetic peptide”
WLSPHHH

SEQ ID NO: 121 - protein, 7 aa, Artificial Sequence
  source: /note=“Description of Artificial Sequence Synthetic peptide”
WLSPKKK

SEQ ID NO: 122 - protein, 6 aa, Artificial Sequence
  source: /note=“Description of Artificial Sequence Synthetic peptide”
ITPKRR

SEQ ID NO: 123 - protein, 5 aa, Artificial Sequence
  source: /note=“Description of Artificial Sequence Synthetic peptide”
ACHHK

SEQ ID NO: 124 - protein, 5 aa, Artificial Sequence
  source: /note=“Description of Artificial Sequence Synthetic peptide”
VCHHK

SEQ ID NO: 125 - protein, 5 aa, Artificial Sequence
  source: /note=“Description of Artificial Sequence Synthetic peptide”
  MOD_RES (1)..(2): Any amino acid
XXRKH

SEQ ID NO: 126 - protein, 5 aa, Artificial Sequence
  source: /note=“Description of Artificial Sequence Synthetic peptide”
  MOD_RES (1)..(2): Any amino acid
XXRRR

SEQ ID NO: 127 - protein, 5 aa, Artificial Sequence
  source: /note=“Description of Artificial Sequence Synthetic peptide”
  MOD_RES (1)..(2): Any amino acid
XXKKK

SEQ ID NO: 128 - protein, 5 aa, Artificial Sequence
  source: /note=“Description of Artificial Sequence Synthetic peptide”
  MOD_RES (1)..(2): Any amino acid
XXHHH

SEQ ID NO: 129 - protein, 5 aa, Artificial Sequence
  source: /note=“Description of Artificial Sequence Synthetic peptide”
  MOD_RES (1)..(2): Any amino acid
XXRKK

SEQ ID NO: 130 - protein, 5 aa, Artificial Sequence
  source: /note=“Description of Artificial Sequence Synthetic peptide”
  MOD_RES (1)..(2): Any amino acid
XXRKR

SEQ ID NO: 131 - protein, 5 aa, Artificial Sequence
  source: /note=“Description of Artificial Sequence Synthetic peptide”
  MOD_RES (1)..(2): Any amino acid
XXKKH

SEQ ID NO: 132 - protein, 5 aa, Artificial Sequence
  source: /note=“Description of Artificial Sequence Synthetic peptide”
  MOD_RES (1)..(2): Any amino acid
XXHKH

SEQ ID NO: 133 - protein, 5 aa, Artificial Sequence
  source: /note=“Description of Artificial Sequence Synthetic peptide”
  MOD_RES (1)..(2): Any amino acid
XXRRH

SEQ ID NO: 134 - protein, 5 aa, Artificial Sequence
  source: /note=“Description of Artificial Sequence Synthetic peptide”
  MOD_RES (1)..(2): Any amino acid
XXRHH

SEQ ID NO: 135 - protein, 5 aa, Artificial Sequence
  source: /note=“Description of Artificial Sequence Synthetic peptide”
  VARIANT (3)..(3): /replace=“Arg” or “His” or “Gln” or “Asn”
  MOD_RES (4)..(4): Any amino acid
  VARIANT (5)..(5): /replace=“Arg” or “His” or “Gln” or “Asn”
  MISC_FEATURE (1)..(5): /note=“Variant residues given in the sequence have no preference with respect to those in the annotations for variant positions”
ITKXK

SEQ ID NO: 136 - protein, 5 aa, Artificial Sequence
  source: /note=“Description of Artificial Sequence Synthetic peptide”
  VARIANT (3)..(3): /replace=“Arg” or “His” or “Gln” or “Asn”
  MOD_RES (4)..(4): Any amino acid
  VARIANT (5)..(5): /replace=“Arg” or “His” or “Gln” or “Asn”
  MISC_FEATURE (1)..(5): /note=“Variant residues given in the sequence have no preference with respect to those in the annotations for variant positions”
ACKXK

SEQ ID NO: 137 - protein, 5 aa, Artificial Sequence
  source: /note=“Description of Artificial Sequence Synthetic peptide”
  VARIANT (3)..(3): /replace=“Arg” or “His” or “Gln” or “Asn”
  MOD_RES (4)..(4): Any amino acid
  VARIANT (5)..(5): /replace=“Arg” or “His” or “Gln” or “Asn”
  MISC_FEATURE (1)..(5): /note=“Variant residues given in the sequence have no preference with respect to those in the annotations for variant positions”
VCKXK

SEQ ID NO: 138 - protein, 5 aa, Artificial Sequence
  source: /note=“Description of Artificial Sequence Synthetic peptide”
  VARIANT (3)..(3): /replace=“Arg” or “His” or “Gln” or “Asn”
  MOD_RES (4)..(4): Any amino acid
  VARIANT (5)..(5): /replace=“Arg” or “His” or “Gln” or “Asn”
  MISC_FEATURE (1)..(5): /note=“Variant residues given in the sequence have no preference with respect to those in the annotations for variant positions”
SPKXK

SEQ ID NO: 139 - protein, 5 aa, Artificial Sequence
  source: /note=“Description of Artificial Sequence Synthetic peptide”
SPRLH

SEQ ID NO: 140 - protein, 9 aa, Artificial Sequence
  source: /note=“Description of Artificial Sequence Synthetic peptide”
RRARIRDRK

SEQ ID NO: 141 - protein, 9 aa, Artificial Sequence
  source: /note=“Description of Artificial Sequence Synthetic peptide”
KKVKVKEKR

SEQ ID NO: 142 - protein, 9 aa, Artificial Sequence
  source: /note=“Description of Artificial Sequence Synthetic peptide”
RRLRLRDRK

SEQ ID NO: 143 - protein, 9 aa, Artificial Sequence
  source: /note=“Description of Artificial Sequence Synthetic peptide”
RRGRGRDRK

SEQ ID NO: 144 - protein, 9 aa, Artificial Sequence
  source: /note=“Description of Artificial Sequence Synthetic peptide”
RRIRARDRK

SEQ ID NO: 145 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 147 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSHIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 148 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSFIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 149 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSLIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 150 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSMIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 151 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGLSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 152 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGSSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 153 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGTSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 154 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGWSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 155 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDVYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 156 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDHYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 157 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDSYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 158 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDQYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 159 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGGTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 160 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGMTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 161 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGNTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 162 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGHTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 163 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGLTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 164 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGFTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 165 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKGPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKQPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 214 aa, Artificial Sequence
  source: /note=“Description of Artificial Sequence Synthetic polypeptide”
DIQMTQSPGTLSLSPGERATLSCRASQSVSSYLAWYQQKPGQAPRLLIYDASNRATGIPARFSGSGSGTEFTLTISSLQSEDFAVYYCQQYDKSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSGIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 174 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSIIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 175 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSDIGPSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 176 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDMYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 177 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDLYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 178 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDTYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 179 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDIYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 180 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGGSGGITKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 181 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGWTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 182 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGYTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 183 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGSTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 184 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGPTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 185 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGVTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 186 - protein, 230 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGTTKYADSVKGRFTISRDNSKNTLYLQMNSLRAEDTATYYCAREGHNDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 187 - protein, 230 aa, Artificial Sequence; identical to SEQ ID NO: 158
  source: /note=“Description of Artificial Sequence Synthetic polypeptide”

SEQ ID NO: 188 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSHIGPSGGITKYADSVKGRFTISRDNSKNTLYLQMNSLRAEDTATYYCAREGHNDWYFDLWGRGTLVTVSS

SEQ ID NO: 189 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSFIGPSGGITKYADSVKGRFTISRDNSKNTLYLQMNSLRAEDTATYYCAREGHNDWYFDLWGRGTLVTVSS

SEQ ID NO: 190 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSLIGPSGGITKYADSVKGRFTISRDNSKNTLYLQMNSLRAEDTATYYCAREGHNDWYFDLWGRGTLVTVSS

SEQ ID NO: 191 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSMIGPSGGITKYADSVKGRFTISRDNSKNTLYLQMNSLRAEDTATYYCAREGHNDWYFDLWGRGTLVTVSS

SEQ ID NO: 192 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGLSGGITKYADSVKGRFTISRDNSKNTLYLQMNSLRAEDTATYYCAREGHNDWYFDLWGRGTLVTVSS

SEQ ID NO: 193 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGSSGGITKYADSVKGRFTISRDNSKNTLYLQMNSLRAEDTATYYCAREGHNDWYFDLWGRGTLVTVSS

SEQ ID NO: 194 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGTSGGITKYADSVKGRFTISRDNSKNTLYLQMNSLRAEDTATYYCAREGHNDWYFDLWGRGTLVTVSS

SEQ ID NO: 195 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGWSGGITKYADSVKGRFTISRDNSKNTLYLQMNSLRAEDTATYYCAREGHNDWYFDLWGRGTLVTVSS

SEQ ID NO: 196 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDVYFDLWGRGTLVTVSS

SEQ ID NO: 197 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDHYFDLWGRGTLVTVSS

SEQ ID NO: 198 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDSYFDLWGRGTLVTVSS

SEQ ID NO: 199 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDQYFDLWGRGTLVTVSS

SEQ ID NO: 200 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGGTKYADSVKGRFTISRDNSKNTLYLQMNSLRAEDTATYYCAREGHNDWYFDLWGRGTLVTVSS

SEQ ID NO: 201 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGMTKYADSVKGRFTISRDNSKNTLYLQMNSLRAEDTATYYCAREGHNDWYFDLWGRGTLVTVSS

SEQ ID NO: 202 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGNTKYADSVKGRFTISRDNSKNTLYLQMNSLRAEDTATYYCAREGHNDWYFDLWGRGTLVTVSS

SEQ ID NO: 203 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGHTKYADSVKGRFTISRDNSKNTLYLQMNSLRAEDTATYYCAREGHNDWYFDLWGRGTLVTVSS

SEQ ID NO: 204 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGLTKYADSVKGRFTISRDNSKNTLYLQMNSLRAEDTATYYCAREGHNDWYFDLWGRGTLVTVSS

SEQ ID NO: 205 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGFTKYADSVKGRFTISRDNSKNTLYLQMNSLRAEDTATYYCAREGHNDWYFDLWGRGTLVTVSS

SEQ ID NO: 206 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSGIGPSGGITKYADSVKGRFTISRDNSKNTLYLQMNSLRAEDTATYYCAREGHNDWYFDLWGRGTLVTVSS

SEQ ID NO: 207 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSIIGPSGGITKYADSVKGRFTISRDNSKNTLYLQMNSLRAEDTATYYCAREGHNDWYFDLWGRGTLVTVSS

SEQ ID NO: 208 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSDIGPSGGITKYADSVKGRFTISRDNSKNTLYLQMNSLRAEDTATYYCAREGHNDWYFDLWGRGTLVTVSS

SEQ ID NO: 209 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDMYFDLWGRGTLVTVSS

SEQ ID NO: 210 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDLYFDLWGRGTLVTVSS

SEQ ID NO: 211 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDTYFDLWGRGTLVTVSS

SEQ ID NO: 212 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDIYFDLWGRGTLVTVSS

SEQ ID NO: 213 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGGSGGITKYADSVKGRFTISRDNSKNTLYLQMNSLRAEDTATYYCAREGHNDWYFDLWGRGTLVTVSS

SEQ ID NO: 214 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGWTKYADSVKGRFTISRDNSKNTLYLQMNSLRAEDTATYYCAREGHNDWYFDLWGRGTLVTVSS

SEQ ID NO: 215 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGYTKYADSVKGRFTISRDNSKNTLYLQMNSLRAEDTATYYCAREGHNDWYFDLWGRGTLVTVSS

SEQ ID NO: 216 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGSTKYADSVKGRFTISRDNSKNTLYLQMNSLRAEDTATYYCAREGHNDWYFDLWGRGTLVTVSS

SEQ ID NO: 217 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGPTKYADSVKGRFTISRDNSKNTLYLQMNSLRAEDTATYYCAREGHNDWYFDLWGRGTLVTVSS

SEQ ID NO: 218 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGVTKYADSVKGRFTISRDNSKNTLYLQMNSLRAEDTATYYCAREGHNDWYFDLWGRGTLVTVSS

SEQ ID NO: 219 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGTTKYADSVKGRFTISRDNSKNTLYLQMNSLRAEDTATYYCAREGHNDWYFDLWGRGTLVTVSS

SEQ ID NO: 220 - protein, 119 aa, Artificial Sequence; identical to SEQ ID NO: 199
  source: /note=“Description of Artificial Sequence Synthetic polypeptide”

SEQ ID NO: 221 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPLYTFGQGTKLEIK

SEQ ID NO: 222 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPIYTFGQGTKLEIK

SEQ ID NO: 223 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPQYTFGQGTKLEIK

SEQ ID NO: 224 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPKYTFGQGTKLEIK

SEQ ID NO: 225 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPAYTFGQGTKLEIK

SEQ ID NO: 226 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPVYTFGQGTKLEIK

SEQ ID NO: 227 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPYYTFGQGTKLEIK

SEQ ID NO: 228 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPFYTFGQGTKLEIK

SEQ ID NO: 229 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGLSGGFTFYADSVKGRFTISRDNSKNTLYLQMNSLRAEDTAVYYCATEGDNDAFDIWGQGTTVTVSS

SEQ ID NO: 230 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGSSGGFTFYADSVKGRFTISRDNSKNTLYLQMNSLRAEDTAVYYCATEGDNDAFDIWGQGTTVTVSS

SEQ ID NO: 231 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGTSGGFTFYADSVKGRFTISRDNSKNTLYLQMNSLRAEDTAVYYCATEGDNDAFDIWGQGTTVTVSS

SEQ ID NO: 232 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGWSGGFTFYADSVKGRFTISRDNSKNTLYLQMNSLRAEDTAVYYCATEGDNDAFDIWGQGTTVTVSS

SEQ ID NO: 233 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGGSGGFTFYADSVKGRFTISRDNSKNTLYLQMNSLRAEDTAVYYCATEGDNDAFDIWGQGTTVTVSS

SEQ ID NO: 234 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNAPMYTFGQGTKLEIK

SEQ ID NO: 235 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNDPMYTFGQGTKLEIK

SEQ ID NO: 236 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNLPMYTFGQGTKLEIK

SEQ ID NO: 237 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNNPMYTFGQGTKLEIK

SEQ ID NO: 238 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNGPMYTFGQGTKLEIK

SEQ ID NO: 239 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNQPMYTFGQGTKLEIK

SEQ ID NO: 240 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNVPMYTFGQGTKLEIK

SEQ ID NO: 241 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNSPMYTFGQGTKLEIK

SEQ ID NO: 242 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSHIGPSGGITKYADSVKGRFTISRDNSKNTLYLQMNSLRAEDTATYYCAREGTYDWYLDLWGRGTLVTVSS

SEQ ID NO: 243 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSFIGPSGGITKYADSVKGRFTISRDNSKNTLYLQMNSLRAEDTATYYCAREGTYDWYLDLWGRGTLVTVSS

SEQ ID NO: 244 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSLIGPSGGITKYADSVKGRFTISRDNSKNTLYLQMNSLRAEDTATYYCAREGTYDWYLDLWGRGTLVTVSS

SEQ ID NO: 245 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSMIGPSGGITKYADSVKGRFTISRDNSKNTLYLQMNSLRAEDTATYYCAREGTYDWYLDLWGRGTLVTVSS

SEQ ID NO: 246 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGLSGGITKYADSVKGRFTISRDNSKNTLYLQMNSLRAEDTATYYCAREGTYDWYLDLWGRGTLVTVSS

SEQ ID NO: 247 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGSSGGITKYADSVKGRFTISRDNSKNTLYLQMNSLRAEDTATYYCAREGTYDWYLDLWGRGTLVTVSS

SEQ ID NO: 248 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGTSGGITKYADSVKGRFTISRDNSKNTLYLQMNSLRAEDTATYYCAREGTYDWYLDLWGRGTLVTVSS

SEQ ID NO: 249 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGWSGGITKYADSVKGRFTISRDNSKNTLYLQMNSLRAEDTATYYCAREGTYDWYLDLWGRGTLVTVSS

SEQ ID NO: 250 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDVYLDLWGRGTLVTVSS

SEQ ID NO: 251 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDHYLDLWGRGTLVTVSS

SEQ ID NO: 252 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDSYLDLWGRGTLVTVSS

SEQ ID NO: 253 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDQYLDLWGRGTLVTVSS

SEQ ID NO: 254 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGGTKYADSVKGRFTISRDNSKNTLYLQMNSLRAEDTATYYCAREGTYDWYLDLWGRGTLVTVSS

SEQ ID NO: 255 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGMTKYADSVKGRFTISRDNSKNTLYLQMNSLRAEDTATYYCAREGTYDWYLDLWGRGTLVTVSS

SEQ ID NO: 256 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGNTKYADSVKGRFTISRDNSKNTLYLQMNSLRAEDTATYYCAREGTYDWYLDLWGRGTLVTVSS

SEQ ID NO: 257 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGHTKYADSVKGRFTISRDNSKNTLYLQMNSLRAEDTATYYCAREGTYDWYLDLWGRGTLVTVSS

SEQ ID NO: 258 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGLTKYADSVKGRFTISRDNSKNTLYLQMNSLRAEDTATYYCAREGTYDWYLDLWGRGTLVTVSS

SEQ ID NO: 259 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGFTKYADSVKGRFTISRDNSKNTLYLQMNSLRAEDTATYYCAREGTYDWYLDLWGRGTLVTVSS

SEQ ID NO: 260 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSGIGPSGGITKYADSVKGRFTISRDNSKNTLYLQMNSLRAEDTATYYCAREGTYDWYLDLWGRGTLVTVSS

SEQ ID NO: 261 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSIIGPSGGITKYADSVKGRFTISRDNSKNTLYLQMNSLRAEDTATYYCAREGTYDWYLDLWGRGTLVTVSS

SEQ ID NO: 262 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSDIGPSGGITKYADSVKGRFTISRDNSKNTLYLQMNSLRAEDTATYYCAREGTYDWYLDLWGRGTLVTVSS

SEQ ID NO: 263 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDMYLDLWGRGTLVTVSS

SEQ ID NO: 264 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDLYLDLWGRGTLVTVSS

SEQ ID NO: 265 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDTYLDLWGRGTLVTVSS

SEQ ID NO: 266 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDIYLDLWGRGTLVTVSS

SEQ ID NO: 267 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGGSGGITKYADSVKGRFTISRDNSKNTLYLQMNSLRAEDTATYYCAREGTYDWYLDLWGRGTLVTVSS

SEQ ID NO: 268 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGWTKYADSVKGRFTISRDNSKNTLYLQMNSLRAEDTATYYCAREGTYDWYLDLWGRGTLVTVSS

SEQ ID NO: 269 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGYTKYADSVKGRFTISRDNSKNTLYLQMNSLRAEDTATYYCAREGTYDWYLDLWGRGTLVTVSS

SEQ ID NO: 270 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGSTKYADSVKGRFTISRDNSKNTLYLQMNSLRAEDTATYYCAREGTYDWYLDLWGRGTLVTVSS

SEQ ID NO: 271 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGPTKYADSVKGRFTISRDNSKNTLYLQMNSLRAEDTATYYCAREGTYDWYLDLWGRGTLVTVSS

SEQ ID NO: 272 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGVTKYADSVKGRFTISRDNSKNTLYLQMNSLRAEDTATYYCAREGTYDWYLDLWGRGTLVTVSS

SEQ ID NO: 273 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGTTKYADSVKGRFTISRDNSKNTLYLQMNSLRAEDTATYYCAREGTYDWYLDLWGRGTLVTVSS

SEQ ID NO: 274 - protein, 119 aa, Artificial Sequence; identical to SEQ ID NO: 253
  source: /note=“Description of Artificial Sequence Synthetic polypeptide”

SEQ ID NO: 275 - protein, 447 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 276 - protein, 215 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN